(19) United States
(12) Patent Application Publication
(10) Pub. No.: US 2026/0251664 A1
(43) Pub. Date: Aug. 27, 2026

(54) Methods of Assessing Dementia Risk

(71) Applicant: SomaLogic Operating Co., Inc., Boulder, CO (US)

(72) Inventors: Kelsey M. Loupy, Boulder, CO (US); Rachel M. Ostroff, Boulder, CO (US); Lori K. Bogren, Boulder, CO (US); Laura Mae Sampson, Boulder, CO (US); Amy Zhang, Boulder, CO (US); Natasha Kureshi, Boulder, CO (US); Hannah Biegel, Boulder, CO (US); Clare Paterson, Boulder, CO (US); Yolanda Hagar, Boulder, CO (US); Leigh Alexander, Boulder, CO (US)

(73) Assignee: SomaLogic Operating Co., Inc., Boulder, CO (US)

(21) Appl. No.: 18/875,848

(22) PCT Filed: Jul. 13, 2023
(86) PCT No.: PCT/US2023/027583

§ 371 Date: Dec. 17, 2024

Related U.S. Application Data
(60) Provisional application No. 63/389,214, filed on Jul. 14, 2022.
(60) Provisional application No. 63/446,402, filed on Feb. 17, 2023.

(51) Int. Cl. G01N 33/68; G01N 33/53

(52) U.S. Cl. CPC G01N 33/6896; G01N 33/53; G01N 2333/4703; G01N 2333/91215; G01N 2800/2814; G01N 2800/50; G01N 2800/60

(57) ABSTRACT

The present disclosure includes biomarkers, methods, devices, reagents, systems, and kits for the evaluation of risk of dementia in a middle-aged individual within a specified timeframe, for example 5, 10, 15 and/or 20 years. In one aspect, the disclosure provides biomarkers that can be used alone or in various combinations to evaluate risk of dementia within 5, 10, 15 and/or 20 years. In another aspect, methods are provided for evaluating risk of dementia within 5, 10, 15 and/or 20 years in a middle-aged individual, where the methods include detecting, in a biological sample from an individual, at least one biomarker value corresponding to at least one biomarker selected from the group of biomarkers provided in Table 6.

### CROSS-REFERENCE TO RELATED APPLICATIONS

This application claims the benefit of priority of US Provisional Application Nos. 63/389,214, filed Jul. 14, 2022, and 63/446,402, filed Feb. 17, 2023, each of which is incorporated by reference herein in its entirety for any purpose.

### FIELD

The present application relates generally to the detection of biomarkers and methods of evaluating the risk of dementia in an individual and, more specifically, to one or more biomarkers, methods, devices, reagents, systems, and kits used to assess an individual for the risk of developing dementia, for example, within 5, 10, 15, and/or 20 years.

### BACKGROUND

The following description provides a summary of information relevant to the present application and is not an admission that any of the information provided or publications referenced herein is prior art to the present application.

Dementia is a group of diseases characterized by a deterioration in cognitive ability beyond what is expected to be associated with normal aging. The global prevalence of dementia in men and women over the age of 60 years is estimated at 4.7%, and 6.9%, respectively, within North America. The most common type of dementia is Alzheimer's disease, which accounts for 60-80% of dementia cases, and is the 6^{th} leading cause of death. Alzheimer's disease currently affects over 6 million adults in the United States—a number expected to double in the next 30 years. Early onset or familial dementia generally occurs prior to age 65, and is associated with a high genetic burden, whereas dementia onset after 65 is generally considered “sporadic” in origin.

The symptomatic hallmarks of dementia are clinical changes in memory, thinking, and other cognitive skills as reported by either the individual themselves, or a caregiver. Diagnosis of dementia based on DSM-5 criteria involves an extensive battery of neurocognitive assessments, usually conducted over a span of time to assess decline in function; the screening also takes into account the patient's history of psychiatric disorders, medications, laboratory results, and informant questioning to rule out possible alternative causes of cognitive impairments. To determine the underlying etiology of dementia, additional, more invasive, assessments to identify dementia-subtype-related pathologies are often conducted, including neurological imaging, spinal fluid or serological assessments of biomarkers. For example, Alzheimer's disease is associated with the neurological accumulation of beta-amyloid plaques and tau tangles, whereas Lewy body disease dementia is associated with alpha-synuclein aggregations.

Several treatments are commonly prescribed in individuals with dementia, and can temporarily improve cognition; however, these drugs do not stop, nor slow, the progression of underlying neuropathological damage. New classes of Alzheimer's disease drugs targeting beta-amyloid have recently been fast-tracked for approval by the FDA and have preliminary positive findings in early phase clinical trials in early-stage Alzheimer's disease patients and those with mild cognitive impairment.

The greatest risk factor for dementia is age. Estimates of Alzheimer's disease prevalence increase from 5.3% among persons aged 65 to 74 years to 13.8% among persons aged 75-84 years and greater than 30% in persons over the age of 85. Among both sexes, the average age of onset for any incident dementia in the United States is 83.7 years. With life expectancies increasing, and the aging population expanding, the expected prevalence and number of individuals with Alzheimer's disease projected to significantly increase over the coming decades. (FIG. 1)

Other risk factors for increased likelihood of dementia include family history of dementia, as well as multiple modifiable lifestyle factors including those related to cardiovascular disease, obesity, and diabetes. Common genetic polymorphisms known to increase risk for dementia include polymorphisms in the APOE gene encoding variations in the ε2, ε3, and ε4 alleles, which have been associated with Alzheimer's disease, cerebrovascular dysfunction, and Lewy body disease. The ε2 allele is thought to be neuroprotective, ε3 allele neutral, and ε4 allele risk-associated.

The United States Preventive Services Task Force (USPSTF) determined that there is inconclusive evidence for, or against, recommending regular screening for dementia in asymptomatic adults, although the USPSTF acknowledged the need to identify cognitive impairment early on. Because the onset of dementia is slow with a long prodromal stage known to occur prior to the presentation of cognitive symptoms, the neuropathophysiology of Alzheimer's disease (termed preclinical Alzheimer's disease) can begin to take shape 15-20 years in advance with plaques and tangles evident in the brain decades before cognitive symptom onset. The current screening tests are limited to detecting current dementia pathophysiology or progression when symptoms are present, at which point it may be too late to prevent or provide adequate treatment.

Considering the lack of evidence-based treatment options available for dementia, risk intervention (healthy diet, exercise, social and cognitive stimulation, lowering vascular disease risk factors) is thought to be the most effective way to reduce future dementia prevalence.

There are currently few long-term predictive screening tests for dementia. Recently, the FDA approved a direct-to-consumer test provided by 23andMe®, which reports an individual's risk for late-onset Alzheimer's disease by assessing the number of APOE4 gene variants that the individual carries. There are several clinical risk calculators based upon a combination of demographic, personal and family history, and lifestyle factors; however, these risk calculators and genetic factors do not change over time and often require self-reported variables. No clinical risk calculators are currently widely used as standard of care in clinical practice.

The development of a proteomic model for determining risk of dementia would be highly desirable. Accordingly, a need exists for biomarkers, methods, devices, reagents, systems, and kits that enable the prediction of the development of dementia within a specified timeframe, such as a 5, 10, 15, and/or 20 year period.

### SUMMARY

The present application includes biomarkers, methods, reagents, devices, systems, and kits for the prediction of risk of developing dementia within a specified timeframe, such as a 20 year period. In some embodiments, methods of identifying subjects at risk for developing dementia are provided. In some embodiments, methods of detecting levels of N biomarker proteins in a sample are provided.

In some embodiments, methods of determining whether a subject is at risk for developing dementia within a 20 year period, comprising detecting a level of CILP2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of detecting levels of N biomarker proteins in a sample, comprising obtaining the sample from the subject, and detecting the level of each of the N biomarker proteins in the sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of PTN biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of PH biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of Notch1 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of NADK biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of CDON biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of MP2K2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of H2A3 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of IGFALS biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 20 year period, comprising detecting a level of S100A13 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, IGFALS, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of CILP2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of PTN biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of PH biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of Notch1 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of NADK biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of CDON biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of MP2K2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of H2A3 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of IGFALS biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, methods of determining whether a subject is at risk for developing dementia within a 5, 10, and/or 15 year period, comprising detecting a level of S100A13 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, IGFALS, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, N is 2 to 25, or N is 3 to 25, or N is 4 to 25, or N is 5 to 25, or N is 6 to 25, or N is 7 to 25, or N is 8 to 25, or N is 9 to 25, or N is 10 to 25, or N is 11 to 25, or N is 12 to 25, or N is 13 to 25, or N is 14 to 25, or N is 15 to 25. In some embodiments, N is 2, N is 3, or N is 4, or N is 5, or N is 6, or N is 7, or N is 8, or N is 9, or N is 10, or N is 11, or N is 12, or N is 13, or N is 14, or N is 15, or N is 16, or N is 17, or N is 18, or N is 19, or N is 20, or N is 21, or N is 22, or N is 23, or N is 24, or N is 25.

In some embodiments, each of the N biomarker proteins is selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG. In some embodiments, at least one of the N biomarker proteins is selected from CILP2, PTN, PH, Notch1, NADK, CDON, MP2K2, H2A3, IGFALS, and S100A13. In some embodiments, at least 2, at least 3, at least 4, at least 5, at least 6, at least 7, at least 8, at least 9, or at least 10 of the N protein biomarkers are selected from CILP2, PTN, PH, Notch1, NADK, CDON, MP2K2, H2A3, IGFALS, and S100A13. In some embodiments, two of the N biomarker proteins are CILP2 and H2A3, or two of the N biomarker proteins are CILP2 and IGFALS, or two of the N biomarker proteins are CILP2 and MP2K2, or two of the N biomarker proteins are CILP2 and NADK, or two of the N biomarker proteins are CILP2 and Notch1, or two of the N biomarker proteins are CILP2 and PH, or two of the N biomarker proteins are CILP2 and PTN, or two of the N biomarker proteins are CILP2 and S100A13, or two of the N biomarker proteins are CILP2 and CDON. In some embodiments, two of the N biomarker proteins are PTN and PH, or two of the N biomarker proteins are PTN and S100A13, or two of the N biomarker proteins are PTN and Notch1, or two of the N biomarker proteins are PTN and NADK, or two of the N biomarker proteins are PTN and MP2K2, or two of the N biomarker proteins are PTN and IGFALS, or two of the N biomarker proteins are PTN and H2A3, or two of the N biomarker proteins are PTN and CDON. In some embodiments, two of the N biomarker proteins are PH and Notch1, or two of the N biomarker proteins are PH and NADK, or two of the N biomarker proteins are PH and CDON, or two of the N biomarker proteins are PH and MP2K2, or two of the N biomarker proteins are PH and H2A3, or two of the N biomarker proteins are PH and IGFALS, or two of the N biomarker proteins are PH and S100A13. In some embodiments, two of the N biomarker proteins are Notch1 and NADK, or two of the N biomarker proteins are Notch1 and CDON, or two of the N biomarker proteins are Notch1 and MP2K2, or two of the N biomarker proteins are Notch1 and H2A3, or two of the N biomarker proteins are Notch1 and IGFALS, or two of the N biomarker proteins are Notch1 and S100A13. In some embodiments, two of the N biomarker proteins are NADK and CDON, or two of the N biomarker proteins are NADK and MP2K2, or two of the N biomarker proteins are NADK and H2A3, or two of the N biomarker proteins are NADK and IGFALS, or two of the N biomarker proteins are NADK and S100A13. In some embodiments, two of the N biomarker proteins are CDON and MP2K2, or two of the N biomarker proteins are CDON and H2A3, or two of the N biomarker proteins are CDON and IGFALS, or two of the N biomarker proteins are CDON and S100A13. In some embodiments, two of the N biomarker proteins are MP2K2 and H2A3, or two of the N biomarker proteins are MP2K2 and IGFALS, or two of the N biomarker proteins are MP2K2 and S100A13. In some embodiments, two of the N biomarker proteins are H2A3 and IGFALS, or two of the N biomarker proteins are H2A3 and S100A13. In some embodiments, two of the N biomarker proteins are IGFALS and S100A13.

In some embodiments, the sample is a blood sample, a plasma sample, or a serum sample. In some embodiments, the subject is 39, 40, 41, 42, 43, 44, 45, 46, 47, 48, 49, 50, 51, 52, 53, 54, 55, 56, 57, 58, 59, 60, 61, 62, 63, 64, or 65 years of age or older. In some embodiments, detecting is performed using mass spectrometry, an aptamer based assay and/or an antibody based assay. In some embodiments, the method includes contacting biomarker proteins of the sample or samples with a set of biomarker capture reagents, wherein each biomarker capture reagent of the set of biomarker capture reagents specifically binds to a different biomarker protein being detected. In some embodiments, each biomarker capture reagent is an antibody or an aptamer. In some embodiments, each biomarker capture reagent is an aptamer. In some embodiments, at least one aptamer is a slow off-rate aptamer. In some embodiments, at least one slow off-rate aptamer includes at least one, at least two, at least three, at least four, at least five, at least six, at least seven, at least eight, at least nine, or at least 10 nucleotides with modifications. In some embodiments, each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes. In some embodiments, the level of each biomarker protein measured is determined from a relative florescence unit (RFU) or a protein concentration.

In some embodiments, determining the risk for developing dementia within a 5, 10, 15, and/or 20 year period is based on input of the levels of the N biomarker proteins measured in a statistical model. In some embodiments, the determining comprises analyzing the levels of the N biomarker protein using an accelerated failure time (AFT) model with a Weibull distribution. In some embodiments, the model has an area under the curve (AUC) selected from at least 0.65, at least 0.66, at least 0.67, at least 0.68, at least 0.69, at least 0.7, at least 0.75, at least 0.8, at least 0.85, at least 0.9, or at least 0.95. In some embodiments, the model provides an absolute risk probability of a dementia diagnosis within 5, 10, 15, and/or 20 years. In some embodiments, the model provides a relative risk probability calculation of dementia diagnosis within 5, 10, 15, and/or 20 years.

In some embodiments, the relative risk is a value range used to predict the development of dementia within 5, 10, 15, and/or 20 years. In some embodiments, the relative risk is selective from low to high. In some embodiments, the range of relative risk is 0.25-6.67. In some embodiments, the model provides the absolute probability or the relative risk prediction for developing dementia within a 5, 10, 15, and/or 20 year period based on the level of each of the proteins selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, the subject is identified as at risk for developing dementia within the 5, 10, 15, and/or 20 year period. In some embodiments the at-risk subject is administered a treatment. In some embodiments, the treatment comprises implementing a healthy diet, exercise, social and cognitive stimulation, and/or lowering vascular disease risk factors. In some embodiments, the at risk subject is monitored at one or more additional time points to determine the risk of developing dementia within the 5, 10, 15, and/or 20 year period. In some embodiments, the at-risk subject is stratified in a preventive therapeutic trial. In some embodiments, the at-risk subject is administered a second diagnostic test. In some embodiments, the second diagnostic test is an APOE diagnostic test. In some embodiments, the subject at risk for developing dementia within the 5, 10, 15, and/or 20 year period is identified for a purpose of determining a life and/or financial plan. In some embodiments, one or more additional biomarkers associated with dementia are identified in a subject at risk for dementia or having dementia.

In some embodiments, the risk is determined within a 5 year period. In some embodiments, the risk is determined within a 10 year period. In some embodiments, the risk is determined within a 15 year period. In some embodiments, the risk is determined within a 20 year period.

In some embodiments, a kit is provided wherein the kit includes N biomarker protein capture reagents, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker protein capture reagents specifically binds to a biomarker protein selected from CILP2, PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

In some embodiments, N is 2 to 25, or N is 3 to 25, or N is 4 to 25, or N is 5 to 25, or N is 6 to 25, or N is 7 to 25, or N is 8 to 25, or N is 9 to 25, or N is 10 to 25, or N is 11 to 25, or N is 12 to 25, or N is 13 to 25, or N is 14 to 25, or N is 15 to 25. In some embodiments, N is 2, N is 3, or N is 4, or N is 5, or N is 6, or N is 7, or N is 8, or N is 9, or N is 10, or N is 11, or N is 12, or N is 13, or N is 14, or N is 15, or N is 16, or N is 17, or N is 18, or N is 19, or N is 20, or N is 21, or N is 22, or N is 23, or N is 24, or N is 25. In some embodiments, each of the N biomarker protein capture reagents specifically binds to a different biomarker protein. In some embodiments, each of the N biomarker protein capture reagents specifically binds to a biomarker protein selected from CILP2, PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG. In some embodiments, at least 1 of the N biomarker protein capture reagents specifically binds to a biomarker protein selected from CILP2, PTN, PH, Notch1, NADK, CDON, MP2K2, H2A3, IGFALS, and S100A13.

In some embodiments, at least 2, at least 3, at least 4, at least 5, at least 6, at least 7, at least 8, at least 9, or at least 10 of the N biomarker protein capture reagents specifically binds to a biomarker protein selected from CILP2, PTN, PH, Notch1, NADK, CDON, MP2K2, H2A3, IGFALS, and S100A13.

In some embodiments, two of the N biomarker protein capture reagents specifically bind CILP2 and H2A3, or two of the N biomarker protein capture reagents specifically bind CILP2 and IGFALS, or two of the N biomarker protein capture reagents specifically bind CILP2 and MP2K2, or two of the N biomarker protein capture reagents specifically bind CILP2 and NADK, or two of the N biomarker protein capture reagents specifically bind CILP2 and Notch1, or two of the N biomarker protein capture reagents specifically bind CILP2 and PH, or two of the N biomarker protein capture reagents specifically bind CILP2 and PTN, or two of the N biomarker protein capture reagents specifically bind CILP2 and S100A13, or two of the N biomarker protein capture reagents specifically bind CILP2 and CDON.

In some embodiments, two of the N biomarker protein capture reagents specifically bind PTN and PH, or two of the N biomarker protein capture reagents specifically bind PTN and S100A13, or two of the N biomarker protein capture reagents specifically bind PTN and Notch1, or two of the N biomarker protein capture reagents specifically bind PTN and NADK, or two of the N biomarker protein capture reagents specifically bind PTN and MP2K2, or two of the N biomarker protein capture reagents specifically bind PTN and IGFALS, or two of the N biomarker protein capture reagents specifically bind PTN and H2A3, or two of the N biomarker protein capture reagents specifically bind PTN and CDON.

In some embodiments, two of the N biomarker protein capture reagents specifically bind PH and Notch1, or two of the N biomarker protein capture reagents specifically bind PH and NADK, or two of the N biomarker protein capture reagents specifically bind PH and CDON, or two of the N biomarker protein capture reagents specifically bind PH and MP2K2, or two of the N biomarker protein capture reagents specifically bind PH and H2A3, or two of the N biomarker protein capture reagents specifically bind PH and IGFALS, or two of the N biomarker protein capture reagents specifically bind PH and S100A13.

In some embodiments, two of the N biomarker protein capture reagents specifically bind Notch1 and NADK, or two of the N biomarker protein capture reagents specifically bind Notch1 and CDON, or two of the N biomarker protein capture reagents specifically bind Notch1 and MP2K2, or two of the N biomarker protein capture reagents specifically bind Notch1 and H2A3, or two of the N biomarker protein capture reagents specifically bind Notch1 and IGFALS, or two of the N biomarker protein capture reagents specifically bind Notch1 and S100A13.

In some embodiments, two of the N biomarker protein capture reagents specifically bind NADK and CDON, or two of the N biomarker protein capture reagents specifically bind NADK and MP2K2, or two of the N biomarker protein capture reagents specifically bind NADK and H2A3, or two of the N biomarker protein capture reagents specifically bind NADK and IGFALS, or two of the N biomarker protein capture reagents specifically bind NADK and S100A13.

In some embodiments, two of the N biomarker protein capture reagents specifically bind CDON and MP2K2, or two of the N biomarker protein capture reagents specifically bind CDON and H2A3, or two of the N biomarker protein capture reagents specifically bind CDON and IGFALS, or two of the N biomarker protein capture reagents specifically bind CDON and S100A13.

In some embodiments, two of the N biomarker protein capture reagents specifically bind MP2K2 and H2A3, or two of the N biomarker protein capture reagents specifically bind MP2K2 and IGFALS, or two of the N biomarker protein capture reagents specifically bind MP2K2 and S100A13. In some embodiments, two of the N biomarker protein capture reagents specifically bind H2A3 and IGFALS, or two of the N biomarker protein capture reagents specifically bind H2A3 and S100A13. In some embodiments, two of the N biomarker protein capture reagents specifically bind IGFALS and S100A13.

In some embodiments a kit including N biomarker protein capture reagents is provided, wherein the kit includes biomarker protein capture reagents for carrying out any of the methods described herein. In some embodiments, each of the N biomarker protein capture reagents is an antibody or an aptamer. In some embodiments, each biomarker protein capture reagent is an aptamer. In some embodiments, at least one aptamer is a slow off-rate aptamer. In some embodiments, at least one slow off-rate aptamer comprises at least one, at least two, at least three, at least four, at least five, at least six, at least seven, at least eight, at least nine, or at least 10 nucleotides with modifications. In some embodiments, each slow off-rate aptamer binds to its target protein with an off rate (tr) of ≥20 minutes, ≥30 minutes, ≥60 minutes, ≥90 minutes, ≥120 minutes, ≥150 minutes, ≥180 minutes, ≥210 minutes, or ≥240 minutes. In some embodiments, the kit is for use in detecting the N biomarker proteins in a sample from a subject. In some embodiments, the kit is for use in determining whether the subject is at risk for developing dementia within a 5, 10, 15 and/or 20 year period.

### BRIEF DESCRIPTION OF THE DRAWINGS

FIG. 1 shows the projected number of people in the U.S. (by age bracket) with Alzheimer's Disease from 2020-2060.

FIG. 2 shows a calibration plot of Kaplan Meier estimates for observed event rate against model predicted probabilities, stratified into deciles of risk.

FIG. 3 shows certain nucleobase modifications that can be used in an aptamer.

FIG. 4 illustrates an exemplary computer system for use with various computer-implemented methods described herein.

FIG. 5 is a flowchart for a method of indicating evaluating risk of dementia in accordance with one or more embodiments.

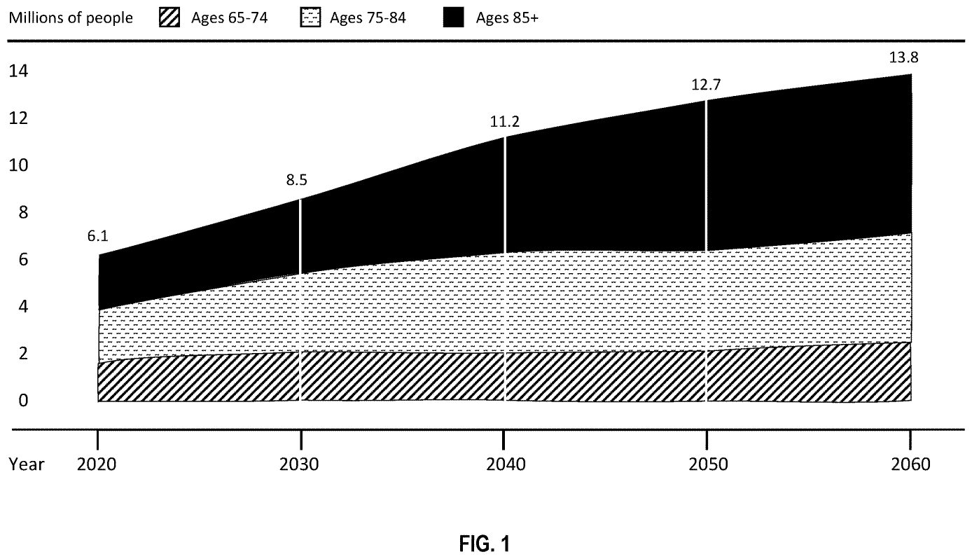

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, certain methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “a SOMAmer” includes mixtures of SOMAmers, reference to “a probe” includes mixtures of probes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The present application includes biomarkers, methods, devices, reagents, systems, and kits for the prediction of risk of dementia within a defined period of time, such as 5, 10, 15, and/or 20 years. In some aspects, the prediction of risk of dementia is for a middle-aged subject, such as 39, 40, 41, 42, 43, 44, 45, 46, 47, 48, 49, 50, 51, 52, 53, 54, 55, 56, 57, 58, 59, 60, 61, 62, 63, 64, or 65 years of age or older.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), dried blood spots (e.g., obtained from infants), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, it should be realized that a biological sample can be derived by taking biological samples from a number of individuals and pooling them or pooling an aliquot of each individual's biological sample. The pooled sample can be treated as a sample from a single individual and if an increased or decreased risk of a dementia is established in the pooled sample, then each individual biological sample can be re-tested to determine which individual/s have an increased or decreased risk of a dementia.

For purposes of this specification, the phrase “data attributed to a biological sample from an individual” is intended to mean that the data in some form derived from, or were generated using, the biological sample of the individual. The data may have been reformatted, revised, or mathematically altered to some degree after having been generated, such as by conversion from units in one measurement system to units in another measurement system; but, the data are understood to have been derived from, or were generated using, the biological sample.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” is a set of copies of one type or species of molecule or multi-molecular structure. “Target molecules”, “targets”, and “analytes” refer to more than one such set of molecules. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent” or “capture reagent” refers to a molecule that is capable of binding specifically to a biomarker. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. In some embodiments, a capture reagent is selected from an aptamer and an antibody.

As used herein, “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

The term “antibody” refers to full-length antibodies of any species and fragments and derivatives of such antibodies, including Fab fragments, F(ab′)2 fragments, single chain antibodies, Fv fragments, and single chain Fv fragments. The term “antibody” also refers to synthetically-derived antibodies, such as phage display-derived antibodies and fragments, affybodies, nanobodies, etc.

As used herein, “marker” and “biomarker” and “feature” are used interchangeably to refer to a target molecule that indicates or is a sign of a normal or abnormal process in an individual or of a disease or other condition in an individual. More specifically, a “marker” or “biomarker” or “feature” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker. In certain aspects, a feature is an analyte/SOMAmer reagent of other predictors in a statistical model.

As used herein, “biomarker value”, “value”, “biomarker level”, “feature level” and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease or condition, relative to its expression in a normal or control subject. The terms also include genes (or the corresponding protein expression products) whose expression is activated to a higher or lower level at different stages of the same disease or condition. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

A “control level” of a target molecule refers to the level of the target molecule in a properly handled sample of the same sample type. Control level may refer to the average level of the target molecule in properly handled samples from a population of individuals.

As used herein, “individual” refers to a test subject or patient. The individual can be a mammal or a non-mammal. In various embodiments, the individual is a mammal. A mammalian individual can be a human or non-human. In various embodiments, the individual is a human. A healthy or normal individual is an individual in which the disease or condition of interest (including, for example, dementia) is not detectable by conventional diagnostic methods. A middle-aged individual is an individual 49 years of age or older.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The prediction of risk of a dementia includes distinguishing individuals who have an increased risk of dementia from individuals who do not.

“Prognose”, “prognosing”, “prognosis”, and variations thereof refer to the prediction of a future course of a disease or condition in an individual who has the disease or condition (e.g., predicting patient survival), and such terms encompass the evaluation of disease or condition response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the risk that a disease or condition will recur in an individual who apparently has been cured of the disease or has had the condition resolved. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” risk of dementia can include, for example, any of the following: predicting the future risk of dementia in an individual; predicting the risk of dementia in an individual who apparently has no dementia issues. Evaluation of risk of dementia can include embodiments such as the assessment of risk of dementia on a continuous scale, or classification of risk of dementia in escalating classifications. Classification of risk includes, for example, classification into two or more classifications such as “No Elevated Risk of Dementia” and “Elevated Risk of Dementia.” The evaluation of risk of dementia is for a defined period; such period can be, for example, 5, 10, 15, and/or 20 years. In some aspects, evaluation of risk of dementia is for subjects aged 39, 40, 41, 42, 43, 44, 45, 46, 47, 48, 49, 50, 51, 52, 53, 54, 55, 56, 57, 58, 59, 60, 61, 62, 63, 64, or 65 years of age or older.

As used herein, “additional biomedical information” refers to one or more evaluations of an individual, other than using any of the biomarkers described herein, that are associated with dementia risk. “Additional biomedical information” includes any of the following: physical descriptors of an individual, including the height and/or weight of an individual; the age of an individual; the gender of an individual; change in weight; the ethnicity of an individual; occupational history; family history of dementia; the presence of a genetic marker(s) correlating with a higher risk of dementia in the individual; clinical symptoms such as abdominal pain, weight gain or loss gene expression values; physical descriptors of an individual, including physical descriptors observed by radiologic imaging; smoking status; alcohol use history; occupational history; dietary habits—salt, saturated fat and cholesterol intake; caffeine consumption; and imaging information. Testing of biomarker levels in combination with an evaluation of any additional biomedical information, including other laboratory tests, may, for example, improve sensitivity, specificity, and/or AUC for prediction of dementia as compared to biomarker testing alone or evaluating any particular item of additional biomedical information alone (e.g., carotid intima thickness imaging alone). Additional biomedical information can be obtained from an individual using routine techniques known in the art, such as from the individual themselves by use of a routine patient questionnaire or health history questionnaire, etc., or from a medical practitioner, etc. Testing of biomarker levels in combination with an evaluation of any additional biomedical information may, for example, improve sensitivity, specificity, and/or thresholds for prediction of dementia as compared to biomarker testing alone or evaluating any particular item of additional biomedical information alone (e.g., CT imaging alone).

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

As used herein, “analyte” is the protein target of a capture reagent. In certain aspects, the capture reagent is an aptamer. In certain further aspects, the capture reagent is a SOMAmer.

As used herein, “Lin's CCC” means concordance correlation coefficient which measures the concordance between a new test and an existing test that is considered the gold standard.

As used herein, “study”, means a set of samples and clinical data that are analyzed to derive the test.

As used herein, “training dataset”, means a subset of data from a study used to fit a model.

As used herein, “validation dataset”, means a final subset of data used to assess the performance of a selected model developed on a verification dataset.

As used herein, “verification dataset”, means a separate subset of data used to provide an unbiased evaluation of a model fit on the training dataset while tuning model parameters.

As used herein, the term “need” or “needed” refers to a judgement made by a health care provider regarding treatment of a patient which is considered by the health care provider to be beneficial to the health status of the patient.

### Risk Analysis

In some aspects, an objective test for prediction of risk of dementia within 20 years is disclosed herein. In some aspects, an objective test for prediction of risk of dementia within 15 years is disclosed herein. In some aspects, an objective test for prediction of risk of dementia within 10 years is disclosed herein. In some aspects, an objective test for prediction of risk of dementia within 5 years is disclosed herein.

The risk analysis profile may be described as in Table 1.

TABLE 1
Risk analysis

| | | Likelihood | | Need for |
|---|---|---|---|---|
| | | relative to | | Mitigating |
| Hazard | Hazardous Situation(s) | SoC | Harm(s) | Measures |
| False | HCP recommends onset of | [ ] Lower | Emotional stress; | [✓] Low |
| Positive | more frequent cognition | [✓] Equivalent | unneeded inconvenience | [ ] Med |
| Result | screening. | [ ] Higher | for patient. Highly | [ ] High |
| (erroneous | | | unlikely for any invasive | |
| overprediction | | | testing to result without | |
| of dementia | | | initial non-invasive | |
| risk) | | | assessments. | |
| | Patient thinks their | | Patient may initiate | |
| | dementia risk is higher than | | changes in life planning | |
| | actual risk. | | (care, finances etc.) that | |
| | | | are unneeded | |
| | Patient initiates lifestyle | [✓] Lower | No harm, as these lifestyle | |
| | changes to lower risks of | [ ] Equivalent | choices are known to | |
| | future dementia diagnosis | [ ] Higher | improve overall health | |
| | unnecessarily (e.g., | | and adhere to general | |
| | smoking cessation, weight | | health | |
| | loss, increases social | | guidelines. | |
| | interactions) | | | |
| False Negative | Patient thinks | [ ] Lower | Patient may be more | [✓] Low |
| Result | their dementia risk is | [✓] Equivalent | reluctant to alter risk- | [ ] Med |
| (erroneous | lower than actual risk. | [ ] Higher | related behaviors or plan | [ ] High |
| underprediction | | | for long-term care. | |
| of dementia | Physician uses this test to | [ ] Lower | Misguided | [ ] Low |
| risk) | diagnose early-stage | [✓] Equivalent | information; HCP | [✓] Med |
| Off Label Use | dementia | [ ] Higher | may recommend | [ ] High |
| | | | dementia | |
| | | | biomarker | |
| | | | analysis with | |
| | | | higher level of | |
| | | | invasiveness and | |
| | | | discomfort (e.g., | |
| | | | CSF draw or brain | |
| | | | imaging) earlier | |
| | | | than current SoC | |
| | Physician uses this test in | [ ] Lower | Prediction results | [ ] Low |
| | patients not at known | [✓] Equivalent | may | [✓] Med |
| False Negative | average risk (e.g., high- | [ ] Higher | be inaccurate | [ ] High |
| Result | risk individuals) | | | |
| (erroneous | Patient thinks | | Patient may be more | [✓] Low |
| underprediction | their dementia risk is | | reluctant to alter risk- | [ ] Med |
| of dementia | lower than actual risk. | | related behaviors or plan | [ ] High |
| risk) | | | for long-term care. | |

The testing methods disclosed herein provide convenience for health care providers in assessment and monitoring of the risk dementia.

TABLE 2
Model Performance Target

| Validation Metric(s) | Performance Target |
|---|---|
| Area Under the Curve (AUC) | Model AUC at 20 years ≥ 0.678 |
| Area Under the Curve (AUC) | Model AUC at 5 years ≥ 0.678 |

The performance target was based on the performance of the leading genetic risk variant for Alzheimer's Disease (APOE ε4) in predicting future risk for dementia. (Escott-Price V, Sims R, Bannister C, et al. Common polygenic variation enhances risk prediction for Alzheimer's disease. Brain. 2015; 138(12): 3673-3684.)

There are currently no dementia risk prediction tests used as standard of care in routine clinical practice. There are multiple clinical risk calculators that have been developed to predict future risk for dementia, for example the Cardiovascular Risk Factors, Aging, and Incidence of Dementia (CAIDE) risk score calculator. (Kivipelto M, Ngandu T, Laatikainen T, et al. Risk score for the prediction of dementia risk in 20 years among middle aged people: a longitudinal, population-based study. Lancet Neurol. 2006; 5(9): 735-741.) This risk calculator (and other similar risk calculators) rely on demographic, lifestyle, genetic, and clinical information that are 1) immutable to change so have limited viability to monitor dementia risk over time, 2) rely on self-reported data, 3) have limited validation in independent cohorts, and 4) have not demonstrated reproducible associations with current or future pathophysiological markers of dementia etiology, such as Aβ deposition. (Hooshmand B, Polvikoski T, Kivipelto M, et al. CAIDE Dementia Risk Score, Alzheimer and cerebrovascular pathology: a population-based autopsy study. J Intern Med. 2018; 283(6): 597-603. Stephen R, Liu Y, Ngandu T et al. Associations of CAIDE Dementia Risk Score with MRI, PIB-PET measures, and cognition. J Alzheimers Dis. 2017; 59(2): 695-705.) There are multiple clinical risk calculators that have been developed to predict near-term risk for dementia, such as the Dementia Population Risk Tool (DemPoRT). The near-term risk calculators rely on similar factors to longer term risk calculators. Other blood-based biomarker tests are under investigation for their ability to diagnose Alzheimer's disease-specific etiology; for example, Alzosure® by Diadem was granted Breakthrough Device designation by the FDA and shows potential to predict risk for Alzheimer's disease diagnosis within 6 years. However, to date only APOE genotyping is commonly accepted into clinical practice for assessing risk. Therefore, instead of comparing model performance to a clinically unused risk calculator, model performance was compared against the ability of risk alleles of the APOE gene to accurately predict those at future risk of dementia.

The number of APOE ε4 risk alleles an individual carries is the most commonly used genetic risk determinant for Alzheimer's disease, with this risk allele also being linked to risk for other dementia types including cerebrovascular disease-related dementia, and Lewy body disease. (Tai L M, Thomas R, Marottoli F M, et al. The role of APOE in cerebrovascular dysfunction. Acta Neuropathol 2016; 131(5): 709-723.) In 2017, the FDA approved the Late-Onset Alzheimer's Disease Genetic Health Risk report. (U.S. Food and Drug Administration. “FDA allows marketing of first direct-to-consumer tests that provide genetic risk information for certain conditions” [press release]. April 2017.). The highest documented AUC for APOE ε4 prediction of dementia risk is 0.678. (Escott-Price V, Sims R, Bannister C, et al. Common polygenic variation enhances risk prediction for Alzheimer's disease. Brain. 2015; 138(12): 3673-3684.) Therefore, an AUC greater than or equal to 0.678 was used as a performance threshold during model development for the mid-life dementia risk test. Furthermore, since a subset of participants of the ARIC study had known APOE genotype, the direct within-study comparison of proteomic model to genetic model performance was enabled.

In some embodiments, the number of biomarkers useful for a biomarker subset or panel is based on the sensitivity and specificity value for the particular combination of biomarker values. The terms “sensitivity” and “specificity” are used herein with respect to the ability to correctly classify an individual, based on one or more biomarker values detected in their biological sample, as having an increased risk of dementia within 20 years or not having increased risk of dementia within the same time period. “Sensitivity” indicates the performance of the biomarker(s) with respect to correctly classifying individuals that have increased risk of dementia. “Specificity” indicates the performance of the biomarker(s) with respect to correctly classifying individuals who do not have increased relative risk of dementia.

In an alternate method, scores may be reported on a continuous range, with a threshold of high, intermediate or low risk of dementia, with thresholds determined based on clinical findings.

In some embodiments, overall performance of a panel of one or more biomarkers is represented by the area-under-the-curve (AUC) value. The AUC value is derived from a receiver operating characteristic (ROC) curve. The ROC curve is the plot of the true positive rate (sensitivity) of a test against the false positive rate (1-specificity) of the test. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., normal individuals and individuals at risk for dementia). ROC curves are useful for plotting the performance of a particular feature (e.g., any of the biomarkers described herein and/or any item of additional biomedical information) in distinguishing between two populations. Typically, the feature data across the entire population are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve.

### Exemplary Uses of Biomarkers

In various exemplary embodiments, methods are provided for evaluating risk of dementia in an individual by detecting one or more biomarker values corresponding to one or more biomarkers that are present in the circulation of an individual, such as in blood, serum or plasma, by any number of analytical methods, including any of the analytical methods described herein. These biomarkers are, for example, differentially expressed in individuals with increased risk of dementia as compared to individuals without increased risk of dementia. Detection of the differential expression of a biomarker in an individual can be used, for example, to permit the prediction of risk of dementia of a middle-aged adult (49 years of age or older) within a 20 year time frame.

In addition to testing biomarker levels as a stand-alone diagnostic test, biomarker levels can also be done in conjunction with determination of SNPs or other genetic lesions or variability that are indicative of increased risk of susceptibility of disease or condition. (See, e.g., Amos et al., Nature Genetics 40, 616-622 (2009)).

Any of the described biomarkers may also be used in imaging tests. For example, an imaging agent can be coupled to any of the described biomarkers, which can be used to aid in prediction of risk of dementia, to monitor response to therapeutic interventions, to select for target populations in a clinical trial among other uses.

### Detection and Determination of Biomarkers and Biomarker Levels

A biomarker level for the biomarkers described herein can be detected using any of a variety of known analytical methods. In one embodiment, a biomarker level is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to SOMAmers, antibodies, adnectins, ankyrins, other antibody mimetics and other protein scaffolds, autoantibodies, chimeras, small molecules, a F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker level is detected using a biomarker/capture reagent complex.

In other embodiments, the biomarker level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker level is detected directly from the biomarker in a biological sample.

In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In yet other embodiments, the detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker levels for the biomarkers described herein can be detected using known analytical methods including, singleplex SOMAmer assays, multiplexed SOMAmer assays, singleplex or multiplexed immunoassays, mRNA expression profiling, miRNA expression profiling, mass spectrometric analysis, histological/cytological methods, etc. as detailed below.

Determination of Biomarker Levels using Aptamer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of a biomarker value corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

As used herein, a “SOMAmer” or Slow Off-Rate Modified Aptamer refers to an aptamer having improved off-rate characteristics. SOMAmers can be generated using the improved SELEX methods described in U.S. Publication No. 2009/0004667, entitled “Method for Generating Aptamers with Improved Off-Rates.”

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker value corresponding to a biomarker in the test sample.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to effect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e, the SOMAmer) for detecting and quantifying a non-nucleic acid target, thus allowing the wide variety of nucleic acid technologies, including amplification, to be applied to a broader range of desired targets, including protein targets.

SOMAmers can be constructed to facilitate the separation of the assay components from a SOMAmer biomarker complex (or photoSOMAmer biomarker covalent complex) and permit isolation of the SOMAmer for detection and/or quantification. In some embodiments, these constructs can include a cleavable or releasable element within the SOMAmer sequence. In other embodiments, additional functionality can be introduced into the SOMAmer, for example, a labeled or detectable component, a spacer component, or a specific binding tag or immobilization element. For example, the SOMAmer can include a tag connected to the SOMAmer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. For prediction of dementia, the molecular capture reagents would be an aptamer or an antibody or the like and the specific target would be a dementia biomarker as in Table 6.

In some embodiments, a method for signal generation takes advantage of anisotropy signal change due to the interaction of a fluorophore-labeled capture reagent with its specific biomarker target. When the labeled capture reagent reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker value corresponding to a biomarker in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex.

Any means known in the art can be used to detect a biomarker value by detecting the aptamer component of an aptamer affinity complex. A number of different detection methods can be used to detect the aptamer component of an affinity complex, such as, for example, hybridization assays, mass spectroscopy, or QPCR. In some embodiments, nucleic acid sequencing methods can be used to detect the aptamer component of an aptamer affinity complex and thereby detect a biomarker value. Briefly, a test sample can be subjected to any kind of nucleic acid sequencing method to identify and quantify the sequence or sequences of one or more aptamers present in the test sample. In some embodiments, the sequence includes the entire aptamer molecule or any portion of the molecule that may be used to uniquely identify the molecule. In other embodiments, the identifying sequencing is a specific sequence added to the aptamer; such sequences are often referred to as “tags,” “barcodes,” or “zipcodes.” In some embodiments, the sequencing method includes enzymatic steps to amplify the aptamer sequence or to convert any kind of nucleic acid, including RNA and DNA that contain chemical modifications to any position, to any other kind of nucleic acid appropriate for sequencing.

In some embodiments, the sequencing method includes one or more cloning steps. In other embodiments the sequencing method includes a direct sequencing method without cloning.

In some embodiments, the sequencing method includes a directed approach with specific primers that target one or more aptamer in the test sample. In other embodiments, the sequencing method includes a shotgun approach that targets all aptamer in the test sample.

In some embodiments, the sequencing method includes enzymatic steps to amplify the molecule targeted for sequencing. In other embodiments, the sequencing method directly sequences single molecules. An exemplary nucleic acid sequencing-based method that can be used to detect a biomarker value corresponding to a biomarker in a biological sample includes the following: (a) converting a mixture of aptamers that contain chemically modified nucleotides to unmodified nucleic acids with an enzymatic step; (b) shotgun sequencing the resulting unmodified nucleic acids with a massively parallel sequencing platform such as, for example, the 454 Sequencing System (454 Life Sciences/Roche), the Illumina Sequencing System (Illumina), the ABI SOLiD Sequencing System (Applied Biosystems), the HeliScope Single Molecule Sequencer (Helicos Biosciences), or the Pacific Biosciences Real Time Single-Molecule Sequencing System (Pacific BioSciences) or the Polonator G Sequencing System (Dover Systems); and (c) identifying and quantifying the SOMAmers present in the mixture by specific sequence and sequence count.

### Determination of Biomarker Values Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Values Using Gene Expression Profiling

Measuring mRNA in a biological sample may be used as a surrogate for detection of the level of the corresponding protein in the biological sample. Thus, any of the biomarkers or biomarker panels described herein can also be detected by detecting the appropriate RNA.

mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

miRNA molecules are small RNAs that are non-coding but may regulate gene expression. Any of the methods suited to the measurement of mRNA expression levels can also be used for the corresponding miRNA. Recently many laboratories have investigated the use of miRNAs as biomarkers for disease. Many diseases involve wide-spread transcriptional regulation, and it is not surprising that miRNAs might find a role as biomarkers. The connection between miRNA concentrations and disease is often even less clear than the connections between protein levels and disease, yet the value of miRNA biomarkers might be substantial. Of course, as with any RNA expressed differentially during disease, the problems facing the development of an in vitro diagnostic product will include the requirement that the miRNAs survive in the diseased cell and are easily extracted for analysis, or that the miRNAs are released into blood or other matrices where they must survive long enough to be measured. Protein biomarkers have similar requirements, although many potential protein biomarkers are secreted intentionally at the site of pathology and function, during disease, in a paracrine fashion. Many potential protein biomarkers are designed to function outside the cells within which those proteins are synthesized.

### Detection of Biomarkers Using In Vivo Molecular Imaging Technologies

Any of the described biomarkers (see Table 6) may also be used in molecular imaging tests. For example, an imaging agent can be coupled to any of the described biomarkers, which can be used to aid in prediction of risk of dementia, to monitor response to therapeutic interventions, to select a population for clinical trials among other uses.

In vivo imaging technologies provide non-invasive methods for determining the state of a particular disease or condition in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the dementia status of an individual.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning (coronary calcium score), positron emission tomography (PET), single photon emission computed tomography (SPECT), computed tomography angiography, and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Labeled antibodies which specifically bind any of the biomarkers in Table 6 can be injected into an individual suspected of having an increased risk of dementia, detectable according to the particular biomarker used, for the purpose of diagnosing or evaluating the disease status or condition of the individual. The label used will be selected in accordance with the imaging modality to be used, as previously described. Localization of the label permits determination of the tissue damage or other indications related to the risk of dementia. The amount of label within an organ or tissue also allows determination of the involvement of the dementia biomarkers due to the risk of dementia in that organ or tissue.

Similarly, aptamers may be used for such in vivo imaging diagnostic methods. For example, an aptamer that was used to identify a particular biomarker described in Table 6 (and therefore binds specifically to that particular biomarker) may be appropriately labeled and injected into an individual being evaluated for dementia, detectable according to the particular biomarker, for the purpose of diagnosing or evaluating the levels of tissue damage, atherosclerotic plaques, components of inflammatory response and other factors associated with the risk of dementia in the individual. The label used will be selected in accordance with the imaging modality to be used, as previously described. Localization of the label permits determination of the site of the processes leading to increased risk. The amount of label within an organ or tissue also allows determination of the infiltration of the pathological process in that organ or tissue. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

The use of in vivo molecular biomarker imaging is increasing, including for clinical trials, for example, to more rapidly measure clinical efficacy in trials for new disease or condition therapies and/or to avoid prolonged treatment with a placebo for those diseases, such as multiple sclerosis, in which such prolonged treatment may be considered to be ethically questionable.

For a review of other techniques, see N. Blow, Nature Methods, 6, 465-469, 2009.

### Determination of Biomarker Values Using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Determination of Biomarker Values Using a Proximity Ligation Assay

A proximity ligation assay can be used to determine biomarker values. Briefly, a test sample is contacted with a pair of affinity probes that may be a pair of antibodies or a pair of aptamers, with each member of the pair extended with an oligonucleotide. The targets for the pair of affinity probes may be two distinct determinates on one protein or one determinate on each of two different proteins, which may exist as homo- or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein, as well as, information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker levels that are useful in methods for prediction of dementia, where the methods comprise detecting, in a biological sample from an individual, biomarker levels that each correspond to a biomarker selected from the group consisting of the biomarkers provided in Table 6, wherein a classification, as described in detail below, using the biomarker levels indicates whether the middle-aged individual has elevated risk of developing dementia within a 5, 10, 15, and/or 20 year time period. While certain of the described dementia biomarkers are useful alone for predicting risk of dementia, methods are also described herein for the grouping of multiple subsets of the dementia biomarkers that are each useful as a panel of two or more biomarkers. In accordance with any of the methods described herein, biomarker levels can be detected and classified individually or they can be detected and classified collectively, as for example in a multiplex assay format.

### Classification of Biomarkers and Calculation of Disease Scores

In some embodiments, biomarker “signature” for a given diagnostic or predictive test contains a set of markers, each marker having different levels in the populations of interest. Different levels, in this context, may refer to different means of the marker levels for the individuals in two or more groups, or different variances in the two or more groups, or a combination of both. For the simplest form of a diagnostic test, these markers can be used to assign an unknown sample from an individual into one of two groups, either at increased risk of dementia or not. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker values. In general, classification methods are most easily performed using supervised learning techniques where a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging, boosting, forests and random forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009; each of which is incorporated by reference in its entirety.

To produce a classifier using supervised learning techniques, a set of samples called training data are obtained. In the context of diagnostic tests, training data includes samples from the distinct groups (classes) to which unknown samples will later be assigned. For example, samples collected from individuals in a control population and individuals in a particular disease, condition or event population can constitute training data to develop a classifier that can classify unknown samples (or, more particularly, the individuals from whom the samples were obtained) as either having the disease, condition or elevated risk of an event or being free from the disease, condition or elevated risk of an event. The development of the classifier from the training data is known as training the classifier. Specific details on classifier training depend on the nature of the supervised learning technique (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009).

Since typically there are many more potential biomarker values than samples in a training set, care must be used to avoid over-fitting. Over-fitting occurs when a statistical model describes random error or noise instead of the underlying relationship. Over-fitting can be avoided in a variety of ways, including, for example, by limiting the number of markers used in developing the classifier, by assuming that the marker responses are independent of one another, by limiting the complexity of the underlying statistical model employed, and by ensuring that the underlying statistical model conforms to the data.

In order to identify a set of biomarkers associated with occurrence of events, the combined set of control and early event samples were analyzed using Principal Component Analysis (PCA). PCA displays the samples with respect to the axes defined by the strongest variations between all the samples, without regard to the case or control outcome, thus mitigating the risk of overfitting the distinction between case and control. Since the occurrence of serious thrombotic events has a strong component of chance involved, requiring unstable plaque to rupture in vital vessels to be reported, one would not expect to see a clear separation between the control and event sample sets. While the observed separation between case and control is not large, it occurs on the second principal component, corresponding to around 10% of the total variation in this set of samples, which indicates that the underlying biological variation is relatively simple to quantify.

In the next set of analyses, biomarkers can be analyzed for those components of difference between samples which were specific to the separation between the control samples and early event samples. One method that may be employed is the use of DSGA (Bair, E. and Tibshirani, R. (2004) Semi-supervised methods to predict patient survival from gene expression data. PLOS Biol., 2, 511-522) to remove (deflate) the first three principal component directions of variation between the samples in the control set. Although the dimensionality reduction is performed on the control set to discover, both the samples in the control and the samples from the early event samples are run through the PCA. Separation of cases from early events can be observed along the horizontal axis.

### Cross Validated Selection of Proteins Relevant to Dementia

In order to avoid over-fitting of protein predictive power to idiosyncratic features of a particular selection of samples, a cross-validation and dimensional reduction approach can be taken. Cross-validation involves the multiple selection of sets of samples to determine the association of risk by protein combined with the use of the unselected samples to monitor the ability of the method to apply to samples which were not used in producing the model of risk (The Elements of Statistical Learning—Data Mining, Inference, and Prediction, T. Hastie, et al., editors, Springer Science+Business Media, LLC, 2nd edition, 2009). We applied the supervised PCA method of Tibshirani et al (Bair, E. and Tibshirani, R. (2004) Semi-supervised methods to predict patient survival from gene expression data. PLOS Biol., 2, 511-522.) which is applicable to high dimensional datasets in the modeling of risk of dementia. The supervised PCA (SPCA) method involves the univariate selection of a set of proteins statistically associated with the observed event hazard in the data and the determination of the correlated component which combines information from all of these proteins. This determination of the correlated component is a dimensionality reduction step which not only combines information across proteins, but also mitigates the likelihood of overfitting by reducing the number of independent variables from the full protein menu of over 1000 proteins down to a few principal components (in this work, we only examined the first principal component).

### Univariate Analysis and Multivariate Analysis of the Relationship of Individual Proteins to Time to Event

The Cox proportional hazard model (Cox, David R (1972). “Regression Models and Life-Tables”. Journal of the Royal Statistical Society. Series B (Methodological) 34 (2): 187-220.)) is widely used in medical statistics. Cox regression avoids fitting a specific function of time to the cumulative survival, and instead employs a model of relative risk referred to a baseline hazard function (which may vary with time). The baseline hazard function describes the common shape of the survival time distribution for all individuals, while the relative risk gives the level of the hazard for a set of covariate values (such as a single individual or group), as a multiple of the baseline hazard. The relative risk is constant with time in the Cox model.

Accelerated failure time (AFT) models are a sub-class of survival models. Survival models predict time-to-event data under partial information. For example, in the data for the dementia model, the event is dementia diagnosis, but time-to-diagnosis event data is available for a fraction of the subjects in the study. For the rest of the subjects, the available information is that the subjects were not diagnosed with dementia from the time of the blood draw up to the end of the study. This second category is partial information, called “censoring”, because it is uncertain if or when they would ever be diagnosed with dementia.

Because survival models account for censoring, they can still use the data from those censored subjects, where other longitudinal models trying to predict when an event occurs can only use the information from subjects with dementia diagnoses. And because survival models take into account time-to-event, they can produce predicted probabilities of the event occurring within any time frame, which is different from most classification models (logistic regression, random forest).

AFT survival models in particular are a regression model which specifies/assumes a linear relationship between the model's covariates and log(time-to-event). So, a subject with 2× higher covariates (protein RFU counts) than baseline may be predicted to “survive” a dementia diagnosis 2× longer than baseline.

The two most common survival models are AFT models and proportional hazards models, and an AFT Weibull model is both. The definition of a proportional hazards model is a little more complicated than that of an AFT model—in a proportional hazards model, a subject with 2× higher covariates than baseline may have a 2× higher hazard at any time point, where hazard is the negative derivative of the survival curve over time.

Other common proportional hazards models are exponential and Cox models. Exponential models are a sub-type of Weibull model. Cox models are more limited in use—predicted probabilities of time-to-event are not available from Cox models, only relative risk. AFT models can give both absolute and relative risk.

### Kits

Any combination of the biomarkers of Table 6 can be detected using a suitable kit, such as for use in performing the methods disclosed herein. Furthermore, any kit can contain one or more detectable labels as described herein, such as a fluorescent moiety, etc.

In one embodiment, a kit includes (a) one or more capture reagents (such as, for example, at least one aptamer or antibody) for detecting one or more biomarkers in a biological sample, wherein the biomarkers include any of the biomarkers set forth in Table 6 and optionally (b) one or more software or computer program products for classifying the individual from whom the biological sample was obtained as either having or not having increased risk of dementia or for determining the likelihood that the individual has increased risk of dementia, as further described herein. Alternatively, rather than one or more computer program products, one or more instructions for manually performing the above steps by a human can be provided.

The combination of a solid support with a corresponding capture reagent having a signal generating material is referred to herein as a “detection device” or “kit”. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In one aspect, the invention provides kits for the analysis of dementia risk status. The kits include PCR primers for one or more aptamers specific to biomarkers selected from Table 6. The kit may further include instructions for use and correlation of the biomarkers with prediction of risk of dementia. The kit may also include a DNA array containing the complement of one or more of the aptamers specific for the biomarkers selected from Table 6, reagents, and/or enzymes for amplifying or isolating sample DNA. The kits may include reagents for real-time PCR, for example, TaqMan probes and/or primers, and enzymes.

For example, a kit can comprise (a) reagents comprising at least capture reagent for quantifying one or more biomarkers in a test sample, wherein said biomarkers comprise the biomarkers set forth in Table 6, or any other biomarkers or biomarkers panels described herein, and optionally (b) one or more algorithms or computer programs for performing the steps of comparing the amount of each biomarker quantified in the test sample to one or more predetermined cutoffs and assigning a score for each biomarker quantified based on said comparison, combining the assigned scores for each biomarker quantified to obtain a total score, comparing the total score with a predetermined score, and using said comparison to determine whether an individual has an increased risk of dementia. Alternatively, rather than one or more algorithms or computer programs, one or more instructions for manually performing the above steps by a human can be provided.

### Computer Methods and Software

Once a biomarker or biomarker panel is selected, a method for diagnosing an individual can comprise the following: 1) collect or otherwise obtain a biological sample; 2) perform an analytical method to detect and measure the biomarker or biomarkers in the panel in the biological sample; 3) perform any data normalization or standardization required for the method used to collect biomarker levels; 4) calculate the marker score; 5) combine the marker scores to obtain a total diagnostic or predictive score; and 6) report the individual's diagnostic or predictive score. In this approach, the diagnostic or predictive score may be a single number determined from the sum of all the marker calculations that is compared to a preset threshold value that is an indication of the presence or absence of disease. Or the diagnostic or predictive score may be a series of bars that each represent a biomarker level and the pattern of the responses may be compared to a pre-set pattern for determination of the presence or absence of disease, condition or the increased risk (or not) of an event.

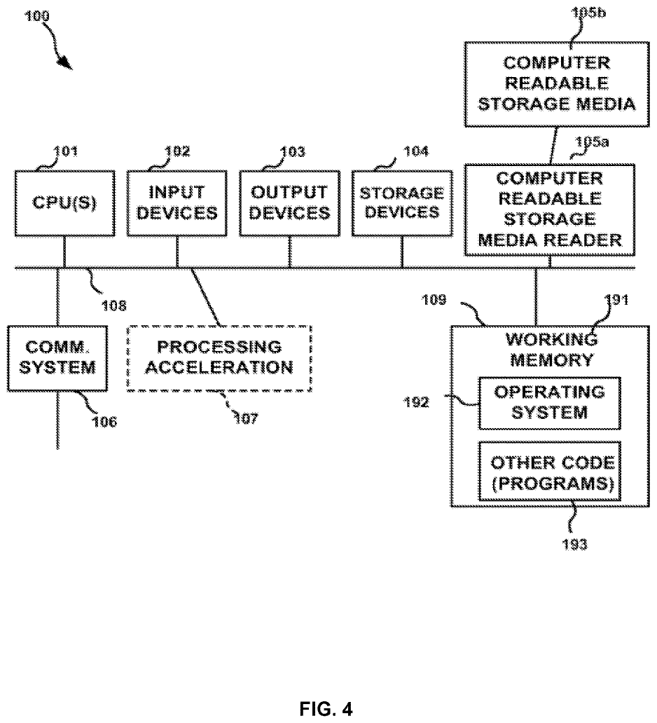

At least some embodiments of the methods described herein can be implemented with the use of a computer. An example of a computer system 100 is shown in FIG. 4. With reference to FIG. 4, system 100 is shown comprised of hardware elements that are electrically coupled via bus 108, including a processor 101, input device 102, output device 103, storage device 104, computer-readable storage media reader 105a, communications system 106, processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

With respect to FIG. 4, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of prediction of risk of dementia. The biomarker data (or biomarker information) can be utilized as an input to the computer for use as part of a computer implemented method. The biomarker data can include the data as described herein.

In one aspect, the system further comprises one or more devices for providing input data to the one or more processors.

The system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing dementia risk prediction biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The dementia risk prediction biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the dementia risk prediction biomarkers. The computer program may comprise multiple modules performing various functions or operations, such as a processing module for processing raw data and generating supplemental data and an analysis module for analyzing raw data and supplemental data to generate a dementia risk prediction status and/or diagnosis or risk calculation. Calculation of risk status for dementia may optionally comprise generating or collecting any other information, including additional biomedical information, regarding the condition of the individual relative to the disease, condition or event, identifying whether further tests may be desirable, or otherwise evaluating the health status of the individual.

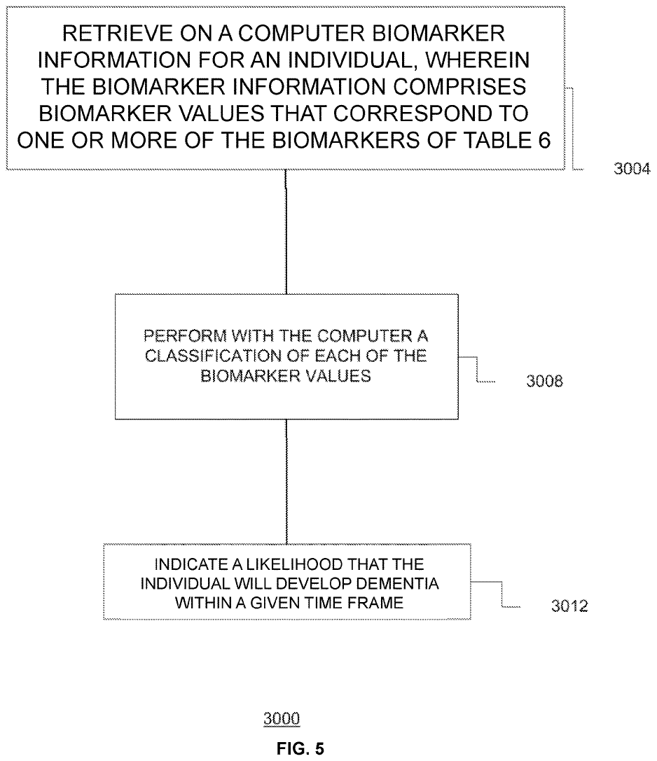

Referring now to FIG. 5, an example of a method of utilizing a computer in accordance with principles of a disclosed embodiment can be seen. In FIG. 5, a flowchart 3000 is shown. In block 3004, biomarker information can be retrieved for an individual. The biomarker information can be retrieved from a computer database, for example, after testing of the individual's biological sample is performed. The biomarker information can comprise biomarker levels that each correspond to one or more of the biomarkers of Table 6. In block 3008, a computer can be utilized to classify each of the biomarker levels. And, in block 3012, a determination can be made as to the likelihood that an individual has increased risk of dementia based upon a plurality of classifications. The indication can be output to a display or other indicating device so that it is viewable by a person. Thus, for example, it can be displayed on a display screen of a computer or other output device.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for evaluation of the risk of dementia. The computer program product includes a computer readable medium embodying program code executable by a processor of a computing device or system, the program code comprising: code that retrieves data attributed to a biological sample from an individual, wherein the data comprises biomarker values that each correspond to one or more of the biomarkers of Table 6; and code that executes a classification method that indicates a dementia risk status of the individual as a function of the biomarker values.

In still another aspect, a computer program product is provided for indicating a likelihood of risk of dementia. The computer program product includes a computer readable medium embodying program code executable by a processor of a computing device or system, the program code comprising: code that retrieves data attributed to a biological sample from an individual, wherein the data comprises a biomarker value corresponding to one or more of the biomarkers of Table 6; and code that executes a classification method that indicates a dementia risk status of the individual as a function of the biomarker value.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, PLAs, or ASICs.

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The biomarker identification process, the utilization of the biomarkers disclosed herein, and the various methods for determining biomarker values are described in detail above with respect to evaluation of risk of a dementia. However, the application of the process, the use of identified biomarkers, and the methods for determining biomarker values are fully applicable to other specific types of diseases or medical conditions, or to the identification of individuals who may or may not be benefited by an ancillary medical treatment.

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1. Multiplex Aptamer Assay and Statistical Approaches for Biomarker Identification

A multiplex aptamer assay was used to analyze test samples and control samples to identify biomarkers predictive of risk of dementia within a 20 year period. The multiplexed analysis used in this experiment included aptamers to detect approximately 5,000 proteins in blood from small sample volumes (~65 μl of serum or plasma), with low limits of detection (1 pM median), ~7 logs of dynamic range, and ~5% median coefficient of variation. The multiplex aptamer assay is described, generally, e.g., in Gold et al. (2010) Aptamer-Based Multiplexed Proteomic Technology for Biomarker Discovery. PLoS ONE 5(12): e15004; and U.S. Publication Nos: 2012/0101002 and 2012/0077695.

### Example 2. Model Specification

The endpoints for these analyses are dementia time-to-event outcomes, which have two components. 1) Time, in number of days from the blood draw to a dementia diagnosis (via level 3 adjudication methods including cognitive assessment tests, telephone screening, informant ratings, hospital records, and death record review) or exiting/completing the study. 2) A binary variable denoting whether a level 3 dementia diagnosis was observed during the study period or not.

The selected model is a 25-feature protein-only (see Table 6) accelerated failure time (AFT) model with a Weibull distribution. The model was trained on the entire study period, with performance maximized at 20 years. Table 3 shows the performance metrics for the model.

TABLE 3
Performance of selected dementia risk model at 20 years
on training, verification, and validation data.

| | | | Sensi- | Speci- | | |
|---|---|---|---|---|---|---|
| | | AUC at | tivity at | ficity at | C- | PEC at |
| | | 20 years | 20 years | 20 years | Index* | 20 years |
| | N per | (95% | (95% | (95% | (95% | (95% |
| Dataset | group | CI) | CI) | CI) | CI) | CI) |
| Training | 7895 | 0.73 | 0.62 | 0.72 | 0.70 | 0.13 |
| | | (0.71- | (0.58- | (0.70- | (0.68- | (0.13- |
| | | 0.75) | 0.65) | 0.73) | 0.71) | 0.14) |
| Verifi- | 1691 | 0.73 | 0.61 | 0.74 | 0.69 | 0.14 |
| cation | | (0.69- | (0.54, | (0.71- | (0.65- | (0.12- |
| | | 0.76) | 0.67) | 0.77) | 0.72) | 0.15) |
| Validation | 1691 | 0.70 | 0.60 | 0.68 | 0.67 | 0.14 |
| | | (0.66- | (0.54- | (0.65- | (0.64- | (0.12- |
| | | 0.74) | 0.67) | 0.71) | 0.70) | 0.15) |

Training, verification, and validation results exceed performance criteria of an AUC greater than or equal to 0.678. Sensitivity and specificity values were determined based on the cut-off value in the training dataset with the highest total Jouden's J measure (0.16). AUC, Area Under the Curve; PEC, Prediction Error Curve; CI, Confidence Intervals.
*C-Index is not time specific.

The model provides two predictions:

1) Absolute risk: This output is the absolute probability of not having a dementia diagnosis (heretofore Pr(Dementia-free), a value between 0 and 1, within 20 years. When delivered as a result and assessed by business rules, the resulting predicted probability will be subtracted from 1, to provide an absolute 20-year probability for a dementia diagnosis.

2) Relative risk: This is a continuous variable that is calculated using the absolute risk probability generated by the model (described above), divided by the “baseline” absolute probability (defined below) in the training cohort. This approach allows for the model risk probability prediction to be interpreted such that higher values indicate a greater likelihood of a dementia diagnosis within the next twenty years.

The baseline risk probability score represents the absolute risk for the “average” person in the training cohort based on the model algorithm. A “baseline” individual is defined as an individual with model feature values set to zero. All features in the model are centered on the overall mean, which means a value of 0 for any given feature is equal to the mean (i.e., the average). The baseline value is calculated by setting all the features to zero and then generating the absolute risk probability on those “zeroed” features. As such, a score less than 1 represents lower than average risk and a score greater than 1 represents higher risk than average risk. The rate of 20-year dementia diagnosis in the ARIC dataset is consistent with U.S. population dementia event rate age-matched for the intended use population.

The baseline absolute risk score in the training data is 0.15 or 15%.

Relative risk scores (where relative risk is absolute risk divided by the baseline absolute risk of 0.15) and absolute risk scores were stratified into four clinically meaningful risk categories in the training dataset. Kaplan-Meier (KM) plots were generated for these risk bins for the training and verification datasets across the entire time span of the study (30 years). Note that the first two risk bins (Low and Medium-Low) correspond to people whose predicted risk of a dementia diagnosis is lower than the average risk based on our data (baseline score, 0.15 relative risk equal to or lower than 1), while Medium-High and High risk bins correspond to predicted risk that is higher than the average/baseline.

The summary of absolute probability and relative risk stratification and corresponding event rates is shown in Table 4. Both unadjusted event rates and KM event rates are shown, KM event rates consider censoring.

TABLE 4
Summary of observed twenty year event rate by absolute
probability and relative risk bins in training data.

| | Relative | Absolute | | | | Event Rate | |
|---|---|---|---|---|---|---|---|
| Predicted | Risk | Probability | N | N | N | | Kaplan - |
| Class | Cutoffs | Cutoffs | Total | Censored* | Events | Unadjusted | Meier |
| Low | 0 ≤ x ≤ | 0 ≤ x ≤ | 880 | 182 | 28 | 3.18% | 3.79% |
| | 0.5 | 0.07 | | (20.7%) | | | |
| Medium- | 0.5 < x ≤ | 0.07 < x ≤ | 3,516 | 980 | 304 | 8.65% | 10.89% |
| Low | 1 | 0.15 | | (27.9%) | | | |
| Medium- | 1 < x ≤ | 0.15 < x ≤ | 1,750 | 694 | 225 | 12.86% | 18.4% |
| High | 1.5 | 0.22 | | (39.7%) | | | |
| High | 1.5 < x ≤ | 0.22 < x ≤ | 1,749 | 962 | 346 | 19.78% | 35.58% |
| | 6.67 | 1 | | (55.0%) | | | |

*Individuals were considered censored if last follow-up occurred before the end of time interval (twenty years from blood draw).

Based on this stratification, the scoring rules for absolute risk and relative risk for the mid-life dementia risk test are shown in Table 5.

TABLE 5
Scoring rules for relative risk and absolute risk probability

| Test Outcome | Test Outcome | | |
|---|---|---|---|
| x (relative risk | x (absolute risk | Predicted | |
| probability) | probability) | Class | Plain Language |
| 0 ≤ x ≤ 0.5 | 0 ≤ x ≤ 0.07 | Low | Relative risk predictions between 0 and |
| | | | 0.5 (inclusive) AND Absolute risk |
| | | | probabilities between 0 and 0.07 |
| | | | (inclusive) are labeled as “Low”. |
| 0.5 < x ≤ 1.0 | 0.07 < x ≤ 0.15 | Medium- | Relative risk predictions between 0.5 and |
| | | Low | 1 (inclusive of 1) AND Absolute risk |
| | | | probabilities between 0.07 and 0.15 |
| | | | (inclusive of 0.15) are labeled as |
| | | | “Medium-Low”. |
| 1.0 < x ≤ 1.5 | 0.15 < x ≤ 0.22 | Medium- | Relative risk predictions between 1 and 1.5 |
| | | High | (inclusive of 1.5) AND Absolute risk |
| | | | probabilities between 0.15 and 0.22 |
| | | | (inclusive of 0.22) are labeled as “Medium- |
| | | | High”. |
| 1.5 < x ≤ 6.67 | 0.22 < x ≤ 1 | High | Relative risk predictions greater than 1.5 |
| | | | AND Absolute risk probabilities greater than |
| | | | 0.22 are labeled as “High”. |

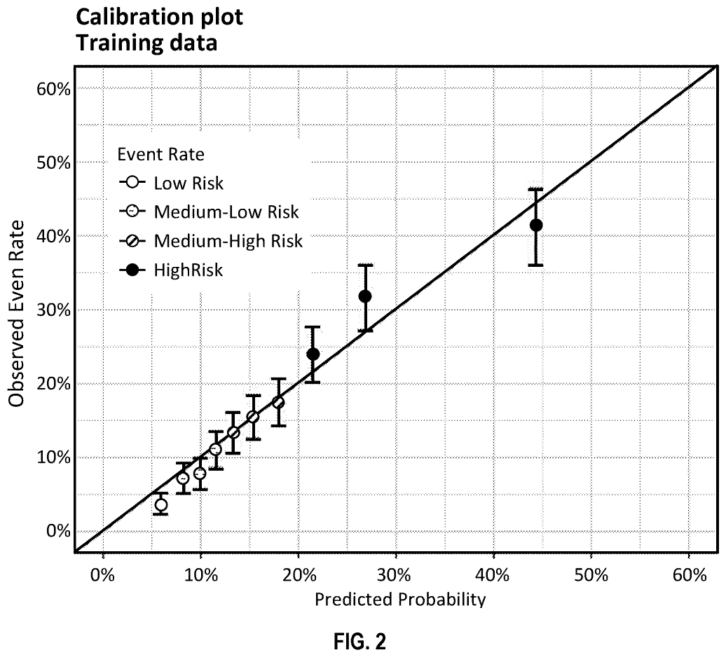

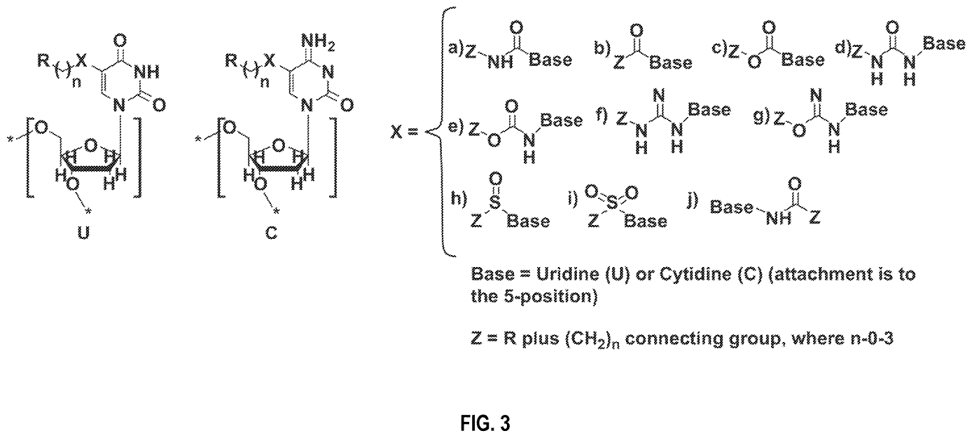

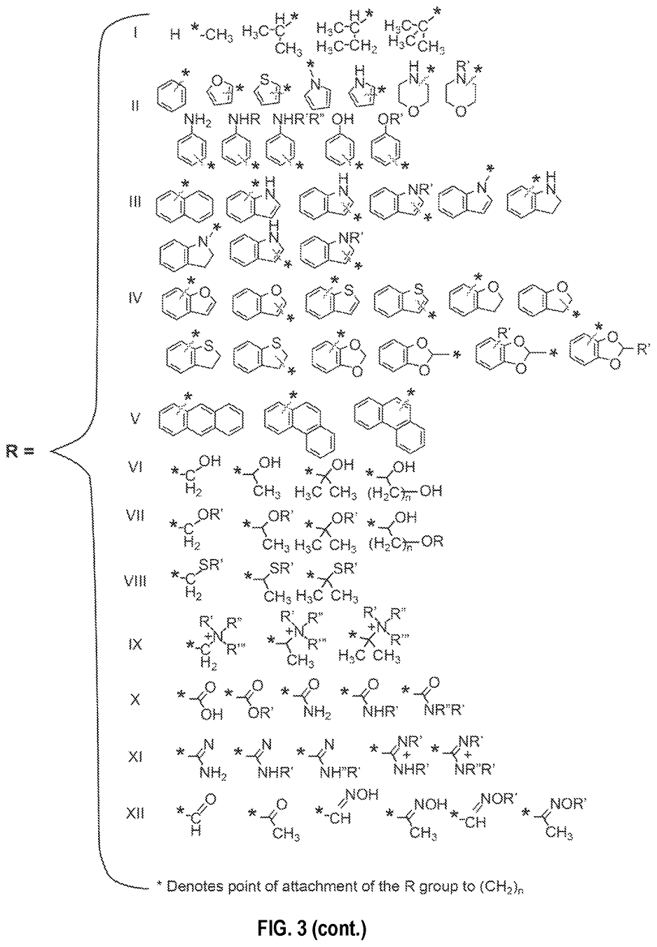

To assess the calibration of the mid-life dementia risk test model, KM plots comparing KM-estimates for observed event rate to the selected model's predicted probabilities, stratified into deciles of risk were generated (FIG. 2). Note that the error bars generally lie close to the 45-degree line, indicating that the mean predicted probability by the model accurately represents the event rate observed in the training data and the model is well calibrated.

TABLE 6
Features included in the selected model

| Entrez | Target | Target Full Name | Uniprot |
|---|---|---|---|
| PTN | PTN | Pleiotrophin | P21246 |
| NPTXR | NPTXR | Neuronal pentraxin receptor | O95502 |
| TBCA | TBCA | Tubulin-specific chaperone A | O75347 |
| CDCP1 | CDCP1 | CUB domain-containing protein 1 | Q9H5V8 |
| AGER | sRAGE | Advanced glycosylation end product-specific | B5A982 |
| | | receptor, soluble | |
| SVEP1 | SVEP1 | Sushi, von Willebrand factor type A, EGF | Q4LDE5 |
| | | and pentraxin domain-containing protein 1 | |
| NDST1 | NDST1 | Bifunctional heparan sulfate N- | P52848 |
| | | deacetylase/N-sulfotransferase 1 | |
| PPY | PH | Pancreatic hormone | P01298 |
| ASB9 | ASB9 | Ankyrin repeat and SOCS box protein 9 | Q96DX5 |
| NEFL | NFL | Neurofilament light polypeptide | P07196 |
| CCL18 | PARC | C-C motif chemokine 18 | P55774 |
| NOTCH1 | Notch1 | Neurogenic locus notch homolog protein 1 | P46531 |
| CILP2 | CILP2 | Cartilage intermediate layer protein 2 | Q8IUL8 |
| NADK | NADK | NAD kinase | O95544 |
| HAVCR1 | TIM-1 | Hepatitis A virus cellular receptor 1 | Q96D42 |
| S100A9 | calgranulin | Protein S100-A9 | P06702 |
| | B | | |
| CDON | CDON | Cell adhesion molecule-related/down- | Q4KMG0 |
| | | regulated by oncogenes | |
| MAP2K2 | MP2K2 | Dual specificity mitogen-activated protein | P36507 |
| | | kinase kinase 2 | |
| HIST3H2A | H2A3 | Histone H2A type 3 | Q7L7L0 |
| CHI3L1 | YKL-40 | Chitinase-3-like protein 1 | P36222 |
| IGFALS | IGFALS | Insulin-like growth factor-binding protein | P35858 |
| | | complex acid labile subunit | |
| S100A13 | S100A13 | Protein S100-A13 | Q99584 |
| IL18 | IL-18 | Interleukin-18 | Q14116 |
| ADAMTS13 | ATS13 | A disintegrin and metalloproteinase with | Q76LX8 |
| | | thrombospondin motifs 13 | |
| TNFRSF11B | OPG | Tumor necrosis factor receptor superfamily | O00300 |
| | | member 11B | |

The model output is the Pr(dementia-free) at 20 years. The output will be reported as the probability of a dementia diagnosis, which is (1−Pr(dementia-free)). The event probability at 20 years will be reported as a continuous variable. Because the output of this model is a probability, values outside of the range [0,1] are failures and will not be reported.

| Usage | Rule | Rationale |
|---|---|---|
| LDT | Mid-life dementia diagnosis risk | of the verification dataset) |
| | will be reported as a relative risk | This information will help guide |
| | compared to an average in ARIC | clinical decision making by |
| | visit 3 training data set. Possible | providing health care providers and |
| | values for relative risk are 0.25- | patients with a tool to understand |
| | 6.67 whereby scores >1 represent | the risk for dementia compared to |
| | a higher risk than the reference | other people with a similar disease |
| | population average and may be | process. Reporting absolute values |
| | described as an “x” times | of risk <4x will not contribute |
| | higher/lower risk than the | additional value to clinical decision |
| | reference population average | making. Moreover, very few (0.2% |
| | risk. Individuals with relative risk | of the training dataset, and 0.35% |
| | scores less than 0.25 will be | individuals in the development |
| | reported as <4x. | cohorts displayed relative risk |
| | | scores <0.25 and is therefore likely |
| | | extremely rare in the general |
| | | intended use population. |
| | Mid-life dementia diagnosis risk | This information will help guide |
| | will be reported as an absolute | clinical decision making by |
| | risk probability score. Possible | providing health care providers and |
| | values for absolute risk are 0-1. | patients with a tool to understand |
| | | the absolute numerical risk for |
| | | dementia. |
| | Dementia risk will also be | Providing risk bins will allow a |
| | reported in risk bins based on our | simple interpretation of dementia |
| | reference population (See Table | risk compared to other people with |
| | 4). | a similar disease process. |
| RUO | Dementia risk will be reported as | Reporting the absolute probability |
| | the absolute risk probability up to | of dementia diagnosis risk is |
| | 3 decimal places. | necessary for research use. |
| | Possible values for absolute risk | |
| | are 0-1. | |

Example for LDT usage: A hypothetical patient has an absolute predicted probability equal to 0.76 based on the proteomic model. This patient's relative risk is 5.07, putting them in the high-risk bin. This relative risk in the example is interpreted as follows: this patient has 5.07 times or a 407% higher risk for a dementia diagnosis within the next twenty years compared to the average individual in our reference population.

$$R R = \frac{0.76}{0.15} = 5.07$$

### Example 3. Datasets for Test Development and Validation

Development and validation cohort(s). The Atherosclerosis Risk in Communities (ARIC) Study is a prospective epidemiologic study conducted in four U.S. communities: Forsyth County, NC; Jackson, MS; the northwest suburbs of Minneapolis, MN; and Washington County, MD. The ARIC study enrolled 15,792 participants aged 45-64. Enrollment took place from 1987 to 1989 and now has over 30 years of follow-up through study visit 7, between 2018 and 2019. While the ARIC Study was originally designed to investigate the etiology and natural history of atherosclerosis, the etiology of clinical atherosclerotic diseases, and variation in cardiovascular risk factors, medical care and disease by race, gender, location, and date, expansion of the study to facilitate dementia epidemiology research has been implemented. Dementia diagnosis (including Alzheimer's disease, cerebrovascular disease-related dementia, Lewy-body related dementia, or undefined dementia) were adjudicated through cognitive assessment tests, telephone screening, informant ratings, hospital records, and death record review. The mid-life dementia risk model has been developed from documented dementia diagnoses (level 3 adjudication) starting from the third visit conducted in 1993-1995 through a thirty year follow up period, however this model is intended to perform on dementia diagnoses up to 20-years from follow up. (Knopman D S, et al. Mild cognitive impairment and dementia prevalence: The Atherosclerosis Risk in Communities Neurocognitive Study (ARIC-NCS). Alzheimers Dement (Amst). 2016; 2:1-11.)

Recent estimates suggest that prevalence of Alzheimer's disease dementia for persons aged 75-84 years is approximately 13.8%.5 The average absolute probability for developing dementia in this dataset within the span of 20 years, from visit 3 (average age of 60 years) up to visit 6 (average age of 79 years) was 0.15, or 15%, and therefore corresponded within the range of US prevalence estimates.

Dataset Stratification. For this test, the data was split independently into three sets (training 70%/verification 15%/validation 15%) stratified by dementia diagnosis as the endpoint as described in Escott-Price et al., allowing identification of a robust model while mitigating overfitting issues. (Escott-Price V, et al. Brain. 2015; 138(12): 3673-3684.) The validation data set was not used in the POC or refinement stages.

### Model Development Data

TABLE 7a
Demographic information for training data used for mid-life
dementia risk model development in ARIC visit 3
through to the end of follow up period stratified by
the occurrence of a level 3 dementia diagnosis (N/Y).

| Covariate | Measure | Total | | N | | Y | |
|---|---|---|---|---|---|---|---|
| Sample Size | N | 7895 | | 6539 | | 1356 | |
| Subject | Female | 4307 | (54.6%) | 3530 | (54%) | 777 | (57.3%) |
| Gender | Male | 3588 | (45.4%) | 3009 | (46%) | 579 | (42.7%) |
| Subject | Black | 1667 | (21.1%) | 1326 | (20.3%) | 341 | (25.1%) |
| Ethnicity | White | 6228 | (78.9%) | 5213 | (79.7%) | 1015 | (74.9%) |
| Age (years) | Mean (SD) | 60.16 | (5.694) | 59.59 | (5.614) | 62.92 | (5.252) |
| | Median | 60 | | 59 | | 64 | |
| | Range | 4-73 | | 49-73 | | 50-72 | |
| Time (Days) | Mean (SD) | 6612 | (2337) | 6664 | (2436) | 6363 | (1761) |

| | | | | |
|---|---|---|---|---|
| To Diagnosis/ | Median | 7639 | 8102 | 6722 |
| Censoring | Range | 3-9056 | 3-9056 | 540-8917 |

TABLE 7b
Cumulative Dementia diagnosis event rate by 5-year time
intervals post-Visit 3 blood draw for training data.

| Status | Years 0-5 | Years 5-10 | Years 10-15 | Years 10-20 |
|---|---|---|---|---|
| Censored after | 7487 | 6731 | 5702 | 4174 |
| time interval | | | | |
| Censored during | 375 | 1034 | 1857 | 2818 |
| time interval* | | | | |
| Event | 33 | 130 | 336 | 903 |
| Crude event rate | 0.42% | 1.65% | 4.26% | 11.44% |
| Kaplan-Meier | 0.44% | 1.89% | 5.56% | 17.79% |
| event rate# | | | | |

*Individuals with last follow-up before the end of time interval.
# Kaplan-Meier event rate takes into account the loss of individuals censored before the end of the time interval.

### Model Verification Data

TABLE 8a
Demographic information for verification data used for
mid-life dementia risk model development in ARIC
visit 3 through to the end of follow up period stratified
by the occurrence of a level 3 dementia diagnosis (N/Y).

| Covariate | Measure | Total | | N | | Y | |
|---|---|---|---|---|---|---|---|
| Sample Size | N | 1691 | | 1400 | | 291 | |
| Subject | Female | 922 | (54.5%) | 750 | (53.6%) | 172 | (59.1%) |
| Gender | Male | 769 | (45.5%) | 650 | (46.4%) | 119 | (40.9%) |
| Subject | Black | 344 | (20.3%) | 262 | (18.7%) | 82 | (28.2%) |
| Ethnicity | White | 1347 | (79.7%) | 1138 | (81.3%) | 209 | (71.8%) |
| Age (years) | Mean | 60.39 | (5.717) | 59.77 | (5.638) | 63.38 | (5.127) |
| | (SD) | | | | | | |
| | Median | 60 | | 59 | | 64 | |
| | Range | 50-72 | | 50-71 | | 50-72 | |
| Time (Days) | Mean | 6442 | (2455) | 6475 | (2562) | 6283 | (1851) |

| | | | | |
|---|---|---|---|---|
| To | (SD) | | | |
| Diagnosis/ | Median | 7378 | 8052 | 6728 |
| Censoring | Range | 10-9056 | 10-9056 | 37-8859 |

TABLE 8b
Cumulative Dementia diagnosis event rate by 5-year time intervals
post-Visit 3 blood draw for verification data

| | Years | Years | Years | Years |
|---|---|---|---|---|
| Status | 0-5 | 5-10 | 10-15 | 15-20 |
| Censored after time interval | 1570 | 1408 | 1177 | 857 |
| Censored during time | 106 | 253 | 444 | 634 |
| interval* | | | | |
| Event | 15 | 30 | 70 | 200 |
| Crude event rate | 0.89% | 1.77% | 4.14% | 11.83% |
| Kaplan-Meier event rate* | 0.95% | 2.09% | 5.61% | 18.92% |

*Individuals with last follow-up before the end of time interval.
# Kaplan-Meier event rate takes into account the loss of individuals censored before the end of the time interval.

### Model Validation Data

TABLE 9a
Demographic information for validation data to be used for mid-life dementia
risk model validation in ARIC visit 3 through to the end of follow up period stratified by
the occurrence of a level 3 dementia diagnosis (N/Y).

| Covariate | Measure | Total | | N | | Y | |
|---|---|---|---|---|---|---|---|
| Sample Size | N | 1691 | | 1401 | | 290 | |
| Subject Gender | Female | 908 | (53.7%) | 754 | (53.8%) | 154 | (53.1%) |
| | Male | 783 | (46.3%) | 647 | (46.2%) | 136 | (46.9%) |
| Subject Ethnicity | Black | 336 | (19.9%) | 261 | (18.6%) | 75 | (25.9%) |
| | White | 1355 | (80.1%) | 1140 | (81.4%) | 215 | (74.1%) |
| | Mean | 60.07 | (5.754) | 59.53 | (5.656) | 62.73 | (5.487) |
| | (SD) | | | | | | |
| Age (Years) | Median | 60 | | 59 | | 63 | |
| | Range | 50 - 72 | | 50 - 71 | | 51 - 72 | |
| | Mean | 6649 | (2339) | 6692 | (2463) | 6439 | (1592) |
| | (SD) | | | | | | |
| Time (Days) To | Median | 7764 | | 8110 | | 6738 | |
| Diagnosis/ | Range | 91 - 9050 | | 91 - 9050 | | 1279 - 8773 | |
| Censoring | | | | | | | |

TABLE 9b
Cumulative Dementia diagnosis event rate by 5-year time intervals
post-Visit 3 blood draw for validation data

| | Years | Years | | |
|---|---|---|---|---|
| Status | 0 - 5 | 5 - 10 | Years 10 - 15 | Years 15 - 20 |
| Censored after time | 1598 | 1450 | 1237 | 916 |
| interval | | | | |
| Censored during time | 89 | 219 | 391 | 573 |
| interval* | | | | |
| Event | 4 | 22 | 63 | 202 |
| Crude event rate | 0.24% | 1.30% | 3.73% | 11.95% |
| Kaplan-Meier event | 0.25% | 1.49% | 4.85% | 18.07% |
| rate# | | | | |

*Individuals with last follow-up before the end of time interval.
#Kaplan-Meier event rate takes into account the loss of individuals censored before the end of the time interval.

### Example 4. Results from Development

Data QC and Pre-Analytics Results. The original clinical dataset included samples. The following number of samples were removed based on various flags. The removals are detailed in Table 10.

TABLE 10
Number of samples removed at each data cleaning step and
corresponding number of samples remaining.

| | N | N |
|---|---|---|
| Step | (removed) | (remaining) |
| Baseline | | 11,361 |
| Prevalent dementia | 6 | 11,355 |
| Outside range normalization scale factors | 31 | 11,324 |
| Outliers | 45 | 11,279 |
| Missing event/time | 0 | 11,279 |
| Time to event = 0 | 2 | 11,277 |

Note: Outliers are samples with >5% of SOMAmer measurements exceeding 6 median absolute deviations from the median. RowCheck failures are samples with at least one scale factor outside the acceptable range of 0.4 to 2.5.

Additionally, 363 analytes were removed before analyses began as they did not pass target confirmation specificity testing leaving 4921 analytes available for analyses. No other issues were identified during Data QC or Pre-Analytics. The samples for this test were run on an analyte assay from May 13, 2019 until Jul. 8, 2019.

POC Approach and Results. The model performance requirement was met with an AUC at 20 years≥0.678.

### Refinement Approach and Results.

The selected model for the Mid-life Dementia Risk Test is a 25-analyte, AFT survival model using a Weibull distribution. The model was trained on 70% of the ARIC visit 3 dataset with no prevalent dementia. Verification metrics were calculated on a separated 15% dataset, and an additional 15% dataset was held-out for use in validation. The primary model output is absolute risk (%) (i.e., probability of event (level 3 dementia diagnosis)) within twenty years of blood draw.

Observations over the entire course of the study period were used, with model performance optimized for 20 years. Analytes were selected for the selected model through a series of feature filtering and feature reduction methods. Initial feature filtering removed analytes where there was evidence they violated assumptions of proportional hazards, using FDR¹⁶≤0.05 for a proportional hazards test as the criteria for removing the analyte, as feature selection was mainly performed using Cox proportional hazards elastic net models, which require the proportionality assumption to be satisfied.

Additionally, features with low concordance (Lin's CCC<0.85) between V4 and lifted V4.1 BioIVT verification data were excluded from model development. An important requirement for the selected model was that it perform well on both V4 and V4.1 SomaScan data; the ARIC SomaScan data is on the V4 version, but in application, the test will be used on V4.1 data. To develop a model that performs well for both, only analytes with higher concordance were used in refinement.

After the removal of the analytes listed above, the top 50 analytes, ranked according to univariate tests, were then selected for model development and cross-validation was performed to confirm the features contained enough signal to effectively predict dementia outcomes. A single Cox LASSO iteration reduced the feature set to 25 (of the 50 initial features), which was followed by 10-fold cross-validation using an elastic net Cox PH model. These results showed that models with a ridge penalty performed the best, suggesting that further reduction of the feature space would result in worse predictive performance.

Using the 25 features selected in the above routine, both an AFT Weibull and an elastic net Cox PH model were evaluated for robustness using our model assessment tools. Both models performed similarly in terms of predictive performance (i.e., AUC), but the AFT Weibull model passed the V4.0 to V4.1 verification criteria while the Cox model did not. The Weibull model was chosen as the selected model.

Performance metrics for the training data and verification data assessed at 20 years is presented in Table 11. The training and verification AUC values meet the requirement of AUC greater than or equal to 0.678. The lower bound of the confidence interval for AUC is likewise greater than 0.678 in the verification data, suggesting that our results are robust. AUC over the verification data set is slightly higher than over the training data; however, the difference is mild and non-significant, lying within 95% confidence interval.

TABLE 11
Performance metrics and 95% CI for the training
and verification data from the final Mid-life
Dementia Risk model evaluated at 20 years.

| | | | Sensi- | Speci- | | |
|---|---|---|---|---|---|---|
| | | AUC at | tivity at | ficity at | C- | PEC at |
| | | 20 years | 20 years | 20 years | Index* | 20 years |
| | N per | (95% | (95% | (95% | (95% | (95% |
| Dataset | group | CI) | CI) | CI) | CI) | CI) |
| Training | 7895 | 0.73 | 0.62 | 0.72 | 0.70 | 0.13 |
| | | (0.71- | (0.58- | (0.70- | (0.68- | (0.13- |
| | | 0.75) | 0.65) | 0.73) | 0.71) | 0.14) |
| Verification | 1691 | 0.73 | 0.61 | 0.74 | 0.69 | 0.14 |
| | | (0.69- | (0.54- | (0.71- | (0.65- | (0.12- |
| | | 0.76) | 0.67) | 0.77) | 0.72) | 0.15) |

The cut-off value used to calculate sensitivity and specificity metrics was 0.16, based on the cut-off with the highest Youden's J in the training data. *C-Index is not time specific.

Performance at 5, 10, and 15 years. The selected model was additionally used to predict the risk of a dementia diagnosis from mid-life at 5-, 10-, and 15-years post-blood draw and performance metrics were calculated. The performance metrics are detailed in Table 12.

TABLE 12
Performance metrics for the mid-life dementia risk model evaluated
at 5-, 10-, and 15-years post Visit 3 blood-draw. specific.

| Dataset | Timepoint | | AUC | Sensitivity | Specificity | C-Index* | PEC |
|---|---|---|---|---|---|---|---|
| Training | 5 | years | 0.67 | 0.54 | 0.81 | 0.70 | 0.004 |
| | 10 | years | 0.78 | 0.72 | 0.70 | | 0.018 |
| | 15 | years | 0.76 | 0.60 | 0.787 | | 0.049 |
| Verifi- | 5 | years | 0.78 | 0.73 | 0.72 | 0.69 | 0.009 |

| | | | | | |
|---|---|---|---|---|---|
| cation | 10-years | 0.75 | 0.73 | 0.71 | 0.020 |
| | 15-years | 0.76 | 0.81 | 0.64 | 0.050 |

*C-Index is not time

Performance at defined sensitivity thresholds. The selected model performance was assessed in training and verification datasets at defined sensitivity thresholds (0.8, 0.90, and 0.95) as shown in Table 13.

TABLE 13
Model performance metrics at year 20 for sensitivity levels 0.8, 0.9,
and 0.95. AUC for training data is 0.732 and for verification data is
0.733.

| Sensitivity | Dataset | Specificity | Cut-off point |
|---|---|---|---|
| 0.8 | Training | 0.51 | 0.12 |
| | Verification | 0.48 | 0.12 |
| 0.9 | Training | 0.33 | 0.10 |
| | Verification | 0.33 | 0.10 |
| 0.95 | Training | 0.20 | 0.08 |
| | Verification | 0.13 | 0.07 |

Performance compared to APOE. The performance of the selected model was compared to the predictive performance of APOE genotype to predict an individual's dementia risk. The following assessments were performed:

1. Using the selected model fit to the training data, assess model performance on subset of training samples with APOE genotype information (APOE-only data).

2. Using the selected model fit to the APOE-only data, assess model performance on APOE-only data.

3. Using the selected model feature and including APOE as a covariate, fit the model to the APOE-only data, assess model performance on APOE-only data.

4. Using a model with only APOE as a covariate, fit the model to the APOE-only data, assess model performance on APOE-only data.

Since APOE genotyping for ε2, ε3, and ε4 alleles was only conducted in a subset of ARIC participants model performance could be conducted on a subset of samples.

44% of individuals in the ARIC visit 3 dataset have an assigned APOE genotype (designated by the combination of three alleles: ε2, ε3, and ε4). Homozygotes for the ε2 allele were uncommon, with only 2 samples of this genotype diagnosed with dementia within 20 years. Therefore, samples with E2/E2 and E2/E3 genotypes were collapsed into one category. The resulting five-level factor was added as a feature to the selected model for our model comparisons. The distribution of APOE allele combinations in the training dataset and the verification dataset are shown in Tables 14 and 15, respectively, split by dementia diagnosis status.

TABLE 14
Number of samples within the training dataset with APOE genotype,
stratified by dementia diagnosis status at 20 years.

| | | | | Dementia diagnosis | | | |
|---|---|---|---|---|---|---|---|
| Covariate | Measure | Total | | within 20 years | | | |
| Sample Size | N | 7896 | | 6539 | | 1357 | |
| APOE | E2/E2 | 30 | (0.4%) | 28 | (0.4%) | 2 | (0.1%) |
| genotype | E2/E3 | 428 | (5.4%) | 367 | (5.6%) | 61 | (4.5%) |
| | E2/E4 | 92 | (1.2%) | 81 | (1.2%) | 11 | (0.8%) |
| | E3/E3 | 1842 | (23.3%) | 1637 | (25%) | 205 | (15.1%) |
| | E3/E4 | 755 | (9.6%) | 601 | (9.2%) | 154 | (11.3%) |
| | E4/E4 | 72 | (0.9%) | 45 | (0.7%) | 27 | (2%) |
| | Unknown | 4677 | (59.2%) | 3780 | (57.8%) | 897 | (66.1%) |

TABLE 15
Number of samples within the verification dataset with APOE
genotype, stratified by dementia diagnosis status at 20 years.

| | | | | Dementia diagnosis | | | |
|---|---|---|---|---|---|---|---|
| | | | | within 20 years | | | |
| Covariate | Measure | Total | | N | | Y | |
| Sample | N | 1692 | | 1400 | | 292 | |
| Size | E2/E2 | 3 | (0.2%) | 2 | (0.1%) | 1 | (0.3%) |
| APOE | E2/E3 | 94 | (5.6%) | 83 | (5.9%) | 11 | (3.8%) |
| genotype | E2/E4 | 18 | (1.1%) | 17 | (1.2%) | 1 | (0.3%) |
| | E3/E3 | 363 | (21.5%) | 321 | (22.9%) | 42 | (14.4%) |
| | E3/E4 | 140 | (8.3%) | 107 | (7.6%) | 33 | (11.3%) |
| | E4/E4 | 15 | (0.9%) | 9 | (0.6%) | 6 | (2.1%) |
| | Unknown | 1059 | (62.6%) | 861 | (61.5%) | 198 | (67.8%) |

The demographics of individuals with and without known APOE genotype stratified by dementia diagnosis status in training and verification datasets are shown in Table 16 and 17, respectively.

TABLE 16
Demographics for training data subsets with and without APOE status
information.

| Subset | Covariate | Measure | Total | | N | | Y | |
|---|---|---|---|---|---|---|---|---|
| APOE | Sample Size | N | 3219 | | 2759 | | 460 | |
| Only | Subject Gender | Female | 1788 | (55.5%) | 1543 | (55.9%) | 245 | (53.3%) |
| training | | Male | 1431 | (44.5%) | 1216 | (44.1%) | 215 | (46.7%) |
| | Subject Ethnicity | Black | 535 | (16.6%) | 421 | (15.3%) | 114 | (24.8%) |
| | | White | 2684 | (83.4%) | 2338 | (84.7%) | 346 | (75.2%) |
| | Age | Mean | 58 | (5.065) | 57.43 | (4.791) | 61.42 | (5.322) |
| | | (SD) | | | | | | |
| | | Median | 57 | | 57 | | 62 | |
| | | Range | 49 - 71 | | 49 - 71 | | 50 - 71 | |
| | Time To Diagnosis/ | Mean | 8261 | (649.4) | 8399 | (483.5) | 7435 | (867.4) |
| | Censoring | (SD) | | | | | | |
| | | Median | 8391 | | 8466 | | 7626 | |
| | | Range | 3394 - 9056 | | 6099 - 9056 | | 3394 - 8917 | |
| No APOE | Sample Size | N | 4677 | | 3780 | | 897 | |
| data | Subject Gender | Female | 2520 | (53.9%) | 1987 | (52.6%) | 533 | (59.4%) |
| training | | Male | 2157 | (46.1%) | 1793 | (47.4%) | 364 | (40.6%) |
| | Subject Ethnicity | Black | 1132 | (24.2%) | 905 | (23.9%) | 227 | (25.3%) |
| | | White | 3545 | (75.8%) | 2875 | (76.1%) | 670 | (74.7%) |
| | Age | Mean | 61.65 | (5.626) | 61.16 | (5.648) | 63.7 | (5.047) |
| | | (SD) | | | | | | |
| | | Median | 62 | | 615 | | 65 | |
| | | Range | 50 - 73 | | 50 - 73 | | 50 - 72 | |
| | Time To Diagnosis/ | Mean | 5477 | (2402) | 5397 | (2509) | 5815 | (1848) |
| | Censoring | (SD) | | | | | | |

| | | | |
|---|---|---|---|
| Median | 5727 | 5538 | 6294 |
| Range | 3 - 9056 | 3 - 9056 | 540 - 8811 |

TABLE 17
Demographics for verification data subsets with and without APOE
status information.

| Subset | Covariate | Measure | Total | | N | | Y | |
|---|---|---|---|---|---|---|---|---|
| APOE-only | Sample Size | N | 633 | | 539 | | 94 | |
| verification | Subject Gender | Female | 388 | (61.3%) | 333 | (61.8%) | 55 | (58.5%) |
| | | Male | 245 | (38.7%) | 206 | (38.2%) | 39 | (41.5%) |
| | Subject Ethnicity | Black | 107 | (16.9%) | 81 | (15%) | 26 | (27.7%) |
| | | White | 526 | (83.1%) | 458 | (85%) | 68 | (72.3%) |
| | | Mean (SD) | 58.32 | (5.278) | 57.65 | (5.017) | 62.17 | (5.113) |
| | Age | Median | 58 | | 57 | | 62 | |
| | | Range | 50 - 71 | | 50 - 71 | | 52 - 70 | |
| | Time To Diagnosis/ | Mean (SD) | 8229 | (683.6) | 8386 | (509) | 7325 | (840.5) |
| | Censoring | Median | 8376 | | 8487 | | 7258 | |
| | | Range | 3327 - 9056 | | 6145 - 9056 | | 3327 - 8859 | |
| No APOE data | Sample Size | N | 1059 | | 861 | | 198 | |
| verification | Subject Gender | Female | 535 | (50.5%) | 417 | (48.4%) | 118 | (59.6%) |
| | | Male | 524 | (49.5%) | 444 | (51.6%) | 80 | (40.4%) |
| | Subject Ethnicity | Black | 237 | (22.4%) | 181 | (21%) | 56 | (28.3%) |
| | | White | 822 | (77.6%) | 680 | (79%) | 142 | (71.7%) |
| | | Mean (SD) | 61.63 | (5.617) | 61.09 | (5.605) | 63.98 | (5.045) |
| | Age | Median | 62 | | 61 | | 65 | |
| | | Range | 50 - 72 | | 50 - 71 | | 50 - 72 | |
| | Time To Diagnosis/ | Mean (SD) | 5376 | (2511) | 5279 | (2607) | 5797 | (1993) |

| | | | | |
|---|---|---|---|---|
| Censoring | Median | 5570 | 5327 | 6242 |
| | Range | 10 - 9050 | 10 - 9050 | 37 - 8627 |

Model performance metrics for models fit with and without APOE genotype are shown in Table 18. The final proteomic model outperformed the model consisting of APOE genotype only (AUC 0.718 vs 0.609). Over the training data, the final model including APO genotype performs the best for nearly all metrics, with a 0.737 AUC in comparison to 0.718 without the APOE genotype.

TABLE 18
Model performance metrics over the subset of training data samples
(N = 3219) with subject APOE genotype information. The cut-off
value used to calculate sensitivity and specificity was 0.16, based on
highest Youden's J for the training data.

| Model (Fit to data) | AUC | Sensitivity | Specificity | C-Index | PEC |
|---|---|---|---|---|---|
| Final | 0.73 | 0.60 | 0.75 | 0.69 | 0.07 |
| (Training) | (0.69 - 0.76) | (0.54 - 0.67) | (0.73 - 0.76) | (0.67 - 0.71) | (0.06 - 0.07) |
| Final | 0.72 | 0.05 | 0.99 | 0.69 | 0.06 |
| (APOE-only | (0.68 - 0.75) | (0.02 - 0.08) | (0.98 - 0.99) | (0.66 - 0.71) | (0.05 - 0.07) |
| training) | | | | | |
| Final + APOE | 0.74 | 0.13 | 0.98 | 0.70 | 0.06 |
| (APOE-only | (0.70 - 0.77) | (0.08 - 0.17) | (0.97 - 0.98) | (0.67 - 0.73) | (0.05 - 0.07) |
| training) | | | | | |
| APOE | 0.61 | 0.00 | 1.00 | 0.59 | 0.06 |
| (APOE-only | (0.56 - 0.65) | (0.00 - 0.00) | (1.00 - 1.00) | (0.56 - 0.61) | (0.05 - 0.07) |
| training) | | | | | |

The models which were fit on the APOE-only training data consistently have low sensitivity values when using a cut-off value of 0.16 (Table 18, above). This indicates that the predicted probabilities based on the APOE-only training data are lower than those based on the full data. For comparison, Table 19 provides the sensitivity and specificity metrics when using the cut-off value with highest Youden's J over the APOE-only training data.

TABLE 19
Sensitivity and specificity over APOE training data using cut-off
values based on highest Youden's J.

| Model (Fit to data) | Cut-off value | Sensitivity | Specificity |
|---|---|---|---|
| Final | 0.14 | 0.71 | 0.65 |
| (Training) | | (0.65 - 0.77) | (0.63 - 0.66) |
| Final | 0.05 | 0.72 | 0.64 |
| (APOE-only training) | | (0.67 - 0.78) | (0.62 - 0.66) |
| Final + APOE | 0.06 | 0.61 | 0.76 |
| (APOE-only training) | | (0.55 - 0.68) | (0.74 - 0.77) |
| APOE | 0.05 | 0.45 | 0.76 |
| (APOE-only training) | | (0.39 - 0.53) | (0.74 - 0.77) |

The model with only APOE as a feature has the lowest AUC by a large margin, with a difference of 0.109 between the APOE-only model and the final model fit only to the APOE training data. In comparison, the difference in performance between the final model with and without APOE is small, at 0.019. Both differences are statistically significant, as shown in Table 20, which presents results from paired Delong's tests.

TABLE 20
Paired Delong's hypothesis test results over APOE-only training data
for difference in AUC values

| Alternative | Difference in AUC | Statistic | p-value |
|---|---|---|---|
| AUCFinal-AUCAPOE > 0 | 0.11 | 4.62 | 0.00 |
| AUCFinal-AUCFinal + | −0.02 | −2.36 | 0.01 |
| APOE < 0 | | | |

Performance metrics for models fit with and without APOE genotype over the verification data using a cut-off value of 0.16 are also provided in Table 21. Table 22 provides sensitivity and specificity metrics for the verification data when using the cut-off values with highest Youden's J, evaluated over the APOE-only subset of training data.

Note that performance over the verification data exhibits the same pattern as over the training data, where the APOE-only model has the lowest AUC and the Final+APOE model has the highest AUC. There is insufficient evidence in the verification data that these differences are statistically significant, as shown in Table 23 with results of paired Delong's hypothesis tests.

TABLE 21
Model performance metrics over the subset of verification data samples with
subject APOE genotype information (N = 1692). The cut-off value used to calculate sensitivity
and specificity was 0.16, based on highest Youden's J for the training data.

| Model | | | | | |
|---|---|---|---|---|---|
| (Fit to data) | AUC | Sensitivity | Specificity | C-Index | PEC |
| Final | 0.64 | 0.40 | 0.75 | 0.66 | 0.64 |
| (Training) | (0.56 - 0.73) | (0.25 - 0.54) | (0.70 - 0.77) | (0.61 - 0.71) | (0.56 - 0.73) |
| Final | 0.64 | 0.10 | 0.97 | 0.67 | 0.64 |
| (APOE- | (0.55 - 0.73) | (0.02 - 0.20) | (0.95 - 0.98) | (0.61 - 0.73) | (0.55 - 0.73) |
| only | | | | | |
| training) | | | | | |
| Final + | 0.70 | 0.10 | 0.97 | 0.71 | 0.70 |
| APOE | (0.64 - 0.76) | (0.02 - 0.18) | (0.95 - 0.98) | (0.66 - 0.75) | (0.64 - 0.76) |
| (APOE-only | | | | | |
| training) | | | | | |
| APOE | 0.61 | 0.00 | 1.00 | 0.59 | 0.61 |
| (APOE-only | (0.53 - 0.69) | (0.00 - 0.00) | (1.00 - 1.00) | (0.52 - 0.64) | (0.53 - 0.69) |
| training) | | | | | |

TABLE 22
Sensitivity and specificity over APOE verification data using cut-off
values based on APOE training data (as in Table 20).

| Model (Fit to data) | Cut-off value | Sensitivity | Specificity |
|---|---|---|---|
| Final | 0.14 | 0.56 | 0.65 |
| (Training) | | (0.40 - 0.72) | (0.61 - 0.69) |
| Final | 0.05 | 0.54 | 0.64 |
| (APOE-only training) | | (0.38 - 0.67) | (0.61 - 0.68) |
| Final + APOE | 0.06 | 0.54 | 0.76 |
| (APOE-only training) | | (0.40 - 0.71) | (0.73 - 0.80) |
| APOE | 0.05 | 0.40 | 0.76 |
| (APOE-only training) | | (0.27 - 0.53) | (0.73 - 0.80) |

TABLE 23
Paired Delong's hypothesis test results over the APOE-only
verification data for difference in AUC values.

| Alternative | Difference | Statistic | p-value |
|---|---|---|---|
| AUCFinal-AUCAPOE > 0 | 0.03 | 0.48 | 0.31 |
| AUC Final-AUCFinal + APOE < 0 | −0.06 | −0.51 | 0.31 |

### Example 5. Model Validation Plan and Results

Clinical Validation Plan. Validation will be assessed on the 15% hold out dataset of the ARIC Visit 3 data that have not yet been used.

The selected model from refinement is a 25-feature AFT model with a Weibull distribution. Predicted absolute probabilities for the risk of a dementia diagnosis at 20-years post-blood draw will be calculated using the selected model. Predictive performance of the model will need to have an AUC of at least 0.65, 0.66, 0.67, 0.678, 0.68, 0.69, 0.7 or greater at 20 years post-blood draw on the validation dataset.

Clinical Results on Validation Data. The required passing criteria for validation of this model was that the mid-life dementia risk model have an AUC of at least 0.65, 0.66, 0. 67, 0.678, 0.68, 0.69, 0.7 or greater at 20 years post-blood draw on the validation dataset. The model passed validation with a 20-year AUC equal to 0.70 as shown in Table 24.

TABLE 24
Performance metrics and 95% confidence intervals for
the mid-life dementia model over the validation dataset,
evaluated at 20 years post-visit 3 blood draw.

| Evalu- | | AUC | | | | PEC |
|---|---|---|---|---|---|---|
| ation | | (95% | Sensitivity | Specificity | C-Index* | (95% |
| Time | N | CI) | (95% CI) | (95% CI) | (95% CI) | CI) |
| 20 | 1691 | 0.70 | 0.60 | 0.68 | 0.67 | 0.14 |
| years | | (0.66- | (0.54- | (0.65- | (0.64- | (0.12- |
| | | 0.74) | 0.67) | 0.71) | 0.70) | 0.15) |

*C-Index is not time-specific.

Model performance metrics are slightly lower over the validation data than in the training or verification data sets. However, the AUC is sufficiently high to pass the performance criterion and in all cases the metrics are close to those in the training or verification data sets.

Results from simulations, in Table 25 below, show that the best approach for handling out-of-range aptamers is imputation with zero replacement. For this reason, it is recommended to use the average, zero, for imputation.

TABLE 25
The impact of winsorization and zero imputation on the validation
data. The table presents Lin's CCC between the original predictions
and predictions based on imputed data.

| Imputation Method | Lin's CCC |
|---|---|
| Validation data truth | 1 |
| standard | |
| Winsorization | 0.97 |
| Replacement with zero | 1 |

Ninety-five percent and 99% upper and lower bounds for variation in the model predictions due to the assay from the training data are listed in Table 26 below.

TABLE 26
95% and 99% upper and lower bounds for variation
in the model predictions on training data.

| Error | | |
|---|---|---|
| Bounds | Lower Bound | Upper Bound |
| 95% | Prediction −0.07 | Prediction +0.07 |
| 99% | Prediction −0.09 | Prediction +0.09 |

### Example 6: Multiplex Aptamer Assay and Statistical Approaches for Biomarker Identification

A multiplex aptamer assay was used to analyze test samples and control samples to identify biomarkers predictive of risk of dementia within a 5 year period. The multiplexed analysis used in this experiment included aptamers to detect approximately 5,000 proteins in blood from small sample volumes (~65 μl of serum or plasma), with low limits of detection (1 pM median), ~7 logs of dynamic range, and ~5% median coefficient of variation. The multiplex aptamer assay is described, generally, e.g., in Gold et al. (2010) Aptamer-Based Multiplexed Proteomic Technology for Biomarker Discovery. PLoS ONE 5(12): e15004; and U.S. Publication Nos: 2012/0101002 and 2012/0077695.

### Example 7. Model Specification

The endpoints for these analyses are dementia time-to-event outcomes, which have two components. 1) Time, in number of days from the blood draw to a dementia diagnosis (via level 3 adjudication methods including cognitive assessment tests, telephone screening, informant ratings, hospital records, and death record review) or exiting/completing the study. 2) A binary variable denoting whether a level 3 dementia diagnosis was observed during the study period or not.

The selected model measures 25-feature proteins (see Table 6) accelerated failure time (AFT) model with a Weibull distribution, applied to predict risk for a dementia diagnosis within 5 years. The uncalibrated model was applied to the entire study period from ARIC visit 5, and performance was maximized at five years. The final calibrated risk (CR) is a piecewise linear transformation of 5-year predictions from the 20-Year Mid-Life dementia risk model (uncalibrated risk; UCR) bounding the predicted probabilities between 0 and 1.

The model provides two predictions:

### 1. Absolute Risk:

This output is the calibrated absolute risk produced by the Mid-Life Dementia risk model. The absolute risk is calculated by generating the survival model output, which is the absolute probability of not having a dementia diagnosis (heretofore Pr[Dementia-free]), a value between 0 and 1, within 5 years. The resulting predicted probability is then subtracted from 1, to provide an absolute 5-year probability for a dementia diagnosis. This risk probability is then calibrated according to the calibration equation, described previously. The absolute risk probability is capped exactly at 0.000001 (rounded to 0.000) and 0.999999 (rounded to 1.000).

### 2. Relative Risk:

This is a continuous variable that is calculated using the absolute risk probability generated by the model (described above), divided by the “baseline” absolute probability (defined below) in the training cohort. This approach allows for the model risk probability prediction to be interpreted such that higher values indicate a greater likelihood of a dementia diagnosis within the next five years.

The baseline risk probability for the calibrated model represents the absolute risk for the “average” person in the training cohort. Using the training dataset, an average protein measurement (log₁₀[RFU]) was derived for each model protein, representing the proteomic profile of a “baseline” individual. The baseline absolute risk probability was calculated by applying the “baseline” proteomic profile to the calibrated 5-year dementia risk model. Thus, a relative risk score less than 1 represents lower than average risk and a relative risk score greater than 1 represents higher risk than average risk, based on the “baseline” proteomic profile. The rate of 5-year dementia diagnosis in the ARIC dataset for individuals over the age of 65 years is consistent with U.S. population dementia event rate age-matched for the intended use population.

The baseline absolute risk score in the calibrated training data is 0.062842 or approximately 6.3%.

Relative risk scores (where relative risk is the absolute risk divided by the baseline absolute risk of 0.062842) and absolute risk scores were stratified into four corresponding and clinically meaningful risk categories, corresponding to the binning used for the 20-year mid-life dementia risk test, in the training dataset. Kaplan-Meier (KM) plots were generated for these risk bins for the training and verification datasets across the entire time span of the study from ARIC visit 5 (6.5 years). Note that the first two risk bins (“Low” and “Medium-Low”) correspond to people whose predicted risk of a dementia diagnosis is lower than the average risk based on our data (baseline score 0.062842, relative risk equal to or lower than 1), while “Medium-High” and “High” risk bins correspond to predicted risk that is higher than the average/baseline.

The summary of relative risk stratification and corresponding event rates is shown in Table 27. Both unadjusted event rates and KM event rates are shown, KM event rates consider censoring.

TABLE 27
Summary of observed five-year event rate by relative risk bins in training data.

| | Relative | | | | Event Rate | |
|---|---|---|---|---|---|---|
| Predicted | Risk Bin | N | N | N | | Kaplan- |
| Class | Cutoffs | Total | Censored* | Events | Crude | Meie [symbol] |
| Low | 0.00 ≤ x ≤ | 817 | 121 | 12 | 1.469% | 1.510% |
| | 0.50 | | (15%) | | | |
| Medium- | 0.50 < x ≤ | 1,100 | 202 | 42 | 3.818% | 4.060% |
| Low | 1.0 | | (18%) | | | |
| Medium- | 1.0 < x ≤ | 609 | 137 | 52 | 8.539% | 9.427% |
| High | 1.5 | | (22%) | | | |
| High | 1.5 < x ≤ | 962 | 288 | 164 | 17.048% | 20.337% |
| | 15.9 | | (30%) | | | |

*Individuals were considered censored if last follow-up occurred before the end of time interval (five years from blood draw). [symbol]
aplan-Meier event rate accounts for the loss of individuals censored before the end of the time interval.

The scoring rules for risk binning based on relative risk for the 5-Year Dementia Risk Test are shown in Table 28a and scoring rules for LDT reported values are shown in Table 28b.

TABLE 28a
Scoring rules for Predicted Class (“Low,” “Medium-
Low,” “Medium-High,” and “High”
risk) using relative risk. “[RR]” denotes
the relative risk value (rounded according to the
reporting rules) for that individual.

| Test Outcome RR* (Rounded | |
|---|---|
| Relative Risk) | Predicted Class |
| 0.00 ≤ RR ≤ 0.50 | Low |
| 0.50 < RR ≤ 1.0 | Medium-Low |
| 1.0 < RR ≤ 1.5 | Medium-High |
| 1.5 < RR ≤ 15.9 | High |

TABLE 28b
Scoring rules for LDT reported values using relative
risk. “[RR]” denotes the relative risk value
(rounded according to the reporting rules) for that individual.

| Test Outcome RR | |
|---|---|
| (Rounded Relative Risk) | LDT Reported Relative Risk Values |
| 0.00 ≤ RR < 0.25 | “More than 75% lower risk compared to |
| | average” |
| 0.25 ≤ RR < 1.0 | “[1-RR] × 100% lower risk compared to |
| | average” |
| RR = 1.0 | “Average risk” |
| 1.0 < RR ≤ 15.9 | “[RR]-fold higher risk compared to average” |

To assess the calibration of the 5-year dementia risk model, KM plots comparing KM-estimates for observed event rate to the final model's absolute predicted probabilities, stratified into deciles of risk were generated. Note that the error bars generally lie close to the 45-degree line, indicating that the mean predicted probability by the model accurately represents the event rate observed in the training data and the model is well calibrated. In addition, goodness-of-fit for the calibrated 5-year dementia risk model was assessed with the Hosmer-Lemeshow (HL) test. Large p-values indicate lack of evidence of poor calibration. Calculated using quintiles, the model had HL test p-values of 0.080 and 0.273 on the training and verification sets, respectively, suggesting overall good calibration of the model.

The model output is the calibrated value of the absolute risk (1−Pr(dementia-free)), derived from the 20-year mid-life dementia risk model and applied at 5 years. The event probability at 5 years will be reported as a continuous variable. Because the output of this model is a probability, values outside of the range [0,1] are failures and will not be reported.

| Usage | Rule | Rationale |
|---|---|---|
| LDT | 5 -year dementia risk will be | This information will help |
| | reported as a relative risk | guide clinical decision making |
| | compared to an average in | by providing health care |
| | ARIC visit 5 training dataset | providers and patients with a |
| | rounded to three decimal | tool to understand the risk for |
| | places, which will be | dementia compared to other |
| | converted into a percent | people with a similar disease |
| | relative higher/lower risk with | process. Provider may use this |
| | one decimal place. Possible | information to refer patient |
| | values for relative risk are | to a specialist for additional |
| | 0.250-15.913 (reported range | recommendations/medical |
| | 75.0% lower risk to 1481.3% | advising. Reporting relative |
| | higher risk), whereby | risk values of risk <0.250 |
| | scores >1.000 represent a higher | (i.e., more than 75.0% lower |
| | risk than the reference | risk) will not contribute |
| | population average. Individuals | additional value to clinical |
| | with a relative risk score equal | decision making. |
| | to 1.000 will be reported as | |
| | “0.0% higher/lower risk.” | |
| | Individuals with relative risk | |
| | scores less than | |
| | 0.250 will be reported as | |
| | “more than 75.0% lower risk.” | |
| | 5-year dementia risk will be | This information will help |
| | reported as an absolute risk | guide clinical decision making |
| | probability rounded to three | by providing health care |
| | places, which will be | providers and patients with a |
| | converted into a percent risk | tool to understand the absolute |
| | probability rounded to one | numerical risk for dementia. |
| | decimal place. Possible values | |
| | for absolute risk | |
| | are 0.000-1.000 (reported | |
| | range 0.0%-100.0%). | |
| | Dementia risk will also be | Providing risk bins will allow |
| | reported in risk bins based on | a simple interpretation of |
| | our reference population. | dementia |
| | | risk compared to other people |
| | | with a similar disease process. |
| RUO | Dementia risk will be | Reporting the absolute |
| | reported as the absolute risk | probability of dementia |
| | probability up to 3 decimal | diagnosis risk is necessary for |
| | places. | research use. |
| | Possible values for absolute | |
| | risk are 0.000-1.000. | |

LDT Rules for Risk Binning: By way of example, Relative risk (RR) values will be reported to the patient and are used for binning and assigning risk labels. Relative risk is calculated by taking the absolute risk (AR) before rounding and dividing by the baseline risk (BR, BR=0.062842). Relative risk is then used to stratify predictions into four risk bins (“Low,” “Medium-Low,” “Medium-High,” and “High” risk).

LDT Rules for Reporting Relative Risk: By way of example, for ease of interpretation to the patient, relative risk values less than 1.0 will be reported in a manner different than those greater than 1.0. The binning procedure and reported values are therefore broken down into three components (RR<1.0, RR=1.0, RR>1.0).

For all three cases, the first two steps are:

1. Round RR.
a. For RR less than 1.0, round RR to 2 decimal places.
b. For RR greater than or equal to 1.0, round RR to 1 decimal place.

2. Assign the bin label based on the rules in Table 3a.

The value reported to the clinician/patient will vary based on the three relative risk categories (distinct from relative risk bins), which are as follows and shown in Table 3b.

3. Rounded RR less than 1.0
a. If rounded RR is less than 0.25, reported value to patient is “More than 75% lower risk compared to average”
b. If rounded RR is greater than or equal to 0.25 and less than 1.0, reported value to patient is calculated as

$$Percent lower risk = 100 \% \cdot ( ⊢ 1 - [ rounded RR from ( 1 a ) ] ) , ⊣$$

with “Risk is [percent lower risk] lower risk than average” reported to patient.

4. Rounded RR equals 1.0

a. “Patient has average risk”

5. Rounded RR greater than 1.0

a. Report value to the patient as “[rounded RR from (lb)]-fold higher risk compared to average.”

Example for LDT usage: A hypothetical patient has an absolute predicted probability that is equal to 0.023456 based on the proteomic model. This patient's relative risk rounded to two decimals is 0.37, and, since this is less than 1 is converted to a percentage and will be reported as 63% lower risk compared to average. This relative risk score corresponds to the “Low” risk bin.

$$R R = \frac{0.023456}{0.062842} = 0.37 \to 1 - 0.37 = 0.63 \to = 0.63 \cdot 100 = 63 \%$$

This relative risk in example 1 is interpreted as follows: this patient's 5-year risk for a dementia diagnosis is 63% lower compared to our reference population with an observed event rate of 8.6%.

Example 2 for LDT usage: A hypothetical patient has an absolute predicted probability that is equal to 0.760489 based on the proteomic model. This patient's relative risk rounded to one decimal is 12.1, and since it is greater than 1, will be reported as 12.1-fold higher risk compared to average. This relative risk score corresponds to the “High” risk bin.

$$R R = \frac{0.760489}{0.062842} = 12.1 - fold higher risk compared to average$$

This relative risk in the example may be interpreted as follows: this patient's 5-year risk for a dementia diagnosis is 12.1-fold higher compared to our reference population with an observed event rate of 8.6%.

### Example 8. Datasets for Test Development and Validation

Development and validation cohort(s). The Atherosclerosis Risk in Communities (ARIC) Study is a prospective epidemiologic study conducted in four U.S. communities: Forsyth County, NC; Jackson, MS; the northwest suburbs of Minneapolis, MN; and Washington County, MD. The ARIC study enrolled 15,792 participants aged 45-64. Enrollment took place from 1987 to 1989 and now has over 30 years of follow-up through study visit 7, between 2018 and 2019. The ARIC Study was originally designed to investigate the etiology and natural history of atherosclerosis, the etiology of clinical atherosclerotic diseases, and variation in cardiovascular risk factors, medical care and disease by race, gender, location, and date; however, expansion of the study to facilitate dementia epidemiology research has been implemented. (Knopman et al., Alzheimer's Dement (Amst), 2016; 2:1-11.) Dementia diagnosis (including Alzheimer's disease, cerebrovascular disease-related dementia, Lewy-body related dementia, or undefined dementia) were adjudicated through cognitive assessment tests, telephone screening, informant ratings, hospital records, and death record review (considered level 3 adjudication). The average age of onset for any incident dementia in the United States is 83.7 years. While the 20-Year Mid-Life Dementia Risk model was built using ARIC visit 3 samples (average age of 60.2 years), the 5-Year Dementia Risk model was calibrated from the 20-year Mid-Life Dementia Risk model using ARIC visit 5 samples collected between 2011 and 2013, among individuals aged 66-90 years old (average age of 75.6 years), with 6 years of follow-up data. This model is intended to predict risk for a dementia diagnosis up to 5 years from blood draw, and thus performance metrics for this model were assessed at 5 years.

The prevalence of dementia among persons over the age of 65 in the United States is nearly 11%,⁴ while the age-adjusted prevalence of any dementia among European individuals over age 65 is estimated to be 6.4%. (Lobo et al., Neurology, 2000; 54 (11 Suppl 5): S4-S9.) Rates of dementia increase with age, and thus the prevalence of Alzheimer's disease-specific dementia is 5.3% for individuals between 65 and 74 years old, 13.8% for individuals between 75 and 84 years old, and 34.6% for individuals over age 85. Alzheimer's Association Report, 2021, Alzheimer's Dement, 2021; 17(3): 327-406.) In this dataset comprised of individuals over age 65 (66-90 years old, average age 75.6 years at blood draw), the average event rate for developing dementia within the span of 5 years was 8.6%, and the proteomic model-derived baseline absolute risk was 6.3%. These values generally correspond within the range of prevalence estimates in the United States and across Europe.

Dataset Stratification. For this test, data were split independently into three sets (training 70%/verification 15%/validation 15%) stratified across datasets by dementia diagnosis (events versus non-events) as the endpoint, as described in Escott-Price et al., allowing identification of a robust model while mitigating overfitting issues. (Escott-Price V, et al. Brain. 2015; 138(12): 3673-3684.). The validation data set was not used in the POC or refinement stages.

### Model Development Data

TABLE 29a
Demographic information for training data used for the calibrated 5-year dementia
risk model development in ARIC visit 5 through to the end of follow up period
stratified by the occurrence of a level 3 dementia diagnosis (N/Y).

| | | | | Dementia Diagnosis | | | |
|---|---|---|---|---|---|---|---|
| Covariate | Measure | Total | | No | | Yes | |
| Sample Size | N | 3488 | | 3129 | | 359 | |
| Subject Gender | Female | 1977 | (56.7%) | 1783 | (57.0%) | 194 | (54.0%) |
| | Male | 1511 | (43.3%) | 1346 | (43.0%) | 165 | (46.0%) |
| Subject Ethnicity | Black | 634 | (18.2%) | 534 | (17.1%) | 100 | (27.9%) |
| | White | 2854 | (81.8%) | 2595 | (82.9%) | 259 | (72.1%) |
| APOE Genotype | E2/E2 | 28 | (0.8%) | 27 | (0.9%) | 1 | (0.3%) |
| | E2/E3 | 477 | (13.7%) | 435 | (13.9%) | 42 | (11.7%) |
| | E2/E4 | 102 | (2.9%) | 94 | (3.0%) | 8 | (2.2%) |
| | E3/E3 | 1924 | (55.2%) | 1756 | (56.1%) | 168 | (46.8%) |
| | E3/E4 | 761 | (21.8%) | 648 | (20.7%) | 113 | (31.5%) |
| | E4/E4 | 64 | (1.8%) | 50 | (1.6%) | 14 | (3.9%) |
| | Unknown | 132 | (3.8%) | 119 | (3.8%) | 13 | (3.6%) |
| Age (Years) At Visit 5 | Mean (SD) | 75.524 | (5.147) | 75.134 | (4.955) | 78.928 | (5.533) |
| | Median | 75 | | 74 | | 79 | |
| | Range | 66-90 | | 66-90 | | 67-90 | |
| Time (Days) To | Mean (SD) | 1867.062 | (483.618) | 1923.347 | (445.202) | 1376.487 | (526.572) |

| Diagnosis/ | Median | 1990 | 2030 | 1440 |
|---|---|---|---|---|
| Censoring | Range | 4-2400 | 25-2400 | 4-2270 |

TABLE 29b
Cumulative dementia diagnosis event rate based on
time post-Visit 5 blood draw for training data.

| | Within | Within Follow- |
|---|---|---|
| Status | 5 Years | up Period |
| Censored or had event after time | 2470 | NA |
| interval | | |
| Censored during time interval* | 748 | 3129 |
| Cumulative Number of Events | 270 | 359 |
| Crude event rate | 7.741% | 10.292% |
| Kaplan-Meier event rat [symbol] | 8.460% | 13.411% |

*Individuals were considered censored if last follow-up occurred before the end of time interval (five years from blood draw). [symbol]
aplan-Meier event rate accounts for the loss of individuals censored before the end of the time interval.

### Model Verification Data

TABLE 30a
Demographic information for verification data used for the calibrated 5-Year
Dementia Risk model development in ARIC visit 5 through to the end of follow
up period stratified by the occurrence of a level 3 dementia diagnosis (N/Y).

| | | | | Dementia Diagnosis | | | |
|---|---|---|---|---|---|---|---|
| Covariate | Measure | Total | | No | | Yes | |
| Sample Size | N | 748 | | 671 | | 77 | |
| Subject Gender | Female | 434 | (58.0%) | 400 | (59.6%) | 34 | (44.2%) |
| | Male | 314 | (42.0%) | 271 | (40.4%) | 43 | (55.8%) |
| Subject Ethnicity | Black | 144 | (19.3%) | 117 | (17.4%) | 27 | (35.1%) |
| | White | 604 | (80.7%) | 554 | (82.6%) | 50 | (64.9%) |
| APOE Genotype | E2/E2 | 8 | (1.1%) | 6 | (0.9%) | 2 | (2.6%) |
| | E2/E3 | 88 | (11.8%) | 80 | (11.9%) | 8 | (10.4%) |
| | E2/E4 | 22 | (2.9%) | 21 | (3.1%) | 1 | (1.3%) |
| | E3/E3 | 406 | (54.3%) | 373 | (55.6%) | 33 | (42.9%) |
| | E3/E4 | 172 | (23.0%) | 152 | (22.7%) | 20 | (26.0%) |
| | E4/E4 | 12 | (1.6%) | 7 | (1.0%) | 5 | (6.5%) |
| | Unknown | 40 | (5.3%) | 32 | (4.8%) | 8 | (10.4%) |
| Age (Years) At Visit 5 | Mean (SD) | 75.574 | (5.07) | 75.195 | (4.939) | 78.87 | (5.032) |
| | Median | 75 | | 74 | | 79 | |
| | Range | 67-90 | | 67-90 | | 70-89 | |
| Time (Days)To | Mean (SD) | 1900.636 | (458.629) | 1958.908 | (414.954) | 1392.844 | (508.178) |

| Diagnosis/ | Median | 2020 | 2050 | 1430 |
|---|---|---|---|---|
| Censoring | Range | 26-2400 | 26-2400 | 129-2260 |

TABLE 30b
Cumulative dementia diagnosis event rate based on
time post-Visit 5 blood draw for verification data.

| | Within | Within Follow- |
|---|---|---|
| Status | 5 Years | up Period |
| Censored or had event after time | 554 | NA |
| interval | | |
| Censored during time interval* | 134 | 671 |
| Cumulative Number of Events | 60 | 77 |
| Crude event rate | 8.021% | 10.294% |
| Kaplan-Meier event rat [symbol] | 8.680% | 12.835% |

*Individuals were considered censored if last follow-up occurred before the end of time interval (five years from blood draw). [symbol]
aplan-Meier event rate accounts for the loss of individuals censored before the end of the time interval.

### Model Validation Data

TABLE 31a
Demographic information for validation data used for the calibrated 5-year dementia
risk model development in ARIC visit 5 through to the end of follow up period
stratified by the occurrence of a level 3 dementia diagnosis (N/Y).

| | | | | Dementia Diagnosis | | | |
|---|---|---|---|---|---|---|---|
| Covariate | Measure | Total | | No | | Yes | |
| Sample Size | N | 749 | | 671 | | 78 | |
| Subject Gender | Female | 431 | (57.5%) | 388 | (57.8%) | 43 | (55.1%) |
| | Male | 318 | (42.5%) | 283 | (42.2%) | 35 | (44.9%) |
| Subject Ethnicity | Black | 120 | (16.0%) | 105 | (15.6%) | 15 | (19.2%) |
| | White | 629 | (84.0%) | 566 | (84.4%) | 63 | (80.8%) |
| APOE Genotype | E2/E2 | 6 | (0.8%) | 6 | (0.9%) | 0 | (0.0%) |
| | E2/E3 | 96 | (12.8%) | 82 | (12.2%) | 14 | (17.9%) |
| | E2/E4 | 13 | (1.7%) | 10 | (1.5%) | 3 | (3.8%) |
| | E3/E3 | 415 | (55.4%) | 385 | (57.4%) | 30 | (38.5%) |
| | E3/E4 | 164 | (21.9%) | 141 | (21.0%) | 23 | (29.5%) |
| | E4/E4 | 15 | (2.0%) | 13 | (1.9%) | 2 | (2.6%) |
| | Unknown | 40 | (5.3%) | 34 | (5.1%) | 6 | (7.7%) |
| Age (Years) At Visit 5 | Mean (SD) | 75.706 | (5.138) | 75.323 | (4.943) | 79 | (5.622) |
| | Median | 75 | | 74 | | 79 | |
| | Range | 67-90 | | 67-89 | | 69-90 | |
| Time (Days) To | Mean (SD) | 1892.421 | (439.225) | 1958.507 | (368.724) | 1323.91 | (570.611) |

| Diagnosis/ | Median | 1990 | 2030 | 1450 |
|---|---|---|---|---|
| Censoring | Range | 47-2400 | 151-2400 | 47-2150 |

TABLE 31b
Cumulative dementia diagnosis event rate based on
time post-Visit 5 blood draw for validation data.

| | Within | Within Follow- |
|---|---|---|
| Status | 5 Years | up Period |
| Censored or had event after time | 547 | NA |
| interval | | |
| Censored during time interval* | 141 | 671 |
| Cumulative Number of Events | 61 | 78 |
| Crude event rate | 8.144% | 10.414% |
| Kaplan-Meier event rat [symbol] | 8.709% | 12.860% |

*Individuals were considered censored if last follow-up occurred before the end of time interval (five years from blood draw). [symbol]
aplan-Meier event rate accounts for the loss of individuals censored before the end of the time interval.

Results from Development

Data QC and Pre-Analytics Results. For ARIC visit 5, there were 5,024 samples with clinical endpoint data. There were no proteomic data for 8 (0.1592) of these samples. There was a total of 5,016 samples that had both clinical and v4.0 proteomic data. There was no noticeable association between clinical covariates (gender, age, ethnicity, and APOE genotype) and the normalization scale factors. The following number of samples were removed based on various flags. The removals are detailed in Table 32.

TABLE 32
Number of samples removed at each data cleaning step
and corresponding number of samples remaining.

| | N | N |
|---|---|---|
| Step | (removed) | (remaining) |
| Baseline (samples with clinical endpoint) | NA | 5,024 |
| Missing proteomic information | 8 | 5,016 |
| Outside range normalization scale factors | 0 | 5,016 |
| Outliers | 25 | 4,991 |
| Time-to-event of 0 days | 6 | 4,985 |

Note:
Outliers are samples with >5% of analyte measurements exceeding 6 median absolute deviations from the median. RowCheck failures are samples with at least one scale factor outside the acceptable range of 0.4 to 2.5.

In addition, 363 analytes that did not pass target confirmation specificity testing were removed before analyses began, leaving 4921 analytes available for analyses. No other issues were identified during Data QC or Pre-Analytics. The samples for this test were run on an analyte assay from Sep. 1, 2018 until Oct. 16, 2018.

### POC Approach and Results

The 20-year mid-life dementia risk model is an AFT Weibull survival model developed on the ARIC visit 3 dataset. The 20-year model was used to predict 5-year dementia risk for individuals in the ARIC visit 5 dataset, an older population. While these predictions provided good rankings of risk (e.g., individuals with lower predicted risks tended to have lower event rates), these predictions were not well calibrated.

The calibration method that performed best in POC analysis was linear scaling fit on 20 quantiles with an AUC of 0.799 (confidence interval 0.748 to 0.802), which exceeded our feasibility criterion of an AUC greater than or equal to 0.678. The PEC for this model was 0.081 (confidence interval 0.072 to 0.090) and the Hosmer-Lemeshow test p-value was 0.123 (ap-value>0.05 means lack of evidence against good calibration). These results indicate that the model is well-calibrated. Additionally, the KM event rate 95% confidence intervals in the calibration plot capture the mean predicted risk for all 10 deciles.

### Refinement Approach and Results

The calibrated 5-year dementia risk model developed in refinement was developed using the training and verification splits from POC of the ARIC visit 5 dataset. The individuals in these sets were aged 66 to 90 years old. Individuals with diagnosed dementia at blood draw were excluded from this model development. Individuals with mild cognitive impairment (MCI) at blood draw were not excluded.

Refinement analysis pursued calibration of the 20-year mid-life dementia risk model, which was developed on ARIC visit 3 and predicts risk of dementia within 20 years, on an older population (ARIC visit 5) predicting risk of dementia within 5 years. The 20-year mid-life dementia risk model is an AFT Weibull model with 25 analytes. Uncalibrated predictions for the development data were obtained by predicting dementia risk with the 20-year mid-life dementia risk model at 5 years (1825 days). POC results show that these predictions severely under-predicted the observed event rate.

The calibration function (e.g., the function that transforms an uncalibrated predicted risk into a calibrated predicted risk) was fit by first grouping predicted probabilities into quantiles and then using linear regression to find the line of best fit between the mean predicted risk and the KM event rate for each quantile. The linear regression fit was weighted based on width of KM event rate confidence intervals: quantiles with small confidence intervals were more heavily weighted than quantiles with large confidence intervals.

The final calibration function was fit using 20 quantiles and weights for each quantile defined by

$${w}_{i} = \frac{1}{{( {UB}_{i} - {LB}_{i} )}^{0.75}} .$$

These tuning parameters (power in the weights and number of quantiles) were chosen through 5-repeat, 10-fold cross-validation optimizing on the Hosmer-Lemeshow test p-value; however, the cross-validated performance was very similar across all parameter combinations.

A piecewise defined calibration function was chosen to bound calibrated predictions between 0 and 1. Without the bounds at the top and the bottom of the uncalibrated predicted values, the calibrated predicted values are not guaranteed to be between 0 and 1.

Thus, the final calibrated risk (“CR”) is a piecewise linear transformation of 5-year predictions from the mid-life dementia risk model (uncalibrated risk; “UCR”) bounding the predicted-probabilities between 0 and 1 Specifically,

$$CR = \{ \begin{matrix}0.000001 & if & UCR \leq 0.000513 \\ 32.795126 \cdot UCR − 0.01681402 & if & 0.000513 < UCR < 0.031005 \\ 0.999999 & if & UCR \geq 0.031005\end{matrix}$$

Performance metrics for the training and verification data assessed at 5 years is presented in Table 33. The training and verification AUC values meet the requirement of AUC greater than or equal to 0.678.

TABLE 33
Performance and 95% confidence interval (CI) of calibrated
5-year dementia risk model for training and verification
data, evaluated at 5 years. Training and verification results
exceed performance criteria of an AUC greater than or equal
to 0.678. Sensitivity and specificity values were determined
based on the cut-off value in the training dataset maximizing
Youden's J index (0.073). AUC, Area Under the Curve; CI,
Confidence Intervals; HL, Hosmer-Lemeshow.

| | | | | Sensi- | Speci- | HL |
|---|---|---|---|---|---|---|
| | | AUC at | | tivity at | ficity at | Test for |
| | | 5 Years | C-Inder* | 5 Years | 5 Years | Goodness |
| | N per | (95% | (95% | (95% | (95% | of Fit |
| Dataset | group | CI) | CI) | CI) | CI) | p-value |
| Training | | 0.778 | 0.747 | 0.770 | 0.673 | 0.080 |
| | 3488 | (0.748, | (0.717, | (0.722, | (0.654, | |
| | | 0.802) | 0.769) | 0.819) | 0.690) | |
| Verifi- | | 0.735 | 0.718 | 0.667 | 0.641 | 0.273 |
| cation | 748 | (0.669, | (0.663, | (0.552, | (0.605, | |
| | | 0.794) | 0.769) | 0.783) | 0.679) | |

*C-Index is not time specific. [symbol]
or the Hosmer-Lemeshow (HL) test, p-values less than 0.05 indicate evidence of poor calibration.

### Performance Compared to APOE

The performance of the final model was compared to the performance of APOE genotype to predict an individual's 5-year dementia risk.

An APOE-only AFT Weibull model was fit on the subset of the training data with known APOE genotype. Performance of the APOE-only model was compared to the performance of the final model applied to the subset of training and verification samples with known APOE genotype information.

Since APOE genotyping for ε2, ε3, and ε4 alleles was only conducted in a subset of ARIC participants, model performance could only be conducted on a subset of samples. However, 96.2% of training samples and 94.7% of verification samples derived from the ARIC visit 5 dataset had an assigned APOE genotype (designated by the combination of three alleles: ε2, ε3, and ε4). Homozygotes for the ε2 allele were uncommon (N=28), with only 1 sample of this genotype diagnosed with dementia within 5 years in the training dataset. Therefore, samples with E2/E2 and E2/E3 genotypes were combined into one category for this model.

Model performance metrics are shown in Table 34 and dynamic range is shown in Table 35. The final calibrated 5-year dementia risk proteomic model outperformed the model consisting of APOE genotype only, with AUCs of 0.780 vs 0.575, respectively, for training samples and AUCs of 0.726 vs 0.564, respectively, for verification samples.

TABLE 34
Model performance metrics evaluated at 5 years post-blood
draw for the subset of training and verification data samples
with subject APOE genotype information (training data, N =
3356; verification data, N = 708). Confidence intervals
were estimated via bootstrapping. APOE, Apolipoprotein E;
AUC, Area Under the Curve; CI, Confidence Intervals.

| | | AUC at | | Sensitivity | Specificity |
|---|---|---|---|---|---|
| | | 5 Years | C-Index¹ | at 5 Years | at 5 Years |
| Model | Dataset | (95% CI) | (95% CI) | (95% CI) | (95% CI) |
| APOE-only | Training² | 0.575 | 0.567 | 0.379 | 0.766 |
| Model³ | | (0.542, | (0.537, | (0.321, | (0.752, |
| | | 0.606) | 0.595) | 0.432) | 0.786) |
| | Verifica- | 0.564 | 0.559 | 0.333 | 0.763 |
| | tion² | (0.493, | (0.489, | (0.209, | (0.728, |
| | | 0.633) | 0.617) | 0.464) | 0.797) |
| 5-Year | Training² | 0.780 | 0.748 | 0.774 | 0.672 |
| Dementia | | (0.747, | (0.720, | (0.729, | (0.654, |
| Model⁴ | | 0.806) | 0.773) | 0.826) | 0.688) |
| | Verifica- | 0.726 | 0.709 | 0.648 | 0.639 |
| | tion² | (0.659, | (0.651, | (0.523, | (0.595, |
| | | 0.790) | 0.767) | 0.782) | 0.682) |

¹C-Index is not dependent on evaluation time
²Subset of samples with known APOE genotype
³Sensitivity and specificity were calculated using the cutoff that maximized Youden's J for the APOE-only model on subset of the training data with known APOE genotype (cutoff = 0.076)
⁴Sensitivity and specificity were calculated using the cutoff that maximized Youden's J on the full training data for the 5-year dementia risk model (cutoff = 0.073)

TABLE 35
The dynamic range of the 5-year dementia risk model compared
to the dynamic range of the APOE-only model in training data.

| | | Predicted | Observed |
|---|---|---|---|
| Method | Bins in Ratio | Risk Ratio | Risk Ratio |
| 5-Year Dementia | High Risk over Low | 10.307 | 13.403 |
| Risk Model | Risk | | |
| APOE-only Model* | E4/E4 Genotype over | 2.496 | 2.317 |
| | combined E2/E2 + | | |
| | E2/E3 Genotype | | |

*Event rates calculated on the subset of the training data with known APOE genotype.

### Validation Plan

### Clinical Validation Plan

Validation will be assessed on the 15% hold out dataset of the ARIC Visit 5 data that have not yet been used. The final model from refinement is a calibration function applied to the 20-year mid-life dementia risk model, which is a 25-feature AFT model with a Weibull distribution. Predicted absolute probabilities for the risk of a dementia diagnosis at 5 years post-blood draw will be calculated using the final calibrated model. Predictive performance of the model will need to have an AUC of at least 0.678 at 5 years post-blood draw on the validation dataset.

### Model Validation Results

### Clinical Results on Validation Data

The required passing criteria for validation was that the 5-year dementia risk model have an AUC of at least 0.678 at 5 years post-blood draw using the validation dataset. The model passed validation with a 5-year AUC equal to 0.776 as shown in Table 36.

TABLE 36
Performance metrics and 95% confidence intervals (CI)
for the 5-year dementia risk model over the validation
dataset, evaluated at 5 years post-visit 5 blood draw.

| | | | | Sensi- | Speci- | HL |
|---|---|---|---|---|---|---|
| | | AUC at | | tivity at | ficity at | Test for |
| | | 5 Years | C-Inder* | 5 Years | 5 Years | Goodness |
| | N per | (95% | (95% | (95% | (95% | of Fit |
| Dataset | group | CI) | CI) | CI) | CI) | p-valu [symbol] |
| Validation | 749 | 0.776 | 0.761 | 0.689 | 0.673 | 0.357 |
| | | (0.705, | (0.703, | (0.571, | (0.635, | |
| | | 0.831) | 0.807) | 0.812) | 0.709) | |

*C-Index is not time-specific. [symbol]
or the Hosmer-Lemeshow (HL) test, p-values less than 0.05 indicate evidence of poor calibration.

Model performance metrics over the validation data are about the same or higher than those from the training or verification datasets.

Validation conclusions: The final model is a recalibration of the 25-feature mid-life dementia risk model and predicts risk of a dementia diagnosis within 5 years among adults aged 65 years and older. The model output for RUO is an absolute risk probability, with possible scores ranging from 0.000 to 1.000. The model outputs for LDT are 1) a relative risk for a dementia diagnosis compared to an average person in the reference population and 2) a corresponding risk bin. The range of raw relative risk values is 0.25 to 15.9, which will be converted to a percent lower risk for values less than 1.0 and reported as x-fold higher risk for values greater than 1.0. Validation exceeds the performance metric of an AUC greater than or equal to 0.678.

### Example 9: Analysis of Dementia Model Biomarker Panels

Model biomarker panels comprising various combinations of the biomarkers listed in Table 6 were analyzed to determine the Area Under the Curve (AUC) value for the various combinations. The model biomarker panels may be based on a panel of N biomarker proteins having an AUC value of at least 0.65, at least 0.66, at least 0.67, at least 0.68, at least 0.69, at least 0.7, at least 0.75, at least 0.8, at least 0.85, at least 0.9, or at least 0.95, where N is 1, 2, 3, 4, 5, 6, 7, 8, 9, 10, 11, 12, 13, 14, 15, 16, 17, 18, 19, 20, 21, 22, 23, 24, and/or 25 of the biomarker proteins listed in Table 6. The Tables below show exemplary model results when various combinations comprising 1 to 25 biomarker proteins were measured.

TABLE 37
CILP2 Biomarker Panels-20 Year Model

| AUC | CILP2 | CDON | H2A3 | IGFALS | MP2K2 | NADK | Notch1 | PH | PTN | S100A13 |
|---|---|---|---|---|---|---|---|---|---|---|
| 0.588 | CILP2 | | H2A3 | | | | | | | |
| 0.589 | CILP2 | CDON | | | | | | | | |
| 0.59 | CILP2 | | | | | | Notch1 | | | |
| 0.591 | CILP2 | | | | | | | | | S100A13 |
| 0.596 | CILP2 | CDON | | | | | Notch1 | | | |
| 0.598 | CILP2 | | | | MP2K2 | | | | | |
| 0.599 | CILP2 | | H2A3 | | | | | | | S100A13 |
| 0.6 | CILP2 | CDON | H2A3 | | | | | | | |
| 0.6 | CILP2 | | | | | NADK | | | | |
| 0.601 | CILP2 | CDON | | | | | | | | S100A13 |
| 0.602 | CILP2 | | H2A3 | | | NADK | | | | |
| 0.602 | CILP2 | | H2A3 | | | | Notch1 | | | |
| 0.607 | CILP2 | | | | MP2K2 | | Notch1 | | | |
| 0.607 | CILP2 | | | | | | Notch1 | | | S100A13 |
| 0.608 | CILP2 | CDON | | | MP2K2 | | | | | |
| 0.608 | CILP2 | CDON | H2A3 | | | | Notch1 | | | |
| 0.609 | CILP2 | CDON | | | | NADK | | | | |
| 0.609 | CILP2 | | | | | NADK | Notch1 | | | |
| 0.609 | CILP2 | CDON | H2A3 | | | | | | | S100A13 |
| 0.611 | CILP2 | CDON | H2A3 | | | NADK | | | | |
| 0.612 | CILP2 | CDON | | | MP2K2 | | Notch1 | | | |
| 0.612 | CILP2 | | H2A3 | | | NADK | Notch1 | | | |
| 0.612 | CILP2 | | | | | | | PH | | |
| 0.613 | CILP2 | | | IGFALS | | | | | | |
| 0.613 | CILP2 | CDON | | | | NADK | Notch1 | | | |
| 0.613 | CILP2 | | | | MP2K2 | | | | | S100A13 |
| 0.613 | CILP2 | CDON | | | | | Notch1 | | | S100A13 |
| 0.614 | CILP2 | | H2A3 | | MP2K2 | | | | | |
| 0.614 | CILP2 | | | | | NADK | | | | S100A13 |
| 0.614 | CILP2 | | H2A3 | | | NADK | | | | S100A13 |
| 0.615 | CILP2 | | H2A3 | | | | Notch1 | | | S100A13 |
| 0.616 | CILP2 | | | | MP2K2 | NADK | | | | |
| 0.617 | CILP2 | CDON | H2A3 | | | NADK | Notch1 | | | |
| 0.619 | CILP2 | | H2A3 | IGFALS | | | | | | |
| 0.62 | CILP2 | CDON | | IGFALS | | | | | | |
| 0.62 | CILP2 | | H2A3 | | MP2K2 | NADK | | | | |
| 0.62 | CILP2 | | | | MP2K2 | NADK | Notch1 | | | |
| 0.62 | CILP2 | CDON | | | MP2K2 | | | | | S100A13 |
| 0.621 | CILP2 | CDON | H2A3 | | MP2K2 | | | | | |
| 0.621 | CILP2 | | | IGFALS | | | Notch1 | | | |
| 0.621 | CILP2 | | H2A3 | | MP2K2 | | Notch1 | | | |
| 0.621 | CILP2 | CDON | | | | NADK | | | | S100A13 |
| 0.621 | CILP2 | CDON | H2A3 | | | NADK | | | | S100A13 |
| 0.621 | CILP2 | CDON | H2A3 | | | | Notch1 | | | S100A13 |
| 0.621 | CILP2 | | | | | | | PH | | S100A13 |
| 0.622 | CILP2 | CDON | | | MP2K2 | NADK | | | | |
| 0.622 | CILP2 | | H2A3 | | MP2K2 | | | | | S100A13 |
| 0.623 | CILP2 | CDON | | | | | | PH | | |
| 0.623 | CILP2 | | | | MP2K2 | | Notch1 | | | S100A13 |
| 0.624 | CILP2 | CDON | | IGFALS | | | Notch1 | | | |
| 0.624 | CILP2 | | | | | | Notch1 | PH | | |
| 0.625 | CILP2 | | | IGFALS | MP2K2 | | | | | |
| 0.625 | CILP2 | CDON | | | MP2K2 | NADK | Notch1 | | | |
| 0.625 | CILP2 | | | IGFALS | | | | | | S100A13 |
| 0.625 | CILP2 | | | | | NADK | Notch1 | | | S100A13 |
| 0.626 | CILP2 | CDON | H2A3 | IGFALS | | | | | | |
| 0.626 | CILP2 | CDON | H2A3 | | MP2K2 | | Notch1 | | | |
| 0.626 | CILP2 | | H2A3 | | MP2K2 | NADK | Notch1 | | | |
| 0.627 | CILP2 | CDON | H2A3 | | MP2K2 | NADK | | | | |
| 0.627 | CILP2 | | H2A3 | | | | | PH | | |
| 0.627 | CILP2 | CDON | | | MP2K2 | | Notch1 | | | S100A13 |
| 0.627 | CILP2 | | H2A3 | | | NADK | Notch1 | | | S100A13 |
| 0.628 | CILP2 | | | IGFALS | | NADK | | | | |
| 0.628 | CILP2 | | H2A3 | IGFALS | | NADK | | | | |
| 0.628 | CILP2 | | H2A3 | IGFALS | | | Notch1 | | | |
| 0.628 | CILP2 | CDON | | | | | Notch1 | PH | | |
| 0.628 | CILP2 | | H2A3 | IGFALS | | | | | | S100A13 |
| 0.628 | CILP2 | CDON | H2A3 | | MP2K2 | | | | | S100A13 |
| 0.629 | CILP2 | CDON | | IGFALS | MP2K2 | | | | | |
| 0.629 | CILP2 | CDON | | | | NADK | Notch1 | | | S100A13 |
| 0.63 | CILP2 | CDON | H2A3 | | MP2K2 | NADK | Notch1 | | | |
| 0.63 | CILP2 | | | | MP2K2 | | | PH | | |
| 0.63 | CILP2 | CDON | | IGFALS | | | | | | S100A13 |
| 0.63 | CILP2 | CDON | H2A3 | | | NADK | Notch1 | | | S100A13 |
| 0.63 | CILP2 | CDON | | | | | | PH | | S100A13 |
| 0.63 | CILP2 | | H2A3 | | | | | PH | | S100A13 |
| 0.631 | CILP2 | CDON | H2A3 | IGFALS | | | Notch1 | | | |
| 0.631 | CILP2 | | | IGFALS | MP2K2 | | Notch1 | | | |
| 0.631 | CILP2 | | | | MP2K2 | NADK | | | | S100A13 |
| 0.632 | CILP2 | | H2A3 | IGFALS | MP2K2 | | | | | |
| 0.632 | CILP2 | CDON | | IGFALS | | NADK | | | | |
| 0.632 | CILP2 | CDON | H2A3 | IGFALS | | NADK | | | | |
| 0.632 | CILP2 | | | IGFALS | | NADK | Notch1 | | | |
| 0.632 | CILP2 | | | | | NADK | | PH | | |
| 0.632 | CILP2 | | H2A3 | | MP2K2 | NADK | | | | S100A13 |
| 0.632 | CILP2 | | H2A3 | | MP2K2 | | Notch1 | | | S100A13 |
| 0.633 | CILP2 | CDON | H2A3 | IGFALS | | | | | | S100A13 |
| 0.633 | CILP2 | | | | | | Notch1 | PH | | S100A13 |
| 0.634 | CILP2 | | | IGFALS | MP2K2 | NADK | | | | |
| 0.634 | CILP2 | CDON | | IGFALS | MP2K2 | | Notch1 | | | |
| 0.634 | CILP2 | CDON | | IGFALS | | NADK | Notch1 | | | |
| 0.634 | CILP2 | | H2A3 | IGFALS | | NADK | Notch1 | | | |
| 0.634 | CILP2 | CDON | H2A3 | | | | | PH | | |
| 0.634 | CILP2 | | H2A3 | | | NADK | | PH | | |
| 0.635 | CILP2 | | | | MP2K2 | | Notch1 | PH | | |
| 0.635 | CILP2 | CDON | | | MP2K2 | NADK | | | | S100A13 |
| 0.635 | CILP2 | | | IGFALS | | | Notch1 | | | S100A13 |
| 0.636 | CILP2 | | H2A3 | IGFALS | MP2K2 | NADK | | | | |
| 0.636 | CILP2 | CDON | H2A3 | IGFALS | | NADK | Notch1 | | | |
| 0.636 | CILP2 | | H2A3 | | | | Notch1 | PH | | |
| 0.636 | CILP2 | CDON | H2A3 | | MP2K2 | NADK | | | | S100A13 |
| 0.636 | CILP2 | CDON | H2A3 | | MP2K2 | | Notch1 | | | S100A13 |
| 0.637 | CILP2 | CDON | H2A3 | IGFALS | MP2K2 | | | | | |
| 0.637 | CILP2 | CDON | | | MP2K2 | | | PH | | |
| 0.637 | CILP2 | | | | MP2K2 | NADK | Notch1 | | | S100A13 |
| 0.637 | CILP2 | CDON | H2A3 | | | | | PH | | S100A13 |
| 0.638 | CILP2 | CDON | | IGFALS | MP2K2 | NADK | | | | |
| 0.638 | CILP2 | | H2A3 | IGFALS | MP2K2 | | Notch1 | | | |
| 0.638 | CILP2 | | | IGFALS | MP2K2 | NADK | Notch1 | | | |
| 0.638 | CILP2 | | | | | NADK | Notch1 | PH | | |
| 0.638 | CILP2 | | | IGFALS | MP2K2 | | | | | S100A13 |
| 0.638 | CILP2 | | H2A3 | IGFALS | | NADK | | | | S100A13 |
| 0.638 | CILP2 | CDON | | IGFALS | | | Notch1 | | | S100A13 |
| 0.638 | CILP2 | | | | MP2K2 | | | PH | | S100A13 |
| 0.638 | CILP2 | CDON | | | | | Notch1 | PH | | S100A13 |
| 0.639 | CILP2 | CDON | | | | NADK | | PH | | |
| 0.639 | CILP2 | | | IGFALS | | NADK | | | | S100A13 |
| 0.639 | CILP2 | | H2A3 | IGFALS | | | Notch1 | | | S100A13 |
| 0.64 | CILP2 | CDON | H2A3 | IGFALS | MP2K2 | NADK | | | | |
| 0.64 | CILP2 | CDON | | IGFALS | MP2K2 | NADK | Notch1 | | | |
| 0.64 | CILP2 | CDON | H2A3 | | | | Notch1 | PH | | |
| 0.64 | CILP2 | CDON | | | MP2K2 | | Notch1 | PH | | |
| 0.64 | CILP2 | CDON | | | MP2K2 | NADK | Notch1 | | | S100A13 |
| 0.64 | CILP2 | | H2A3 | | MP2K2 | NADK | Notch1 | | | S100A13 |
| 0.641 | CILP2 | CDON | H2A3 | IGFALS | MP2K2 | | Notch1 | | | |
| 0.641 | CILP2 | | H2A3 | IGFALS | MP2K2 | NADK | Notch1 | | | |
| 0.641 | CILP2 | CDON | H2A3 | | | NADK | | PH | | |
| 0.641 | CILP2 | CDON | | | | NADK | Notch1 | PH | | |
| 0.641 | CILP2 | CDON | H2A3 | IGFALS | | NADK | | | | S100A13 |
| 0.641 | CILP2 | CDON | H2A3 | IGFALS | | | Notch1 | | | S100A13 |
| 0.641 | CILP2 | | | | | NADK | | PH | | S100A13 |
| 0.641 | CILP2 | | H2A3 | | | NADK | | PH | | S100A13 |
| 0.642 | CILP2 | | | IGFALS | | | | PH | | |
| 0.642 | CILP2 | | | | MP2K2 | NADK | | PH | | |
| 0.642 | CILP2 | | H2A3 | | | NADK | Notch1 | PH | | |
| 0.642 | CILP2 | CDON | | IGFALS | MP2K2 | | | | | S100A13 |
| 0.642 | CILP2 | | H2A3 | IGFALS | MP2K2 | | | | | S100A13 |
| 0.642 | CILP2 | CDON | | IGFALS | | NADK | | | | S100A13 |
| 0.642 | CILP2 | | H2A3 | | | | Notch1 | PH | | S100A13 |
| 0.643 | CILP2 | CDON | H2A3 | | MP2K2 | NADK | Notch1 | | | S100A13 |
| 0.644 | CILP2 | CDON | H2A3 | IGFALS | MP2K2 | NADK | Notch1 | | | |
| 0.644 | CILP2 | | H2A3 | | MP2K2 | | | PH | | |
| 0.644 | CILP2 | CDON | | | MP2K2 | | | PH | | S100A13 |
| 0.645 | CILP2 | CDON | H2A3 | | | NADK | Notch1 | PH | | |
| 0.645 | CILP2 | CDON | H2A3 | IGFALS | MP2K2 | | | | | S100A13 |
| 0.645 | CILP2 | | | IGFALS | MP2K2 | | Notch1 | | | S100A13 |
| 0.645 | CILP2 | | | | MP2K2 | | Notch1 | PH | | S100A13 |
| 0.646 | CILP2 | | | | | | | | PTN | |
| 0.646 | CILP2 | | | IGFALS | | | Notch1 | PH | | |
| 0.646 | CILP2 | | | | MP2K2 | NADK | Notch1 | PH | | |
| 0.646 | CILP2 | | | IGFALS | | NADK | Notch1 | | | S100A13 |
| 0.646 | CILP2 | | H2A3 | IGFALS | | NADK | Notch1 | | | S100A13 |
| 0.646 | CILP2 | CDON | | | | NADK | | PH | | S100A13 |
| 0.646 | CILP2 | CDON | H2A3 | | | NADK | | PH | | S100A13 |
| 0.646 | CILP2 | CDON | H2A3 | | | | Notch1 | PH | | S100A13 |
| 0.647 | CILP2 | | H2A3 | | | | | | PTN | |
| 0.647 | CILP2 | CDON | | IGFALS | | | | PH | | |
| 0.647 | CILP2 | | H2A3 | | MP2K2 | NADK | | PH | | |
| 0.647 | CILP2 | CDON | | IGFALS | MP2K2 | | Notch1 | | | S100A13 |
| 0.647 | CILP2 | CDON | | IGFALS | | NADK | Notch1 | | | S100A13 |
| 0.647 | CILP2 | CDON | H2A3 | IGFALS | | NADK | Notch1 | | | S100A13 |
| 0.647 | CILP2 | | | IGFALS | | | | PH | | S100A13 |
| 0.647 | CILP2 | | H2A3 | | MP2K2 | | | PH | | S100A13 |
| 0.648 | CILP2 | CDON | | | | | | | PTN | |
| 0.648 | CILP2 | CDON | | | MP2K2 | NADK | | PH | | |
| 0.648 | CILP2 | | | IGFALS | MP2K2 | NADK | | | | S100A13 |
| 0.648 | CILP2 | | H2A3 | IGFALS | MP2K2 | NADK | | | | S100A13 |
| 0.648 | CILP2 | | | | | NADK | Notch1 | PH | | S100A13 |
| 0.649 | CILP2 | | H2A3 | IGFALS | | | | PH | | |
| 0.649 | CILP2 | CDON | H2A3 | | MP2K2 | | | PH | | |
| 0.649 | CILP2 | | | IGFALS | MP2K2 | | | PH | | |
| 0.649 | CILP2 | CDON | | IGFALS | | | Notch1 | PH | | |
| 0.649 | CILP2 | CDON | | | MP2K2 | NADK | Notch1 | PH | | |
| 0.649 | CILP2 | CDON | | | MP2K2 | | Notch1 | PH | | S100A13 |
| 0.65 | CILP2 | | H2A3 | | MP2K2 | | Notch1 | PH | | |
| 0.65 | CILP2 | | H2A3 | IGFALS | MP2K2 | | Notch1 | | | S100A13 |
| 0.651 | CILP2 | CDON | H2A3 | | | | | | PTN | |
| 0.651 | CILP2 | | | | | | | | PTN | S100A13 |
| 0.651 | CILP2 | CDON | | IGFALS | MP2K2 | NADK | | | | S100A13 |
| 0.651 | CILP2 | CDON | H2A3 | IGFALS | MP2K2 | NADK | | | | S100A13 |
| 0.651 | CILP2 | CDON | H2A3 | | MP2K2 | | | PH | | S100A13 |
| 0.651 | CILP2 | CDON | | | | NADK | Notch1 | PH | | S100A13 |
| 0.651 | CILP2 | | H2A3 | | | NADK | Notch1 | PH | | S100A13 |
| 0.652 | CILP2 | | | | MP2K2 | | | | PTN | |
| 0.652 | CILP2 | | | IGFALS | | NADK | | PH | | |
| 0.652 | CILP2 | CDON | H2A3 | | MP2K2 | NADK | | PH | | |
| 0.652 | CILP2 | | H2A3 | | MP2K2 | NADK | Notch1 | PH | | |
| 0.652 | CILP2 | CDON | H2A3 | IGFALS | MP2K2 | | Notch1 | | | S100A13 |
| 0.652 | CILP2 | CDON | | IGFALS | | | | PH | | S100A13 |
| 0.652 | CILP2 | | H2A3 | IGFALS | | | | PH | | S100A13 |
| 0.653 | CILP2 | CDON | | | MP2K2 | | | | PTN | |
| 0.653 | CILP2 | | H2A3 | IGFALS | | NADK | | PH | | |
| 0.653 | CILP2 | CDON | H2A3 | | MP2K2 | | Notch1 | PH | | |
| 0.653 | CILP2 | | | IGFALS | MP2K2 | | Notch1 | PH | | |
| 0.653 | CILP2 | CDON | | | | | | | PTN | S100A13 |
| 0.653 | CILP2 | | H2A3 | | | | | | PTN | S100A13 |
| 0.653 | CILP2 | | | IGFALS | MP2K2 | NADK | Notch1 | | | S100A13 |
| 0.653 | CILP2 | | | | MP2K2 | NADK | | PH | | S100A13 |
| 0.654 | CILP2 | | H2A3 | | MP2K2 | | | | PTN | |
| 0.654 | CILP2 | CDON | H2A3 | IGFALS | | | | PH | | |
| 0.654 | CILP2 | CDON | | IGFALS | MP2K2 | | | PH | | |
| 0.654 | CILP2 | | H2A3 | IGFALS | | | Notch1 | PH | | |
| 0.654 | CILP2 | | | IGFALS | | NADK | Notch1 | PH | | |
| 0.654 | CILP2 | | H2A3 | IGFALS | MP2K2 | NADK | Notch1 | | | S100A13 |
| 0.654 | CILP2 | | H2A3 | | MP2K2 | NADK | | PH | | S100A13 |
| 0.654 | CILP2 | | | IGFALS | | | Notch1 | PH | | S100A13 |
| 0.654 | CILP2 | CDON | H2A3 | | | NADK | Notch1 | PH | | S100A13 |
| 0.655 | CILP2 | CDON | | IGFALS | MP2K2 | | Notch1 | PH | | |
| 0.655 | CILP2 | CDON | H2A3 | | MP2K2 | NADK | Notch1 | PH | | |
| 0.655 | CILP2 | CDON | H2A3 | | | | | | PTN | S100A13 |
| 0.655 | CILP2 | CDON | | IGFALS | MP2K2 | NADK | Notch1 | | | S100A13 |
| 0.655 | CILP2 | | H2A3 | | MP2K2 | | Notch1 | PH | | S100A13 |
| 0.656 | CILP2 | | H2A3 | | | NADK | | | PTN | |
| 0.656 | CILP2 | CDON | | | | | Notch1 | | PTN | |
| 0.656 | CILP2 | CDON | | IGFALS | | NADK | | PH | | |
| 0.656 | CILP2 | | | IGFALS | MP2K2 | NADK | | PH | | |
| 0.656 | CILP2 | CDON | H2A3 | IGFALS | | | Notch1 | PH | | |
| 0.656 | CILP2 | CDON | | IGFALS | | NADK | Notch1 | PH | | |
| 0.656 | CILP2 | CDON | H2A3 | IGFALS | MP2K2 | NADK | Notch1 | | | S100A13 |
| 0.656 | CILP2 | CDON | H2A3 | IGFALS | | | | PH | | S100A13 |
| 0.656 | CILP2 | CDON | | | MP2K2 | NADK | | PH | | S100A13 |
| 0.657 | CILP2 | CDON | H2A3 | | MP2K2 | | | | PTN | |
| 0.657 | CILP2 | | | | | | Notch1 | | PTN | |
| 0.657 | CILP2 | | H2A3 | IGFALS | MP2K2 | | | PH | | |
| 0.657 | CILP2 | CDON | H2A3 | IGFALS | | NADK | | PH | | |
| 0.657 | CILP2 | | H2A3 | IGFALS | | NADK | Notch1 | PH | | |
| 0.657 | CILP2 | | | | MP2K2 | | | | PTN | S100A13 |
| 0.657 | CILP2 | | | IGFALS | MP2K2 | | | PH | | S100A13 |
| 0.657 | CILP2 | CDON | H2A3 | | MP2K2 | NADK | | PH | | S100A13 |
| 0.657 | CILP2 | CDON | | IGFALS | | | Notch1 | PH | | S100A13 |
| 0.657 | CILP2 | CDON | H2A3 | | MP2K2 | | Notch1 | PH | | S100A13 |
| 0.657 | CILP2 | | | | MP2K2 | NADK | Notch1 | PH | | S100A13 |
| 0.658 | CILP2 | CDON | | | MP2K2 | | | | PTN | S100A13 |
| 0.659 | CILP2 | | | | | NADK | | | PTN | |
| 0.659 | CILP2 | CDON | | | | NADK | | | PTN | |
| 0.659 | CILP2 | CDON | H2A3 | | | NADK | | | PTN | |
| 0.659 | CILP2 | | H2A3 | IGFALS | MP2K2 | NADK | | PH | | |
| 0.659 | CILP2 | CDON | H2A3 | IGFALS | | NADK | Notch1 | PH | | |
| 0.659 | CILP2 | | | IGFALS | MP2K2 | NADK | Notch1 | PH | | |
| 0.659 | CILP2 | | H2A3 | | MP2K2 | | | | PTN | S100A13 |
| 0.659 | CILP2 | | H2A3 | IGFALS | | NADK | | PH | | S100A13 |
| 0.659 | CILP2 | CDON | | | MP2K2 | NADK | Notch1 | PH | | S100A13 |
| 0.66 | CILP2 | CDON | | | MP2K2 | | Notch1 | | PTN | |
| 0.66 | CILP2 | CDON | | IGFALS | MP2K2 | NADK | | PH | | |
| 0.66 | CILP2 | | | IGFALS | | NADK | | PH | | S100A13 |
| 0.66 | CILP2 | | H2A3 | IGFALS | | | Notch1 | PH | | S100A13 |
| 0.661 | CILP2 | | H2A3 | | MP2K2 | NADK | | | PTN | |
| 0.661 | CILP2 | | H2A3 | | | | Notch1 | | PTN | |
| 0.661 | CILP2 | CDON | H2A3 | | | | Notch1 | | PTN | |
| 0.661 | CILP2 | | | | MP2K2 | | Notch1 | | PTN | |
| 0.661 | CILP2 | | | | | | | PH | PTN | |
| 0.661 | CILP2 | CDON | | IGFALS | MP2K2 | NADK | Notch1 | PH | | |
| 0.661 | CILP2 | CDON | H2A3 | | MP2K2 | | | | PTN | S100A13 |
| 0.661 | CILP2 | CDON | | IGFALS | MP2K2 | | | PH | | S100A13 |
| 0.661 | CILP2 | | H2A3 | | MP2K2 | NADK | Notch1 | PH | | S100A13 |
| 0.662 | CILP2 | CDON | H2A3 | IGFALS | MP2K2 | | | PH | | |
| 0.662 | CILP2 | | H2A3 | IGFALS | MP2K2 | | Notch1 | PH | | |
| 0.662 | CILP2 | | H2A3 | | | NADK | | | PTN | S100A13 |
| 0.662 | CILP2 | CDON | | | | | Notch1 | | PTN | S100A13 |
| 0.662 | CILP2 | | H2A3 | IGFALS | MP2K2 | | | PH | | S100A13 |
| 0.662 | CILP2 | CDON | | IGFALS | | NADK | | PH | | S100A13 |
| 0.662 | CILP2 | CDON | H2A3 | IGFALS | | NADK | | PH | | S100A13 |
| 0.662 | CILP2 | CDON | H2A3 | IGFALS | | | Notch1 | PH | | S100A13 |
| 0.662 | CILP2 | CDON | H2A3 | | MP2K2 | NADK | Notch1 | PH | | S100A13 |
| 0.663 | CILP2 | | | | MP2K2 | NADK | | | PTN | |
| 0.663 | CILP2 | CDON | | | MP2K2 | NADK | | | PTN | |
| 0.663 | CILP2 | CDON | H2A3 | | MP2K2 | NADK | | | PTN | |
| 0.663 | CILP2 | CDON | | | | | | PH | PTN | |
| 0.663 | CILP2 | CDON | H2A3 | IGFALS | MP2K2 | NADK | | PH | | |
| 0.663 | CILP2 | | H2A3 | IGFALS | MP2K2 | NADK | Notch1 | PH | | |
| 0.663 | CILP2 | CDON | H2A3 | | | NADK | | | PTN | S100A13 |
| 0.663 | CILP2 | | | | | | Notch1 | | PTN | S100A13 |
| 0.663 | CILP2 | | | IGFALS | MP2K2 | | Notch1 | PH | | S100A13 |
| 0.664 | CILP2 | CDON | | IGFALS | | | | | PTN | |
| 0.664 | CILP2 | | H2A3 | IGFALS | | | | | PTN | |
| 0.664 | CILP2 | | H2A3 | | | | | PH | PTN | |
| 0.664 | CILP2 | CDON | H2A3 | IGFALS | MP2K2 | | Notch1 | PH | | |
| 0.664 | CILP2 | | | | | NADK | | | PTN | S100A13 |
| 0.664 | CILP2 | | | IGFALS | | NADK | Notch1 | PH | | S100A13 |
| 0.665 | CILP2 | | | IGFALS | | | | | PTN | |
| 0.665 | CILP2 | | H2A3 | | MP2K2 | | Notch1 | | PTN | |
| 0.665 | CILP2 | CDON | H2A3 | | MP2K2 | | Notch1 | | PTN | |
| 0.665 | CILP2 | CDON | H2A3 | IGFALS | MP2K2 | NADK | Notch1 | PH | | |
| 0.665 | CILP2 | CDON | | | | NADK | | | PTN | S100A13 |
| 0.665 | CILP2 | | | | | | | PH | PTN | S100A13 |
| 0.665 | CILP2 | CDON | H2A3 | IGFALS | MP2K2 | | | PH | | S100A13 |
| 0.665 | CILP2 | CDON | | IGFALS | MP2K2 | | Notch1 | PH | | S100A13 |
| 0.665 | CILP2 | | H2A3 | IGFALS | | NADK | Notch1 | PH | | S100A13 |
| 0.666 | CILP2 | CDON | H2A3 | IGFALS | | | | | PTN | |
| 0.666 | CILP2 | CDON | | IGFALS | MP2K2 | | | | PTN | |
| 0.666 | CILP2 | CDON | | | | NADK | Notch1 | | PTN | |
| 0.666 | CILP2 | | H2A3 | | MP2K2 | NADK | | | PTN | S100A13 |
| 0.666 | CILP2 | CDON | | | MP2K2 | | Notch1 | | PTN | S100A13 |
| 0.666 | CILP2 | CDON | | | | | | PH | PTN | S100A13 |
| 0.666 | CILP2 | | | IGFALS | MP2K2 | NADK | | PH | | S100A13 |
| 0.666 | CILP2 | CDON | | IGFALS | | NADK | Notch1 | PH | | S100A13 |
| 0.667 | CILP2 | | | IGFALS | MP2K2 | | | | PTN | |
| 0.667 | CILP2 | | H2A3 | IGFALS | MP2K2 | | | | PTN | |
| 0.667 | CILP2 | | H2A3 | | | NADK | Notch1 | | PTN | |
| 0.667 | CILP2 | CDON | H2A3 | | | NADK | Notch1 | | PTN | |
| 0.667 | CILP2 | CDON | H2A3 | | | | | PH | PTN | |
| 0.667 | CILP2 | | | | MP2K2 | | | PH | PTN | |
| 0.667 | CILP2 | | H2A3 | | | | Notch1 | | PTN | S100A13 |
| 0.667 | CILP2 | CDON | H2A3 | | | | Notch1 | | PTN | S100A13 |
| 0.667 | CILP2 | | | | MP2K2 | | Notch1 | | PTN | S100A13 |
| 0.667 | CILP2 | | H2A3 | IGFALS | MP2K2 | NADK | | PH | | S100A13 |
| 0.667 | CILP2 | CDON | H2A3 | IGFALS | | NADK | Notch1 | PH | | S100A13 |
| 0.668 | CILP2 | CDON | H2A3 | IGFALS | MP2K2 | | | | PTN | |
| 0.668 | CILP2 | | | | | NADK | Notch1 | | PTN | |
| 0.668 | CILP2 | CDON | | | MP2K2 | NADK | Notch1 | | PTN | |
| 0.668 | CILP2 | CDON | | | MP2K2 | | | PH | PTN | |
| 0.668 | CILP2 | | | | MP2K2 | NADK | | | PTN | S100A13 |
| 0.668 | CILP2 | CDON | | | MP2K2 | NADK | | | PTN | S100A13 |
| 0.668 | CILP2 | CDON | H2A3 | | MP2K2 | NADK | | | PTN | S100A13 |
| 0.668 | CILP2 | | H2A3 | | | | | PH | PTN | S100A13 |
| 0.668 | CILP2 | CDON | | IGFALS | MP2K2 | NADK | | PH | | S100A13 |
| 0.668 | CILP2 | | H2A3 | IGFALS | MP2K2 | | Notch1 | PH | | S100A13 |
| 0.669 | CILP2 | | | | MP2K2 | NADK | Notch1 | | PTN | |
| 0.669 | CILP2 | CDON | | IGFALS | | | | | PTN | S100A13 |
| 0.669 | CILP2 | | H2A3 | IGFALS | | | | | PTN | S100A13 |
| 0.669 | CILP2 | CDON | H2A3 | IGFALS | MP2K2 | NADK | | PH | | S100A13 |
| 0.669 | CILP2 | CDON | H2A3 | IGFALS | MP2K2 | | Notch1 | PH | | S100A13 |
| 0.67 | CILP2 | | H2A3 | IGFALS | | NADK | | | PTN | |
| 0.67 | CILP2 | CDON | | IGFALS | | | Notch1 | | PTN | |
| 0.67 | CILP2 | | H2A3 | | MP2K2 | NADK | Notch1 | | PTN | |
| 0.67 | CILP2 | CDON | H2A3 | | MP2K2 | NADK | Notch1 | | PTN | |
| 0.67 | CILP2 | | H2A3 | | MP2K2 | | | PH | PTN | |
| 0.67 | CILP2 | | H2A3 | | | NADK | | PH | PTN | |
| 0.67 | CILP2 | CDON | | | | | Notch1 | PH | PTN | |
| 0.67 | CILP2 | | | IGFALS | | | | | PTN | S100A13 |
| 0.67 | CILP2 | CDON | H2A3 | IGFALS | | | | | PTN | S100A13 |
| 0.67 | CILP2 | CDON | H2A3 | | | | | PH | PTN | S100A13 |
| 0.67 | CILP2 | | | | MP2K2 | | | PH | PTN | S100A13 |
| 0.67 | CILP2 | | | IGFALS | MP2K2 | NADK | Notch1 | PH | | S100A13 |
| 0.671 | CILP2 | CDON | H2A3 | IGFALS | | NADK | | | PTN | |
| 0.671 | CILP2 | | H2A3 | IGFALS | MP2K2 | NADK | | | PTN | |
| 0.671 | CILP2 | | | | | NADK | | PH | PTN | |
| 0.671 | CILP2 | | | | | | Notch1 | PH | PTN | |
| 0.671 | CILP2 | | H2A3 | | MP2K2 | | Notch1 | | PTN | S100A13 |
| 0.671 | CILP2 | CDON | H2A3 | | MP2K2 | | Notch1 | | PTN | S100A13 |
| 0.671 | CILP2 | CDON | | | MP2K2 | | | PH | PTN | S100A13 |
| 0.671 | CILP2 | CDON | | IGFALS | MP2K2 | NADK | Notch1 | PH | | S100A13 |
| 0.672 | CILP2 | CDON | | IGFALS | | NADK | | | PTN | |
| 0.672 | CILP2 | CDON | H2A3 | IGFALS | MP2K2 | NADK | | | PTN | |
| 0.672 | CILP2 | | | IGFALS | | | Notch1 | | PTN | |
| 0.672 | CILP2 | CDON | | IGFALS | MP2K2 | | Notch1 | | PTN | |
| 0.672 | CILP2 | CDON | H2A3 | | MP2K2 | | | PH | PTN | |
| 0.672 | CILP2 | CDON | | | | NADK | | PH | PTN | |
| 0.672 | CILP2 | CDON | H2A3 | | | NADK | | PH | PTN | |
| 0.672 | CILP2 | CDON | | IGFALS | MP2K2 | | | | PTN | S100A13 |
| 0.672 | CILP2 | | H2A3 | IGFALS | MP2K2 | NADK | Notch1 | PH | | S100A13 |
| 0.673 | CILP2 | | | IGFALS | | NADK | | | PTN | |
| 0.673 | CILP2 | CDON | | IGFALS | MP2K2 | NADK | | | PTN | |
| 0.673 | CILP2 | CDON | H2A3 | IGFALS | | | Notch1 | | PTN | |
| 0.673 | CILP2 | CDON | | | MP2K2 | | Notch1 | PH | PTN | |
| 0.673 | CILP2 | | | IGFALS | MP2K2 | | | | PTN | S100A13 |
| 0.673 | CILP2 | | H2A3 | IGFALS | MP2K2 | | | | PTN | S100A13 |
| 0.673 | CILP2 | CDON | | | | NADK | Notch1 | | PTN | S100A13 |
| 0.673 | CILP2 | CDON | H2A3 | | | NADK | Notch1 | | PTN | S100A13 |
| 0.673 | CILP2 | | H2A3 | | MP2K2 | | | PH | PTN | S100A13 |
| 0.673 | CILP2 | CDON | H2A3 | IGFALS | MP2K2 | NADK | Notch1 | PH | | S100A13 |
| 0.674 | CILP2 | | | IGFALS | MP2K2 | NADK | | | PTN | |
| 0.674 | CILP2 | | H2A3 | IGFALS | | | Notch1 | | PTN | |
| 0.674 | CILP2 | | | IGFALS | MP2K2 | | Notch1 | | PTN | |
| 0.674 | CILP2 | | H2A3 | | MP2K2 | NADK | | PH | PTN | |
| 0.674 | CILP2 | | | | MP2K2 | | Notch1 | PH | PTN | |
| 0.674 | CILP2 | CDON | H2A3 | IGFALS | MP2K2 | | | | PTN | S100A13 |
| 0.674 | CILP2 | | | | | NADK | Notch1 | | PTN | S100A13 |
| 0.674 | CILP2 | | H2A3 | | | NADK | Notch1 | | PTN | S100A13 |
| 0.674 | CILP2 | | H2A3 | | | NADK | | PH | PTN | S100A13 |
| 0.674 | CILP2 | | | | | | Notch1 | PH | PTN | S100A13 |
| 0.674 | CILP2 | CDON | | | | | Notch1 | PH | PTN | S100A13 |
| 0.675 | CILP2 | | H2A3 | IGFALS | MP2K2 | | Notch1 | | PTN | |
| 0.675 | CILP2 | CDON | H2A3 | IGFALS | MP2K2 | | Notch1 | | PTN | |
| 0.675 | CILP2 | | | | MP2K2 | NADK | | PH | PTN | |
| 0.675 | CILP2 | CDON | | | MP2K2 | NADK | | PH | PTN | |
| 0.675 | CILP2 | | H2A3 | IGFALS | | NADK | | | PTN | S100A13 |
| 0.675 | CILP2 | CDON | H2A3 | | MP2K2 | | | PH | PTN | S100A13 |
| 0.676 | CILP2 | | | IGFALS | | | | PH | PTN | |
| 0.676 | CILP2 | CDON | | IGFALS | | | | PH | PTN | |
| 0.676 | CILP2 | CDON | H2A3 | | MP2K2 | NADK | | PH | PTN | |
| 0.676 | CILP2 | | H2A3 | | | | Notch1 | PH | PTN | |
| 0.676 | CILP2 | CDON | H2A3 | | | | Notch1 | PH | PTN | |
| 0.676 | CILP2 | CDON | H2A3 | IGFALS | | NADK | | | PTN | S100A13 |
| 0.676 | CILP2 | | | | MP2K2 | NADK | Notch1 | | PTN | S100A13 |
| 0.676 | CILP2 | CDON | | | MP2K2 | NADK | Notch1 | | PTN | S100A13 |
| 0.676 | CILP2 | CDON | H2A3 | | MP2K2 | NADK | Notch1 | | PTN | S100A13 |
| 0.676 | CILP2 | | | | | NADK | | PH | PTN | S100A13 |
| 0.676 | CILP2 | CDON | | | | NADK | | PH | PTN | S100A13 |
| 0.676 | CILP2 | CDON | H2A3 | | | NADK | | PH | PTN | S100A13 |
| 0.677 | CILP2 | CDON | | IGFALS | | NADK | Notch1 | | PTN | |
| 0.677 | CILP2 | CDON | H2A3 | IGFALS | | NADK | Notch1 | | PTN | |
| 0.677 | CILP2 | | H2A3 | IGFALS | | | | PH | PTN | |
| 0.677 | CILP2 | CDON | | IGFALS | | NADK | | | PTN | S100A13 |
| 0.677 | CILP2 | CDON | | IGFALS | | | Notch1 | | PTN | S100A13 |
| 0.677 | CILP2 | | H2A3 | | MP2K2 | NADK | Notch1 | | PTN | S100A13 |
| 0.677 | CILP2 | CDON | | | MP2K2 | | Notch1 | PH | PTN | S100A13 |
| 0.678 | CILP2 | | H2A3 | IGFALS | | NADK | Notch1 | | PTN | |
| 0.678 | CILP2 | CDON | | IGFALS | MP2K2 | NADK | Notch1 | | PTN | |
| 0.678 | CILP2 | CDON | H2A3 | IGFALS | MP2K2 | NADK | Notch1 | | PTN | |
| 0.678 | CILP2 | | | IGFALS | MP2K2 | | | PH | PTN | |
| 0.678 | CILP2 | CDON | | | | NADK | Notch1 | PH | PTN | |
| 0.678 | CILP2 | | | IGFALS | | NADK | | | PTN | S100A13 |
| 0.678 | CILP2 | | H2A3 | IGFALS | MP2K2 | NADK | | | PTN | S100A13 |
| 0.678 | CILP2 | CDON | H2A3 | IGFALS | MP2K2 | NADK | | | PTN | S100A13 |
| 0.678 | CILP2 | | | IGFALS | | | Notch1 | | PTN | S100A13 |
| 0.678 | CILP2 | | | | MP2K2 | | Notch1 | PH | PTN | S100A13 |
| 0.679 | CILP2 | | | IGFALS | | NADK | Notch1 | | PTN | |
| 0.679 | CILP2 | | | IGFALS | MP2K2 | NADK | Notch1 | | PTN | |
| 0.679 | CILP2 | | H2A3 | IGFALS | MP2K2 | NADK | Notch1 | | PTN | |
| 0.679 | CILP2 | CDON | H2A3 | IGFALS | | | | PH | PTN | |
| 0.679 | CILP2 | CDON | | IGFALS | MP2K2 | | | PH | PTN | |
| 0.679 | CILP2 | CDON | H2A3 | | MP2K2 | | Notch1 | PH | PTN | |
| 0.679 | CILP2 | | | | | NADK | Notch1 | PH | PTN | |
| 0.679 | CILP2 | CDON | | IGFALS | MP2K2 | NADK | | | PTN | S100A13 |
| 0.679 | CILP2 | | H2A3 | IGFALS | | | Notch1 | | PTN | S100A13 |
| 0.679 | CILP2 | CDON | H2A3 | IGFALS | | | Notch1 | | PTN | S100A13 |
| 0.679 | CILP2 | CDON | | IGFALS | MP2K2 | | Notch1 | | PTN | S100A13 |
| 0.679 | CILP2 | CDON | | | MP2K2 | NADK | | PH | PTN | S100A13 |
| 0.679 | CILP2 | | H2A3 | | MP2K2 | NADK | | PH | PTN | S100A13 |
| 0.679 | CILP2 | CDON | H2A3 | | MP2K2 | NADK | | PH | PTN | S100A13 |
| 0.68 | CILP2 | | H2A3 | IGFALS | MP2K2 | | | PH | PTN | |
| 0.68 | CILP2 | | H2A3 | | MP2K2 | | Notch1 | PH | PTN | |
| 0.68 | CILP2 | | H2A3 | | | NADK | Notch1 | PH | PTN | |
| 0.68 | CILP2 | CDON | H2A3 | | | NADK | Notch1 | PH | PTN | |
| 0.68 | CILP2 | CDON | | | MP2K2 | NADK | Notch1 | PH | PTN | |
| 0.68 | CILP2 | | | IGFALS | MP2K2 | NADK | | | PTN | S100A13 |
| 0.68 | CILP2 | | | IGFALS | MP2K2 | | Notch1 | | PTN | S100A13 |
| 0.68 | CILP2 | | | IGFALS | | | | PH | PTN | S100A13 |
| 0.68 | CILP2 | CDON | | IGFALS | | | | PH | PTN | S100A13 |
| 0.68 | CILP2 | | | | MP2K2 | NADK | | PH | PTN | S100A13 |
| 0.68 | CILP2 | | H2A3 | | | | Notch1 | PH | PTN | S100A13 |
| 0.68 | CILP2 | CDON | H2A3 | | | | Notch1 | PH | PTN | S100A13 |
| 0.681 | CILP2 | | H2A3 | IGFALS | | NADK | | PH | PTN | |
| 0.681 | CILP2 | | | | MP2K2 | NADK | Notch1 | PH | PTN | |
| 0.681 | CILP2 | CDON | H2A3 | IGFALS | MP2K2 | | Notch1 | | PTN | S100A13 |
| 0.681 | CILP2 | | H2A3 | IGFALS | | | | PH | PTN | S100A13 |
| 0.682 | CILP2 | CDON | H2A3 | IGFALS | MP2K2 | | | PH | PTN | |
| 0.682 | CILP2 | | | IGFALS | | NADK | | PH | PTN | |
| 0.682 | CILP2 | CDON | | IGFALS | | NADK | | PH | PTN | |
| 0.682 | CILP2 | CDON | H2A3 | IGFALS | | NADK | | PH | PTN | |
| 0.682 | CILP2 | CDON | | IGFALS | | | Notch1 | PH | PTN | |
| 0.682 | CILP2 | CDON | H2A3 | | MP2K2 | NADK | Notch1 | PH | PTN | |
| 0.682 | CILP2 | | H2A3 | IGFALS | MP2K2 | | Notch1 | | PTN | S100A13 |
| 0.682 | CILP2 | CDON | H2A3 | IGFALS | | | | PH | PTN | S100A13 |
| 0.683 | CILP2 | CDON | | IGFALS | MP2K2 | NADK | | PH | PTN | |
| 0.683 | CILP2 | | H2A3 | IGFALS | MP2K2 | NADK | | PH | PTN | |
| 0.683 | CILP2 | | | IGFALS | | | Notch1 | PH | PTN | |
| 0.683 | CILP2 | | H2A3 | | MP2K2 | NADK | Notch1 | PH | PTN | |
| 0.683 | CILP2 | CDON | | IGFALS | | NADK | Notch1 | | PTN | S100A13 |
| 0.683 | CILP2 | CDON | H2A3 | IGFALS | | NADK | Notch1 | | PTN | S100A13 |
| 0.683 | CILP2 | | | IGFALS | MP2K2 | | | PH | PTN | S100A13 |
| 0.683 | CILP2 | CDON | | IGFALS | MP2K2 | | | PH | PTN | S100A13 |
| 0.683 | CILP2 | CDON | H2A3 | | MP2K2 | | Notch1 | PH | PTN | S100A13 |
| 0.683 | CILP2 | CDON | | | | NADK | Notch1 | PH | PTN | S100A13 |
| 0.684 | CILP2 | | | IGFALS | MP2K2 | NADK | | PH | PTN | |
| 0.684 | CILP2 | CDON | H2A3 | IGFALS | MP2K2 | NADK | | PH | PTN | |
| 0.684 | CILP2 | CDON | | IGFALS | MP2K2 | | Notch1 | PH | PTN | |
| 0.684 | CILP2 | | H2A3 | IGFALS | | NADK | Notch1 | | PTN | S100A13 |
| 0.684 | CILP2 | | H2A3 | IGFALS | MP2K2 | | | PH | PTN | S100A13 |
| 0.684 | CILP2 | | H2A3 | | MP2K2 | | Notch1 | PH | PTN | S100A13 |
| 0.684 | CILP2 | | | | | NADK | Notch1 | PH | PTN | S100A13 |
| 0.685 | CILP2 | | | IGFALS | MP2K2 | | Notch1 | PH | PTN | |
| 0.685 | CILP2 | | | IGFALS | | NADK | Notch1 | | PTN | S100A13 |
| 0.685 | CILP2 | CDON | | IGFALS | MP2K2 | NADK | Notch1 | | PTN | S100A13 |
| 0.685 | CILP2 | CDON | H2A3 | IGFALS | MP2K2 | NADK | Notch1 | | PTN | S100A13 |
| 0.685 | CILP2 | CDON | H2A3 | IGFALS | MP2K2 | | | PH | PTN | S100A13 |
| 0.685 | CILP2 | | H2A3 | | | NADK | Notch1 | PH | PTN | S100A13 |
| 0.685 | CILP2 | CDON | H2A3 | | | NADK | Notch1 | PH | PTN | S100A13 |
| 0.685 | CILP2 | CDON | | | MP2K2 | NADK | Notch1 | PH | PTN | S100A13 |
| 0.686 | CILP2 | | H2A3 | IGFALS | | | Notch1 | PH | PTN | |
| 0.686 | CILP2 | CDON | H2A3 | IGFALS | | | Notch1 | PH | PTN | |
| 0.686 | CILP2 | | H2A3 | IGFALS | MP2K2 | NADK | Notch1 | | PTN | S100A13 |
| 0.686 | CILP2 | | H2A3 | IGFALS | | NADK | | PH | PTN | S100A13 |
| 0.686 | CILP2 | CDON | H2A3 | IGFALS | | NADK | | PH | PTN | S100A13 |
| 0.686 | CILP2 | CDON | | IGFALS | | | Notch1 | PH | PTN | S100A13 |
| 0.686 | CILP2 | | | | MP2K2 | NADK | Notch1 | PH | PTN | S100A13 |
| 0.687 | CILP2 | CDON | | IGFALS | | NADK | Notch1 | PH | PTN | |
| 0.687 | CILP2 | | | IGFALS | MP2K2 | NADK | Notch1 | | PTN | S100A13 |
| 0.687 | CILP2 | | | IGFALS | | NADK | | PH | PTN | S100A13 |
| 0.687 | CILP2 | CDON | | IGFALS | | NADK | | PH | PTN | S100A13 |
| 0.687 | CILP2 | | | IGFALS | | | Notch1 | PH | PTN | S100A13 |
| 0.687 | CILP2 | CDON | H2A3 | | MP2K2 | NADK | Notch1 | PH | PTN | S100A13 |
| 0.688 | CILP2 | | H2A3 | IGFALS | MP2K2 | | Notch1 | PH | PTN | |
| 0.688 | CILP2 | CDON | H2A3 | IGFALS | MP2K2 | | Notch1 | PH | PTN | |
| 0.688 | CILP2 | | | IGFALS | | NADK | Notch1 | PH | PTN | |
| 0.688 | CILP2 | CDON | H2A3 | IGFALS | | NADK | Notch1 | PH | PTN | |
| 0.688 | CILP2 | CDON | | IGFALS | MP2K2 | NADK | Notch1 | PH | PTN | |
| 0.688 | CILP2 | | H2A3 | IGFALS | MP2K2 | NADK | | PH | PTN | S100A13 |
| 0.688 | CILP2 | CDON | H2A3 | IGFALS | MP2K2 | NADK | | PH | PTN | S100A13 |
| 0.688 | CILP2 | CDON | | IGFALS | MP2K2 | | Notch1 | PH | PTN | S100A13 |
| 0.688 | CILP2 | | H2A3 | | MP2K2 | NADK | Notch1 | PH | PTN | S100A13 |
| 0.689 | CILP2 | | H2A3 | IGFALS | | NADK | Notch1 | PH | PTN | |
| 0.689 | CILP2 | | | IGFALS | MP2K2 | NADK | Notch1 | PH | PTN | |
| 0.689 | CILP2 | CDON | H2A3 | IGFALS | MP2K2 | NADK | Notch1 | PH | PTN | |
| 0.689 | CILP2 | | | IGFALS | MP2K2 | NADK | | PH | PTN | S100A13 |
| 0.689 | CILP2 | CDON | | IGFALS | MP2K2 | NADK | | PH | PTN | S100A13 |
| 0.69 | CILP2 | | H2A3 | IGFALS | MP2K2 | NADK | Notch1 | PH | PTN | |
| 0.69 | CILP2 | CDON | H2A3 | IGFALS | | | Notch1 | PH | PTN | S100A13 |
| 0.69 | CILP2 | | | IGFALS | MP2K2 | | Notch1 | PH | PTN | S100A13 |
| 0.691 | CILP2 | | H2A3 | IGFALS | | | Notch1 | PH | PTN | S100A13 |
| 0.692 | CILP2 | CDON | H2A3 | IGFALS | MP2K2 | | Notch1 | PH | PTN | S100A13 |
| 0.692 | CILP2 | CDON | | IGFALS | | NADK | Notch1 | PH | PTN | S100A13 |
| 0.693 | CILP2 | | H2A3 | IGFALS | MP2K2 | | Notch1 | PH | PTN | S100A13 |
| 0.693 | CILP2 | CDON | H2A3 | IGFALS | | NADK | Notch1 | PH | PTN | S100A13 |
| 0.694 | CILP2 | | | IGFALS | | NADK | Notch1 | PH | PTN | S100A13 |
| 0.694 | CILP2 | | H2A3 | IGFALS | | NADK | Notch1 | PH | PTN | S100A13 |
| 0.694 | CILP2 | CDON | | IGFALS | MP2K2 | NADK | Notch1 | PH | PTN | S100A13 |
| 0.694 | CILP2 | CDON | H2A3 | IGFALS | MP2K2 | NADK | Notch1 | PH | PTN | S100A13 |
| 0.695 | CILP2 | | | IGFALS | MP2K2 | NADK | Notch1 | PH | PTN | S100A13 |
| *0.696 | CILP2 | | H2A3 | IGFALS | MP2K2 | NADK | Notch1 | PH | PTN | S100A13 |

*Included in Table 38

TABLE 38
CILP2 Biomarker Panels, Includes CILP2, H2A3, IGFALS, MP2K2, NADK,
Notch1, PH, PTN and S100A13 plus additional markers-20 Year Model

| AUC | * | CDCP1 | NDST1 | TIM-1 | TBCA | ASB9 | OPG | SVEP1 | PARC |
|---|---|---|---|---|---|---|---|---|---|
| 0.696 | * | | | | | | | | |
| 0.706 | * | CDCP1 | | | | | | | |
| 0.699 | * | | NDST1 | | | | | | |
| 0.701 | * | | | TIM-1 | | | | | |
| 0.696 | * | | | | TBCA | | | | |
| 0.7 | * | | | | | ASB9 | | | |
| 0.697 | * | | | | | | OPG | | |
| 0.698 | * | | | | | | | SVEP1 | |
| 0.697 | * | | | | | | | | PARC |
| 0.697 | * | | | | | | | | |
| 0.698 | * | | | | | | | | |
| 0.703 | * | | | | | | | | |
| 0.697 | * | | | | | | | | |
| 0.696 | * | | | | | | | | |
| 0.696 | * | | | | | | | | |
| 0.707 | * | | | | | | | | |

| AUC | ATS13 | YKL-40 | sRAGE | calgranulin B | IL-18 | NFL | NPTXR |
|---|---|---|---|---|---|---|---|
| 0.696 | | | | | | | |
| 0.706 | | | | | | | |
| 0.699 | | | | | | | |
| 0.701 | | | | | | | |
| 0.696 | | | | | | | |
| 0.7 | | | | | | | |
| 0.697 | | | | | | | |
| 0.698 | | | | | | | |
| 0.697 | | | | | | | |
| 0.697 | ATS13 | | | | | | |
| 0.698 | | YKL-40 | | | | | |
| 0.703 | | | sRAGE | | | | |
| 0.697 | | | | calgranulin B | | | |
| 0.696 | | | | | IL-18 | | |
| 0.696 | | | | | | NFL | |
| 0.707 | | | | | | | NPTXR |

Includes CILP2, H2A3, IGFALS, MP2K2, NADK, Notch1, PH, PTN and S100A13

TABLE 39
PTN Biomarker Panels-20 Year Model

| AUC | PTN | CDON | CILP2 | H2A3 | IGFALS | MP2K2 | NADK | Notch1 | PH | S100A13 |
|---|---|---|---|---|---|---|---|---|---|---|
| 0.605 | PTN | | | | | MP2K2 | | | | |
| 0.61 | PTN | | | | | | | | | S100A13 |
| 0.611 | PTN | | | H2A3 | | | | | | |
| 0.613 | PTN | CDON | | | | | | | | |
| 0.616 | PTN | | | | | MP2K2 | | | | S100A13 |
| 0.617 | PTN | | | H2A3 | | MP2K2 | | | | |
| 0.619 | PTN | CDON | | | | MP2K2 | | | | |
| 0.619 | PTN | | | H2A3 | | | | | | S100A13 |
| 0.622 | PTN | | | | | | NADK | | | |
| 0.624 | PTN | | | H2A3 | | | NADK | | | |
| 0.624 | PTN | CDON | | | | | | | | S100A13 |
| 0.625 | PTN | CDON | | H2A3 | | | | | | |
| 0.625 | PTN | | | | | MP2K2 | NADK | | | |
| 0.625 | PTN | | | | | | | | PH | |
| 0.625 | PTN | | | H2A3 | | MP2K2 | | | | S100A13 |
| 0.627 | PTN | | | H2A3 | | MP2K2 | NADK | | | |
| 0.628 | PTN | | | | | | | Notch1 | | |
| 0.628 | PTN | CDON | | | | MP2K2 | | | | S100A13 |
| 0.629 | PTN | CDON | | | | | NADK | | | |
| 0.63 | PTN | CDON | | H2A3 | | MP2K2 | | | | |
| 0.63 | PTN | | | | | MP2K2 | | Notch1 | | |
| 0.63 | PTN | | | | | | NADK | | | S100A13 |
| 0.631 | PTN | CDON | | | | | | Notch1 | | |
| 0.631 | PTN | | | | | MP2K2 | | | PH | |
| 0.631 | PTN | | | H2A3 | | | NADK | | | S100A13 |
| 0.632 | PTN | | | | IGFALS | | | | | |
| 0.632 | PTN | CDON | | | | MP2K2 | NADK | | | |
| 0.632 | PTN | | | | | | | | PH | S100A13 |
| 0.633 | PTN | | | | IGFALS | MP2K2 | | | | |
| 0.633 | PTN | CDON | | H2A3 | | | | | | S100A13 |
| 0.633 | PTN | | | | | MP2K2 | NADK | | | S100A13 |
| 0.634 | PTN | CDON | | H2A3 | | | NADK | | | |
| 0.634 | PTN | CDON | | | | MP2K2 | | Notch1 | | |
| 0.635 | PTN | | | H2A3 | | MP2K2 | NADK | | | S100A13 |
| 0.637 | PTN | CDON | | H2A3 | | MP2K2 | NADK | | | |
| 0.637 | PTN | CDON | | | | | | | PH | |
| 0.637 | PTN | CDON | | H2A3 | | MP2K2 | | | | S100A13 |
| 0.637 | PTN | | | | | MP2K2 | | | PH | S100A13 |
| 0.638 | PTN | | | H2A3 | IGFALS | | | | | |
| 0.638 | PTN | | | H2A3 | | | | | PH | |
| 0.638 | PTN | CDON | | | | | NADK | | | S100A13 |
| 0.639 | PTN | CDON | | | IGFALS | | | | | |
| 0.639 | PTN | | | H2A3 | IGFALS | MP2K2 | | | | |
| 0.64 | PTN | | | H2A3 | | | | Notch1 | | |
| 0.64 | PTN | CDON | | | | MP2K2 | NADK | | | S100A13 |
| 0.641 | PTN | CDON | | | IGFALS | MP2K2 | | | | |
| 0.641 | PTN | CDON | | | | MP2K2 | | | PH | |
| 0.642 | PTN | | | H2A3 | | MP2K2 | | Notch1 | | |
| 0.642 | PTN | | | | | | NADK | Notch1 | | |
| 0.642 | PTN | | | | IGFALS | | | | | S100A13 |
| 0.642 | PTN | CDON | | H2A3 | | | NADK | | | S100A13 |
| 0.642 | PTN | | | | | | | Notch1 | | S100A13 |
| 0.642 | PTN | | | H2A3 | | | | | PH | S100A13 |
| 0.643 | PTN | CDON | | H2A3 | | | | Notch1 | | |
| 0.643 | PTN | | | | | MP2K2 | NADK | Notch1 | | |
| 0.643 | PTN | | | | | | NADK | | PH | |
| 0.643 | PTN | CDON | | | | | | | PH | S100A13 |
| 0.644 | PTN | CDON | | | | | NADK | Notch1 | | |
| 0.644 | PTN | | | H2A3 | | MP2K2 | | | PH | |
| 0.644 | PTN | | | | IGFALS | MP2K2 | | | | S100A13 |
| 0.644 | PTN | CDON | | H2A3 | | MP2K2 | NADK | | | S100A13 |
| 0.644 | PTN | | | | | MP2K2 | | Notch1 | | S100A13 |
| 0.645 | PTN | | | | IGFALS | | NADK | | | |
| 0.645 | PTN | | | H2A3 | IGFALS | | NADK | | | |
| 0.645 | PTN | | | | IGFALS | MP2K2 | NADK | | | |
| 0.645 | PTN | | | H2A3 | IGFALS | MP2K2 | NADK | | | |
| 0.645 | PTN | CDON | | | | MP2K2 | NADK | Notch1 | | |
| 0.646 | PTN | | CILP2 | | | | | | | |
| 0.646 | PTN | CDON | | H2A3 | | MP2K2 | | Notch1 | | |
| 0.646 | PTN | | | H2A3 | | | NADK | Notch1 | | |
| 0.646 | PTN | | | H2A3 | | | NADK | | PH | |
| 0.646 | PTN | | | | | MP2K2 | NADK | | PH | |
| 0.646 | PTN | | | H2A3 | IGFALS | | | | | S100A13 |
| 0.646 | PTN | CDON | | | | | | Notch1 | | S100A13 |
| 0.646 | PTN | CDON | | | | MP2K2 | | | PH | S100A13 |
| 0.647 | PTN | | CILP2 | H2A3 | | | | | | |
| 0.647 | PTN | CDON | | H2A3 | IGFALS | | | | | |
| 0.647 | PTN | | | H2A3 | | MP2K2 | NADK | Notch1 | | |
| 0.648 | PTN | CDON | CILP2 | | | | | | | |
| 0.648 | PTN | CDON | | H2A3 | IGFALS | MP2K2 | | | | |
| 0.648 | PTN | CDON | | H2A3 | | | | | PH | |
| 0.648 | PTN | | | H2A3 | IGFALS | MP2K2 | | | | S100A13 |
| 0.648 | PTN | CDON | | | | MP2K2 | | Notch1 | | S100A13 |
| 0.648 | PTN | | | H2A3 | | MP2K2 | | | PH | S100A13 |
| 0.648 | PTN | | | | | | NADK | | PH | S100A13 |
| 0.649 | PTN | CDON | | | IGFALS | | NADK | | | |
| 0.649 | PTN | CDON | | | IGFALS | MP2K2 | NADK | | | |
| 0.649 | PTN | CDON | | H2A3 | | | NADK | Notch1 | | |
| 0.649 | PTN | | | | IGFALS | | | | PH | |
| 0.649 | PTN | CDON | | | | | NADK | | PH | |
| 0.649 | PTN | CDON | | | IGFALS | | | | | S100A13 |
| 0.65 | PTN | | | H2A3 | | MP2K2 | NADK | | PH | |
| 0.65 | PTN | | | | | | | Notch1 | PH | |
| 0.65 | PTN | | | | | MP2K2 | | Notch1 | PH | |
| 0.651 | PTN | CDON | CILP2 | H2A3 | | | | | | |
| 0.651 | PTN | CDON | | H2A3 | | MP2K2 | NADK | Notch1 | | |
| 0.651 | PTN | | | | IGFALS | MP2K2 | | | PH | |
| 0.651 | PTN | CDON | | | | MP2K2 | NADK | | PH | |
| 0.651 | PTN | CDON | | | | | | Notch1 | PH | |
| 0.651 | PTN | | CILP2 | | | | | | | S100A13 |
| 0.651 | PTN | CDON | | | IGFALS | MP2K2 | | | | S100A13 |
| 0.651 | PTN | | | H2A3 | | | | Notch1 | | S100A13 |
| 0.651 | PTN | | | H2A3 | | | NADK | | PH | S100A13 |
| 0.651 | PTN | | | | | MP2K2 | NADK | | PH | S100A13 |
| 0.652 | PTN | | CILP2 | | | MP2K2 | | | | |
| 0.652 | PTN | CDON | | H2A3 | IGFALS | | NADK | | | |
| 0.652 | PTN | CDON | | H2A3 | IGFALS | MP2K2 | NADK | | | |
| 0.652 | PTN | | | | IGFALS | | | Notch1 | | |
| 0.652 | PTN | | | | IGFALS | MP2K2 | | Notch1 | | |
| 0.652 | PTN | CDON | | H2A3 | | | | | PH | S100A13 |
| 0.653 | PTN | CDON | CILP2 | | | MP2K2 | | | | |
| 0.653 | PTN | CDON | | | IGFALS | | | Notch1 | | |
| 0.653 | PTN | CDON | | H2A3 | | MP2K2 | | | PH | |
| 0.653 | PTN | CDON | | | | MP2K2 | | Notch1 | PH | |
| 0.653 | PTN | CDON | CILP2 | | | | | | | S100A13 |
| 0.653 | PTN | | CILP2 | H2A3 | | | | | | S100A13 |
| 0.653 | PTN | | | H2A3 | IGFALS | | NADK | | | S100A13 |
| 0.653 | PTN | | | H2A3 | | MP2K2 | | Notch1 | | S100A13 |
| 0.654 | PTN | | CILP2 | H2A3 | | MP2K2 | | | | |
| 0.654 | PTN | CDON | | | IGFALS | MP2K2 | | Notch1 | | |
| 0.654 | PTN | CDON | | H2A3 | | | NADK | | PH | |
| 0.654 | PTN | CDON | | H2A3 | IGFALS | | | | | S100A13 |
| 0.654 | PTN | | | | IGFALS | | NADK | | | S100A13 |
| 0.654 | PTN | | | | IGFALS | MP2K2 | NADK | | | S100A13 |
| 0.654 | PTN | | | H2A3 | IGFALS | MP2K2 | NADK | | | S100A13 |
| 0.654 | PTN | CDON | | H2A3 | | | | Notch1 | | S100A13 |
| 0.654 | PTN | | | H2A3 | | MP2K2 | NADK | | PH | S100A13 |
| 0.655 | PTN | CDON | CILP2 | H2A3 | | | | | | S100A13 |
| 0.655 | PTN | | | | | | NADK | Notch1 | | S100A13 |
| 0.655 | PTN | | | | | MP2K2 | NADK | Notch1 | | S100A13 |
| 0.655 | PTN | CDON | | | | | NADK | | PH | S100A13 |
| 0.656 | PTN | | CILP2 | H2A3 | | | NADK | | | |
| 0.656 | PTN | CDON | CILP2 | | | | | Notch1 | | |
| 0.656 | PTN | CDON | | H2A3 | IGFALS | MP2K2 | | | | S100A13 |
| 0.656 | PTN | CDON | | H2A3 | | MP2K2 | | Notch1 | | S100A13 |
| 0.656 | PTN | CDON | | H2A3 | | MP2K2 | | | PH | S100A13 |
| 0.657 | PTN | CDON | CILP2 | H2A3 | | MP2K2 | | | | |
| 0.657 | PTN | | CILP2 | | | | | Notch1 | | |
| 0.657 | PTN | CDON | | | IGFALS | | | | PH | |
| 0.657 | PTN | | | H2A3 | IGFALS | | | | PH | |
| 0.657 | PTN | CDON | | H2A3 | | MP2K2 | NADK | | PH | |
| 0.657 | PTN | | CILP2 | | | MP2K2 | | | | S100A13 |
| 0.657 | PTN | CDON | | | | | NADK | Notch1 | | S100A13 |
| 0.657 | PTN | | | H2A3 | | | NADK | Notch1 | | S100A13 |
| 0.657 | PTN | | | | IGFALS | | | | PH | S100A13 |
| 0.657 | PTN | CDON | | | | MP2K2 | NADK | | PH | S100A13 |
| 0.658 | PTN | | | H2A3 | IGFALS | | | Notch1 | | |
| 0.658 | PTN | | | H2A3 | IGFALS | MP2K2 | | Notch1 | | |
| 0.658 | PTN | CDON | | | IGFALS | MP2K2 | | | PH | |
| 0.658 | PTN | CDON | CILP2 | | | MP2K2 | | | | S100A13 |
| 0.658 | PTN | CDON | | | IGFALS | | NADK | | | S100A13 |
| 0.658 | PTN | CDON | | | | MP2K2 | NADK | Notch1 | | S100A13 |
| 0.658 | PTN | | | H2A3 | | MP2K2 | NADK | Notch1 | | S100A13 |
| 0.658 | PTN | | | | | | | Notch1 | PH | S100A13 |
| 0.659 | PTN | | CILP2 | | | | NADK | | | |
| 0.659 | PTN | CDON | CILP2 | | | | NADK | | | |
| 0.659 | PTN | CDON | CILP2 | H2A3 | | | NADK | | | |
| 0.659 | PTN | | | | IGFALS | MP2K2 | NADK | Notch1 | | |
| 0.659 | PTN | | | H2A3 | IGFALS | MP2K2 | | | PH | |
| 0.659 | PTN | | | | IGFALS | | NADK | | PH | |
| 0.659 | PTN | | CILP2 | H2A3 | | MP2K2 | | | | S100A13 |
| 0.659 | PTN | CDON | | H2A3 | IGFALS | | NADK | | | S100A13 |
| 0.659 | PTN | CDON | | | IGFALS | MP2K2 | NADK | | | S100A13 |
| 0.659 | PTN | CDON | | H2A3 | | | NADK | | PH | S100A13 |
| 0.659 | PTN | | | | | MP2K2 | | Notch1 | PH | S100A13 |
| 0.66 | PTN | CDON | | H2A3 | IGFALS | | | Notch1 | | |
| 0.66 | PTN | CDON | CILP2 | | | MP2K2 | | Notch1 | | |
| 0.66 | PTN | | | | IGFALS | | NADK | Notch1 | | |
| 0.66 | PTN | CDON | | | IGFALS | | NADK | Notch1 | | |
| 0.66 | PTN | CDON | | | IGFALS | MP2K2 | NADK | Notch1 | | |
| 0.66 | PTN | | | | IGFALS | MP2K2 | NADK | | PH | |
| 0.66 | PTN | | | | | | NADK | Notch1 | PH | |
| 0.66 | PTN | | | | | MP2K2 | NADK | Notch1 | PH | |
| 0.66 | PTN | CDON | | H2A3 | IGFALS | MP2K2 | NADK | | | S100A13 |
| 0.66 | PTN | CDON | | H2A3 | | | NADK | Notch1 | | S100A13 |
| 0.66 | PTN | | | | IGFALS | MP2K2 | | | PH | S100A13 |
| 0.661 | PTN | | CILP2 | H2A3 | | MP2K2 | NADK | | | |
| 0.661 | PTN | | CILP2 | H2A3 | | | | Notch1 | | |
| 0.661 | PTN | CDON | CILP2 | H2A3 | | | | Notch1 | | |
| 0.661 | PTN | | CILP2 | | | MP2K2 | | Notch1 | | |
| 0.661 | PTN | CDON | | H2A3 | IGFALS | MP2K2 | | Notch1 | | |
| 0.661 | PTN | | CILP2 | | | | | | PH | |
| 0.661 | PTN | CDON | | | | | NADK | Notch1 | PH | |
| 0.661 | PTN | CDON | CILP2 | H2A3 | | MP2K2 | | | | S100A13 |
| 0.661 | PTN | CDON | | H2A3 | | MP2K2 | NADK | Notch1 | | S100A13 |
| 0.661 | PTN | CDON | | H2A3 | | MP2K2 | NADK | | PH | S100A13 |
| 0.661 | PTN | CDON | | | | | | Notch1 | PH | S100A13 |
| 0.662 | PTN | | | H2A3 | IGFALS | | NADK | Notch1 | | |
| 0.662 | PTN | | | H2A3 | IGFALS | MP2K2 | NADK | Notch1 | | |
| 0.662 | PTN | | | H2A3 | IGFALS | | NADK | | PH | |
| 0.662 | PTN | | | H2A3 | | | | Notch1 | PH | |
| 0.662 | PTN | CDON | | | | MP2K2 | NADK | Notch1 | PH | |
| 0.662 | PTN | | CILP2 | H2A3 | | | NADK | | | S100A13 |
| 0.662 | PTN | CDON | CILP2 | | | | | Notch1 | | S100A13 |
| 0.662 | PTN | CDON | | | | MP2K2 | | Notch1 | PH | S100A13 |
| 0.663 | PTN | | CILP2 | | | MP2K2 | NADK | | | |
| 0.663 | PTN | CDON | CILP2 | | | MP2K2 | NADK | | | |
| 0.663 | PTN | CDON | CILP2 | H2A3 | | MP2K2 | NADK | | | |
| 0.663 | PTN | CDON | CILP2 | | | | | | PH | |
| 0.663 | PTN | | | H2A3 | IGFALS | MP2K2 | NADK | | PH | |
| 0.663 | PTN | CDON | CILP2 | H2A3 | | | NADK | | | S100A13 |
| 0.663 | PTN | | CILP2 | | | | | Notch1 | | S100A13 |
| 0.663 | PTN | CDON | | | IGFALS | | | | PH | S100A13 |
| 0.663 | PTN | | | H2A3 | IGFALS | | | | PH | S100A13 |
| 0.664 | PTN | CDON | CILP2 | | IGFALS | | | | | |
| 0.664 | PTN | | CILP2 | H2A3 | IGFALS | | | | | |
| 0.664 | PTN | CDON | | H2A3 | IGFALS | | NADK | Notch1 | | |
| 0.664 | PTN | CDON | | H2A3 | IGFALS | MP2K2 | NADK | Notch1 | | |
| 0.664 | PTN | | CILP2 | H2A3 | | | | | PH | |
| 0.664 | PTN | CDON | | H2A3 | | | | Notch1 | PH | |
| 0.664 | PTN | | | H2A3 | | MP2K2 | | Notch1 | PH | |
| 0.664 | PTN | | CILP2 | | | | NADK | | | S100A13 |
| 0.664 | PTN | | | | IGFALS | | | Notch1 | | S100A13 |
| 0.664 | PTN | CDON | | | IGFALS | | | Notch1 | | S100A13 |
| 0.665 | PTN | | CILP2 | | IGFALS | | | | | |
| 0.665 | PTN | | CILP2 | H2A3 | | MP2K2 | | Notch1 | | |
| 0.665 | PTN | CDON | CILP2 | H2A3 | | MP2K2 | | Notch1 | | |
| 0.665 | PTN | CDON | | H2A3 | IGFALS | | | | PH | |
| 0.665 | PTN | CDON | | | IGFALS | | NADK | | PH | |
| 0.665 | PTN | CDON | | | IGFALS | MP2K2 | NADK | | PH | |
| 0.665 | PTN | CDON | CILP2 | | | | NADK | | | S100A13 |
| 0.665 | PTN | | | | IGFALS | MP2K2 | | Notch1 | | S100A13 |
| 0.665 | PTN | | CILP2 | | | | | | PH | S100A13 |
| 0.665 | PTN | CDON | | | IGFALS | MP2K2 | | | PH | S100A13 |
| 0.665 | PTN | | | H2A3 | IGFALS | MP2K2 | | | PH | S100A13 |
| 0.666 | PTN | CDON | CILP2 | H2A3 | IGFALS | | | | | |
| 0.666 | PTN | CDON | CILP2 | | IGFALS | MP2K2 | | | | |
| 0.666 | PTN | CDON | CILP2 | | | | NADK | Notch1 | | |
| 0.666 | PTN | CDON | | H2A3 | | MP2K2 | | Notch1 | PH | |
| 0.666 | PTN | | | H2A3 | | | NADK | Notch1 | PH | |
| 0.666 | PTN | | CILP2 | H2A3 | | MP2K2 | NADK | | | S100A13 |
| 0.666 | PTN | CDON | CILP2 | | | MP2K2 | | Notch1 | | S100A13 |
| 0.666 | PTN | CDON | | | IGFALS | MP2K2 | | Notch1 | | S100A13 |
| 0.666 | PTN | CDON | CILP2 | | | | | | PH | S100A13 |
| 0.667 | PTN | | CILP2 | | IGFALS | MP2K2 | | | | |
| 0.667 | PTN | | CILP2 | H2A3 | IGFALS | MP2K2 | | | | |
| 0.667 | PTN | | CILP2 | H2A3 | | | NADK | Notch1 | | |
| 0.667 | PTN | CDON | CILP2 | H2A3 | | | NADK | Notch1 | | |
| 0.667 | PTN | CDON | CILP2 | H2A3 | | | | | PH | |
| 0.667 | PTN | | CILP2 | | | MP2K2 | | | PH | |
| 0.667 | PTN | CDON | | H2A3 | IGFALS | MP2K2 | | | PH | |
| 0.667 | PTN | | | | IGFALS | | | Notch1 | PH | |
| 0.667 | PTN | | | | IGFALS | MP2K2 | | Notch1 | PH | |
| 0.667 | PTN | CDON | | H2A3 | | | NADK | Notch1 | PH | |
| 0.667 | PTN | | | H2A3 | | MP2K2 | NADK | Notch1 | PH | |
| 0.667 | PTN | | CILP2 | H2A3 | | | | Notch1 | | S100A13 |
| 0.667 | PTN | CDON | CILP2 | H2A3 | | | | Notch1 | | S100A13 |
| 0.667 | PTN | | | H2A3 | IGFALS | | | Notch1 | | S100A13 |
| 0.667 | PTN | | CILP2 | | | MP2K2 | | Notch1 | | S100A13 |
| 0.668 | PTN | CDON | CILP2 | H2A3 | IGFALS | MP2K2 | | | | |
| 0.668 | PTN | | CILP2 | | | | NADK | Notch1 | | |
| 0.668 | PTN | CDON | CILP2 | | | MP2K2 | NADK | Notch1 | | |
| 0.668 | PTN | CDON | CILP2 | | | MP2K2 | | | PH | |
| 0.668 | PTN | CDON | | H2A3 | IGFALS | | NADK | | PH | |
| 0.668 | PTN | CDON | | | IGFALS | | | Notch1 | PH | |
| 0.668 | PTN | | CILP2 | | | MP2K2 | NADK | | | S100A13 |
| 0.668 | PTN | CDON | CILP2 | | | MP2K2 | NADK | | | S100A13 |
| 0.668 | PTN | CDON | CILP2 | H2A3 | | MP2K2 | NADK | | | S100A13 |
| 0.668 | PTN | CDON | | H2A3 | IGFALS | | | Notch1 | | S100A13 |
| 0.668 | PTN | | | H2A3 | IGFALS | MP2K2 | | Notch1 | | S100A13 |
| 0.668 | PTN | | CILP2 | H2A3 | | | | | PH | S100A13 |
| 0.668 | PTN | | | | IGFALS | | NADK | | PH | S100A13 |
| 0.668 | PTN | | | H2A3 | | | | Notch1 | PH | S100A13 |
| 0.668 | PTN | | | | | | NADK | Notch1 | PH | S100A13 |
| 0.669 | PTN | | CILP2 | | | MP2K2 | NADK | Notch1 | | |
| 0.669 | PTN | CDON | | H2A3 | IGFALS | MP2K2 | NADK | | PH | |
| 0.669 | PTN | CDON | | | IGFALS | MP2K2 | | Notch1 | PH | |
| 0.669 | PTN | CDON | | H2A3 | | MP2K2 | NADK | Notch1 | PH | |
| 0.669 | PTN | CDON | CILP2 | | IGFALS | | | | | S100A13 |
| 0.669 | PTN | | CILP2 | H2A3 | IGFALS | | | | | S100A13 |
| 0.669 | PTN | CDON | | H2A3 | IGFALS | | | | PH | S100A13 |
| 0.669 | PTN | | | H2A3 | IGFALS | | NADK | | PH | S100A13 |
| 0.669 | PTN | | | | IGFALS | MP2K2 | NADK | | PH | S100A13 |
| 0.669 | PTN | | | | | MP2K2 | NADK | Notch1 | PH | S100A13 |
| 0.67 | PTN | | CILP2 | H2A3 | IGFALS | | NADK | | | |
| 0.67 | PTN | CDON | CILP2 | | IGFALS | | | Notch1 | | |
| 0.67 | PTN | | CILP2 | H2A3 | | MP2K2 | NADK | Notch1 | | |
| 0.67 | PTN | CDON | CILP2 | H2A3 | | MP2K2 | NADK | Notch1 | | |
| 0.67 | PTN | | CILP2 | H2A3 | | MP2K2 | | | PH | |
| 0.67 | PTN | | CILP2 | H2A3 | | | NADK | | PH | |
| 0.67 | PTN | CDON | CILP2 | | | | | Notch1 | PH | |
| 0.67 | PTN | | CILP2 | | IGFALS | | | | | S100A13 |
| 0.67 | PTN | CDON | CILP2 | H2A3 | IGFALS | | | | | S100A13 |
| 0.67 | PTN | CDON | | H2A3 | IGFALS | MP2K2 | | Notch1 | | S100A13 |
| 0.67 | PTN | CDON | CILP2 | H2A3 | | | | | PH | S100A13 |
| 0.67 | PTN | | CILP2 | | | MP2K2 | | | PH | S100A13 |
| 0.67 | PTN | | | H2A3 | IGFALS | MP2K2 | NADK | | PH | S100A13 |
| 0.67 | PTN | CDON | | H2A3 | | | | Notch1 | PH | S100A13 |
| 0.67 | PTN | | | H2A3 | | MP2K2 | | Notch1 | PH | S100A13 |
| 0.67 | PTN | CDON | | | | | NADK | Notch1 | PH | S100A13 |
| 0.67 | PTN | CDON | | | | MP2K2 | NADK | Notch1 | PH | S100A13 |
| 0.671 | PTN | CDON | CILP2 | H2A3 | IGFALS | | NADK | | | |
| 0.671 | PTN | | CILP2 | H2A3 | IGFALS | MP2K2 | NADK | | | |
| 0.671 | PTN | | CILP2 | | | | NADK | | PH | |
| 0.671 | PTN | | CILP2 | | | | | Notch1 | PH | |
| 0.671 | PTN | | CILP2 | H2A3 | | MP2K2 | | Notch1 | | S100A13 |
| 0.671 | PTN | CDON | CILP2 | H2A3 | | MP2K2 | | Notch1 | | S100A13 |
| 0.671 | PTN | | | | IGFALS | | NADK | Notch1 | | S100A13 |
| 0.671 | PTN | CDON | | | IGFALS | | NADK | Notch1 | | S100A13 |
| 0.671 | PTN | | | | IGFALS | MP2K2 | NADK | Notch1 | | S100A13 |
| 0.671 | PTN | CDON | CILP2 | | | MP2K2 | | | PH | S100A13 |
| 0.671 | PTN | CDON | | H2A3 | IGFALS | MP2K2 | | | PH | S100A13 |
| 0.672 | PTN | CDON | CILP2 | | IGFALS | | NADK | | | |
| 0.672 | PTN | CDON | CILP2 | H2A3 | IGFALS | MP2K2 | NADK | | | |
| 0.672 | PTN | | CILP2 | | IGFALS | | | Notch1 | | |
| 0.672 | PTN | CDON | CILP2 | | IGFALS | MP2K2 | | Notch1 | | |
| 0.672 | PTN | CDON | CILP2 | H2A3 | | MP2K2 | | | PH | |
| 0.672 | PTN | CDON | CILP2 | | | | NADK | | PH | |
| 0.672 | PTN | CDON | CILP2 | H2A3 | | | NADK | | PH | |
| 0.672 | PTN | CDON | CILP2 | | IGFALS | MP2K2 | | | | S100A13 |
| 0.672 | PTN | | | H2A3 | IGFALS | | NADK | Notch1 | | S100A13 |
| 0.672 | PTN | CDON | | | IGFALS | MP2K2 | NADK | Notch1 | | S100A13 |
| 0.672 | PTN | | | H2A3 | IGFALS | MP2K2 | NADK | Notch1 | | S100A13 |
| 0.672 | PTN | CDON | | | IGFALS | | NADK | | PH | S100A13 |
| 0.672 | PTN | CDON | | | IGFALS | MP2K2 | NADK | | PH | S100A13 |
| 0.672 | PTN | CDON | | H2A3 | | MP2K2 | | Notch1 | PH | S100A13 |
| 0.672 | PTN | | | H2A3 | | | NADK | Notch1 | PH | S100A13 |
| 0.673 | PTN | | CILP2 | | IGFALS | | NADK | | | |
| 0.673 | PTN | CDON | CILP2 | | IGFALS | MP2K2 | NADK | | | |
| 0.673 | PTN | CDON | CILP2 | H2A3 | IGFALS | | | Notch1 | | |
| 0.673 | PTN | CDON | CILP2 | | | MP2K2 | | Notch1 | PH | |
| 0.673 | PTN | | | | IGFALS | | NADK | Notch1 | PH | |
| 0.673 | PTN | | | | IGFALS | MP2K2 | NADK | Notch1 | PH | |
| 0.673 | PTN | | CILP2 | | IGFALS | MP2K2 | | | | S100A13 |
| 0.673 | PTN | | CILP2 | H2A3 | IGFALS | MP2K2 | | | | S100A13 |
| 0.673 | PTN | CDON | CILP2 | | | | NADK | Notch1 | | S100A13 |
| 0.673 | PTN | CDON | CILP2 | H2A3 | | | NADK | Notch1 | | S100A13 |
| 0.673 | PTN | CDON | | H2A3 | IGFALS | | NADK | Notch1 | | S100A13 |
| 0.673 | PTN | CDON | | H2A3 | IGFALS | MP2K2 | NADK | Notch1 | | S100A13 |
| 0.673 | PTN | | CILP2 | H2A3 | | MP2K2 | | | PH | S100A13 |
| 0.673 | PTN | | | H2A3 | | MP2K2 | NADK | Notch1 | PH | S100A13 |
| 0.674 | PTN | | CILP2 | | IGFALS | MP2K2 | NADK | | | |
| 0.674 | PTN | | CILP2 | H2A3 | IGFALS | | | Notch1 | | |
| 0.674 | PTN | | CILP2 | | IGFALS | MP2K2 | | Notch1 | | |
| 0.674 | PTN | | CILP2 | H2A3 | | MP2K2 | NADK | | PH | |
| 0.674 | PTN | | CILP2 | | | MP2K2 | | Notch1 | PH | |
| 0.674 | PTN | CDON | | | IGFALS | | NADK | Notch1 | PH | |
| 0.674 | PTN | CDON | | | IGFALS | MP2K2 | NADK | Notch1 | PH | |
| 0.674 | PTN | CDON | CILP2 | H2A3 | IGFALS | MP2K2 | | | | S100A13 |
| 0.674 | PTN | | CILP2 | | | | NADK | Notch1 | | S100A13 |
| 0.674 | PTN | | CILP2 | H2A3 | | | NADK | Notch1 | | S100A13 |
| 0.674 | PTN | | CILP2 | H2A3 | | | NADK | | PH | S100A13 |
| 0.674 | PTN | CDON | | H2A3 | IGFALS | | NADK | | PH | S100A13 |
| 0.674 | PTN | | CILP2 | | | | | Notch1 | PH | S100A13 |
| 0.674 | PTN | CDON | CILP2 | | | | | Notch1 | PH | S100A13 |
| 0.674 | PTN | CDON | | H2A3 | | | NADK | Notch1 | PH | S100A13 |
| 0.675 | PTN | | CILP2 | H2A3 | IGFALS | MP2K2 | | Notch1 | | |
| 0.675 | PTN | CDON | CILP2 | H2A3 | IGFALS | MP2K2 | | Notch1 | | |
| 0.675 | PTN | | CILP2 | | | MP2K2 | NADK | | PH | |
| 0.675 | PTN | CDON | CILP2 | | | MP2K2 | NADK | | PH | |
| 0.675 | PTN | | | H2A3 | IGFALS | | | Notch1 | PH | |
| 0.675 | PTN | | CILP2 | H2A3 | IGFALS | | NADK | | | S100A13 |
| 0.675 | PTN | CDON | CILP2 | H2A3 | | MP2K2 | | | PH | S100A13 |
| 0.675 | PTN | CDON | | H2A3 | IGFALS | MP2K2 | NADK | | PH | S100A13 |
| 0.675 | PTN | | | | IGFALS | | | Notch1 | PH | S100A13 |
| 0.675 | PTN | CDON | | H2A3 | | MP2K2 | NADK | Notch1 | PH | S100A13 |
| 0.676 | PTN | | CILP2 | | IGFALS | | | | PH | |
| 0.676 | PTN | CDON | CILP2 | | IGFALS | | | | PH | |
| 0.676 | PTN | CDON | CILP2 | H2A3 | | MP2K2 | NADK | | PH | |
| 0.676 | PTN | | CILP2 | H2A3 | | | | Notch1 | PH | |
| 0.676 | PTN | CDON | CILP2 | H2A3 | | | | Notch1 | PH | |
| 0.676 | PTN | | | H2A3 | IGFALS | MP2K2 | | Notch1 | PH | |
| 0.676 | PTN | CDON | CILP2 | H2A3 | IGFALS | | NADK | | | S100A13 |
| 0.676 | PTN | | CILP2 | | | MP2K2 | NADK | Notch1 | | S100A13 |
| 0.676 | PTN | CDON | CILP2 | | | MP2K2 | NADK | Notch1 | | S100A13 |
| 0.676 | PTN | CDON | CILP2 | H2A3 | | MP2K2 | NADK | Notch1 | | S100A13 |
| 0.676 | PTN | | CILP2 | | | | NADK | | PH | S100A13 |
| 0.676 | PTN | CDON | CILP2 | | | | NADK | | PH | S100A13 |
| 0.676 | PTN | CDON | CILP2 | H2A3 | | | NADK | | PH | S100A13 |
| 0.676 | PTN | CDON | | | IGFALS | | | Notch1 | PH | S100A13 |
| 0.676 | PTN | | | | IGFALS | MP2K2 | | Notch1 | PH | S100A13 |
| 0.677 | PTN | CDON | CILP2 | | IGFALS | | NADK | Notch1 | | |
| 0.677 | PTN | CDON | CILP2 | H2A3 | IGFALS | | NADK | Notch1 | | |
| 0.677 | PTN | | CILP2 | H2A3 | IGFALS | | | | PH | |
| 0.677 | PTN | CDON | | H2A3 | IGFALS | | | Notch1 | PH | |
| 0.677 | PTN | CDON | | H2A3 | IGFALS | MP2K2 | | Notch1 | PH | |
| 0.677 | PTN | | | H2A3 | IGFALS | | NADK | Notch1 | PH | |
| 0.677 | PTN | | | H2A3 | IGFALS | MP2K2 | NADK | Notch1 | PH | |
| 0.677 | PTN | CDON | CILP2 | | IGFALS | | NADK | | | S100A13 |
| 0.677 | PTN | CDON | CILP2 | | IGFALS | | | Notch1 | | S100A13 |
| 0.677 | PTN | | CILP2 | H2A3 | | MP2K2 | NADK | Notch1 | | S100A13 |
| 0.677 | PTN | CDON | CILP2 | | | MP2K2 | | Notch1 | PH | S100A13 |
| 0.677 | PTN | CDON | | | IGFALS | MP2K2 | | Notch1 | PH | S100A13 |
| 0.678 | PTN | | CILP2 | H2A3 | IGFALS | | NADK | Notch1 | | |
| 0.678 | PTN | CDON | CILP2 | | IGFALS | MP2K2 | NADK | Notch1 | | |
| 0.678 | PTN | CDON | CILP2 | H2A3 | IGFALS | MP2K2 | NADK | Notch1 | | |
| 0.678 | PTN | | CILP2 | | IGFALS | MP2K2 | | | PH | |
| 0.678 | PTN | CDON | CILP2 | | | | NADK | Notch1 | PH | |
| 0.678 | PTN | CDON | | H2A3 | IGFALS | | NADK | Notch1 | PH | |
| 0.678 | PTN | | CILP2 | | IGFALS | | NADK | | | S100A13 |
| 0.678 | PTN | | CILP2 | H2A3 | IGFALS | MP2K2 | NADK | | | S100A13 |
| 0.678 | PTN | CDON | CILP2 | H2A3 | IGFALS | MP2K2 | NADK | | | S100A13 |
| 0.678 | PTN | | CILP2 | | IGFALS | | | Notch1 | | S100A13 |
| 0.678 | PTN | | CILP2 | | | MP2K2 | | Notch1 | PH | S100A13 |
| 0.679 | PTN | | CILP2 | | IGFALS | | NADK | Notch1 | | |
| 0.679 | PTN | | CILP2 | | IGFALS | MP2K2 | NADK | Notch1 | | |
| 0.679 | PTN | | CILP2 | H2A3 | IGFALS | MP2K2 | NADK | Notch1 | | |
| 0.679 | PTN | CDON | CILP2 | H2A3 | IGFALS | | | | PH | |
| 0.679 | PTN | CDON | CILP2 | | IGFALS | MP2K2 | | | PH | |
| 0.679 | PTN | CDON | CILP2 | H2A3 | | MP2K2 | | Notch1 | PH | |
| 0.679 | PTN | | CILP2 | | | | NADK | Notch1 | PH | |
| 0.679 | PTN | CDON | | H2A3 | IGFALS | MP2K2 | NADK | Notch1 | PH | |
| 0.679 | PTN | CDON | CILP2 | | IGFALS | MP2K2 | NADK | | | S100A13 |
| 0.679 | PTN | | CILP2 | H2A3 | IGFALS | | | Notch1 | | S100A13 |
| 0.679 | PTN | CDON | CILP2 | H2A3 | IGFALS | | | Notch1 | | S100A13 |
| 0.679 | PTN | CDON | CILP2 | | IGFALS | MP2K2 | | Notch1 | | S100A13 |
| 0.679 | PTN | CDON | CILP2 | | | MP2K2 | NADK | | PH | S100A13 |
| 0.679 | PTN | | CILP2 | H2A3 | | MP2K2 | NADK | | PH | S100A13 |
| 0.679 | PTN | CDON | CILP2 | H2A3 | | MP2K2 | NADK | | PH | S100A13 |
| 0.68 | PTN | | CILP2 | H2A3 | IGFALS | MP2K2 | | | PH | |
| 0.68 | PTN | | CILP2 | H2A3 | | MP2K2 | | Notch1 | PH | |
| 0.68 | PTN | | CILP2 | H2A3 | | | NADK | Notch1 | PH | |
| 0.68 | PTN | CDON | CILP2 | H2A3 | | | NADK | Notch1 | PH | |
| 0.68 | PTN | CDON | CILP2 | | | MP2K2 | NADK | Notch1 | PH | |
| 0.68 | PTN | | CILP2 | | IGFALS | MP2K2 | NADK | | | S100A13 |
| 0.68 | PTN | | CILP2 | | IGFALS | MP2K2 | | Notch1 | | S100A13 |
| 0.68 | PTN | | CILP2 | | IGFALS | | | | PH | S100A13 |
| 0.68 | PTN | CDON | CILP2 | | IGFALS | | | | PH | S100A13 |
| 0.68 | PTN | | CILP2 | | | MP2K2 | NADK | | PH | S100A13 |
| 0.68 | PTN | | CILP2 | H2A3 | | | | Notch1 | PH | S100A13 |
| 0.68 | PTN | CDON | CILP2 | H2A3 | | | | Notch1 | PH | S100A13 |
| 0.681 | PTN | | CILP2 | H2A3 | IGFALS | | NADK | | PH | |
| 0.681 | PTN | | CILP2 | | | MP2K2 | NADK | Notch1 | PH | |
| 0.681 | PTN | CDON | CILP2 | H2A3 | IGFALS | MP2K2 | | Notch1 | | S100A13 |
| 0.681 | PTN | | CILP2 | H2A3 | IGFALS | | | | PH | S100A13 |
| 0.681 | PTN | | | H2A3 | IGFALS | | | Notch1 | PH | S100A13 |
| 0.682 | PTN | CDON | CILP2 | H2A3 | IGFALS | MP2K2 | | | PH | |
| 0.682 | PTN | | CILP2 | | IGFALS | | NADK | | PH | |
| 0.682 | PTN | CDON | CILP2 | | IGFALS | | NADK | | PH | |
| 0.682 | PTN | CDON | CILP2 | H2A3 | IGFALS | | NADK | | PH | |
| 0.682 | PTN | CDON | CILP2 | | IGFALS | | | Notch1 | PH | |
| 0.682 | PTN | CDON | CILP2 | H2A3 | | MP2K2 | NADK | Notch1 | PH | |
| 0.682 | PTN | | CILP2 | H2A3 | IGFALS | MP2K2 | | Notch1 | | S100A13 |
| 0.682 | PTN | CDON | CILP2 | H2A3 | IGFALS | | | | PH | S100A13 |
| 0.682 | PTN | CDON | | H2A3 | IGFALS | | | Notch1 | PH | S100A13 |
| 0.682 | PTN | | | H2A3 | IGFALS | MP2K2 | | Notch1 | PH | S100A13 |
| 0.682 | PTN | | | | IGFALS | | NADK | Notch1 | PH | S100A13 |
| 0.682 | PTN | CDON | | | IGFALS | | NADK | Notch1 | PH | S100A13 |
| 0.682 | PTN | | | | IGFALS | MP2K2 | NADK | Notch1 | PH | S100A13 |
| 0.683 | PTN | CDON | CILP2 | | IGFALS | MP2K2 | NADK | | PH | |
| 0.683 | PTN | | CILP2 | H2A3 | IGFALS | MP2K2 | NADK | | PH | |
| 0.683 | PTN | | CILP2 | | IGFALS | | | Notch1 | PH | |
| 0.683 | PTN | | CILP2 | H2A3 | | MP2K2 | NADK | Notch1 | PH | |
| 0.683 | PTN | CDON | CILP2 | | IGFALS | | NADK | Notch1 | | S100A13 |
| 0.683 | PTN | CDON | CILP2 | H2A3 | IGFALS | | NADK | Notch1 | | S100A13 |
| 0.683 | PTN | | CILP2 | | IGFALS | MP2K2 | | | PH | S100A13 |
| 0.683 | PTN | CDON | CILP2 | | IGFALS | MP2K2 | | | PH | S100A13 |
| 0.683 | PTN | CDON | CILP2 | H2A3 | | MP2K2 | | Notch1 | PH | S100A13 |
| 0.683 | PTN | CDON | | H2A3 | IGFALS | MP2K2 | | Notch1 | PH | S100A13 |
| 0.683 | PTN | CDON | CILP2 | | | | NADK | Notch1 | PH | S100A13 |
| 0.683 | PTN | CDON | | | IGFALS | MP2K2 | NADK | Notch1 | PH | S100A13 |
| 0.684 | PTN | | CILP2 | | IGFALS | MP2K2 | NADK | | PH | |
| 0.684 | PTN | CDON | CILP2 | H2A3 | IGFALS | MP2K2 | NADK | | PH | |
| 0.684 | PTN | CDON | CILP2 | | IGFALS | MP2K2 | | Notch1 | PH | |
| 0.684 | PTN | | CILP2 | H2A3 | IGFALS | | NADK | Notch1 | | S100A13 |
| 0.684 | PTN | | CILP2 | H2A3 | IGFALS | MP2K2 | | | PH | S100A13 |
| 0.684 | PTN | | CILP2 | H2A3 | | MP2K2 | | Notch1 | PH | S100A13 |
| 0.684 | PTN | | CILP2 | | | | NADK | Notch1 | PH | S100A13 |
| 0.685 | PTN | | CILP2 | | IGFALS | MP2K2 | | Notch1 | PH | |
| 0.685 | PTN | | CILP2 | | IGFALS | | NADK | Notch1 | | S100A13 |
| 0.685 | PTN | CDON | CILP2 | | IGFALS | MP2K2 | NADK | Notch1 | | S100A13 |
| 0.685 | PTN | CDON | CILP2 | H2A3 | IGFALS | MP2K2 | NADK | Notch1 | | S100A13 |
| 0.685 | PTN | CDON | CILP2 | H2A3 | IGFALS | MP2K2 | | | PH | S100A13 |
| 0.685 | PTN | | CILP2 | H2A3 | | | NADK | Notch1 | PH | S100A13 |
| 0.685 | PTN | CDON | CILP2 | H2A3 | | | NADK | Notch1 | PH | S100A13 |
| 0.685 | PTN | | | H2A3 | IGFALS | | NADK | Notch1 | PH | S100A13 |
| 0.685 | PTN | CDON | | H2A3 | IGFALS | | NADK | Notch1 | PH | S100A13 |
| 0.685 | PTN | CDON | CILP2 | | | MP2K2 | NADK | Notch1 | PH | S100A13 |
| 0.685 | PTN | | | H2A3 | IGFALS | MP2K2 | NADK | Notch1 | PH | S100A13 |
| 0.685 | PTN | CDON | | H2A3 | IGFALS | MP2K2 | NADK | Notch1 | PH | S100A13 |
| 0.686 | PTN | | CILP2 | H2A3 | IGFALS | | | Notch1 | PH | |
| 0.686 | PTN | CDON | CILP2 | H2A3 | IGFALS | | | Notch1 | PH | |
| 0.686 | PTN | | CILP2 | H2A3 | IGFALS | MP2K2 | NADK | Notch1 | | S100A13 |
| 0.686 | PTN | | CILP2 | H2A3 | IGFALS | | NADK | | PH | S100A13 |
| 0.686 | PTN | CDON | CILP2 | H2A3 | IGFALS | | NADK | | PH | S100A13 |
| 0.686 | PTN | CDON | CILP2 | | IGFALS | | | Notch1 | PH | S100A13 |
| 0.686 | PTN | | CILP2 | | | MP2K2 | NADK | Notch1 | PH | S100A13 |
| 0.687 | PTN | CDON | CILP2 | | IGFALS | | NADK | Notch1 | PH | |
| 0.687 | PTN | | CILP2 | | IGFALS | MP2K2 | NADK | Notch1 | | S100A13 |
| 0.687 | PTN | | CILP2 | | IGFALS | | NADK | | PH | S100A13 |
| 0.687 | PTN | CDON | CILP2 | | IGFALS | | NADK | | PH | S100A13 |
| 0.687 | PTN | | CILP2 | | IGFALS | | | Notch1 | PH | S100A13 |
| 0.687 | PTN | CDON | CILP2 | H2A3 | | MP2K2 | NADK | Notch1 | PH | S100A13 |
| 0.688 | PTN | | CILP2 | H2A3 | IGFALS | MP2K2 | | Notch1 | PH | |
| 0.688 | PTN | CDON | CILP2 | H2A3 | IGFALS | MP2K2 | | Notch1 | PH | |
| 0.688 | PTN | | CILP2 | | IGFALS | | NADK | Notch1 | PH | |
| 0.688 | PTN | CDON | CILP2 | H2A3 | IGFALS | | NADK | Notch1 | PH | |
| 0.688 | PTN | CDON | CILP2 | | IGFALS | MP2K2 | NADK | Notch1 | PH | |
| 0.688 | PTN | | CILP2 | H2A3 | IGFALS | MP2K2 | NADK | | PH | S100A13 |
| 0.688 | PTN | CDON | CILP2 | H2A3 | IGFALS | MP2K2 | NADK | | PH | S100A13 |
| 0.688 | PTN | CDON | CILP2 | | IGFALS | MP2K2 | | Notch1 | PH | S100A13 |
| 0.688 | PTN | | CILP2 | H2A3 | | MP2K2 | NADK | Notch1 | PH | S100A13 |
| 0.689 | PTN | | CILP2 | H2A3 | IGFALS | | NADK | Notch1 | PH | |
| 0.689 | PTN | | CILP2 | | IGFALS | MP2K2 | NADK | Notch1 | PH | |
| 0.689 | PTN | CDON | CILP2 | H2A3 | IGFALS | MP2K2 | NADK | Notch1 | PH | |
| 0.689 | PTN | | CILP2 | | IGFALS | MP2K2 | NADK | | PH | S100A13 |
| 0.689 | PTN | CDON | CILP2 | | IGFALS | MP2K2 | NADK | | PH | S100A13 |
| 0.69 | PTN | | CILP2 | H2A3 | IGFALS | MP2K2 | NADK | Notch1 | PH | |
| 0.69 | PTN | CDON | CILP2 | H2A3 | IGFALS | | | Notch1 | PH | S100A13 |
| 0.69 | PTN | | CILP2 | | IGFALS | MP2K2 | | Notch1 | PH | S100A13 |
| 0.691 | PTN | | CILP2 | H2A3 | IGFALS | | | Notch1 | PH | S100A13 |
| 0.692 | PTN | CDON | CILP2 | H2A3 | IGFALS | MP2K2 | | Notch1 | PH | S100A13 |
| 0.692 | PTN | CDON | CILP2 | | IGFALS | | NADK | Notch1 | PH | S100A13 |
| 0.693 | PTN | | CILP2 | H2A3 | IGFALS | MP2K2 | | Notch1 | PH | S100A13 |
| 0.693 | PTN | CDON | CILP2 | H2A3 | IGFALS | | NADK | Notch1 | PH | S100A13 |
| 0.694 | PTN | | CILP2 | | IGFALS | | NADK | Notch1 | PH | S100A13 |
| 0.694 | PTN | | CILP2 | H2A3 | IGFALS | | NADK | Notch1 | PH | S100A13 |
| 0.694 | PTN | CDON | CILP2 | | IGFALS | MP2K2 | NADK | Notch1 | PH | S100A13 |
| 0.694 | PTN | CDON | CILP2 | H2A3 | IGFALS | MP2K2 | NADK | Notch1 | PH | S100A13 |
| 0.695 | PTN | | CILP2 | | IGFALS | MP2K2 | NADK | Notch1 | PH | S100A13 |
| *0.696 | PTN | | CILP2 | H2A3 | IGFALS | MP2K2 | NADK | Notch1 | PH | S100A13 |

*Included in Table 38

TABLE 40
PH Biomarker Panels-20 Year Model

| AUC | PH | CDON | CILP2 | H2A3 | IGFALS | MP2K2 | NADK | Notch1 | PTN | S100A13 |
|---|---|---|---|---|---|---|---|---|---|---|
| 0.597 | PH | | | | | | | | | S100A13 |
| 0.609 | PH | CDON | | | | | | | | |
| 0.609 | PH | | | | | MP2K2 | | | | |
| 0.611 | PH | | | H2A3 | | | | | | |
| 0.612 | PH | | CILP2 | | | | | | | |
| 0.613 | PH | | | H2A3 | | | | | | S100A13 |
| 0.614 | PH | | | | | | | Notch1 | | |
| 0.616 | PH | | | | | | NADK | | | |
| 0.616 | PH | | | | | MP2K2 | | | | S100A13 |
| 0.617 | PH | CDON | | | | | | | | S100A13 |
| 0.62 | PH | | | H2A3 | | | NADK | | | |
| 0.62 | PH | CDON | | | | | | Notch1 | | |
| 0.621 | PH | | CILP2 | | | | | | | S100A13 |
| 0.622 | PH | CDON | | | | MP2K2 | | | | |
| 0.622 | PH | | | | | MP2K2 | | Notch1 | | |
| 0.622 | PH | | | | | | NADK | | | S100A13 |
| 0.623 | PH | CDON | CILP2 | | | | | | | |
| 0.624 | PH | | CILP2 | | | | | Notch1 | | |
| 0.624 | PH | | | | | | | Notch1 | | S100A13 |
| 0.625 | PH | | | | | | | | PTN | |
| 0.625 | PH | CDON | | H2A3 | | | | | | |
| 0.625 | PH | | | H2A3 | | | NADK | | | S100A13 |
| 0.626 | PH | CDON | | | | | NADK | | | |
| 0.626 | PH | | | | | MP2K2 | NADK | | | |
| 0.627 | PH | | CILP2 | H2A3 | | | | | | |
| 0.627 | PH | | | | IGFALS | | | | | |
| 0.628 | PH | | | H2A3 | | MP2K2 | | | | |
| 0.628 | PH | CDON | CILP2 | | | | | Notch1 | | |
| 0.629 | PH | CDON | | | | MP2K2 | | Notch1 | | |
| 0.629 | PH | | | | | | NADK | Notch1 | | |
| 0.629 | PH | CDON | | H2A3 | | | | | | S100A13 |
| 0.629 | PH | | | H2A3 | | MP2K2 | | | | S100A13 |
| 0.63 | PH | | CILP2 | | | MP2K2 | | | | |
| 0.63 | PH | | | H2A3 | | | | Notch1 | | |
| 0.63 | PH | CDON | CILP2 | | | | | | | S100A13 |
| 0.63 | PH | | CILP2 | H2A3 | | | | | | S100A13 |
| 0.63 | PH | CDON | | | | MP2K2 | | | | S100A13 |
| 0.631 | PH | | | | | MP2K2 | | | PTN | |
| 0.632 | PH | | CILP2 | | | | NADK | | | |
| 0.632 | PH | CDON | | H2A3 | | | NADK | | | |
| 0.632 | PH | | | H2A3 | | MP2K2 | NADK | | | |
| 0.632 | PH | | | | | | | | PTN | S100A13 |
| 0.632 | PH | CDON | | | | | | Notch1 | | S100A13 |
| 0.633 | PH | CDON | | | | | NADK | Notch1 | | |
| 0.633 | PH | | | | | MP2K2 | NADK | | | S100A13 |
| 0.633 | PH | | CILP2 | | | | | Notch1 | | S100A13 |
| 0.634 | PH | CDON | CILP2 | H2A3 | | | | | | |
| 0.634 | PH | | | | IGFALS | MP2K2 | | | | |
| 0.634 | PH | | CILP2 | H2A3 | | | NADK | | | |
| 0.634 | PH | CDON | | | | MP2K2 | NADK | | | |
| 0.634 | PH | | | | | MP2K2 | NADK | Notch1 | | |
| 0.634 | PH | CDON | | | | | NADK | | | S100A13 |
| 0.634 | PH | | | | | MP2K2 | | Notch1 | | S100A13 |
| 0.635 | PH | | CILP2 | | | MP2K2 | | Notch1 | | |
| 0.635 | PH | | | H2A3 | | | NADK | Notch1 | | |
| 0.635 | PH | | | | IGFALS | | | | | S100A13 |
| 0.636 | PH | CDON | | H2A3 | | | | Notch1 | | |
| 0.636 | PH | | CILP2 | H2A3 | | | | Notch1 | | |
| 0.637 | PH | CDON | | | | | | | PTN | |
| 0.637 | PH | CDON | | | IGFALS | | | | | |
| 0.637 | PH | CDON | CILP2 | | | MP2K2 | | | | |
| 0.637 | PH | CDON | CILP2 | H2A3 | | | | | | S100A13 |
| 0.637 | PH | | | | | MP2K2 | | | PTN | S100A13 |
| 0.637 | PH | CDON | | H2A3 | | | NADK | | | S100A13 |
| 0.637 | PH | | | H2A3 | | MP2K2 | NADK | | | S100A13 |
| 0.637 | PH | | | H2A3 | | | | Notch1 | | S100A13 |
| 0.638 | PH | | | H2A3 | | | | | PTN | |
| 0.638 | PH | | | H2A3 | IGFALS | | | | | |
| 0.638 | PH | CDON | | H2A3 | | MP2K2 | | | | |
| 0.638 | PH | | CILP2 | | | | NADK | Notch1 | | |
| 0.638 | PH | | CILP2 | | | MP2K2 | | | | S100A13 |
| 0.638 | PH | CDON | CILP2 | | | | | Notch1 | | S100A13 |
| 0.639 | PH | CDON | CILP2 | | | | NADK | | | |
| 0.639 | PH | | | | IGFALS | | | Notch1 | | |
| 0.639 | PH | CDON | | | | MP2K2 | NADK | Notch1 | | |
| 0.64 | PH | | | | IGFALS | | NADK | | | |
| 0.64 | PH | CDON | CILP2 | H2A3 | | | | Notch1 | | |
| 0.64 | PH | CDON | CILP2 | | | MP2K2 | | Notch1 | | |
| 0.64 | PH | CDON | | H2A3 | | | NADK | Notch1 | | |
| 0.64 | PH | CDON | | H2A3 | | MP2K2 | | | | S100A13 |
| 0.64 | PH | | | | | | NADK | Notch1 | | S100A13 |
| 0.641 | PH | CDON | | | | MP2K2 | | | PTN | |
| 0.641 | PH | CDON | CILP2 | H2A3 | | | NADK | | | |
| 0.641 | PH | CDON | | H2A3 | | MP2K2 | NADK | | | |
| 0.641 | PH | | | H2A3 | | MP2K2 | | Notch1 | | |
| 0.641 | PH | CDON | CILP2 | | | | NADK | Notch1 | | |
| 0.641 | PH | | CILP2 | | | | NADK | | | S100A13 |
| 0.641 | PH | | CILP2 | H2A3 | | | NADK | | | S100A13 |
| 0.641 | PH | CDON | | | | MP2K2 | | Notch1 | | S100A13 |
| 0.642 | PH | | CILP2 | | IGFALS | | | | | |
| 0.642 | PH | | | H2A3 | IGFALS | | NADK | | | |
| 0.642 | PH | | CILP2 | | | MP2K2 | NADK | | | |
| 0.642 | PH | | CILP2 | H2A3 | | | NADK | Notch1 | | |
| 0.642 | PH | | | H2A3 | | | | | PTN | S100A13 |
| 0.642 | PH | CDON | | | | MP2K2 | NADK | | | S100A13 |
| 0.642 | PH | | CILP2 | H2A3 | | | | Notch1 | | S100A13 |
| 0.643 | PH | CDON | | | IGFALS | MP2K2 | | | | |
| 0.643 | PH | | | | | | NADK | | PTN | |
| 0.643 | PH | | | | IGFALS | MP2K2 | NADK | | | |
| 0.643 | PH | CDON | | | IGFALS | | | Notch1 | | |
| 0.643 | PH | | | H2A3 | | MP2K2 | NADK | Notch1 | | |
| 0.643 | PH | CDON | | | | | | | PTN | S100A13 |
| 0.643 | PH | | | H2A3 | IGFALS | | | | | S100A13 |
| 0.643 | PH | CDON | | H2A3 | | | | Notch1 | | S100A13 |
| 0.644 | PH | | | H2A3 | | MP2K2 | | | PTN | |
| 0.644 | PH | | CILP2 | H2A3 | | MP2K2 | | | | |
| 0.644 | PH | | | | IGFALS | MP2K2 | | Notch1 | | |
| 0.644 | PH | CDON | | | IGFALS | | | | | S100A13 |
| 0.644 | PH | CDON | CILP2 | | | MP2K2 | | | | S100A13 |
| 0.644 | PH | | | | IGFALS | MP2K2 | | | | S100A13 |
| 0.644 | PH | | | H2A3 | | | NADK | Notch1 | | S100A13 |
| 0.645 | PH | | | H2A3 | IGFALS | MP2K2 | | | | |
| 0.645 | PH | CDON | CILP2 | H2A3 | | | NADK | Notch1 | | |
| 0.645 | PH | | CILP2 | | | MP2K2 | | Notch1 | | S100A13 |
| 0.645 | PH | CDON | | | | | NADK | Notch1 | | S100A13 |
| 0.646 | PH | | | H2A3 | | | NADK | | PTN | |
| 0.646 | PH | | | | | MP2K2 | NADK | | PTN | |
| 0.646 | PH | | CILP2 | | IGFALS | | | Notch1 | | |
| 0.646 | PH | CDON | | H2A3 | | MP2K2 | | Notch1 | | |
| 0.646 | PH | | CILP2 | | | MP2K2 | NADK | Notch1 | | |
| 0.646 | PH | CDON | | | | MP2K2 | | | PTN | S100A13 |
| 0.646 | PH | CDON | CILP2 | | | | NADK | | | S100A13 |
| 0.646 | PH | CDON | CILP2 | H2A3 | | | NADK | | | S100A13 |
| 0.646 | PH | CDON | | H2A3 | | MP2K2 | NADK | | | S100A13 |
| 0.646 | PH | CDON | CILP2 | H2A3 | | | | Notch1 | | S100A13 |
| 0.646 | PH | | | | | MP2K2 | NADK | Notch1 | | S100A13 |
| 0.647 | PH | CDON | CILP2 | | IGFALS | | | | | |
| 0.647 | PH | CDON | | H2A3 | IGFALS | | | | | |
| 0.647 | PH | CDON | | | IGFALS | | NADK | | | |
| 0.647 | PH | | CILP2 | H2A3 | | MP2K2 | NADK | | | |
| 0.647 | PH | | | H2A3 | IGFALS | MP2K2 | NADK | | | |
| 0.647 | PH | | | | IGFALS | | NADK | Notch1 | | |
| 0.647 | PH | | CILP2 | | IGFALS | | | | | S100A13 |
| 0.647 | PH | | CILP2 | H2A3 | | MP2K2 | | | | S100A13 |
| 0.647 | PH | | | H2A3 | | MP2K2 | | Notch1 | | S100A13 |
| 0.648 | PH | CDON | | H2A3 | | | | | PTN | |
| 0.648 | PH | CDON | CILP2 | | | MP2K2 | NADK | | | |
| 0.648 | PH | CDON | | | IGFALS | MP2K2 | | Notch1 | | |
| 0.648 | PH | CDON | | H2A3 | | MP2K2 | NADK | Notch1 | | |
| 0.648 | PH | | | H2A3 | | MP2K2 | | | PTN | S100A13 |
| 0.648 | PH | | | | | | NADK | | PTN | S100A13 |
| 0.648 | PH | | CILP2 | | | | NADK | Notch1 | | S100A13 |
| 0.649 | PH | | | | IGFALS | | | | PTN | |
| 0.649 | PH | | CILP2 | H2A3 | IGFALS | | | | | |
| 0.649 | PH | CDON | CILP2 | H2A3 | | MP2K2 | | | | |
| 0.649 | PH | | CILP2 | | IGFALS | MP2K2 | | | | |
| 0.649 | PH | CDON | | | | | NADK | | PTN | |
| 0.649 | PH | CDON | CILP2 | | IGFALS | | | Notch1 | | |
| 0.649 | PH | | | H2A3 | IGFALS | | | Notch1 | | |
| 0.649 | PH | CDON | CILP2 | | | MP2K2 | NADK | Notch1 | | |
| 0.649 | PH | | | | IGFALS | MP2K2 | NADK | Notch1 | | |
| 0.649 | PH | | | | IGFALS | | NADK | | | S100A13 |
| 0.649 | PH | | | | IGFALS | | | Notch1 | | S100A13 |
| 0.649 | PH | CDON | CILP2 | | | MP2K2 | | Notch1 | | S100A13 |
| 0.649 | PH | CDON | | H2A3 | | | NADK | Notch1 | | S100A13 |
| 0.65 | PH | CDON | | H2A3 | IGFALS | | NADK | | | |
| 0.65 | PH | | | H2A3 | | MP2K2 | NADK | | PTN | |
| 0.65 | PH | CDON | | | IGFALS | MP2K2 | NADK | | | |
| 0.65 | PH | | | | | | | Notch1 | PTN | |
| 0.65 | PH | | | | | MP2K2 | | Notch1 | PTN | |
| 0.65 | PH | | CILP2 | H2A3 | | MP2K2 | | Notch1 | | |
| 0.65 | PH | CDON | | | IGFALS | | NADK | Notch1 | | |
| 0.65 | PH | | | H2A3 | IGFALS | | NADK | | | S100A13 |
| 0.651 | PH | | | | IGFALS | MP2K2 | | | PTN | |
| 0.651 | PH | CDON | | | | MP2K2 | NADK | | PTN | |
| 0.651 | PH | CDON | | | | | | Notch1 | PTN | |
| 0.651 | PH | | | H2A3 | IGFALS | | NADK | Notch1 | | |
| 0.651 | PH | CDON | | H2A3 | IGFALS | | | | | S100A13 |
| 0.651 | PH | CDON | CILP2 | H2A3 | | MP2K2 | | | | S100A13 |
| 0.651 | PH | | | H2A3 | IGFALS | MP2K2 | | | | S100A13 |
| 0.651 | PH | | | H2A3 | | | NADK | | PTN | S100A13 |
| 0.651 | PH | | | | | MP2K2 | NADK | | PTN | S100A13 |
| 0.651 | PH | CDON | CILP2 | | | | NADK | Notch1 | | S100A13 |
| 0.651 | PH | | CILP2 | H2A3 | | | NADK | Notch1 | | S100A13 |
| 0.651 | PH | CDON | | | | MP2K2 | NADK | Notch1 | | S100A13 |
| 0.652 | PH | | CILP2 | | IGFALS | | NADK | | | |
| 0.652 | PH | CDON | CILP2 | H2A3 | | MP2K2 | NADK | | | |
| 0.652 | PH | | CILP2 | H2A3 | | MP2K2 | NADK | Notch1 | | |
| 0.652 | PH | CDON | | H2A3 | | | | | PTN | S100A13 |
| 0.652 | PH | CDON | CILP2 | | IGFALS | | | | | S100A13 |
| 0.652 | PH | | CILP2 | H2A3 | IGFALS | | | | | S100A13 |
| 0.652 | PH | CDON | | | IGFALS | MP2K2 | | | | S100A13 |
| 0.652 | PH | CDON | | H2A3 | | MP2K2 | | Notch1 | | S100A13 |
| 0.652 | PH | | | H2A3 | | MP2K2 | NADK | Notch1 | | S100A13 |
| 0.653 | PH | CDON | | H2A3 | | MP2K2 | | | PTN | |
| 0.653 | PH | | CILP2 | H2A3 | IGFALS | | NADK | | | |
| 0.653 | PH | CDON | | H2A3 | IGFALS | | | Notch1 | | |
| 0.653 | PH | CDON | | | | MP2K2 | | Notch1 | PTN | |
| 0.653 | PH | CDON | CILP2 | H2A3 | | MP2K2 | | Notch1 | | |
| 0.653 | PH | | CILP2 | | IGFALS | MP2K2 | | Notch1 | | |
| 0.653 | PH | | CILP2 | | | MP2K2 | NADK | | | S100A13 |
| 0.653 | PH | CDON | | | IGFALS | | | Notch1 | | S100A13 |
| 0.654 | PH | CDON | CILP2 | H2A3 | IGFALS | | | | | |
| 0.654 | PH | CDON | CILP2 | | IGFALS | MP2K2 | | | | |
| 0.654 | PH | CDON | | H2A3 | IGFALS | MP2K2 | | | | |
| 0.654 | PH | CDON | | H2A3 | | | NADK | | PTN | |
| 0.654 | PH | | CILP2 | H2A3 | IGFALS | | | Notch1 | | |
| 0.654 | PH | | CILP2 | | IGFALS | | NADK | Notch1 | | |
| 0.654 | PH | CDON | | | IGFALS | MP2K2 | NADK | Notch1 | | |
| 0.654 | PH | | | H2A3 | | MP2K2 | NADK | | PTN | S100A13 |
| 0.654 | PH | | CILP2 | H2A3 | | MP2K2 | NADK | | | S100A13 |
| 0.654 | PH | | CILP2 | | IGFALS | | | Notch1 | | S100A13 |
| 0.654 | PH | CDON | CILP2 | H2A3 | | | NADK | Notch1 | | S100A13 |
| 0.655 | PH | CDON | | H2A3 | IGFALS | MP2K2 | NADK | | | |
| 0.655 | PH | CDON | CILP2 | | IGFALS | MP2K2 | | Notch1 | | |
| 0.655 | PH | | | H2A3 | IGFALS | MP2K2 | | Notch1 | | |
| 0.655 | PH | CDON | | H2A3 | IGFALS | | NADK | Notch1 | | |
| 0.655 | PH | CDON | CILP2 | H2A3 | | MP2K2 | NADK | Notch1 | | |
| 0.655 | PH | CDON | | | | | NADK | | PTN | S100A13 |
| 0.655 | PH | CDON | | | IGFALS | | NADK | | | S100A13 |
| 0.655 | PH | | | | IGFALS | MP2K2 | NADK | | | S100A13 |
| 0.655 | PH | | CILP2 | H2A3 | | MP2K2 | | Notch1 | | S100A13 |
| 0.656 | PH | CDON | CILP2 | | IGFALS | | NADK | | | |
| 0.656 | PH | | CILP2 | | IGFALS | MP2K2 | NADK | | | |
| 0.656 | PH | CDON | CILP2 | H2A3 | IGFALS | | | Notch1 | | |
| 0.656 | PH | CDON | CILP2 | | IGFALS | | NADK | Notch1 | | |
| 0.656 | PH | | | H2A3 | IGFALS | MP2K2 | NADK | Notch1 | | |
| 0.656 | PH | CDON | CILP2 | H2A3 | IGFALS | | | | | S100A13 |
| 0.656 | PH | CDON | | H2A3 | | MP2K2 | | | PTN | S100A13 |
| 0.656 | PH | CDON | CILP2 | | | MP2K2 | NADK | | | S100A13 |
| 0.656 | PH | | | H2A3 | IGFALS | MP2K2 | NADK | | | S100A13 |
| 0.656 | PH | | | H2A3 | IGFALS | | | Notch1 | | S100A13 |
| 0.656 | PH | | | | IGFALS | MP2K2 | | Notch1 | | S100A13 |
| 0.656 | PH | CDON | | H2A3 | | MP2K2 | NADK | Notch1 | | S100A13 |
| 0.657 | PH | CDON | | | IGFALS | | | | PTN | |
| 0.657 | PH | | | H2A3 | IGFALS | | | | PTN | |
| 0.657 | PH | | CILP2 | H2A3 | IGFALS | MP2K2 | | | | |
| 0.657 | PH | CDON | CILP2 | H2A3 | IGFALS | | NADK | | | |
| 0.657 | PH | CDON | | H2A3 | | MP2K2 | NADK | | PTN | |
| 0.657 | PH | | CILP2 | H2A3 | IGFALS | | NADK | Notch1 | | |
| 0.657 | PH | | | | IGFALS | | | | PTN | S100A13 |
| 0.657 | PH | | CILP2 | | IGFALS | MP2K2 | | | | S100A13 |
| 0.657 | PH | CDON | | H2A3 | IGFALS | | NADK | | | S100A13 |
| 0.657 | PH | CDON | | | | MP2K2 | NADK | | PTN | S100A13 |
| 0.657 | PH | CDON | CILP2 | H2A3 | | MP2K2 | NADK | | | S100A13 |
| 0.657 | PH | CDON | CILP2 | | IGFALS | | | Notch1 | | S100A13 |
| 0.657 | PH | CDON | CILP2 | H2A3 | | MP2K2 | | Notch1 | | S100A13 |
| 0.657 | PH | | CILP2 | | | MP2K2 | NADK | Notch1 | | S100A13 |
| 0.658 | PH | CDON | | | IGFALS | MP2K2 | | | PTN | |
| 0.658 | PH | CDON | | H2A3 | IGFALS | MP2K2 | | | | S100A13 |
| 0.658 | PH | | | | | | | Notch1 | PTN | S100A13 |
| 0.658 | PH | | | | IGFALS | | NADK | Notch1 | | S100A13 |
| 0.659 | PH | | | H2A3 | IGFALS | MP2K2 | | | PTN | |
| 0.659 | PH | | | | IGFALS | | NADK | | PTN | |
| 0.659 | PH | | CILP2 | H2A3 | IGFALS | MP2K2 | NADK | | | |
| 0.659 | PH | CDON | | H2A3 | IGFALS | MP2K2 | | Notch1 | | |
| 0.659 | PH | CDON | CILP2 | H2A3 | IGFALS | | NADK | Notch1 | | |
| 0.659 | PH | | CILP2 | | IGFALS | MP2K2 | NADK | Notch1 | | |
| 0.659 | PH | CDON | | H2A3 | IGFALS | MP2K2 | NADK | Notch1 | | |
| 0.659 | PH | CDON | | H2A3 | | | NADK | | PTN | S100A13 |
| 0.659 | PH | | CILP2 | H2A3 | IGFALS | | NADK | | | S100A13 |
| 0.659 | PH | CDON | | | IGFALS | MP2K2 | NADK | | | S100A13 |
| 0.659 | PH | CDON | | H2A3 | IGFALS | | | Notch1 | | S100A13 |
| 0.659 | PH | | | | | MP2K2 | | Notch1 | PTN | S100A13 |
| 0.659 | PH | CDON | | | IGFALS | MP2K2 | | Notch1 | | S100A13 |
| 0.659 | PH | CDON | CILP2 | | | MP2K2 | NADK | Notch1 | | S100A13 |
| 0.66 | PH | | | | IGFALS | MP2K2 | NADK | | PTN | |
| 0.66 | PH | CDON | CILP2 | | IGFALS | MP2K2 | NADK | | | |
| 0.66 | PH | | | | | | NADK | Notch1 | PTN | |
| 0.66 | PH | | | | | MP2K2 | NADK | Notch1 | PTN | |
| 0.66 | PH | | | | IGFALS | MP2K2 | | | PTN | S100A13 |
| 0.66 | PH | | CILP2 | | IGFALS | | NADK | | | S100A13 |
| 0.66 | PH | | CILP2 | H2A3 | IGFALS | | | Notch1 | | S100A13 |
| 0.661 | PH | | CILP2 | | | | | | PTN | |
| 0.661 | PH | CDON | | | | | NADK | Notch1 | PTN | |
| 0.661 | PH | CDON | CILP2 | | IGFALS | MP2K2 | NADK | Notch1 | | |
| 0.661 | PH | CDON | CILP2 | | IGFALS | MP2K2 | | | | S100A13 |
| 0.661 | PH | CDON | | H2A3 | | MP2K2 | NADK | | PTN | S100A13 |
| 0.661 | PH | CDON | | | | | | Notch1 | PTN | S100A13 |
| 0.661 | PH | | | H2A3 | IGFALS | | NADK | Notch1 | | S100A13 |
| 0.661 | PH | | CILP2 | H2A3 | | MP2K2 | NADK | Notch1 | | S100A13 |
| 0.662 | PH | CDON | CILP2 | H2A3 | IGFALS | MP2K2 | | | | |
| 0.662 | PH | | | H2A3 | IGFALS | | NADK | | PTN | |
| 0.662 | PH | | | H2A3 | | | | Notch1 | PTN | |
| 0.662 | PH | | CILP2 | H2A3 | IGFALS | MP2K2 | | Notch1 | | |
| 0.662 | PH | CDON | | | | MP2K2 | NADK | Notch1 | PTN | |
| 0.662 | PH | | CILP2 | H2A3 | IGFALS | MP2K2 | | | | S100A13 |
| 0.662 | PH | CDON | CILP2 | | IGFALS | | NADK | | | S100A13 |
| 0.662 | PH | CDON | CILP2 | H2A3 | IGFALS | | NADK | | | S100A13 |
| 0.662 | PH | CDON | | H2A3 | IGFALS | MP2K2 | NADK | | | S100A13 |
| 0.662 | PH | CDON | CILP2 | H2A3 | IGFALS | | | Notch1 | | S100A13 |
| 0.662 | PH | CDON | | | | MP2K2 | | Notch1 | PTN | S100A13 |
| 0.662 | PH | CDON | | | IGFALS | | NADK | Notch1 | | S100A13 |
| 0.662 | PH | CDON | CILP2 | H2A3 | | MP2K2 | NADK | Notch1 | | S100A13 |
| 0.663 | PH | CDON | CILP2 | | | | | | PTN | |
| 0.663 | PH | | | H2A3 | IGFALS | MP2K2 | NADK | | PTN | |
| 0.663 | PH | CDON | CILP2 | H2A3 | IGFALS | MP2K2 | NADK | | | |
| 0.663 | PH | | CILP2 | H2A3 | IGFALS | MP2K2 | NADK | Notch1 | | |
| 0.663 | PH | CDON | | | IGFALS | | | | PTN | S100A13 |
| 0.663 | PH | | | H2A3 | IGFALS | | | | PTN | S100A13 |
| 0.663 | PH | | CILP2 | | IGFALS | MP2K2 | | Notch1 | | S100A13 |
| 0.663 | PH | | | H2A3 | IGFALS | MP2K2 | | Notch1 | | S100A13 |
| 0.663 | PH | | | | IGFALS | MP2K2 | NADK | Notch1 | | S100A13 |
| 0.664 | PH | | CILP2 | H2A3 | | | | | PTN | |
| 0.664 | PH | CDON | | H2A3 | | | | Notch1 | PTN | |
| 0.664 | PH | | | H2A3 | | MP2K2 | | Notch1 | PTN | |
| 0.664 | PH | CDON | CILP2 | H2A3 | IGFALS | MP2K2 | | Notch1 | | |
| 0.664 | PH | | CILP2 | | IGFALS | | NADK | Notch1 | | S100A13 |
| 0.664 | PH | CDON | | H2A3 | IGFALS | | NADK | Notch1 | | S100A13 |
| 0.665 | PH | CDON | | H2A3 | IGFALS | | | | PTN | |
| 0.665 | PH | CDON | | | IGFALS | | NADK | | PTN | |
| 0.665 | PH | CDON | | | IGFALS | MP2K2 | NADK | | PTN | |
| 0.665 | PH | CDON | CILP2 | H2A3 | IGFALS | MP2K2 | NADK | Notch1 | | |
| 0.665 | PH | | CILP2 | | | | | | PTN | S100A13 |
| 0.665 | PH | CDON | | | IGFALS | MP2K2 | | | PTN | S100A13 |
| 0.665 | PH | | | H2A3 | IGFALS | MP2K2 | | | PTN | S100A13 |
| 0.665 | PH | CDON | CILP2 | H2A3 | IGFALS | MP2K2 | | | | S100A13 |
| 0.665 | PH | CDON | CILP2 | | IGFALS | MP2K2 | | Notch1 | | S100A13 |
| 0.665 | PH | | CILP2 | H2A3 | IGFALS | | NADK | Notch1 | | S100A13 |
| 0.665 | PH | CDON | | | IGFALS | MP2K2 | NADK | Notch1 | | S100A13 |
| 0.666 | PH | CDON | | H2A3 | | MP2K2 | | Notch1 | PTN | |
| 0.666 | PH | | | H2A3 | | | NADK | Notch1 | PTN | |
| 0.666 | PH | CDON | CILP2 | | | | | | PTN | S100A13 |
| 0.666 | PH | | CILP2 | | IGFALS | MP2K2 | NADK | | | S100A13 |
| 0.666 | PH | CDON | | H2A3 | IGFALS | MP2K2 | | Notch1 | | S100A13 |
| 0.666 | PH | CDON | CILP2 | | IGFALS | | NADK | Notch1 | | S100A13 |
| 0.666 | PH | | | H2A3 | IGFALS | MP2K2 | NADK | Notch1 | | S100A13 |
| 0.667 | PH | CDON | CILP2 | H2A3 | | | | | PTN | |
| 0.667 | PH | | CILP2 | | | MP2K2 | | | PTN | |
| 0.667 | PH | CDON | | H2A3 | IGFALS | MP2K2 | | | PTN | |
| 0.667 | PH | | | | IGFALS | | | Notch1 | PTN | |
| 0.667 | PH | | | | IGFALS | MP2K2 | | Notch1 | PTN | |
| 0.667 | PH | CDON | | H2A3 | | | NADK | Notch1 | PTN | |
| 0.667 | PH | | | H2A3 | | MP2K2 | NADK | Notch1 | PTN | |
| 0.667 | PH | | CILP2 | H2A3 | IGFALS | MP2K2 | NADK | | | S100A13 |
| 0.667 | PH | CDON | CILP2 | H2A3 | IGFALS | | NADK | Notch1 | | S100A13 |
| 0.668 | PH | CDON | CILP2 | | | MP2K2 | | | PTN | |
| 0.668 | PH | CDON | | H2A3 | IGFALS | | NADK | | PTN | |
| 0.668 | PH | CDON | | | IGFALS | | | Notch1 | PTN | |
| 0.668 | PH | | CILP2 | H2A3 | | | | | PTN | S100A13 |
| 0.668 | PH | | | | IGFALS | | NADK | | PTN | S100A13 |
| 0.668 | PH | CDON | CILP2 | | IGFALS | MP2K2 | NADK | | | S100A13 |
| 0.668 | PH | | | H2A3 | | | | Notch1 | PTN | S100A13 |
| 0.668 | PH | | CILP2 | H2A3 | IGFALS | MP2K2 | | Notch1 | | S100A13 |
| 0.668 | PH | | | | | | NADK | Notch1 | PTN | S100A13 |
| 0.668 | PH | CDON | | H2A3 | IGFALS | MP2K2 | NADK | Notch1 | | S100A13 |
| 0.669 | PH | CDON | | H2A3 | IGFALS | MP2K2 | NADK | | PTN | |
| 0.669 | PH | CDON | | | IGFALS | MP2K2 | | Notch1 | PTN | |
| 0.669 | PH | CDON | | H2A3 | | MP2K2 | NADK | Notch1 | PTN | |
| 0.669 | PH | CDON | | H2A3 | IGFALS | | | | PTN | S100A13 |
| 0.669 | PH | | | H2A3 | IGFALS | | NADK | | PTN | S100A13 |
| 0.669 | PH | | | | IGFALS | MP2K2 | NADK | | PTN | S100A13 |
| 0.669 | PH | CDON | CILP2 | H2A3 | IGFALS | MP2K2 | NADK | | | S100A13 |
| 0.669 | PH | CDON | CILP2 | H2A3 | IGFALS | MP2K2 | | Notch1 | | S100A13 |
| 0.669 | PH | | | | | MP2K2 | NADK | Notch1 | PTN | S100A13 |
| 0.67 | PH | | CILP2 | H2A3 | | MP2K2 | | | PTN | |
| 0.67 | PH | | CILP2 | H2A3 | | | NADK | | PTN | |
| 0.67 | PH | CDON | CILP2 | | | | | Notch1 | PTN | |
| 0.67 | PH | CDON | CILP2 | H2A3 | | | | | PTN | S100A13 |
| 0.67 | PH | | CILP2 | | | MP2K2 | | | PTN | S100A13 |
| 0.67 | PH | | | H2A3 | IGFALS | MP2K2 | NADK | | PTN | S100A13 |
| 0.67 | PH | CDON | | H2A3 | | | | Notch1 | PTN | S100A13 |
| 0.67 | PH | | | H2A3 | | MP2K2 | | Notch1 | PTN | S100A13 |
| 0.67 | PH | CDON | | | | | NADK | Notch1 | PTN | S100A13 |
| 0.67 | PH | CDON | | | | MP2K2 | NADK | Notch1 | PTN | S100A13 |
| 0.67 | PH | | CILP2 | | IGFALS | MP2K2 | NADK | Notch1 | | S100A13 |
| 0.671 | PH | | CILP2 | | | | NADK | | PTN | |
| 0.671 | PH | | CILP2 | | | | | Notch1 | PTN | |
| 0.671 | PH | CDON | CILP2 | | | MP2K2 | | | PTN | S100A13 |
| 0.671 | PH | CDON | | H2A3 | IGFALS | MP2K2 | | | PTN | S100A13 |
| 0.671 | PH | CDON | CILP2 | | IGFALS | MP2K2 | NADK | Notch1 | | S100A13 |
| 0.672 | PH | CDON | CILP2 | H2A3 | | MP2K2 | | | PTN | |
| 0.672 | PH | CDON | CILP2 | | | | NADK | | PTN | |
| 0.672 | PH | CDON | CILP2 | H2A3 | | | NADK | | PTN | |
| 0.672 | PH | CDON | | | IGFALS | | NADK | | PTN | S100A13 |
| 0.672 | PH | CDON | | | IGFALS | MP2K2 | NADK | | PTN | S100A13 |
| 0.672 | PH | CDON | | H2A3 | | MP2K2 | | Notch1 | PTN | S100A13 |
| 0.672 | PH | | | H2A3 | | | NADK | Notch1 | PTN | S100A13 |
| 0.672 | PH | | CILP2 | H2A3 | IGFALS | MP2K2 | NADK | Notch1 | | S100A13 |
| 0.673 | PH | CDON | CILP2 | | | MP2K2 | | Notch1 | PTN | |
| 0.673 | PH | | | | IGFALS | | NADK | Notch1 | PTN | |
| 0.673 | PH | | | | IGFALS | MP2K2 | NADK | Notch1 | PTN | |
| 0.673 | PH | | CILP2 | H2A3 | | MP2K2 | | | PTN | S100A13 |
| 0.673 | PH | | | H2A3 | | MP2K2 | NADK | Notch1 | PTN | S100A13 |
| 0.673 | PH | CDON | CILP2 | H2A3 | IGFALS | MP2K2 | NADK | Notch1 | | S100A13 |
| 0.674 | PH | | CILP2 | H2A3 | | MP2K2 | NADK | | PTN | |
| 0.674 | PH | | CILP2 | | | MP2K2 | | Notch1 | PTN | |
| 0.674 | PH | CDON | | | IGFALS | | NADK | Notch1 | PTN | |
| 0.674 | PH | CDON | | | IGFALS | MP2K2 | NADK | Notch1 | PTN | |
| 0.674 | PH | | CILP2 | H2A3 | | | NADK | | PTN | S100A13 |
| 0.674 | PH | CDON | | H2A3 | IGFALS | | NADK | | PTN | S100A13 |
| 0.674 | PH | | CILP2 | | | | | Notch1 | PTN | S100A13 |
| 0.674 | PH | CDON | CILP2 | | | | | Notch1 | PTN | S100A13 |
| 0.674 | PH | CDON | | H2A3 | | | NADK | Notch1 | PTN | S100A13 |
| 0.675 | PH | | CILP2 | | | MP2K2 | NADK | | PTN | |
| 0.675 | PH | CDON | CILP2 | | | MP2K2 | NADK | | PTN | |
| 0.675 | PH | | | H2A3 | IGFALS | | | Notch1 | PTN | |
| 0.675 | PH | CDON | CILP2 | H2A3 | | MP2K2 | | | PTN | S100A13 |
| 0.675 | PH | CDON | | H2A3 | IGFALS | MP2K2 | NADK | | PTN | S100A13 |
| 0.675 | PH | | | | IGFALS | | | Notch1 | PTN | S100A13 |
| 0.675 | PH | CDON | | H2A3 | | MP2K2 | NADK | Notch1 | PTN | S100A13 |
| 0.676 | PH | | CILP2 | | IGFALS | | | | PTN | |
| 0.676 | PH | CDON | CILP2 | | IGFALS | | | | PTN | |
| 0.676 | PH | CDON | CILP2 | H2A3 | | MP2K2 | NADK | | PTN | |
| 0.676 | PH | | CILP2 | H2A3 | | | | Notch1 | PTN | |
| 0.676 | PH | CDON | CILP2 | H2A3 | | | | Notch1 | PTN | |
| 0.676 | PH | | | H2A3 | IGFALS | MP2K2 | | Notch1 | PTN | |
| 0.676 | PH | | CILP2 | | | | NADK | | PTN | S100A13 |
| 0.676 | PH | CDON | CILP2 | | | | NADK | | PTN | S100A13 |
| 0.676 | PH | CDON | CILP2 | H2A3 | | | NADK | | PTN | S100A13 |
| 0.676 | PH | CDON | | | IGFALS | | | Notch1 | PTN | S100A13 |
| 0.676 | PH | | | | IGFALS | MP2K2 | | Notch1 | PTN | S100A13 |
| 0.677 | PH | | CILP2 | H2A3 | IGFALS | | | | PTN | |
| 0.677 | PH | CDON | | H2A3 | IGFALS | | | Notch1 | PTN | |
| 0.677 | PH | CDON | | H2A3 | IGFALS | MP2K2 | | Notch1 | PTN | |
| 0.677 | PH | | | H2A3 | IGFALS | | NADK | Notch1 | PTN | |
| 0.677 | PH | | | H2A3 | IGFALS | MP2K2 | NADK | Notch1 | PTN | |
| 0.677 | PH | CDON | CILP2 | | | MP2K2 | | Notch1 | PTN | S100A13 |
| 0.677 | PH | CDON | | | IGFALS | MP2K2 | | Notch1 | PTN | S100A13 |
| 0.678 | PH | | CILP2 | | IGFALS | MP2K2 | | | PTN | |
| 0.678 | PH | CDON | CILP2 | | | | NADK | Notch1 | PTN | |
| 0.678 | PH | CDON | | H2A3 | IGFALS | | NADK | Notch1 | PTN | |
| 0.678 | PH | | CILP2 | | | MP2K2 | | Notch1 | PTN | S100A13 |
| 0.679 | PH | CDON | CILP2 | H2A3 | IGFALS | | | | PTN | |
| 0.679 | PH | CDON | CILP2 | | IGFALS | MP2K2 | | | PTN | |
| 0.679 | PH | CDON | CILP2 | H2A3 | | MP2K2 | | Notch1 | PTN | |
| 0.679 | PH | | CILP2 | | | | NADK | Notch1 | PTN | |
| 0.679 | PH | CDON | | H2A3 | IGFALS | MP2K2 | NADK | Notch1 | PTN | |
| 0.679 | PH | CDON | CILP2 | | | MP2K2 | NADK | | PTN | S100A13 |
| 0.679 | PH | | CILP2 | H2A3 | | MP2K2 | NADK | | PTN | S100A13 |
| 0.679 | PH | CDON | CILP2 | H2A3 | | MP2K2 | NADK | | PTN | S100A13 |
| 0.68 | PH | | CILP2 | H2A3 | IGFALS | MP2K2 | | | PTN | |
| 0.68 | PH | | CILP2 | H2A3 | | MP2K2 | | Notch1 | PTN | |
| 0.68 | PH | | CILP2 | H2A3 | | | NADK | Notch1 | PTN | |
| 0.68 | PH | CDON | CILP2 | H2A3 | | | NADK | Notch1 | PTN | |
| 0.68 | PH | CDON | CILP2 | | | MP2K2 | NADK | Notch1 | PTN | |
| 0.68 | PH | | CILP2 | | IGFALS | | | | PTN | S100A13 |
| 0.68 | PH | CDON | CILP2 | | IGFALS | | | | PTN | S100A13 |
| 0.68 | PH | | CILP2 | | | MP2K2 | NADK | | PTN | S100A13 |
| 0.68 | PH | | CILP2 | H2A3 | | | | Notch1 | PTN | S100A13 |
| 0.68 | PH | CDON | CILP2 | H2A3 | | | | Notch1 | PTN | S100A13 |
| 0.681 | PH | | CILP2 | H2A3 | IGFALS | | NADK | | PTN | |
| 0.681 | PH | | CILP2 | | | MP2K2 | NADK | Notch1 | PTN | |
| 0.681 | PH | | CILP2 | H2A3 | IGFALS | | | | PTN | S100A13 |
| 0.681 | PH | | | H2A3 | IGFALS | | | Notch1 | PTN | S100A13 |
| 0.682 | PH | CDON | CILP2 | H2A3 | IGFALS | MP2K2 | | | PTN | |
| 0.682 | PH | | CILP2 | | IGFALS | | NADK | | PTN | |
| 0.682 | PH | CDON | CILP2 | | IGFALS | | NADK | | PTN | |
| 0.682 | PH | CDON | CILP2 | H2A3 | IGFALS | | NADK | | PTN | |
| 0.682 | PH | CDON | CILP2 | | IGFALS | | | Notch1 | PTN | |
| 0.682 | PH | CDON | CILP2 | H2A3 | | MP2K2 | NADK | Notch1 | PTN | |
| 0.682 | PH | CDON | CILP2 | H2A3 | IGFALS | | | | PTN | S100A13 |
| 0.682 | PH | CDON | | H2A3 | IGFALS | | | Notch1 | PTN | S100A13 |
| 0.682 | PH | | | H2A3 | IGFALS | MP2K2 | | Notch1 | PTN | S100A13 |
| 0.682 | PH | | | | IGFALS | | NADK | Notch1 | PTN | S100A13 |
| 0.682 | PH | CDON | | | IGFALS | | NADK | Notch1 | PTN | S100A13 |
| 0.682 | PH | | | | IGFALS | MP2K2 | NADK | Notch1 | PTN | S100A13 |
| 0.683 | PH | CDON | CILP2 | | IGFALS | MP2K2 | NADK | | PTN | |
| 0.683 | PH | | CILP2 | H2A3 | IGFALS | MP2K2 | NADK | | PTN | |
| 0.683 | PH | | CILP2 | | IGFALS | | | Notch1 | PTN | |
| 0.683 | PH | | CILP2 | H2A3 | | MP2K2 | NADK | Notch1 | PTN | |
| 0.683 | PH | | CILP2 | | IGFALS | MP2K2 | | | PTN | S100A13 |
| 0.683 | PH | CDON | CILP2 | | IGFALS | MP2K2 | | | PTN | S100A13 |
| 0.683 | PH | CDON | CILP2 | H2A3 | | MP2K2 | | Notch1 | PTN | S100A13 |
| 0.683 | PH | CDON | | H2A3 | IGFALS | MP2K2 | | Notch1 | PTN | S100A13 |
| 0.683 | PH | CDON | CILP2 | | | | NADK | Notch1 | PTN | S100A13 |
| 0.683 | PH | CDON | | | IGFALS | MP2K2 | NADK | Notch1 | PTN | S100A13 |
| 0.684 | PH | | CILP2 | | IGFALS | MP2K2 | NADK | | PTN | |
| 0.684 | PH | CDON | CILP2 | H2A3 | IGFALS | MP2K2 | NADK | | PTN | |
| 0.684 | PH | CDON | CILP2 | | IGFALS | MP2K2 | | Notch1 | PTN | |
| 0.684 | PH | | CILP2 | H2A3 | IGFALS | MP2K2 | | | PTN | S100A13 |
| 0.684 | PH | | CILP2 | H2A3 | | MP2K2 | | Notch1 | PTN | S100A13 |
| 0.684 | PH | | CILP2 | | | | NADK | Notch1 | PTN | S100A13 |
| 0.685 | PH | | CILP2 | | IGFALS | MP2K2 | | Notch1 | PTN | |
| 0.685 | PH | CDON | CILP2 | H2A3 | IGFALS | MP2K2 | | | PTN | S100A13 |
| 0.685 | PH | | CILP2 | H2A3 | | | NADK | Notch1 | PTN | S100A13 |
| 0.685 | PH | CDON | CILP2 | H2A3 | | | NADK | Notch1 | PTN | S100A13 |
| 0.685 | PH | | | H2A3 | IGFALS | | NADK | Notch1 | PTN | S100A13 |
| 0.685 | PH | CDON | | H2A3 | IGFALS | | NADK | Notch1 | PTN | S100A13 |
| 0.685 | PH | CDON | CILP2 | | | MP2K2 | NADK | Notch1 | PTN | S100A13 |
| 0.685 | PH | | | H2A3 | IGFALS | MP2K2 | NADK | Notch1 | PTN | S100A13 |
| 0.685 | PH | CDON | | H2A3 | IGFALS | MP2K2 | NADK | Notch1 | PTN | S100A13 |
| 0.686 | PH | | CILP2 | H2A3 | IGFALS | | | Notch1 | PTN | |
| 0.686 | PH | CDON | CILP2 | H2A3 | IGFALS | | | Notch1 | PTN | |
| 0.686 | PH | | CILP2 | H2A3 | IGFALS | | NADK | | PTN | S100A13 |
| 0.686 | PH | CDON | CILP2 | H2A3 | IGFALS | | NADK | | PTN | S100A13 |
| 0.686 | PH | CDON | CILP2 | | IGFALS | | | Notch1 | PTN | S100A13 |
| 0.686 | PH | | CILP2 | | | MP2K2 | NADK | Notch1 | PTN | S100A13 |
| 0.687 | PH | CDON | CILP2 | | IGFALS | | NADK | Notch1 | PTN | |
| 0.687 | PH | | CILP2 | | IGFALS | | NADK | | PTN | S100A13 |
| 0.687 | PH | CDON | CILP2 | | IGFALS | | NADK | | PTN | S100A13 |
| 0.687 | PH | | CILP2 | | IGFALS | | | Notch1 | PTN | S100A13 |
| 0.687 | PH | CDON | CILP2 | H2A3 | | MP2K2 | NADK | Notch1 | PTN | S100A13 |
| 0.688 | PH | | CILP2 | H2A3 | IGFALS | MP2K2 | | Notch1 | PTN | |
| 0.688 | PH | CDON | CILP2 | H2A3 | IGFALS | MP2K2 | | Notch1 | PTN | |
| 0.688 | PH | | CILP2 | | IGFALS | | NADK | Notch1 | PTN | |
| 0.688 | PH | CDON | CILP2 | H2A3 | IGFALS | | NADK | Notch1 | PTN | |
| 0.688 | PH | CDON | CILP2 | | IGFALS | MP2K2 | NADK | Notch1 | PTN | |
| 0.688 | PH | | CILP2 | H2A3 | IGFALS | MP2K2 | NADK | | PTN | S100A13 |
| 0.688 | PH | CDON | CILP2 | H2A3 | IGFALS | MP2K2 | NADK | | PTN | S100A13 |
| 0.688 | PH | CDON | CILP2 | | IGFALS | MP2K2 | | Notch1 | PTN | S100A13 |
| 0.688 | PH | | CILP2 | H2A3 | | MP2K2 | NADK | Notch1 | PTN | S100A13 |
| 0.689 | PH | | CILP2 | H2A3 | IGFALS | | NADK | Notch1 | PTN | |
| 0.689 | PH | | CILP2 | | IGFALS | MP2K2 | NADK | Notch1 | PTN | |
| 0.689 | PH | CDON | CILP2 | H2A3 | IGFALS | MP2K2 | NADK | Notch1 | PTN | |
| 0.689 | PH | | CILP2 | | IGFALS | MP2K2 | NADK | | PTN | S100A13 |
| 0.689 | PH | CDON | CILP2 | | IGFALS | MP2K2 | NADK | | PTN | S100A13 |
| 0.69 | PH | | CILP2 | H2A3 | IGFALS | MP2K2 | NADK | Notch1 | PTN | |
| 0.69 | PH | CDON | CILP2 | H2A3 | IGFALS | | | Notch1 | PTN | S100A13 |
| 0.69 | PH | | CILP2 | | IGFALS | MP2K2 | | Notch1 | PTN | S100A13 |
| 0.691 | PH | | CILP2 | H2A3 | IGFALS | | | Notch1 | PTN | S100A13 |
| 0.692 | PH | CDON | CILP2 | H2A3 | IGFALS | MP2K2 | | Notch1 | PTN | S100A13 |
| 0.692 | PH | CDON | CILP2 | | IGFALS | | NADK | Notch1 | PTN | S100A13 |
| 0.693 | PH | | CILP2 | H2A3 | IGFALS | MP2K2 | | Notch1 | PTN | S100A13 |
| 0.693 | PH | CDON | CILP2 | H2A3 | IGFALS | | NADK | Notch1 | PTN | S100A13 |
| 0.694 | PH | | CILP2 | | IGFALS | | NADK | Notch1 | PTN | S100A13 |
| 0.694 | PH | | CILP2 | H2A3 | IGFALS | | NADK | Notch1 | PTN | S100A13 |
| 0.694 | PH | CDON | CILP2 | | IGFALS | MP2K2 | NADK | Notch1 | PTN | S100A13 |
| 0.694 | PH | CDON | CILP2 | H2A3 | IGFALS | MP2K2 | NADK | Notch1 | PTN | S100A13 |
| 0.695 | PH | | CILP2 | | IGFALS | MP2K2 | NADK | Notch1 | PTN | S100A13 |
| *0.696 | PH | | CILP2 | H2A3 | IGFALS | MP2K2 | NADK | Notch1 | PTN | S100A13 |

*Included in Table 38

TABLE 41
Notch1 Biomarker Panels-20 Year Model

| AUC | Notch1 | CDON | CILP2 | H2A3 | IGFALS | MP2K2 | NADK | PH | PTN | S100A13 |
|---|---|---|---|---|---|---|---|---|---|---|
| 0.583 | Notch1 | CDON | | | | | | | | |
| 0.588 | Notch1 | | | | | MP2K2 | | | | |
| 0.589 | Notch1 | | | H2A3 | | | | | | |
| 0.59 | Notch1 | | CILP2 | | | | | | | |
| 0.593 | Notch1 | | | | | | NADK | | | |
| 0.594 | Notch1 | | | | | | | | | S100A13 |
| 0.596 | Notch1 | CDON | CILP2 | | | | | | | |
| 0.598 | Notch1 | CDON | | | | MP2K2 | | | | |
| 0.599 | Notch1 | CDON | | H2A3 | | | | | | |
| 0.599 | Notch1 | | | H2A3 | | | NADK | | | |
| 0.6 | Notch1 | CDON | | | | | NADK | | | |
| 0.602 | Notch1 | | CILP2 | H2A3 | | | | | | |
| 0.603 | Notch1 | | | | | MP2K2 | NADK | | | |
| 0.605 | Notch1 | CDON | | | | | | | | S100A13 |
| 0.606 | Notch1 | | | H2A3 | | | | | | S100A13 |
| 0.607 | Notch1 | | CILP2 | | | MP2K2 | | | | |
| 0.607 | Notch1 | | | H2A3 | | MP2K2 | | | | |
| 0.607 | Notch1 | CDON | | H2A3 | | | NADK | | | |
| 0.607 | Notch1 | | CILP2 | | | | | | | S100A13 |
| 0.608 | Notch1 | CDON | CILP2 | H2A3 | | | | | | |
| 0.608 | Notch1 | | | | | MP2K2 | | | | S100A13 |
| 0.609 | Notch1 | | CILP2 | | | | NADK | | | |
| 0.61 | Notch1 | | | | IGFALS | | | | | |
| 0.61 | Notch1 | CDON | | | | MP2K2 | NADK | | | |
| 0.611 | Notch1 | | | H2A3 | | MP2K2 | NADK | | | |
| 0.612 | Notch1 | CDON | CILP2 | | | MP2K2 | | | | |
| 0.612 | Notch1 | | CILP2 | H2A3 | | | NADK | | | |
| 0.613 | Notch1 | CDON | CILP2 | | | | NADK | | | |
| 0.613 | Notch1 | CDON | CILP2 | | | | | | | S100A13 |
| 0.613 | Notch1 | | | | | | NADK | | | S100A13 |
| 0.614 | Notch1 | | | | | | | PH | | |
| 0.615 | Notch1 | CDON | | H2A3 | | MP2K2 | | | | |
| 0.615 | Notch1 | CDON | | H2A3 | | | | | | S100A13 |
| 0.615 | Notch1 | | CILP2 | H2A3 | | | | | | S100A13 |
| 0.616 | Notch1 | CDON | | | IGFALS | | | | | |
| 0.617 | Notch1 | CDON | CILP2 | H2A3 | | | NADK | | | |
| 0.617 | Notch1 | CDON | | | | MP2K2 | | | | S100A13 |
| 0.617 | Notch1 | | | H2A3 | | | NADK | | | S100A13 |
| 0.619 | Notch1 | | | H2A3 | IGFALS | | | | | |
| 0.619 | Notch1 | | | | IGFALS | MP2K2 | | | | |
| 0.619 | Notch1 | CDON | | H2A3 | | MP2K2 | NADK | | | |
| 0.62 | Notch1 | | CILP2 | | | MP2K2 | NADK | | | |
| 0.62 | Notch1 | CDON | | | | | | PH | | |
| 0.621 | Notch1 | | CILP2 | | IGFALS | | | | | |
| 0.621 | Notch1 | | CILP2 | H2A3 | | MP2K2 | | | | |
| 0.621 | Notch1 | | | | IGFALS | | NADK | | | |
| 0.621 | Notch1 | CDON | CILP2 | H2A3 | | | | | | S100A13 |
| 0.621 | Notch1 | | | H2A3 | | MP2K2 | | | | S100A13 |
| 0.621 | Notch1 | CDON | | | | | NADK | | | S100A13 |
| 0.622 | Notch1 | | | | | MP2K2 | | PH | | |
| 0.622 | Notch1 | | | | | MP2K2 | NADK | | | S100A13 |
| 0.623 | Notch1 | | CILP2 | | | MP2K2 | | | | S100A13 |
| 0.624 | Notch1 | CDON | CILP2 | | IGFALS | | | | | |
| 0.624 | Notch1 | CDON | | | IGFALS | MP2K2 | | | | |
| 0.624 | Notch1 | | CILP2 | | | | | PH | | |
| 0.624 | Notch1 | CDON | | H2A3 | | | NADK | | | S100A13 |
| 0.624 | Notch1 | | | | | | | PH | | S100A13 |
| 0.625 | Notch1 | CDON | | H2A3 | IGFALS | | | | | |
| 0.625 | Notch1 | | | H2A3 | IGFALS | | NADK | | | |
| 0.625 | Notch1 | CDON | CILP2 | | | MP2K2 | NADK | | | |
| 0.625 | Notch1 | | | | IGFALS | MP2K2 | NADK | | | |
| 0.625 | Notch1 | | CILP2 | | | | NADK | | | S100A13 |
| 0.626 | Notch1 | CDON | CILP2 | H2A3 | | MP2K2 | | | | |
| 0.626 | Notch1 | CDON | | | IGFALS | | NADK | | | |
| 0.626 | Notch1 | | CILP2 | H2A3 | | MP2K2 | NADK | | | |
| 0.627 | Notch1 | CDON | CILP2 | | | MP2K2 | | | | S100A13 |
| 0.627 | Notch1 | CDON | | H2A3 | | MP2K2 | | | | S100A13 |
| 0.627 | Notch1 | | CILP2 | H2A3 | | | NADK | | | S100A13 |
| 0.627 | Notch1 | | | H2A3 | | MP2K2 | NADK | | | S100A13 |
| 0.628 | Notch1 | | | | | | | | PTN | |
| 0.628 | Notch1 | | CILP2 | H2A3 | IGFALS | | | | | |
| 0.628 | Notch1 | | | H2A3 | IGFALS | MP2K2 | | | | |
| 0.628 | Notch1 | CDON | CILP2 | | | | | PH | | |
| 0.628 | Notch1 | | | | IGFALS | | | | | S100A13 |
| 0.628 | Notch1 | CDON | | | | MP2K2 | NADK | | | S100A13 |
| 0.629 | Notch1 | CDON | | H2A3 | IGFALS | | NADK | | | |
| 0.629 | Notch1 | CDON | | | | MP2K2 | | PH | | |
| 0.629 | Notch1 | | | | | | NADK | PH | | |
| 0.629 | Notch1 | CDON | CILP2 | | | | NADK | | | S100A13 |
| 0.63 | Notch1 | | | | | MP2K2 | | | PTN | |
| 0.63 | Notch1 | CDON | CILP2 | H2A3 | | MP2K2 | NADK | | | |
| 0.63 | Notch1 | | | H2A3 | | | | PH | | |
| 0.63 | Notch1 | CDON | CILP2 | H2A3 | | | NADK | | | S100A13 |
| 0.631 | Notch1 | CDON | | | | | | | PTN | |
| 0.631 | Notch1 | CDON | CILP2 | H2A3 | IGFALS | | | | | |
| 0.631 | Notch1 | | CILP2 | | IGFALS | MP2K2 | | | | |
| 0.631 | Notch1 | CDON | | | IGFALS | MP2K2 | NADK | | | |
| 0.631 | Notch1 | | | H2A3 | IGFALS | MP2K2 | NADK | | | |
| 0.632 | Notch1 | | CILP2 | | IGFALS | | NADK | | | |
| 0.632 | Notch1 | | | H2A3 | IGFALS | | | | | S100A13 |
| 0.632 | Notch1 | | CILP2 | H2A3 | | MP2K2 | | | | S100A13 |
| 0.632 | Notch1 | CDON | | | | | | PH | | S100A13 |
| 0.633 | Notch1 | CDON | | | | | NADK | PH | | |
| 0.633 | Notch1 | CDON | | | IGFALS | | | | | S100A13 |
| 0.633 | Notch1 | CDON | | H2A3 | | MP2K2 | NADK | | | S100A13 |
| 0.633 | Notch1 | | CILP2 | | | | | PH | | S100A13 |
| 0.634 | Notch1 | CDON | | | | MP2K2 | | | PTN | |
| 0.634 | Notch1 | CDON | CILP2 | | IGFALS | MP2K2 | | | | |
| 0.634 | Notch1 | CDON | | H2A3 | IGFALS | MP2K2 | | | | |
| 0.634 | Notch1 | CDON | CILP2 | | IGFALS | | NADK | | | |
| 0.634 | Notch1 | | CILP2 | H2A3 | IGFALS | | NADK | | | |
| 0.634 | Notch1 | | | | | MP2K2 | NADK | PH | | |
| 0.634 | Notch1 | | | | | MP2K2 | | PH | | S100A13 |
| 0.635 | Notch1 | | CILP2 | | | MP2K2 | | PH | | |
| 0.635 | Notch1 | | | H2A3 | | | NADK | PH | | |
| 0.635 | Notch1 | | CILP2 | | IGFALS | | | | | S100A13 |
| 0.636 | Notch1 | CDON | CILP2 | H2A3 | IGFALS | | NADK | | | |
| 0.636 | Notch1 | CDON | | H2A3 | IGFALS | MP2K2 | NADK | | | |
| 0.636 | Notch1 | CDON | | H2A3 | | | | PH | | |
| 0.636 | Notch1 | | CILP2 | H2A3 | | | | PH | | |
| 0.636 | Notch1 | CDON | CILP2 | H2A3 | | MP2K2 | | | | S100A13 |
| 0.637 | Notch1 | CDON | | H2A3 | IGFALS | | | | | S100A13 |
| 0.637 | Notch1 | | | | IGFALS | MP2K2 | | | | S100A13 |
| 0.637 | Notch1 | | CILP2 | | | MP2K2 | NADK | | | S100A13 |
| 0.637 | Notch1 | | | H2A3 | | | | PH | | S100A13 |
| 0.638 | Notch1 | | CILP2 | H2A3 | IGFALS | MP2K2 | | | | |
| 0.638 | Notch1 | | CILP2 | | IGFALS | MP2K2 | NADK | | | |
| 0.638 | Notch1 | | CILP2 | | | | NADK | PH | | |
| 0.638 | Notch1 | CDON | CILP2 | | IGFALS | | | | | S100A13 |
| 0.638 | Notch1 | | | | IGFALS | | NADK | | | S100A13 |
| 0.638 | Notch1 | CDON | CILP2 | | | | | PH | | S100A13 |
| 0.639 | Notch1 | | | | IGFALS | | | PH | | |
| 0.639 | Notch1 | CDON | | | | MP2K2 | NADK | PH | | |
| 0.639 | Notch1 | | CILP2 | H2A3 | IGFALS | | | | | S100A13 |
| 0.639 | Notch1 | | | H2A3 | IGFALS | | NADK | | | S100A13 |
| 0.64 | Notch1 | | | H2A3 | | | | | PTN | |
| 0.64 | Notch1 | CDON | CILP2 | | IGFALS | MP2K2 | NADK | | | |
| 0.64 | Notch1 | CDON | CILP2 | H2A3 | | | | PH | | |
| 0.64 | Notch1 | CDON | CILP2 | | | MP2K2 | | PH | | |
| 0.64 | Notch1 | CDON | | H2A3 | | | NADK | PH | | |
| 0.64 | Notch1 | CDON | CILP2 | | | MP2K2 | NADK | | | S100A13 |
| 0.64 | Notch1 | | CILP2 | H2A3 | | MP2K2 | NADK | | | S100A13 |
| 0.64 | Notch1 | | | | | | NADK | PH | | S100A13 |
| 0.641 | Notch1 | CDON | CILP2 | H2A3 | IGFALS | MP2K2 | | | | |
| 0.641 | Notch1 | | CILP2 | H2A3 | IGFALS | MP2K2 | NADK | | | |
| 0.641 | Notch1 | | | H2A3 | | MP2K2 | | PH | | |
| 0.641 | Notch1 | CDON | CILP2 | | | | NADK | PH | | |
| 0.641 | Notch1 | CDON | CILP2 | H2A3 | IGFALS | | | | | S100A13 |
| 0.641 | Notch1 | CDON | | | IGFALS | MP2K2 | | | | S100A13 |
| 0.641 | Notch1 | CDON | | | IGFALS | | NADK | | | S100A13 |
| 0.641 | Notch1 | CDON | | | | MP2K2 | | PH | | S100A13 |
| 0.642 | Notch1 | | | H2A3 | | MP2K2 | | | PTN | |
| 0.642 | Notch1 | | | | | | NADK | | PTN | |
| 0.642 | Notch1 | | CILP2 | H2A3 | | | NADK | PH | | |
| 0.642 | Notch1 | | | | | | | | PTN | S100A13 |
| 0.642 | Notch1 | | | H2A3 | IGFALS | MP2K2 | | | | S100A13 |
| 0.642 | Notch1 | | CILP2 | H2A3 | | | | PH | | S100A13 |
| 0.643 | Notch1 | CDON | | H2A3 | | | | | PTN | |
| 0.643 | Notch1 | | | | | MP2K2 | NADK | | PTN | |
| 0.643 | Notch1 | CDON | | | IGFALS | | | PH | | |
| 0.643 | Notch1 | | | H2A3 | | MP2K2 | NADK | PH | | |
| 0.643 | Notch1 | CDON | | H2A3 | IGFALS | | NADK | | | S100A13 |
| 0.643 | Notch1 | CDON | CILP2 | H2A3 | | MP2K2 | NADK | | | S100A13 |
| 0.643 | Notch1 | CDON | | H2A3 | | | | PH | | S100A13 |
| 0.644 | Notch1 | CDON | | | | | NADK | | PTN | |
| 0.644 | Notch1 | CDON | CILP2 | H2A3 | IGFALS | MP2K2 | NADK | | | |
| 0.644 | Notch1 | | | | IGFALS | MP2K2 | | PH | | |
| 0.644 | Notch1 | | | | | MP2K2 | | | PTN | S100A13 |
| 0.644 | Notch1 | | | | IGFALS | MP2K2 | NADK | | | S100A13 |
| 0.644 | Notch1 | | | H2A3 | | | NADK | PH | | S100A13 |
| 0.645 | Notch1 | CDON | | | | MP2K2 | NADK | | PTN | |
| 0.645 | Notch1 | CDON | CILP2 | H2A3 | | | NADK | PH | | |
| 0.645 | Notch1 | | CILP2 | | IGFALS | MP2K2 | | | | S100A13 |
| 0.645 | Notch1 | CDON | | H2A3 | IGFALS | MP2K2 | | | | S100A13 |
| 0.645 | Notch1 | | | H2A3 | IGFALS | MP2K2 | NADK | | | S100A13 |
| 0.645 | Notch1 | | CILP2 | | | MP2K2 | | PH | | S100A13 |
| 0.645 | Notch1 | CDON | | | | | NADK | PH | | S100A13 |
| 0.646 | Notch1 | CDON | | H2A3 | | MP2K2 | | | PTN | |
| 0.646 | Notch1 | | | H2A3 | | | NADK | | PTN | |
| 0.646 | Notch1 | | CILP2 | | IGFALS | | | PH | | |
| 0.646 | Notch1 | CDON | | H2A3 | | MP2K2 | | PH | | |
| 0.646 | Notch1 | | CILP2 | | | MP2K2 | NADK | PH | | |
| 0.646 | Notch1 | CDON | | | | | | | PTN | S100A13 |
| 0.646 | Notch1 | | CILP2 | | IGFALS | | NADK | | | S100A13 |
| 0.646 | Notch1 | | CILP2 | H2A3 | IGFALS | | NADK | | | S100A13 |
| 0.646 | Notch1 | CDON | CILP2 | H2A3 | | | | PH | | S100A13 |
| 0.646 | Notch1 | | | | | MP2K2 | NADK | PH | | S100A13 |
| 0.647 | Notch1 | | | H2A3 | | MP2K2 | NADK | | PTN | |
| 0.647 | Notch1 | | | | IGFALS | | NADK | PH | | |
| 0.647 | Notch1 | CDON | CILP2 | | IGFALS | MP2K2 | | | | S100A13 |
| 0.647 | Notch1 | CDON | CILP2 | | IGFALS | | NADK | | | S100A13 |
| 0.647 | Notch1 | CDON | CILP2 | H2A3 | IGFALS | | NADK | | | S100A13 |
| 0.647 | Notch1 | CDON | | | IGFALS | MP2K2 | NADK | | | S100A13 |
| 0.647 | Notch1 | | | H2A3 | | MP2K2 | | PH | | S100A13 |
| 0.648 | Notch1 | CDON | | | IGFALS | MP2K2 | | PH | | |
| 0.648 | Notch1 | CDON | | H2A3 | | MP2K2 | NADK | PH | | |
| 0.648 | Notch1 | CDON | | | | MP2K2 | | | PTN | S100A13 |
| 0.648 | Notch1 | | CILP2 | | | | NADK | PH | | S100A13 |
| 0.649 | Notch1 | CDON | | H2A3 | | | NADK | | PTN | |
| 0.649 | Notch1 | CDON | CILP2 | | IGFALS | | | PH | | |
| 0.649 | Notch1 | | | H2A3 | IGFALS | | | PH | | |
| 0.649 | Notch1 | CDON | CILP2 | | | MP2K2 | NADK | PH | | |
| 0.649 | Notch1 | | | | IGFALS | MP2K2 | NADK | PH | | |
| 0.649 | Notch1 | CDON | | H2A3 | IGFALS | MP2K2 | NADK | | | S100A13 |
| 0.649 | Notch1 | | | | IGFALS | | | PH | | S100A13 |
| 0.649 | Notch1 | CDON | CILP2 | | | MP2K2 | | PH | | S100A13 |
| 0.649 | Notch1 | CDON | | H2A3 | | | NADK | PH | | S100A13 |
| 0.65 | Notch1 | | | | | | | PH | PTN | |
| 0.65 | Notch1 | | | | | MP2K2 | | PH | PTN | |
| 0.65 | Notch1 | | CILP2 | H2A3 | | MP2K2 | | PH | | |
| 0.65 | Notch1 | CDON | | | IGFALS | | NADK | PH | | |
| 0.65 | Notch1 | | CILP2 | H2A3 | IGFALS | MP2K2 | | | | S100A13 |
| 0.651 | Notch1 | CDON | | H2A3 | | MP2K2 | NADK | | PTN | |
| 0.651 | Notch1 | CDON | | | | | | PH | PTN | |
| 0.651 | Notch1 | | | H2A3 | IGFALS | | NADK | PH | | |
| 0.651 | Notch1 | | | H2A3 | | | | | PTN | S100A13 |
| 0.651 | Notch1 | CDON | CILP2 | | | | NADK | PH | | S100A13 |
| 0.651 | Notch1 | | CILP2 | H2A3 | | | NADK | PH | | S100A13 |
| 0.651 | Notch1 | CDON | | | | MP2K2 | NADK | PH | | S100A13 |
| 0.652 | Notch1 | | | | IGFALS | | | | PTN | |
| 0.652 | Notch1 | | | | IGFALS | MP2K2 | | | PTN | |
| 0.652 | Notch1 | | CILP2 | H2A3 | | MP2K2 | NADK | PH | | |
| 0.652 | Notch1 | CDON | CILP2 | H2A3 | IGFALS | MP2K2 | | | | S100A13 |
| 0.652 | Notch1 | CDON | | H2A3 | | MP2K2 | | PH | | S100A13 |
| 0.652 | Notch1 | | | H2A3 | | MP2K2 | NADK | PH | | S100A13 |
| 0.653 | Notch1 | CDON | | | IGFALS | | | | PTN | |
| 0.653 | Notch1 | CDON | | H2A3 | IGFALS | | | PH | | |
| 0.653 | Notch1 | CDON | | | | MP2K2 | | PH | PTN | |
| 0.653 | Notch1 | CDON | CILP2 | H2A3 | | MP2K2 | | PH | | |
| 0.653 | Notch1 | | CILP2 | | IGFALS | MP2K2 | | PH | | |
| 0.653 | Notch1 | | | H2A3 | | MP2K2 | | | PTN | S100A13 |
| 0.653 | Notch1 | | CILP2 | | IGFALS | MP2K2 | NADK | | | S100A13 |
| 0.653 | Notch1 | CDON | | | IGFALS | | | PH | | S100A13 |
| 0.654 | Notch1 | CDON | | | IGFALS | MP2K2 | | | PTN | |
| 0.654 | Notch1 | | CILP2 | H2A3 | IGFALS | | | PH | | |
| 0.654 | Notch1 | | CILP2 | | IGFALS | | NADK | PH | | |
| 0.654 | Notch1 | CDON | | | IGFALS | MP2K2 | NADK | PH | | |
| 0.654 | Notch1 | CDON | | H2A3 | | | | | PTN | S100A13 |
| 0.654 | Notch1 | | CILP2 | H2A3 | IGFALS | MP2K2 | NADK | | | S100A13 |
| 0.654 | Notch1 | | CILP2 | | IGFALS | | | PH | | S100A13 |
| 0.654 | Notch1 | CDON | CILP2 | H2A3 | | | NADK | PH | | S100A13 |
| 0.655 | Notch1 | CDON | CILP2 | | IGFALS | MP2K2 | | PH | | |
| 0.655 | Notch1 | | | H2A3 | IGFALS | MP2K2 | | PH | | |
| 0.655 | Notch1 | CDON | | H2A3 | IGFALS | | NADK | PH | | |
| 0.655 | Notch1 | CDON | CILP2 | H2A3 | | MP2K2 | NADK | PH | | |
| 0.655 | Notch1 | | | | | | NADK | | PTN | S100A13 |
| 0.655 | Notch1 | | | | | MP2K2 | NADK | | PTN | S100A13 |
| 0.655 | Notch1 | CDON | CILP2 | | IGFALS | MP2K2 | NADK | | | S100A13 |
| 0.655 | Notch1 | | CILP2 | H2A3 | | MP2K2 | | PH | | S100A13 |
| 0.656 | Notch1 | CDON | CILP2 | | | | | | PTN | |
| 0.656 | Notch1 | CDON | CILP2 | H2A3 | IGFALS | | | PH | | |
| 0.656 | Notch1 | CDON | CILP2 | | IGFALS | | NADK | PH | | |
| 0.656 | Notch1 | | | H2A3 | IGFALS | MP2K2 | NADK | PH | | |
| 0.656 | Notch1 | CDON | | H2A3 | | MP2K2 | | | PTN | S100A13 |
| 0.656 | Notch1 | CDON | CILP2 | H2A3 | IGFALS | MP2K2 | NADK | | | S100A13 |
| 0.656 | Notch1 | | | H2A3 | IGFALS | | | PH | | S100A13 |
| 0.656 | Notch1 | | | | IGFALS | MP2K2 | | PH | | S100A13 |
| 0.656 | Notch1 | CDON | | H2A3 | | MP2K2 | NADK | PH | | S100A13 |
| 0.657 | Notch1 | | CILP2 | | | | | | PTN | |
| 0.657 | Notch1 | | CILP2 | H2A3 | IGFALS | | NADK | PH | | |
| 0.657 | Notch1 | CDON | | | | | NADK | | PTN | S100A13 |
| 0.657 | Notch1 | | | H2A3 | | | NADK | | PTN | S100A13 |
| 0.657 | Notch1 | CDON | CILP2 | | IGFALS | | | PH | | S100A13 |
| 0.657 | Notch1 | CDON | CILP2 | H2A3 | | MP2K2 | | PH | | S100A13 |
| 0.657 | Notch1 | | CILP2 | | | MP2K2 | NADK | PH | | S100A13 |
| 0.658 | Notch1 | | | H2A3 | IGFALS | | | | PTN | |
| 0.658 | Notch1 | | | H2A3 | IGFALS | MP2K2 | | | PTN | |
| 0.658 | Notch1 | CDON | | | | MP2K2 | NADK | | PTN | S100A13 |
| 0.658 | Notch1 | | | H2A3 | | MP2K2 | NADK | | PTN | S100A13 |
| 0.658 | Notch1 | | | | | | | PH | PTN | S100A13 |
| 0.658 | Notch1 | | | | IGFALS | | NADK | PH | | S100A13 |
| 0.659 | Notch1 | | | | IGFALS | MP2K2 | NADK | | PTN | |
| 0.659 | Notch1 | CDON | | H2A3 | IGFALS | MP2K2 | | PH | | |
| 0.659 | Notch1 | CDON | CILP2 | H2A3 | IGFALS | | NADK | PH | | |
| 0.659 | Notch1 | | CILP2 | | IGFALS | MP2K2 | NADK | PH | | |
| 0.659 | Notch1 | CDON | | H2A3 | IGFALS | MP2K2 | NADK | PH | | |
| 0.659 | Notch1 | CDON | | H2A3 | IGFALS | | | PH | | S100A13 |
| 0.659 | Notch1 | | | | | MP2K2 | | PH | PTN | S100A13 |
| 0.659 | Notch1 | CDON | | | IGFALS | MP2K2 | | PH | | S100A13 |
| 0.659 | Notch1 | CDON | CILP2 | | | MP2K2 | NADK | PH | | S100A13 |
| 0.66 | Notch1 | CDON | | H2A3 | IGFALS | | | | PTN | |
| 0.66 | Notch1 | CDON | CILP2 | | | MP2K2 | | | PTN | |
| 0.66 | Notch1 | | | | IGFALS | | NADK | | PTN | |
| 0.66 | Notch1 | CDON | | | IGFALS | | NADK | | PTN | |
| 0.66 | Notch1 | CDON | | | IGFALS | MP2K2 | NADK | | PTN | |
| 0.66 | Notch1 | | | | | | NADK | PH | PTN | |
| 0.66 | Notch1 | | | | | MP2K2 | NADK | PH | PTN | |
| 0.66 | Notch1 | CDON | | H2A3 | | | NADK | | PTN | S100A13 |
| 0.66 | Notch1 | | CILP2 | H2A3 | IGFALS | | | PH | | S100A13 |
| 0.661 | Notch1 | | CILP2 | H2A3 | | | | | PTN | |
| 0.661 | Notch1 | CDON | CILP2 | H2A3 | | | | | PTN | |
| 0.661 | Notch1 | | CILP2 | | | MP2K2 | | | PTN | |
| 0.661 | Notch1 | CDON | | H2A3 | IGFALS | MP2K2 | | | PTN | |
| 0.661 | Notch1 | CDON | | | | | NADK | PH | PTN | |
| 0.661 | Notch1 | CDON | CILP2 | | IGFALS | MP2K2 | NADK | PH | | |
| 0.661 | Notch1 | CDON | | H2A3 | | MP2K2 | NADK | | PTN | S100A13 |
| 0.661 | Notch1 | CDON | | | | | | PH | PTN | S100A13 |
| 0.661 | Notch1 | | | H2A3 | IGFALS | | NADK | PH | | S100A13 |
| 0.661 | Notch1 | | CILP2 | H2A3 | | MP2K2 | NADK | PH | | S100A13 |
| 0.662 | Notch1 | | | H2A3 | IGFALS | | NADK | | PTN | |
| 0.662 | Notch1 | | | H2A3 | IGFALS | MP2K2 | NADK | | PTN | |
| 0.662 | Notch1 | | | H2A3 | | | | PH | PTN | |
| 0.662 | Notch1 | | CILP2 | H2A3 | IGFALS | MP2K2 | | PH | | |
| 0.662 | Notch1 | CDON | | | | MP2K2 | NADK | PH | PTN | |
| 0.662 | Notch1 | CDON | CILP2 | | | | | | PTN | S100A13 |
| 0.662 | Notch1 | CDON | CILP2 | H2A3 | IGFALS | | | PH | | S100A13 |
| 0.662 | Notch1 | CDON | | | | MP2K2 | | PH | PTN | S100A13 |
| 0.662 | Notch1 | CDON | | | IGFALS | | NADK | PH | | S100A13 |
| 0.662 | Notch1 | CDON | CILP2 | H2A3 | | MP2K2 | NADK | PH | | S100A13 |
| 0.663 | Notch1 | | CILP2 | H2A3 | IGFALS | MP2K2 | NADK | PH | | |
| 0.663 | Notch1 | | CILP2 | | | | | | PTN | S100A13 |
| 0.663 | Notch1 | | CILP2 | | IGFALS | MP2K2 | | PH | | S100A13 |
| 0.663 | Notch1 | | | H2A3 | IGFALS | MP2K2 | | PH | | S100A13 |
| 0.663 | Notch1 | | | | IGFALS | MP2K2 | NADK | PH | | S100A13 |
| 0.664 | Notch1 | CDON | | H2A3 | IGFALS | | NADK | | PTN | |
| 0.664 | Notch1 | CDON | | H2A3 | IGFALS | MP2K2 | NADK | | PTN | |
| 0.664 | Notch1 | CDON | | H2A3 | | | | PH | PTN | |
| 0.664 | Notch1 | | | H2A3 | | MP2K2 | | PH | PTN | |
| 0.664 | Notch1 | CDON | CILP2 | H2A3 | IGFALS | MP2K2 | | PH | | |
| 0.664 | Notch1 | | | | IGFALS | | | | PTN | S100A13 |
| 0.664 | Notch1 | CDON | | | IGFALS | | | | PTN | S100A13 |
| 0.664 | Notch1 | | CILP2 | | IGFALS | | NADK | PH | | S100A13 |
| 0.664 | Notch1 | CDON | | H2A3 | IGFALS | | NADK | PH | | S100A13 |
| 0.665 | Notch1 | | CILP2 | H2A3 | | MP2K2 | | | PTN | |
| 0.665 | Notch1 | CDON | CILP2 | H2A3 | | MP2K2 | | | PTN | |
| 0.665 | Notch1 | CDON | CILP2 | H2A3 | IGFALS | MP2K2 | NADK | PH | | |
| 0.665 | Notch1 | | | | IGFALS | MP2K2 | | | PTN | S100A13 |
| 0.665 | Notch1 | CDON | CILP2 | | IGFALS | MP2K2 | | PH | | S100A13 |
| 0.665 | Notch1 | | CILP2 | H2A3 | IGFALS | | NADK | PH | | S100A13 |
| 0.665 | Notch1 | CDON | | | IGFALS | MP2K2 | NADK | PH | | S100A13 |
| 0.666 | Notch1 | CDON | CILP2 | | | | NADK | | PTN | |
| 0.666 | Notch1 | CDON | | H2A3 | | MP2K2 | | PH | PTN | |
| 0.666 | Notch1 | | | H2A3 | | | NADK | PH | PTN | |
| 0.666 | Notch1 | CDON | CILP2 | | | MP2K2 | | | PTN | S100A13 |
| 0.666 | Notch1 | CDON | | | IGFALS | MP2K2 | | | PTN | S100A13 |
| 0.666 | Notch1 | CDON | | H2A3 | IGFALS | MP2K2 | | PH | | S100A13 |
| 0.666 | Notch1 | CDON | CILP2 | | IGFALS | | NADK | PH | | S100A13 |
| 0.666 | Notch1 | | | H2A3 | IGFALS | MP2K2 | NADK | PH | | S100A13 |
| 0.667 | Notch1 | | CILP2 | H2A3 | | | NADK | | PTN | |
| 0.667 | Notch1 | CDON | CILP2 | H2A3 | | | NADK | | PTN | |
| 0.667 | Notch1 | | | | IGFALS | | | PH | PTN | |
| 0.667 | Notch1 | | | | IGFALS | MP2K2 | | PH | PTN | |
| 0.667 | Notch1 | CDON | | H2A3 | | | NADK | PH | PTN | |
| 0.667 | Notch1 | | | H2A3 | | MP2K2 | NADK | PH | PTN | |
| 0.667 | Notch1 | | CILP2 | H2A3 | | | | | PTN | S100A13 |
| 0.667 | Notch1 | CDON | CILP2 | H2A3 | | | | | PTN | S100A13 |
| 0.667 | Notch1 | | | H2A3 | IGFALS | | | | PTN | S100A13 |
| 0.667 | Notch1 | | CILP2 | | | MP2K2 | | | PTN | S100A13 |
| 0.667 | Notch1 | CDON | CILP2 | H2A3 | IGFALS | | NADK | PH | | S100A13 |
| 0.668 | Notch1 | | CILP2 | | | | NADK | | PTN | |
| 0.668 | Notch1 | CDON | CILP2 | | | MP2K2 | NADK | | PTN | |
| 0.668 | Notch1 | CDON | | | IGFALS | | | PH | PTN | |
| 0.668 | Notch1 | CDON | | H2A3 | IGFALS | | | | PTN | S100A13 |
| 0.668 | Notch1 | | | H2A3 | IGFALS | MP2K2 | | | PTN | S100A13 |
| 0.668 | Notch1 | | | H2A3 | | | | PH | PTN | S100A13 |
| 0.668 | Notch1 | | CILP2 | H2A3 | IGFALS | MP2K2 | | PH | | S100A13 |
| 0.668 | Notch1 | | | | | | NADK | PH | PTN | S100A13 |
| 0.668 | Notch1 | CDON | | H2A3 | IGFALS | MP2K2 | NADK | PH | | S100A13 |
| 0.669 | Notch1 | | CILP2 | | | MP2K2 | NADK | | PTN | |
| 0.669 | Notch1 | CDON | | | IGFALS | MP2K2 | | PH | PTN | |
| 0.669 | Notch1 | CDON | | H2A3 | | MP2K2 | NADK | PH | PTN | |
| 0.669 | Notch1 | CDON | CILP2 | H2A3 | IGFALS | MP2K2 | | PH | | S100A13 |
| 0.669 | Notch1 | | | | | MP2K2 | NADK | PH | PTN | S100A13 |
| 0.67 | Notch1 | CDON | CILP2 | | IGFALS | | | | PTN | |
| 0.67 | Notch1 | | CILP2 | H2A3 | | MP2K2 | NADK | | PTN | |
| 0.67 | Notch1 | CDON | CILP2 | H2A3 | | MP2K2 | NADK | | PTN | |
| 0.67 | Notch1 | CDON | CILP2 | | | | | PH | PTN | |
| 0.67 | Notch1 | CDON | | H2A3 | IGFALS | MP2K2 | | | PTN | S100A13 |
| 0.67 | Notch1 | CDON | | H2A3 | | | | PH | PTN | S100A13 |
| 0.67 | Notch1 | | | H2A3 | | MP2K2 | | PH | PTN | S100A13 |
| 0.67 | Notch1 | CDON | | | | | NADK | PH | PTN | S100A13 |
| 0.67 | Notch1 | CDON | | | | MP2K2 | NADK | PH | PTN | S100A13 |
| 0.67 | Notch1 | | CILP2 | | IGFALS | MP2K2 | NADK | PH | | S100A13 |
| 0.671 | Notch1 | | CILP2 | | | | | PH | PTN | |
| 0.671 | Notch1 | | CILP2 | H2A3 | | MP2K2 | | | PTN | S100A13 |
| 0.671 | Notch1 | CDON | CILP2 | H2A3 | | MP2K2 | | | PTN | S100A13 |
| 0.671 | Notch1 | | | | IGFALS | | NADK | | PTN | S100A13 |
| 0.671 | Notch1 | CDON | | | IGFALS | | NADK | | PTN | S100A13 |
| 0.671 | Notch1 | | | | IGFALS | MP2K2 | NADK | | PTN | S100A13 |
| 0.671 | Notch1 | CDON | CILP2 | | IGFALS | MP2K2 | NADK | PH | | S100A13 |
| 0.672 | Notch1 | | CILP2 | | IGFALS | | | | PTN | |
| 0.672 | Notch1 | CDON | CILP2 | | IGFALS | MP2K2 | | | PTN | |
| 0.672 | Notch1 | | | H2A3 | IGFALS | | NADK | | PTN | S100A13 |
| 0.672 | Notch1 | CDON | | | IGFALS | MP2K2 | NADK | | PTN | S100A13 |
| 0.672 | Notch1 | | | H2A3 | IGFALS | MP2K2 | NADK | | PTN | S100A13 |
| 0.672 | Notch1 | CDON | | H2A3 | | MP2K2 | | PH | PTN | S100A13 |
| 0.672 | Notch1 | | | H2A3 | | | NADK | PH | PTN | S100A13 |
| 0.672 | Notch1 | | CILP2 | H2A3 | IGFALS | MP2K2 | NADK | PH | | S100A13 |
| 0.673 | Notch1 | CDON | CILP2 | H2A3 | IGFALS | | | | PTN | |
| 0.673 | Notch1 | CDON | CILP2 | | | MP2K2 | | PH | PTN | |
| 0.673 | Notch1 | | | | IGFALS | | NADK | PH | PTN | |
| 0.673 | Notch1 | | | | IGFALS | MP2K2 | NADK | PH | PTN | |
| 0.673 | Notch1 | CDON | CILP2 | | | | NADK | | PTN | S100A13 |
| 0.673 | Notch1 | CDON | CILP2 | H2A3 | | | NADK | | PTN | S100A13 |
| 0.673 | Notch1 | CDON | | H2A3 | IGFALS | | NADK | | PTN | S100A13 |
| 0.673 | Notch1 | CDON | | H2A3 | IGFALS | MP2K2 | NADK | | PTN | S100A13 |
| 0.673 | Notch1 | | | H2A3 | | MP2K2 | NADK | PH | PTN | S100A13 |
| 0.673 | Notch1 | CDON | CILP2 | H2A3 | IGFALS | MP2K2 | NADK | PH | | S100A13 |
| 0.674 | Notch1 | | CILP2 | H2A3 | IGFALS | | | | PTN | |
| 0.674 | Notch1 | | CILP2 | | IGFALS | MP2K2 | | | PTN | |
| 0.674 | Notch1 | | CILP2 | | | MP2K2 | | PH | PTN | |
| 0.674 | Notch1 | CDON | | | IGFALS | | NADK | PH | PTN | |
| 0.674 | Notch1 | CDON | | | IGFALS | MP2K2 | NADK | PH | PTN | |
| 0.674 | Notch1 | | CILP2 | | | | NADK | | PTN | S100A13 |
| 0.674 | Notch1 | | CILP2 | H2A3 | | | NADK | | PTN | S100A13 |
| 0.674 | Notch1 | | CILP2 | | | | | PH | PTN | S100A13 |
| 0.674 | Notch1 | CDON | CILP2 | | | | | PH | PTN | S100A13 |
| 0.674 | Notch1 | CDON | | H2A3 | | | NADK | PH | PTN | S100A13 |
| 0.675 | Notch1 | | CILP2 | H2A3 | IGFALS | MP2K2 | | | PTN | |
| 0.675 | Notch1 | CDON | CILP2 | H2A3 | IGFALS | MP2K2 | | | PTN | |
| 0.675 | Notch1 | | | H2A3 | IGFALS | | | PH | PTN | |
| 0.675 | Notch1 | | | | IGFALS | | | PH | PTN | S100A13 |
| 0.675 | Notch1 | CDON | | H2A3 | | MP2K2 | NADK | PH | PTN | S100A13 |
| 0.676 | Notch1 | | CILP2 | H2A3 | | | | PH | PTN | |
| 0.676 | Notch1 | CDON | CILP2 | H2A3 | | | | PH | PTN | |
| 0.676 | Notch1 | | | H2A3 | IGFALS | MP2K2 | | PH | PTN | |
| 0.676 | Notch1 | | CILP2 | | | MP2K2 | NADK | | PTN | S100A13 |
| 0.676 | Notch1 | CDON | CILP2 | | | MP2K2 | NADK | | PTN | S100A13 |
| 0.676 | Notch1 | CDON | CILP2 | H2A3 | | MP2K2 | NADK | | PTN | S100A13 |
| 0.676 | Notch1 | CDON | | | IGFALS | | | PH | PTN | S100A13 |
| 0.676 | Notch1 | | | | IGFALS | MP2K2 | | PH | PTN | S100A13 |
| 0.677 | Notch1 | CDON | CILP2 | | IGFALS | | NADK | | PTN | |
| 0.677 | Notch1 | CDON | CILP2 | H2A3 | IGFALS | | NADK | | PTN | |
| 0.677 | Notch1 | CDON | | H2A3 | IGFALS | | | PH | PTN | |
| 0.677 | Notch1 | CDON | | H2A3 | IGFALS | MP2K2 | | PH | PTN | |
| 0.677 | Notch1 | | | H2A3 | IGFALS | | NADK | PH | PTN | |
| 0.677 | Notch1 | | | H2A3 | IGFALS | MP2K2 | NADK | PH | PTN | |
| 0.677 | Notch1 | CDON | CILP2 | | IGFALS | | | | PTN | S100A13 |
| 0.677 | Notch1 | | CILP2 | H2A3 | | MP2K2 | NADK | | PTN | S100A13 |
| 0.677 | Notch1 | CDON | CILP2 | | | MP2K2 | | PH | PTN | S100A13 |
| 0.677 | Notch1 | CDON | | | IGFALS | MP2K2 | | PH | PTN | S100A13 |
| 0.678 | Notch1 | | CILP2 | H2A3 | IGFALS | | NADK | | PTN | |
| 0.678 | Notch1 | CDON | CILP2 | | IGFALS | MP2K2 | NADK | | PTN | |
| 0.678 | Notch1 | CDON | CILP2 | H2A3 | IGFALS | MP2K2 | NADK | | PTN | |
| 0.678 | Notch1 | CDON | CILP2 | | | | NADK | PH | PTN | |
| 0.678 | Notch1 | CDON | | H2A3 | IGFALS | | NADK | PH | PTN | |
| 0.678 | Notch1 | | CILP2 | | IGFALS | | | | PTN | S100A13 |
| 0.678 | Notch1 | | CILP2 | | | MP2K2 | | PH | PTN | S100A13 |
| 0.679 | Notch1 | | CILP2 | | IGFALS | | NADK | | PTN | |
| 0.679 | Notch1 | | CILP2 | | IGFALS | MP2K2 | NADK | | PTN | |
| 0.679 | Notch1 | | CILP2 | H2A3 | IGFALS | MP2K2 | NADK | | PTN | |
| 0.679 | Notch1 | CDON | CILP2 | H2A3 | | MP2K2 | | PH | PTN | |
| 0.679 | Notch1 | | CILP2 | | | | NADK | PH | PTN | |
| 0.679 | Notch1 | CDON | | H2A3 | IGFALS | MP2K2 | NADK | PH | PTN | |
| 0.679 | Notch1 | | CILP2 | H2A3 | IGFALS | | | | PTN | S100A13 |
| 0.679 | Notch1 | CDON | CILP2 | H2A3 | IGFALS | | | | PTN | S100A13 |
| 0.679 | Notch1 | CDON | CILP2 | | IGFALS | MP2K2 | | | PTN | S100A13 |
| 0.68 | Notch1 | | CILP2 | H2A3 | | MP2K2 | | PH | PTN | |
| 0.68 | Notch1 | | CILP2 | H2A3 | | | NADK | PH | PTN | |
| 0.68 | Notch1 | CDON | CILP2 | H2A3 | | | NADK | PH | PTN | |
| 0.68 | Notch1 | CDON | CILP2 | | | MP2K2 | NADK | PH | PTN | |
| 0.68 | Notch1 | | CILP2 | | IGFALS | MP2K2 | | | PTN | S100A13 |
| 0.68 | Notch1 | | CILP2 | H2A3 | | | | PH | PTN | S100A13 |
| 0.68 | Notch1 | CDON | CILP2 | H2A3 | | | | PH | PTN | S100A13 |
| 0.681 | Notch1 | | CILP2 | | | MP2K2 | NADK | PH | PTN | |
| 0.681 | Notch1 | CDON | CILP2 | H2A3 | IGFALS | MP2K2 | | | PTN | S100A13 |
| 0.681 | Notch1 | | | H2A3 | IGFALS | | | PH | PTN | S100A13 |
| 0.682 | Notch1 | CDON | CILP2 | | IGFALS | | | PH | PTN | |
| 0.682 | Notch1 | CDON | CILP2 | H2A3 | | MP2K2 | NADK | PH | PTN | |
| 0.682 | Notch1 | | CILP2 | H2A3 | IGFALS | MP2K2 | | | PTN | S100A13 |
| 0.682 | Notch1 | CDON | | H2A3 | IGFALS | | | PH | PTN | S100A13 |
| 0.682 | Notch1 | | | H2A3 | IGFALS | MP2K2 | | PH | PTN | S100A13 |
| 0.682 | Notch1 | | | | IGFALS | | NADK | PH | PTN | S100A13 |
| 0.682 | Notch1 | CDON | | | IGFALS | | NADK | PH | PTN | S100A13 |
| 0.682 | Notch1 | | | | IGFALS | MP2K2 | NADK | PH | PTN | S100A13 |
| 0.683 | Notch1 | | CILP2 | | IGFALS | | | PH | PTN | |
| 0.683 | Notch1 | | CILP2 | H2A3 | | MP2K2 | NADK | PH | PTN | |
| 0.683 | Notch1 | CDON | CILP2 | | IGFALS | | NADK | | PTN | S100A13 |
| 0.683 | Notch1 | CDON | CILP2 | H2A3 | IGFALS | | NADK | | PTN | S100A13 |
| 0.683 | Notch1 | CDON | CILP2 | H2A3 | | MP2K2 | | PH | PTN | S100A13 |
| 0.683 | Notch1 | CDON | | H2A3 | IGFALS | MP2K2 | | PH | PTN | S100A13 |
| 0.683 | Notch1 | CDON | CILP2 | | | | NADK | PH | PTN | S100A13 |
| 0.683 | Notch1 | CDON | | | IGFALS | MP2K2 | NADK | PH | PTN | S100A13 |
| 0.684 | Notch1 | CDON | CILP2 | | IGFALS | MP2K2 | | PH | PTN | |
| 0.684 | Notch1 | | CILP2 | H2A3 | IGFALS | | NADK | | PTN | S100A13 |
| 0.684 | Notch1 | | CILP2 | H2A3 | | MP2K2 | | PH | PTN | S100A13 |
| 0.684 | Notch1 | | CILP2 | | | | NADK | PH | PTN | S100A13 |
| 0.685 | Notch1 | | CILP2 | | IGFALS | MP2K2 | | PH | PTN | |
| 0.685 | Notch1 | | CILP2 | | IGFALS | | NADK | | PTN | S100A13 |
| 0.685 | Notch1 | CDON | CILP2 | | IGFALS | MP2K2 | NADK | | PTN | S100A13 |
| 0.685 | Notch1 | CDON | CILP2 | H2A3 | IGFALS | MP2K2 | NADK | | PTN | S100A13 |
| 0.685 | Notch1 | | CILP2 | H2A3 | | | NADK | PH | PTN | S100A13 |
| 0.685 | Notch1 | CDON | CILP2 | H2A3 | | | NADK | PH | PTN | S100A13 |
| 0.685 | Notch1 | | | H2A3 | IGFALS | | NADK | PH | PTN | S100A13 |
| 0.685 | Notch1 | CDON | | H2A3 | IGFALS | | NADK | PH | PTN | S100A13 |
| 0.685 | Notch1 | CDON | CILP2 | | | MP2K2 | NADK | PH | PTN | S100A13 |
| 0.685 | Notch1 | | | H2A3 | IGFALS | MP2K2 | NADK | PH | PTN | S100A13 |
| 0.685 | Notch1 | CDON | | H2A3 | IGFALS | MP2K2 | NADK | PH | PTN | S100A13 |
| 0.686 | Notch1 | | CILP2 | H2A3 | IGFALS | | | PH | PTN | |
| 0.686 | Notch1 | CDON | CILP2 | H2A3 | IGFALS | | | PH | PTN | |
| 0.686 | Notch1 | | CILP2 | H2A3 | IGFALS | MP2K2 | NADK | | PTN | S100A13 |
| 0.686 | Notch1 | CDON | CILP2 | | IGFALS | | | PH | PTN | S100A13 |
| 0.686 | Notch1 | | CILP2 | | | MP2K2 | NADK | PH | PTN | S100A13 |
| 0.687 | Notch1 | CDON | CILP2 | | IGFALS | | NADK | PH | PTN | |
| 0.687 | Notch1 | | CILP2 | | IGFALS | MP2K2 | NADK | | PTN | S100A13 |
| 0.687 | Notch1 | | CILP2 | | IGFALS | | | PH | PTN | S100A13 |
| 0.687 | Notch1 | CDON | CILP2 | H2A3 | | MP2K2 | NADK | PH | PTN | S100A13 |
| 0.688 | Notch1 | | CILP2 | H2A3 | IGFALS | MP2K2 | | PH | PTN | |
| 0.688 | Notch1 | CDON | CILP2 | H2A3 | IGFALS | MP2K2 | | PH | PTN | |
| 0.688 | Notch1 | | CILP2 | | IGFALS | | NADK | PH | PTN | |
| 0.688 | Notch1 | CDON | CILP2 | H2A3 | IGFALS | | NADK | PH | PTN | |
| 0.688 | Notch1 | CDON | CILP2 | | IGFALS | MP2K2 | NADK | PH | PTN | |
| 0.688 | Notch1 | CDON | CILP2 | | IGFALS | MP2K2 | | PH | PTN | S100A13 |
| 0.688 | Notch1 | | CILP2 | H2A3 | | MP2K2 | NADK | PH | PTN | S100A13 |
| 0.689 | Notch1 | | CILP2 | H2A3 | IGFALS | | NADK | PH | PTN | |
| 0.689 | Notch1 | | CILP2 | | IGFALS | MP2K2 | NADK | PH | PTN | |
| 0.689 | Notch1 | CDON | CILP2 | H2A3 | IGFALS | MP2K2 | NADK | PH | PTN | |
| 0.69 | Notch1 | | CILP2 | H2A3 | IGFALS | MP2K2 | NADK | PH | PTN | |
| 0.69 | Notch1 | CDON | CILP2 | H2A3 | IGFALS | | | PH | PTN | S100A13 |
| 0.69 | Notch1 | | CILP2 | | IGFALS | MP2K2 | | PH | PTN | S100A13 |
| 0.691 | Notch1 | | CILP2 | H2A3 | IGFALS | | | PH | PTN | S100A13 |
| 0.692 | Notch1 | CDON | CILP2 | H2A3 | IGFALS | MP2K2 | | PH | PTN | S100A13 |
| 0.692 | Notch1 | CDON | CILP2 | | IGFALS | | NADK | PH | PTN | S100A13 |
| 0.693 | Notch1 | | CILP2 | H2A3 | IGFALS | MP2K2 | | PH | PTN | S100A13 |
| 0.693 | Notch1 | CDON | CILP2 | H2A3 | IGFALS | | NADK | PH | PTN | S100A13 |
| 0.694 | Notch1 | | CILP2 | | IGFALS | | NADK | PH | PTN | S100A13 |
| 0.694 | Notch1 | | CILP2 | H2A3 | IGFALS | | NADK | PH | PTN | S100A13 |
| 0.694 | Notch1 | CDON | CILP2 | | IGFALS | MP2K2 | NADK | PH | PTN | S100A13 |
| 0.694 | Notch1 | CDON | CILP2 | H2A3 | IGFALS | MP2K2 | NADK | PH | PTN | S100A13 |
| 0.695 | Notch1 | | CILP2 | | IGFALS | MP2K2 | NADK | PH | PTN | S100A13 |
| *0.696 | Notch1 | | CILP2 | H2A3 | IGFALS | MP2K2 | NADK | PH | PTN | S100A13 |

*Included in Table 38

TABLE 42
NADK Biomarker Panels-20 Year Model

| AUC | NADK | CDON | CILP2 | H2A3 | IGFALS | MP2K2 | Notch1 | PH | PTN | S100A13 |
|---|---|---|---|---|---|---|---|---|---|---|
| 0.574 | NADK | | | H2A3 | | | | | | |
| 0.587 | NADK | | | | | | | | | S100A13 |
| 0.588 | NADK | | | | | MP2K2 | | | | |
| 0.589 | NADK | CDON | | | | | | | | |
| 0.591 | NADK | | | H2A3 | | | | | | S100A13 |
| 0.593 | NADK | | | | | | Notch1 | | | |
| 0.594 | NADK | CDON | | H2A3 | | | | | | |
| 0.594 | NADK | | | H2A3 | | MP2K2 | | | | |
| 0.599 | NADK | | | H2A3 | | | Notch1 | | | |
| 0.6 | NADK | | CILP2 | | | | | | | |
| 0.6 | NADK | CDON | | | | | Notch1 | | | |
| 0.602 | NADK | | CILP2 | H2A3 | | | | | | |
| 0.602 | NADK | | | | | MP2K2 | | | | S100A13 |
| 0.603 | NADK | CDON | | | | MP2K2 | | | | |
| 0.603 | NADK | | | | | MP2K2 | Notch1 | | | |
| 0.605 | NADK | CDON | | | | | | | | S100A13 |
| 0.606 | NADK | | | H2A3 | | MP2K2 | | | | S100A13 |
| 0.607 | NADK | CDON | | H2A3 | | | Notch1 | | | |
| 0.608 | NADK | CDON | | H2A3 | | | | | | S100A13 |
| 0.609 | NADK | CDON | CILP2 | | | | | | | |
| 0.609 | NADK | CDON | | H2A3 | | MP2K2 | | | | |
| 0.609 | NADK | | CILP2 | | | | Notch1 | | | |
| 0.61 | NADK | CDON | | | | MP2K2 | Notch1 | | | |
| 0.611 | NADK | CDON | CILP2 | H2A3 | | | | | | |
| 0.611 | NADK | | | | IGFALS | | | | | |
| 0.611 | NADK | | | H2A3 | | MP2K2 | Notch1 | | | |
| 0.612 | NADK | | | H2A3 | IGFALS | | | | | |
| 0.612 | NADK | | CILP2 | H2A3 | | | Notch1 | | | |
| 0.613 | NADK | CDON | CILP2 | | | | Notch1 | | | |
| 0.613 | NADK | | | | | | Notch1 | | | S100A13 |
| 0.614 | NADK | | CILP2 | | | | | | | S100A13 |
| 0.614 | NADK | | CILP2 | H2A3 | | | | | | S100A13 |
| 0.615 | NADK | CDON | | | | MP2K2 | | | | S100A13 |
| 0.616 | NADK | | CILP2 | | | MP2K2 | | | | |
| 0.616 | NADK | | | | | | | PH | | |
| 0.617 | NADK | | | | IGFALS | MP2K2 | | | | |
| 0.617 | NADK | CDON | CILP2 | H2A3 | | | Notch1 | | | |
| 0.617 | NADK | | | H2A3 | | | Notch1 | | | S100A13 |
| 0.619 | NADK | | | H2A3 | IGFALS | MP2K2 | | | | |
| 0.619 | NADK | CDON | | H2A3 | | MP2K2 | Notch1 | | | |
| 0.62 | NADK | CDON | | | IGFALS | | | | | |
| 0.62 | NADK | | CILP2 | H2A3 | | MP2K2 | | | | |
| 0.62 | NADK | | CILP2 | | | MP2K2 | Notch1 | | | |
| 0.62 | NADK | | | H2A3 | | | | PH | | |
| 0.62 | NADK | CDON | | H2A3 | | MP2K2 | | | | S100A13 |
| 0.621 | NADK | | | | IGFALS | | Notch1 | | | |
| 0.621 | NADK | CDON | CILP2 | | | | | | | S100A13 |
| 0.621 | NADK | CDON | CILP2 | H2A3 | | | | | | S100A13 |
| 0.621 | NADK | CDON | | | | | Notch1 | | | S100A13 |
| 0.622 | NADK | | | | | | | | PTN | |
| 0.622 | NADK | CDON | | H2A3 | IGFALS | | | | | |
| 0.622 | NADK | CDON | CILP2 | | | MP2K2 | | | | |
| 0.622 | NADK | | | | | MP2K2 | Notch1 | | | S100A13 |
| 0.622 | NADK | | | | | | | PH | | S100A13 |
| 0.624 | NADK | | | H2A3 | | | | | PTN | |
| 0.624 | NADK | CDON | | H2A3 | | | Notch1 | | | S100A13 |
| 0.625 | NADK | | | | | MP2K2 | | | PTN | |
| 0.625 | NADK | CDON | | | IGFALS | MP2K2 | | | | |
| 0.625 | NADK | | | H2A3 | IGFALS | | Notch1 | | | |
| 0.625 | NADK | CDON | CILP2 | | | MP2K2 | Notch1 | | | |
| 0.625 | NADK | | | | IGFALS | MP2K2 | Notch1 | | | |
| 0.625 | NADK | | CILP2 | | | | Notch1 | | | S100A13 |
| 0.625 | NADK | | | H2A3 | | | | PH | | S100A13 |
| 0.626 | NADK | CDON | | | IGFALS | | Notch1 | | | |
| 0.626 | NADK | | CILP2 | H2A3 | | MP2K2 | Notch1 | | | |
| 0.626 | NADK | CDON | | | | | | PH | | |
| 0.626 | NADK | | | | | MP2K2 | | PH | | |
| 0.626 | NADK | | | | IGFALS | | | | | S100A13 |
| 0.626 | NADK | | | H2A3 | IGFALS | | | | | S100A13 |
| 0.627 | NADK | | | H2A3 | | MP2K2 | | | PTN | |
| 0.627 | NADK | CDON | CILP2 | H2A3 | | MP2K2 | | | | |
| 0.627 | NADK | | CILP2 | H2A3 | | | Notch1 | | | S100A13 |
| 0.627 | NADK | | | H2A3 | | MP2K2 | Notch1 | | | S100A13 |
| 0.628 | NADK | | CILP2 | | IGFALS | | | | | |
| 0.628 | NADK | | CILP2 | H2A3 | IGFALS | | | | | |
| 0.628 | NADK | CDON | | | | MP2K2 | Notch1 | | | S100A13 |
| 0.629 | NADK | CDON | | | | | | | PTN | |
| 0.629 | NADK | CDON | | H2A3 | IGFALS | MP2K2 | | | | |
| 0.629 | NADK | CDON | | H2A3 | IGFALS | | Notch1 | | | |
| 0.629 | NADK | | | | | | Notch1 | PH | | |
| 0.629 | NADK | CDON | CILP2 | | | | Notch1 | | | S100A13 |
| 0.63 | NADK | CDON | CILP2 | H2A3 | | MP2K2 | Notch1 | | | |
| 0.63 | NADK | | | | | | | | PTN | S100A13 |
| 0.63 | NADK | CDON | CILP2 | H2A3 | | | Notch1 | | | S100A13 |
| 0.631 | NADK | CDON | | | IGFALS | MP2K2 | Notch1 | | | |
| 0.631 | NADK | | | H2A3 | IGFALS | MP2K2 | Notch1 | | | |
| 0.631 | NADK | | | H2A3 | | | | | PTN | S100A13 |
| 0.631 | NADK | | CILP2 | | | MP2K2 | | | | S100A13 |
| 0.632 | NADK | CDON | CILP2 | | IGFALS | | | | | |
| 0.632 | NADK | CDON | CILP2 | H2A3 | IGFALS | | | | | |
| 0.632 | NADK | CDON | | | | MP2K2 | | | PTN | |
| 0.632 | NADK | | CILP2 | | IGFALS | | Notch1 | | | |
| 0.632 | NADK | | CILP2 | | | | | PH | | |
| 0.632 | NADK | CDON | | H2A3 | | | | PH | | |
| 0.632 | NADK | | | H2A3 | | MP2K2 | | PH | | |
| 0.632 | NADK | | CILP2 | H2A3 | | MP2K2 | | | | S100A13 |
| 0.632 | NADK | | | | IGFALS | MP2K2 | | | | S100A13 |
| 0.632 | NADK | | | H2A3 | IGFALS | MP2K2 | | | | S100A13 |
| 0.633 | NADK | CDON | | | | | Notch1 | PH | | |
| 0.633 | NADK | CDON | | | IGFALS | | | | | S100A13 |
| 0.633 | NADK | | | | | MP2K2 | | | PTN | S100A13 |
| 0.633 | NADK | CDON | | H2A3 | | MP2K2 | Notch1 | | | S100A13 |
| 0.633 | NADK | | | | | MP2K2 | | PH | | S100A13 |
| 0.634 | NADK | CDON | | H2A3 | | | | | PTN | |
| 0.634 | NADK | | CILP2 | | IGFALS | MP2K2 | | | | |
| 0.634 | NADK | CDON | CILP2 | | IGFALS | | Notch1 | | | |
| 0.634 | NADK | | CILP2 | H2A3 | IGFALS | | Notch1 | | | |
| 0.634 | NADK | | CILP2 | H2A3 | | | | PH | | |
| 0.634 | NADK | CDON | | | | MP2K2 | | PH | | |
| 0.634 | NADK | | | | | MP2K2 | Notch1 | PH | | |
| 0.634 | NADK | CDON | | H2A3 | IGFALS | | | | | S100A13 |
| 0.634 | NADK | CDON | | | | | | PH | | S100A13 |
| 0.635 | NADK | | | H2A3 | | | Notch1 | PH | | |
| 0.635 | NADK | CDON | CILP2 | | | MP2K2 | | | | S100A13 |
| 0.635 | NADK | | | H2A3 | | MP2K2 | | | PTN | S100A13 |
| 0.636 | NADK | | CILP2 | H2A3 | IGFALS | MP2K2 | | | | |
| 0.636 | NADK | CDON | CILP2 | H2A3 | IGFALS | | Notch1 | | | |
| 0.636 | NADK | CDON | | H2A3 | IGFALS | MP2K2 | Notch1 | | | |
| 0.636 | NADK | CDON | CILP2 | H2A3 | | MP2K2 | | | | S100A13 |
| 0.637 | NADK | CDON | | H2A3 | | MP2K2 | | | PTN | |
| 0.637 | NADK | | CILP2 | | | MP2K2 | Notch1 | | | S100A13 |
| 0.637 | NADK | CDON | | H2A3 | | | | PH | | S100A13 |
| 0.637 | NADK | | | H2A3 | | MP2K2 | | PH | | S100A13 |
| 0.638 | NADK | CDON | CILP2 | | IGFALS | MP2K2 | | | | |
| 0.638 | NADK | | CILP2 | | IGFALS | MP2K2 | Notch1 | | | |
| 0.638 | NADK | | CILP2 | | | | Notch1 | PH | | |
| 0.638 | NADK | CDON | | | | | | | PTN | S100A13 |
| 0.638 | NADK | | CILP2 | H2A3 | IGFALS | | | | | S100A13 |
| 0.638 | NADK | | | | IGFALS | | Notch1 | | | S100A13 |
| 0.639 | NADK | CDON | CILP2 | | | | | PH | | |
| 0.639 | NADK | CDON | | | | MP2K2 | Notch1 | PH | | |
| 0.639 | NADK | | CILP2 | | IGFALS | | | | | S100A13 |
| 0.639 | NADK | CDON | | | IGFALS | MP2K2 | | | | S100A13 |
| 0.639 | NADK | | | H2A3 | IGFALS | | Notch1 | | | S100A13 |
| 0.64 | NADK | CDON | CILP2 | H2A3 | IGFALS | MP2K2 | | | | |
| 0.64 | NADK | CDON | CILP2 | | IGFALS | MP2K2 | Notch1 | | | |
| 0.64 | NADK | | | | IGFALS | | | PH | | |
| 0.64 | NADK | CDON | | H2A3 | | | Notch1 | PH | | |
| 0.64 | NADK | CDON | | | | MP2K2 | | | PTN | S100A13 |
| 0.64 | NADK | CDON | | H2A3 | IGFALS | MP2K2 | | | | S100A13 |
| 0.64 | NADK | CDON | CILP2 | | | MP2K2 | Notch1 | | | S100A13 |
| 0.64 | NADK | | CILP2 | H2A3 | | MP2K2 | Notch1 | | | S100A13 |
| 0.64 | NADK | | | | | | Notch1 | PH | | S100A13 |
| 0.641 | NADK | | CILP2 | H2A3 | IGFALS | MP2K2 | Notch1 | | | |
| 0.641 | NADK | CDON | CILP2 | H2A3 | | | | PH | | |
| 0.641 | NADK | CDON | | H2A3 | | MP2K2 | | PH | | |
| 0.641 | NADK | CDON | CILP2 | | | | Notch1 | PH | | |
| 0.641 | NADK | CDON | CILP2 | H2A3 | IGFALS | | | | | S100A13 |
| 0.641 | NADK | CDON | | | IGFALS | | Notch1 | | | S100A13 |
| 0.641 | NADK | | CILP2 | | | | | PH | | S100A13 |
| 0.641 | NADK | | CILP2 | H2A3 | | | | PH | | S100A13 |
| 0.642 | NADK | | | | | | Notch1 | | PTN | |
| 0.642 | NADK | | | H2A3 | IGFALS | | | PH | | |
| 0.642 | NADK | | CILP2 | | | MP2K2 | | PH | | |
| 0.642 | NADK | | CILP2 | H2A3 | | | Notch1 | PH | | |
| 0.642 | NADK | CDON | | H2A3 | | | | | PTN | S100A13 |
| 0.642 | NADK | CDON | CILP2 | | IGFALS | | | | | S100A13 |
| 0.642 | NADK | CDON | | | | MP2K2 | | PH | | S100A13 |
| 0.643 | NADK | | | | | MP2K2 | Notch1 | | PTN | |
| 0.643 | NADK | | | | | | | PH | PTN | |
| 0.643 | NADK | | | | IGFALS | MP2K2 | | PH | | |
| 0.643 | NADK | | | H2A3 | | MP2K2 | Notch1 | PH | | |
| 0.643 | NADK | CDON | | H2A3 | IGFALS | | Notch1 | | | S100A13 |
| 0.643 | NADK | CDON | CILP2 | H2A3 | | MP2K2 | Notch1 | | | S100A13 |
| 0.644 | NADK | CDON | | | | | Notch1 | | PTN | |
| 0.644 | NADK | CDON | CILP2 | H2A3 | IGFALS | MP2K2 | Notch1 | | | |
| 0.644 | NADK | CDON | | H2A3 | | MP2K2 | | | PTN | S100A13 |
| 0.644 | NADK | | | | IGFALS | MP2K2 | Notch1 | | | S100A13 |
| 0.644 | NADK | | | H2A3 | | | Notch1 | PH | | S100A13 |
| 0.645 | NADK | | | | IGFALS | | | | PTN | |
| 0.645 | NADK | | | H2A3 | IGFALS | | | | PTN | |
| 0.645 | NADK | | | | IGFALS | MP2K2 | | | PTN | |
| 0.645 | NADK | | | H2A3 | IGFALS | MP2K2 | | | PTN | |
| 0.645 | NADK | CDON | | | | MP2K2 | Notch1 | | PTN | |
| 0.645 | NADK | CDON | CILP2 | H2A3 | | | Notch1 | PH | | |
| 0.645 | NADK | | | H2A3 | IGFALS | MP2K2 | Notch1 | | | S100A13 |
| 0.645 | NADK | CDON | | | | | Notch1 | PH | | S100A13 |
| 0.646 | NADK | | | H2A3 | | | Notch1 | | PTN | |
| 0.646 | NADK | | | H2A3 | | | | PH | PTN | |
| 0.646 | NADK | | | | | MP2K2 | | PH | PTN | |
| 0.646 | NADK | | CILP2 | | | MP2K2 | Notch1 | PH | | |
| 0.646 | NADK | | CILP2 | | IGFALS | | Notch1 | | | S100A13 |
| 0.646 | NADK | | CILP2 | H2A3 | IGFALS | | Notch1 | | | S100A13 |
| 0.646 | NADK | CDON | CILP2 | | | | | PH | | S100A13 |
| 0.646 | NADK | CDON | CILP2 | H2A3 | | | | PH | | S100A13 |
| 0.646 | NADK | CDON | | H2A3 | | MP2K2 | | PH | | S100A13 |
| 0.646 | NADK | | | | | MP2K2 | Notch1 | PH | | S100A13 |
| 0.647 | NADK | | | H2A3 | | MP2K2 | Notch1 | | PTN | |
| 0.647 | NADK | CDON | | | IGFALS | | | PH | | |
| 0.647 | NADK | | CILP2 | H2A3 | | MP2K2 | | PH | | |
| 0.647 | NADK | | | H2A3 | IGFALS | MP2K2 | | PH | | |
| 0.647 | NADK | | | | IGFALS | | Notch1 | PH | | |
| 0.647 | NADK | CDON | CILP2 | | IGFALS | | Notch1 | | | S100A13 |
| 0.647 | NADK | CDON | CILP2 | H2A3 | IGFALS | | Notch1 | | | S100A13 |
| 0.647 | NADK | CDON | | | IGFALS | MP2K2 | Notch1 | | | S100A13 |
| 0.648 | NADK | CDON | CILP2 | | | MP2K2 | | PH | | |
| 0.648 | NADK | CDON | | H2A3 | | MP2K2 | Notch1 | PH | | |
| 0.648 | NADK | | CILP2 | | IGFALS | MP2K2 | | | | S100A13 |
| 0.648 | NADK | | CILP2 | H2A3 | IGFALS | MP2K2 | | | | S100A13 |
| 0.648 | NADK | | | | | | | PH | PTN | S100A13 |
| 0.648 | NADK | | CILP2 | | | | Notch1 | PH | | S100A13 |
| 0.649 | NADK | CDON | | | IGFALS | | | | PTN | |
| 0.649 | NADK | CDON | | | IGFALS | MP2K2 | | | PTN | |
| 0.649 | NADK | CDON | | H2A3 | | | Notch1 | | PTN | |
| 0.649 | NADK | CDON | | | | | | PH | PTN | |
| 0.649 | NADK | CDON | CILP2 | | | MP2K2 | Notch1 | PH | | |
| 0.649 | NADK | | | | IGFALS | MP2K2 | Notch1 | PH | | |
| 0.649 | NADK | CDON | | H2A3 | IGFALS | MP2K2 | Notch1 | | | S100A13 |
| 0.649 | NADK | | | | IGFALS | | | PH | | S100A13 |
| 0.649 | NADK | CDON | | H2A3 | | | Notch1 | PH | | S100A13 |
| 0.65 | NADK | CDON | | H2A3 | IGFALS | | | PH | | |
| 0.65 | NADK | | | H2A3 | | MP2K2 | | PH | PTN | |
| 0.65 | NADK | CDON | | | IGFALS | MP2K2 | | PH | | |
| 0.65 | NADK | CDON | | | IGFALS | | Notch1 | PH | | |
| 0.65 | NADK | | | H2A3 | IGFALS | | | PH | | S100A13 |
| 0.651 | NADK | CDON | | H2A3 | | MP2K2 | Notch1 | | PTN | |
| 0.651 | NADK | CDON | | | | MP2K2 | | PH | PTN | |
| 0.651 | NADK | | | H2A3 | IGFALS | | Notch1 | PH | | |
| 0.651 | NADK | CDON | CILP2 | | IGFALS | MP2K2 | | | | S100A13 |
| 0.651 | NADK | CDON | CILP2 | H2A3 | IGFALS | MP2K2 | | | | S100A13 |
| 0.651 | NADK | | | H2A3 | | | | PH | PTN | S100A13 |
| 0.651 | NADK | | | | | MP2K2 | | PH | PTN | S100A13 |
| 0.651 | NADK | CDON | CILP2 | | | | Notch1 | PH | | S100A13 |
| 0.651 | NADK | | CILP2 | H2A3 | | | Notch1 | PH | | S100A13 |
| 0.651 | NADK | CDON | | | | MP2K2 | Notch1 | PH | | S100A13 |
| 0.652 | NADK | CDON | | H2A3 | IGFALS | | | | PTN | |
| 0.652 | NADK | CDON | | H2A3 | IGFALS | MP2K2 | | | PTN | |
| 0.652 | NADK | | CILP2 | | IGFALS | | | PH | | |
| 0.652 | NADK | CDON | CILP2 | H2A3 | | MP2K2 | | PH | | |
| 0.652 | NADK | | CILP2 | H2A3 | | MP2K2 | Notch1 | PH | | |
| 0.652 | NADK | | | H2A3 | | MP2K2 | Notch1 | PH | | S100A13 |
| 0.653 | NADK | | CILP2 | H2A3 | IGFALS | | | PH | | |
| 0.653 | NADK | | | H2A3 | IGFALS | | | | PTN | S100A13 |
| 0.653 | NADK | | CILP2 | | IGFALS | MP2K2 | Notch1 | | | S100A13 |
| 0.653 | NADK | | CILP2 | | | MP2K2 | | PH | | S100A13 |
| 0.654 | NADK | CDON | | H2A3 | | | | PH | PTN | |
| 0.654 | NADK | | CILP2 | | IGFALS | | Notch1 | PH | | |
| 0.654 | NADK | CDON | | | IGFALS | MP2K2 | Notch1 | PH | | |
| 0.654 | NADK | | | | IGFALS | | | | PTN | S100A13 |
| 0.654 | NADK | | | | IGFALS | MP2K2 | | | PTN | S100A13 |
| 0.654 | NADK | | | H2A3 | IGFALS | MP2K2 | | | PTN | S100A13 |
| 0.654 | NADK | | CILP2 | H2A3 | IGFALS | MP2K2 | Notch1 | | | S100A13 |
| 0.654 | NADK | | | H2A3 | | MP2K2 | | PH | PTN | S100A13 |
| 0.654 | NADK | | CILP2 | H2A3 | | MP2K2 | | PH | | S100A13 |
| 0.654 | NADK | CDON | CILP2 | H2A3 | | | Notch1 | PH | | S100A13 |
| 0.655 | NADK | CDON | | H2A3 | IGFALS | MP2K2 | | PH | | |
| 0.655 | NADK | CDON | | H2A3 | IGFALS | | Notch1 | PH | | |
| 0.655 | NADK | CDON | CILP2 | H2A3 | | MP2K2 | Notch1 | PH | | |
| 0.655 | NADK | | | | | | Notch1 | | PTN | S100A13 |
| 0.655 | NADK | | | | | MP2K2 | Notch1 | | PTN | S100A13 |
| 0.655 | NADK | CDON | CILP2 | | IGFALS | MP2K2 | Notch1 | | | S100A13 |
| 0.655 | NADK | CDON | | | | | | PH | PTN | S100A13 |
| 0.655 | NADK | CDON | | | IGFALS | | | PH | | S100A13 |
| 0.655 | NADK | | | | IGFALS | MP2K2 | | PH | | S100A13 |
| 0.656 | NADK | | CILP2 | H2A3 | | | | | PTN | |
| 0.656 | NADK | CDON | CILP2 | | IGFALS | | | PH | | |
| 0.656 | NADK | | CILP2 | | IGFALS | MP2K2 | | PH | | |
| 0.656 | NADK | CDON | CILP2 | | IGFALS | | Notch1 | PH | | |
| 0.656 | NADK | | | H2A3 | IGFALS | MP2K2 | Notch1 | PH | | |
| 0.656 | NADK | CDON | CILP2 | H2A3 | IGFALS | MP2K2 | Notch1 | | | S100A13 |
| 0.656 | NADK | CDON | CILP2 | | | MP2K2 | | PH | | S100A13 |
| 0.656 | NADK | | | H2A3 | IGFALS | MP2K2 | | PH | | S100A13 |
| 0.656 | NADK | CDON | | H2A3 | | MP2K2 | Notch1 | PH | | S100A13 |
| 0.657 | NADK | CDON | CILP2 | H2A3 | IGFALS | | | PH | | |
| 0.657 | NADK | CDON | | H2A3 | | MP2K2 | | PH | PTN | |
| 0.657 | NADK | | CILP2 | H2A3 | IGFALS | | Notch1 | PH | | |
| 0.657 | NADK | CDON | | | | | Notch1 | | PTN | S100A13 |
| 0.657 | NADK | | | H2A3 | | | Notch1 | | PTN | S100A13 |
| 0.657 | NADK | CDON | | H2A3 | IGFALS | | | PH | | S100A13 |
| 0.657 | NADK | CDON | | | | MP2K2 | | PH | PTN | S100A13 |
| 0.657 | NADK | CDON | CILP2 | H2A3 | | MP2K2 | | PH | | S100A13 |
| 0.657 | NADK | | CILP2 | | | MP2K2 | Notch1 | PH | | S100A13 |
| 0.658 | NADK | CDON | | | IGFALS | | | | PTN | S100A13 |
| 0.658 | NADK | CDON | | | | MP2K2 | Notch1 | | PTN | S100A13 |
| 0.658 | NADK | | | H2A3 | | MP2K2 | Notch1 | | PTN | S100A13 |
| 0.658 | NADK | | | | IGFALS | | Notch1 | PH | | S100A13 |
| 0.659 | NADK | | CILP2 | | | | | | PTN | |
| 0.659 | NADK | CDON | CILP2 | | | | | | PTN | |
| 0.659 | NADK | CDON | CILP2 | H2A3 | | | | | PTN | |
| 0.659 | NADK | | | | IGFALS | MP2K2 | Notch1 | | PTN | |
| 0.659 | NADK | | | | IGFALS | | | PH | PTN | |
| 0.659 | NADK | | CILP2 | H2A3 | IGFALS | MP2K2 | | PH | | |
| 0.659 | NADK | CDON | CILP2 | H2A3 | IGFALS | | Notch1 | PH | | |
| 0.659 | NADK | | CILP2 | | IGFALS | MP2K2 | Notch1 | PH | | |
| 0.659 | NADK | CDON | | H2A3 | IGFALS | MP2K2 | Notch1 | PH | | |
| 0.659 | NADK | CDON | | H2A3 | IGFALS | | | | PTN | S100A13 |
| 0.659 | NADK | CDON | | | IGFALS | MP2K2 | | | PTN | S100A13 |
| 0.659 | NADK | CDON | | H2A3 | | | | PH | PTN | S100A13 |
| 0.659 | NADK | | CILP2 | H2A3 | IGFALS | | | PH | | S100A13 |
| 0.659 | NADK | CDON | | | IGFALS | MP2K2 | | PH | | S100A13 |
| 0.659 | NADK | CDON | CILP2 | | | MP2K2 | Notch1 | PH | | S100A13 |
| 0.66 | NADK | | | | IGFALS | | Notch1 | | PTN | |
| 0.66 | NADK | CDON | | | IGFALS | | Notch1 | | PTN | |
| 0.66 | NADK | CDON | | | IGFALS | MP2K2 | Notch1 | | PTN | |
| 0.66 | NADK | | | | IGFALS | MP2K2 | | PH | PTN | |
| 0.66 | NADK | CDON | CILP2 | | IGFALS | MP2K2 | | PH | | |
| 0.66 | NADK | | | | | | Notch1 | PH | PTN | |
| 0.66 | NADK | | | | | MP2K2 | Notch1 | PH | PTN | |
| 0.66 | NADK | CDON | | H2A3 | IGFALS | MP2K2 | | | PTN | S100A13 |
| 0.66 | NADK | CDON | | H2A3 | | | Notch1 | | PTN | S100A13 |
| 0.66 | NADK | | CILP2 | | IGFALS | | | PH | | S100A13 |
| 0.661 | NADK | | CILP2 | H2A3 | | MP2K2 | | | PTN | |
| 0.661 | NADK | CDON | | | | | Notch1 | PH | PTN | |
| 0.661 | NADK | CDON | CILP2 | | IGFALS | MP2K2 | Notch1 | PH | | |
| 0.661 | NADK | CDON | | H2A3 | | MP2K2 | Notch1 | | PTN | S100A13 |
| 0.661 | NADK | CDON | | H2A3 | | MP2K2 | | PH | PTN | S100A13 |
| 0.661 | NADK | | | H2A3 | IGFALS | | Notch1 | PH | | S100A13 |
| 0.661 | NADK | | CILP2 | H2A3 | | MP2K2 | Notch1 | PH | | S100A13 |
| 0.662 | NADK | | | H2A3 | IGFALS | | Notch1 | | PTN | |
| 0.662 | NADK | | | H2A3 | IGFALS | MP2K2 | Notch1 | | PTN | |
| 0.662 | NADK | | | H2A3 | IGFALS | | | PH | PTN | |
| 0.662 | NADK | CDON | | | | MP2K2 | Notch1 | PH | PTN | |
| 0.662 | NADK | | CILP2 | H2A3 | | | | | PTN | S100A13 |
| 0.662 | NADK | CDON | CILP2 | | IGFALS | | | PH | | S100A13 |
| 0.662 | NADK | CDON | CILP2 | H2A3 | IGFALS | | | PH | | S100A13 |
| 0.662 | NADK | CDON | | H2A3 | IGFALS | MP2K2 | | PH | | S100A13 |
| 0.662 | NADK | CDON | | | IGFALS | | Notch1 | PH | | S100A13 |
| 0.662 | NADK | CDON | CILP2 | H2A3 | | MP2K2 | Notch1 | PH | | S100A13 |
| 0.663 | NADK | | CILP2 | | | MP2K2 | | | PTN | |
| 0.663 | NADK | CDON | CILP2 | | | MP2K2 | | | PTN | |
| 0.663 | NADK | CDON | CILP2 | H2A3 | | MP2K2 | | | PTN | |
| 0.663 | NADK | | | H2A3 | IGFALS | MP2K2 | | PH | PTN | |
| 0.663 | NADK | CDON | CILP2 | H2A3 | IGFALS | MP2K2 | | PH | | |
| 0.663 | NADK | | CILP2 | H2A3 | IGFALS | MP2K2 | Notch1 | PH | | |
| 0.663 | NADK | CDON | CILP2 | H2A3 | | | | | PTN | S100A13 |
| 0.663 | NADK | | | | IGFALS | MP2K2 | Notch1 | PH | | S100A13 |
| 0.664 | NADK | CDON | | H2A3 | IGFALS | | Notch1 | | PTN | |
| 0.664 | NADK | CDON | | H2A3 | IGFALS | MP2K2 | Notch1 | | PTN | |
| 0.664 | NADK | | CILP2 | | | | | | PTN | S100A13 |
| 0.664 | NADK | | CILP2 | | IGFALS | | Notch1 | PH | | S100A13 |
| 0.664 | NADK | CDON | | H2A3 | IGFALS | | Notch1 | PH | | S100A13 |
| 0.665 | NADK | CDON | | | IGFALS | | | PH | PTN | |
| 0.665 | NADK | CDON | | | IGFALS | MP2K2 | | PH | PTN | |
| 0.665 | NADK | CDON | CILP2 | H2A3 | IGFALS | MP2K2 | Notch1 | PH | | |
| 0.665 | NADK | CDON | CILP2 | | | | | | PTN | S100A13 |
| 0.665 | NADK | | CILP2 | H2A3 | IGFALS | | Notch1 | PH | | S100A13 |
| 0.665 | NADK | CDON | | | IGFALS | MP2K2 | Notch1 | PH | | S100A13 |
| 0.666 | NADK | CDON | CILP2 | | | | Notch1 | | PTN | |
| 0.666 | NADK | | | H2A3 | | | Notch1 | PH | PTN | |
| 0.666 | NADK | | CILP2 | H2A3 | | MP2K2 | | | PTN | S100A13 |
| 0.666 | NADK | | CILP2 | | IGFALS | MP2K2 | | PH | | S100A13 |
| 0.666 | NADK | CDON | CILP2 | | IGFALS | | Notch1 | PH | | S100A13 |
| 0.666 | NADK | | | H2A3 | IGFALS | MP2K2 | Notch1 | PH | | S100A13 |
| 0.667 | NADK | | CILP2 | H2A3 | | | Notch1 | | PTN | |
| 0.667 | NADK | CDON | CILP2 | H2A3 | | | Notch1 | | PTN | |
| 0.667 | NADK | CDON | | H2A3 | | | Notch1 | PH | PTN | |
| 0.667 | NADK | | | H2A3 | | MP2K2 | Notch1 | PH | PTN | |
| 0.667 | NADK | | CILP2 | H2A3 | IGFALS | MP2K2 | | PH | | S100A13 |
| 0.667 | NADK | CDON | CILP2 | H2A3 | IGFALS | | Notch1 | PH | | S100A13 |
| 0.668 | NADK | | CILP2 | | | | Notch1 | | PTN | |
| 0.668 | NADK | CDON | CILP2 | | | MP2K2 | Notch1 | | PTN | |
| 0.668 | NADK | CDON | | H2A3 | IGFALS | | | PH | PTN | |
| 0.668 | NADK | | CILP2 | | | MP2K2 | | | PTN | S100A13 |
| 0.668 | NADK | CDON | CILP2 | | | MP2K2 | | | PTN | S100A13 |
| 0.668 | NADK | CDON | CILP2 | H2A3 | | MP2K2 | | | PTN | S100A13 |
| 0.668 | NADK | | | | IGFALS | | | PH | PTN | S100A13 |
| 0.668 | NADK | CDON | CILP2 | | IGFALS | MP2K2 | | PH | | S100A13 |
| 0.668 | NADK | | | | | | Notch1 | PH | PTN | S100A13 |
| 0.668 | NADK | CDON | | H2A3 | IGFALS | MP2K2 | Notch1 | PH | | S100A13 |
| 0.669 | NADK | | CILP2 | | | MP2K2 | Notch1 | | PTN | |
| 0.669 | NADK | CDON | | H2A3 | IGFALS | MP2K2 | | PH | PTN | |
| 0.669 | NADK | CDON | | H2A3 | | MP2K2 | Notch1 | PH | PTN | |
| 0.669 | NADK | | | H2A3 | IGFALS | | | PH | PTN | S100A13 |
| 0.669 | NADK | | | | IGFALS | MP2K2 | | PH | PTN | S100A13 |
| 0.669 | NADK | CDON | CILP2 | H2A3 | IGFALS | MP2K2 | | PH | | S100A13 |
| 0.669 | NADK | | | | | MP2K2 | Notch1 | PH | PTN | S100A13 |
| 0.67 | NADK | | CILP2 | H2A3 | IGFALS | | | | PTN | |
| 0.67 | NADK | | CILP2 | H2A3 | | MP2K2 | Notch1 | | PTN | |
| 0.67 | NADK | CDON | CILP2 | H2A3 | | MP2K2 | Notch1 | | PTN | |
| 0.67 | NADK | | CILP2 | H2A3 | | | | PH | PTN | |
| 0.67 | NADK | | | H2A3 | IGFALS | MP2K2 | | PH | PTN | S100A13 |
| 0.67 | NADK | CDON | | | | | Notch1 | PH | PTN | S100A13 |
| 0.67 | NADK | CDON | | | | MP2K2 | Notch1 | PH | PTN | S100A13 |
| 0.67 | NADK | | CILP2 | | IGFALS | MP2K2 | Notch1 | PH | | S100A13 |
| 0.671 | NADK | CDON | CILP2 | H2A3 | IGFALS | | | | PTN | |
| 0.671 | NADK | | CILP2 | H2A3 | IGFALS | MP2K2 | | | PTN | |
| 0.671 | NADK | | CILP2 | | | | | PH | PTN | |
| 0.671 | NADK | | | | IGFALS | | Notch1 | | PTN | S100A13 |
| 0.671 | NADK | CDON | | | IGFALS | | Notch1 | | PTN | S100A13 |
| 0.671 | NADK | | | | IGFALS | MP2K2 | Notch1 | | PTN | S100A13 |
| 0.671 | NADK | CDON | CILP2 | | IGFALS | MP2K2 | Notch1 | PH | | S100A13 |
| 0.672 | NADK | CDON | CILP2 | | IGFALS | | | | PTN | |
| 0.672 | NADK | CDON | CILP2 | H2A3 | IGFALS | MP2K2 | | | PTN | |
| 0.672 | NADK | CDON | CILP2 | | | | | PH | PTN | |
| 0.672 | NADK | CDON | CILP2 | H2A3 | | | | PH | PTN | |
| 0.672 | NADK | | | H2A3 | IGFALS | | Notch1 | | PTN | S100A13 |
| 0.672 | NADK | CDON | | | IGFALS | MP2K2 | Notch1 | | PTN | S100A13 |
| 0.672 | NADK | | | H2A3 | IGFALS | MP2K2 | Notch1 | | PTN | S100A13 |
| 0.672 | NADK | CDON | | | IGFALS | | | PH | PTN | S100A13 |
| 0.672 | NADK | CDON | | | IGFALS | MP2K2 | | PH | PTN | S100A13 |
| 0.672 | NADK | | | H2A3 | | | Notch1 | PH | PTN | S100A13 |
| 0.672 | NADK | | CILP2 | H2A3 | IGFALS | MP2K2 | Notch1 | PH | | S100A13 |
| 0.673 | NADK | | CILP2 | | IGFALS | | | | PTN | |
| 0.673 | NADK | CDON | CILP2 | | IGFALS | MP2K2 | | | PTN | |
| 0.673 | NADK | | | | IGFALS | | Notch1 | PH | PTN | |
| 0.673 | NADK | | | | IGFALS | MP2K2 | Notch1 | PH | PTN | |
| 0.673 | NADK | CDON | CILP2 | | | | Notch1 | | PTN | S100A13 |
| 0.673 | NADK | CDON | CILP2 | H2A3 | | | Notch1 | | PTN | S100A13 |
| 0.673 | NADK | CDON | | H2A3 | IGFALS | | Notch1 | | PTN | S100A13 |
| 0.673 | NADK | CDON | | H2A3 | IGFALS | MP2K2 | Notch1 | | PTN | S100A13 |
| 0.673 | NADK | | | H2A3 | | MP2K2 | Notch1 | PH | PTN | S100A13 |
| 0.673 | NADK | CDON | CILP2 | H2A3 | IGFALS | MP2K2 | Notch1 | PH | | S100A13 |
| 0.674 | NADK | | CILP2 | | IGFALS | MP2K2 | | | PTN | |
| 0.674 | NADK | | CILP2 | H2A3 | | MP2K2 | | PH | PTN | |
| 0.674 | NADK | CDON | | | IGFALS | | Notch1 | PH | PTN | |
| 0.674 | NADK | CDON | | | IGFALS | MP2K2 | Notch1 | PH | PTN | |
| 0.674 | NADK | | CILP2 | | | | Notch1 | | PTN | S100A13 |
| 0.674 | NADK | | CILP2 | H2A3 | | | Notch1 | | PTN | S100A13 |
| 0.674 | NADK | | CILP2 | H2A3 | | | | PH | PTN | S100A13 |
| 0.674 | NADK | CDON | | H2A3 | IGFALS | | | PH | PTN | S100A13 |
| 0.674 | NADK | CDON | | H2A3 | | | Notch1 | PH | PTN | S100A13 |
| 0.675 | NADK | | CILP2 | | | MP2K2 | | PH | PTN | |
| 0.675 | NADK | CDON | CILP2 | | | MP2K2 | | PH | PTN | |
| 0.675 | NADK | | CILP2 | H2A3 | IGFALS | | | | PTN | S100A13 |
| 0.675 | NADK | CDON | | H2A3 | IGFALS | MP2K2 | | PH | PTN | S100A13 |
| 0.675 | NADK | CDON | | H2A3 | | MP2K2 | Notch1 | PH | PTN | S100A13 |
| 0.676 | NADK | CDON | CILP2 | H2A3 | | MP2K2 | | PH | PTN | |
| 0.676 | NADK | CDON | CILP2 | H2A3 | IGFALS | | | | PTN | S100A13 |
| 0.676 | NADK | | CILP2 | | | MP2K2 | Notch1 | | PTN | S100A13 |
| 0.676 | NADK | CDON | CILP2 | | | MP2K2 | Notch1 | | PTN | S100A13 |
| 0.676 | NADK | CDON | CILP2 | H2A3 | | MP2K2 | Notch1 | | PTN | S100A13 |
| 0.676 | NADK | | CILP2 | | | | | PH | PTN | S100A13 |
| 0.676 | NADK | CDON | CILP2 | | | | | PH | PTN | S100A13 |
| 0.676 | NADK | CDON | CILP2 | H2A3 | | | | PH | PTN | S100A13 |
| 0.677 | NADK | CDON | CILP2 | | IGFALS | | Notch1 | | PTN | |
| 0.677 | NADK | CDON | CILP2 | H2A3 | IGFALS | | Notch1 | | PTN | |
| 0.677 | NADK | | | H2A3 | IGFALS | | Notch1 | PH | PTN | |
| 0.677 | NADK | | | H2A3 | IGFALS | MP2K2 | Notch1 | PH | PTN | |
| 0.677 | NADK | CDON | CILP2 | | IGFALS | | | | PTN | S100A13 |
| 0.677 | NADK | | CILP2 | H2A3 | | MP2K2 | Notch1 | | PTN | S100A13 |
| 0.678 | NADK | | CILP2 | H2A3 | IGFALS | | Notch1 | | PTN | |
| 0.678 | NADK | CDON | CILP2 | | IGFALS | MP2K2 | Notch1 | | PTN | |
| 0.678 | NADK | CDON | CILP2 | H2A3 | IGFALS | MP2K2 | Notch1 | | PTN | |
| 0.678 | NADK | CDON | CILP2 | | | | Notch1 | PH | PTN | |
| 0.678 | NADK | CDON | | H2A3 | IGFALS | | Notch1 | PH | PTN | |
| 0.678 | NADK | | CILP2 | | IGFALS | | | | PTN | S100A13 |
| 0.678 | NADK | | CILP2 | H2A3 | IGFALS | MP2K2 | | | PTN | S100A13 |
| 0.678 | NADK | CDON | CILP2 | H2A3 | IGFALS | MP2K2 | | | PTN | S100A13 |
| 0.679 | NADK | | CILP2 | | IGFALS | | Notch1 | | PTN | |
| 0.679 | NADK | | CILP2 | | IGFALS | MP2K2 | Notch1 | | PTN | |
| 0.679 | NADK | | CILP2 | H2A3 | IGFALS | MP2K2 | Notch1 | | PTN | |
| 0.679 | NADK | | CILP2 | | | | Notch1 | PH | PTN | |
| 0.679 | NADK | CDON | | H2A3 | IGFALS | MP2K2 | Notch1 | PH | PTN | |
| 0.679 | NADK | CDON | CILP2 | | IGFALS | MP2K2 | | | PTN | S100A13 |
| 0.679 | NADK | CDON | CILP2 | | | MP2K2 | | PH | PTN | S100A13 |
| 0.679 | NADK | | CILP2 | H2A3 | | MP2K2 | | PH | PTN | S100A13 |
| 0.679 | NADK | CDON | CILP2 | H2A3 | | MP2K2 | | PH | PTN | S100A13 |
| 0.68 | NADK | | CILP2 | H2A3 | | | Notch1 | PH | PTN | |
| 0.68 | NADK | CDON | CILP2 | H2A3 | | | Notch1 | PH | PTN | |
| 0.68 | NADK | CDON | CILP2 | | | MP2K2 | Notch1 | PH | PTN | |
| 0.68 | NADK | | CILP2 | | IGFALS | MP2K2 | | | PTN | S100A13 |
| 0.68 | NADK | | CILP2 | | | MP2K2 | | PH | PTN | S100A13 |
| 0.681 | NADK | | CILP2 | H2A3 | IGFALS | | | PH | PTN | |
| 0.681 | NADK | | CILP2 | | | MP2K2 | Notch1 | PH | PTN | |
| 0.682 | NADK | | CILP2 | | IGFALS | | | PH | PTN | |
| 0.682 | NADK | CDON | CILP2 | | IGFALS | | | PH | PTN | |
| 0.682 | NADK | CDON | CILP2 | H2A3 | IGFALS | | | PH | PTN | |
| 0.682 | NADK | CDON | CILP2 | H2A3 | | MP2K2 | Notch1 | PH | PTN | |
| 0.682 | NADK | | | | IGFALS | | Notch1 | PH | PTN | S100A13 |
| 0.682 | NADK | CDON | | | IGFALS | | Notch1 | PH | PTN | S100A13 |
| 0.682 | NADK | | | | IGFALS | MP2K2 | Notch1 | PH | PTN | S100A13 |
| 0.683 | NADK | CDON | CILP2 | | IGFALS | MP2K2 | | PH | PTN | |
| 0.683 | NADK | | CILP2 | H2A3 | IGFALS | MP2K2 | | PH | PTN | |
| 0.683 | NADK | | CILP2 | H2A3 | | MP2K2 | Notch1 | PH | PTN | |
| 0.683 | NADK | CDON | CILP2 | | IGFALS | | Notch1 | | PTN | S100A13 |
| 0.683 | NADK | CDON | CILP2 | H2A3 | IGFALS | | Notch1 | | PTN | S100A13 |
| 0.683 | NADK | CDON | CILP2 | | | | Notch1 | PH | PTN | S100A13 |
| 0.683 | NADK | CDON | | | IGFALS | MP2K2 | Notch1 | PH | PTN | S100A13 |
| 0.684 | NADK | | CILP2 | | IGFALS | MP2K2 | | PH | PTN | |
| 0.684 | NADK | CDON | CILP2 | H2A3 | IGFALS | MP2K2 | | PH | PTN | |
| 0.684 | NADK | | CILP2 | H2A3 | IGFALS | | Notch1 | | PTN | S100A13 |
| 0.684 | NADK | | CILP2 | | | | Notch1 | PH | PTN | S100A13 |
| 0.685 | NADK | | CILP2 | | IGFALS | | Notch1 | | PTN | S100A13 |
| 0.685 | NADK | CDON | CILP2 | | IGFALS | MP2K2 | Notch1 | | PTN | S100A13 |
| 0.685 | NADK | CDON | CILP2 | H2A3 | IGFALS | MP2K2 | Notch1 | | PTN | S100A13 |
| 0.685 | NADK | | CILP2 | H2A3 | | | Notch1 | PH | PTN | S100A13 |
| 0.685 | NADK | CDON | CILP2 | H2A3 | | | Notch1 | PH | PTN | S100A13 |
| 0.685 | NADK | | | H2A3 | IGFALS | | Notch1 | PH | PTN | S100A13 |
| 0.685 | NADK | CDON | | H2A3 | IGFALS | | Notch1 | PH | PTN | S100A13 |
| 0.685 | NADK | CDON | CILP2 | | | MP2K2 | Notch1 | PH | PTN | S100A13 |
| 0.685 | NADK | | | H2A3 | IGFALS | MP2K2 | Notch1 | PH | PTN | S100A13 |
| 0.685 | NADK | CDON | | H2A3 | IGFALS | MP2K2 | Notch1 | PH | PTN | S100A13 |
| 0.686 | NADK | | CILP2 | H2A3 | IGFALS | MP2K2 | Notch1 | | PTN | S100A13 |
| 0.686 | NADK | | CILP2 | H2A3 | IGFALS | | | PH | PTN | S100A13 |
| 0.686 | NADK | CDON | CILP2 | H2A3 | IGFALS | | | PH | PTN | S100A13 |
| 0.686 | NADK | | CILP2 | | | MP2K2 | Notch1 | PH | PTN | S100A13 |
| 0.687 | NADK | CDON | CILP2 | | IGFALS | | Notch1 | PH | PTN | |
| 0.687 | NADK | | CILP2 | | IGFALS | MP2K2 | Notch1 | | PTN | S100A13 |
| 0.687 | NADK | | CILP2 | | IGFALS | | | PH | PTN | S100A13 |
| 0.687 | NADK | CDON | CILP2 | | IGFALS | | | PH | PTN | S100A13 |
| 0.687 | NADK | CDON | CILP2 | H2A3 | | MP2K2 | Notch1 | PH | PTN | S100A13 |
| 0.688 | NADK | | CILP2 | | IGFALS | | Notch1 | PH | PTN | |
| 0.688 | NADK | CDON | CILP2 | H2A3 | IGFALS | | Notch1 | PH | PTN | |
| 0.688 | NADK | CDON | CILP2 | | IGFALS | MP2K2 | Notch1 | PH | PTN | |
| 0.688 | NADK | | CILP2 | H2A3 | IGFALS | MP2K2 | | PH | PTN | S100A13 |
| 0.688 | NADK | CDON | CILP2 | H2A3 | IGFALS | MP2K2 | | PH | PTN | S100A13 |
| 0.688 | NADK | | CILP2 | H2A3 | | MP2K2 | Notch1 | PH | PTN | S100A13 |
| 0.689 | NADK | | CILP2 | H2A3 | IGFALS | | Notch1 | PH | PTN | |
| 0.689 | NADK | | CILP2 | | IGFALS | MP2K2 | Notch1 | PH | PTN | |
| 0.689 | NADK | CDON | CILP2 | H2A3 | IGFALS | MP2K2 | Notch1 | PH | PTN | |
| 0.689 | NADK | | CILP2 | | IGFALS | MP2K2 | | PH | PTN | S100A13 |
| 0.689 | NADK | CDON | CILP2 | | IGFALS | MP2K2 | | PH | PTN | S100A13 |
| 0.69 | NADK | | CILP2 | H2A3 | IGFALS | MP2K2 | Notch1 | PH | PTN | |
| 0.692 | NADK | CDON | CILP2 | | IGFALS | | Notch1 | PH | PTN | S100A13 |
| 0.693 | NADK | CDON | CILP2 | H2A3 | IGFALS | | Notch1 | PH | PTN | S100A13 |
| 0.694 | NADK | | CILP2 | | IGFALS | | Notch1 | PH | PTN | S100A13 |
| 0.694 | NADK | | CILP2 | H2A3 | IGFALS | | Notch1 | PH | PTN | S100A13 |
| 0.694 | NADK | CDON | CILP2 | | IGFALS | MP2K2 | Notch1 | PH | PTN | S100A13 |
| 0.694 | NADK | CDON | CILP2 | H2A3 | IGFALS | MP2K2 | Notch1 | PH | PTN | S100A13 |
| 0.695 | NADK | | CILP2 | | IGFALS | MP2K2 | Notch1 | PH | PTN | S100A13 |
| *0.696 | NADK | | CILP2 | H2A3 | IGFALS | MP2K2 | Notch1 | PH | PTN | S100A13 |

*Included in Table 38

TABLE 43
CDON Biomarker Panels-20 Year Model

| AUC | CDON | CILP2 | H2A3 | IGFALS | MP2K2 | NADK | Notch1 | PH | PTN | S100A13 |
|---|---|---|---|---|---|---|---|---|---|---|
| 0.583 | CDON | | H2A3 | | | | | | | |
| 0.583 | CDON | | | | | | Notch1 | | | |
| 0.583 | CDON | | | | | | | | | S100A13 |
| 0.587 | CDON | | | | MP2K2 | | | | | |
| 0.589 | CDON | CILP2 | | | | | | | | |
| 0.589 | CDON | | | | | NADK | | | | |
| 0.594 | CDON | | H2A3 | | | NADK | | | | |
| 0.595 | CDON | | H2A3 | | | | | | | S100A13 |
| 0.596 | CDON | CILP2 | | | | | Notch1 | | | |
| 0.598 | CDON | | | | MP2K2 | | Notch1 | | | |
| 0.599 | CDON | | H2A3 | | | | Notch1 | | | |
| 0.6 | CDON | CILP2 | H2A3 | | | | | | | |
| 0.6 | CDON | | | | | NADK | Notch1 | | | |
| 0.6 | CDON | | | | MP2K2 | | | | | S100A13 |
| 0.601 | CDON | CILP2 | | | | | | | | S100A13 |
| 0.603 | CDON | | | | MP2K2 | NADK | | | | |
| 0.604 | CDON | | H2A3 | | MP2K2 | | | | | |
| 0.605 | CDON | | | | | NADK | | | | S100A13 |
| 0.605 | CDON | | | | | | Notch1 | | | S100A13 |
| 0.607 | CDON | | | IGFALS | | | | | | |
| 0.607 | CDON | | H2A3 | | | NADK | Notch1 | | | |
| 0.608 | CDON | CILP2 | | | MP2K2 | | | | | |
| 0.608 | CDON | CILP2 | H2A3 | | | | Notch1 | | | |
| 0.608 | CDON | | H2A3 | | | NADK | | | | S100A13 |
| 0.609 | CDON | CILP2 | | | | NADK | | | | |
| 0.609 | CDON | | H2A3 | | MP2K2 | NADK | | | | |
| 0.609 | CDON | | | | | | | PH | | |
| 0.609 | CDON | CILP2 | H2A3 | | | | | | | S100A13 |
| 0.61 | CDON | | | | MP2K2 | NADK | Notch1 | | | |
| 0.611 | CDON | CILP2 | H2A3 | | | NADK | | | | |
| 0.612 | CDON | CILP2 | | | MP2K2 | | Notch1 | | | |
| 0.612 | CDON | | H2A3 | | MP2K2 | | | | | S100A13 |
| 0.613 | CDON | | | | | | | | PTN | |
| 0.613 | CDON | CILP2 | | | | NADK | Notch1 | | | |
| 0.613 | CDON | CILP2 | | | | | Notch1 | | | S100A13 |
| 0.615 | CDON | | H2A3 | | MP2K2 | | Notch1 | | | |
| 0.615 | CDON | | | | MP2K2 | NADK | | | | S100A13 |
| 0.615 | CDON | | H2A3 | | | | Notch1 | | | S100A13 |
| 0.616 | CDON | | H2A3 | IGFALS | | | | | | |
| 0.616 | CDON | | | IGFALS | MP2K2 | | | | | |
| 0.616 | CDON | | | IGFALS | | | Notch1 | | | |
| 0.617 | CDON | CILP2 | H2A3 | | | NADK | Notch1 | | | |
| 0.617 | CDON | | | | MP2K2 | | Notch1 | | | S100A13 |
| 0.617 | CDON | | | | | | | PH | | S100A13 |
| 0.619 | CDON | | | | MP2K2 | | | | PTN | |
| 0.619 | CDON | | H2A3 | | MP2K2 | NADK | Notch1 | | | |
| 0.62 | CDON | CILP2 | | IGFALS | | | | | | |
| 0.62 | CDON | | | IGFALS | | NADK | | | | |
| 0.62 | CDON | | | | | | Notch1 | PH | | |
| 0.62 | CDON | CILP2 | | | MP2K2 | | | | | S100A13 |
| 0.62 | CDON | | H2A3 | | MP2K2 | NADK | | | | S100A13 |
| 0.621 | CDON | CILP2 | H2A3 | | MP2K2 | | | | | |
| 0.621 | CDON | | | IGFALS | | | | | | S100A13 |
| 0.621 | CDON | CILP2 | | | | NADK | | | | S100A13 |
| 0.621 | CDON | CILP2 | H2A3 | | | NADK | | | | S100A13 |
| 0.621 | CDON | CILP2 | H2A3 | | | | Notch1 | | | S100A13 |
| 0.621 | CDON | | | | | NADK | Notch1 | | | S100A13 |
| 0.622 | CDON | | H2A3 | IGFALS | | NADK | | | | |
| 0.622 | CDON | CILP2 | | | MP2K2 | NADK | | | | |
| 0.622 | CDON | | | | MP2K2 | | | PH | | |
| 0.623 | CDON | CILP2 | | | | | | PH | | |
| 0.624 | CDON | CILP2 | | IGFALS | | | Notch1 | | | |
| 0.624 | CDON | | | IGFALS | MP2K2 | | Notch1 | | | |
| 0.624 | CDON | | | | | | | | PTN | S100A13 |
| 0.624 | CDON | | H2A3 | | | NADK | Notch1 | | | S100A13 |
| 0.625 | CDON | | H2A3 | | | | | | PTN | |
| 0.625 | CDON | | H2A3 | IGFALS | MP2K2 | | | | | |
| 0.625 | CDON | | | IGFALS | MP2K2 | NADK | | | | |
| 0.625 | CDON | | H2A3 | IGFALS | | | Notch1 | | | |
| 0.625 | CDON | CILP2 | | | MP2K2 | NADK | Notch1 | | | |
| 0.625 | CDON | | H2A3 | | | | | PH | | |
| 0.626 | CDON | CILP2 | H2A3 | IGFALS | | | | | | |
| 0.626 | CDON | CILP2 | H2A3 | | MP2K2 | | Notch1 | | | |
| 0.626 | CDON | | | IGFALS | | NADK | Notch1 | | | |
| 0.626 | CDON | | | | | NADK | | PH | | |
| 0.626 | CDON | | H2A3 | IGFALS | | | | | | S100A13 |
| 0.627 | CDON | CILP2 | H2A3 | | MP2K2 | NADK | | | | |
| 0.627 | CDON | CILP2 | | | MP2K2 | | Notch1 | | | S100A13 |
| 0.627 | CDON | | H2A3 | | MP2K2 | | Notch1 | | | S100A13 |
| 0.628 | CDON | CILP2 | | | | | Notch1 | PH | | |
| 0.628 | CDON | | | | MP2K2 | | | | PTN | S100A13 |
| 0.628 | CDON | CILP2 | H2A3 | | MP2K2 | | | | | S100A13 |
| 0.628 | CDON | | | | MP2K2 | NADK | Notch1 | | | S100A13 |
| 0.629 | CDON | CILP2 | | IGFALS | MP2K2 | | | | | |
| 0.629 | CDON | | | | | NADK | | | PTN | |
| 0.629 | CDON | | H2A3 | IGFALS | MP2K2 | NADK | | | | |
| 0.629 | CDON | | H2A3 | IGFALS | | NADK | Notch1 | | | |
| 0.629 | CDON | | | | MP2K2 | | Notch1 | PH | | |
| 0.629 | CDON | CILP2 | | | | NADK | Notch1 | | | S100A13 |
| 0.629 | CDON | | H2A3 | | | | | PH | | S100A13 |
| 0.63 | CDON | | H2A3 | | MP2K2 | | | | PTN | |
| 0.63 | CDON | CILP2 | H2A3 | | MP2K2 | NADK | Notch1 | | | |
| 0.63 | CDON | CILP2 | | IGFALS | | | | | | S100A13 |
| 0.63 | CDON | | | IGFALS | MP2K2 | | | | | S100A13 |
| 0.63 | CDON | CILP2 | H2A3 | | | NADK | Notch1 | | | S100A13 |
| 0.63 | CDON | CILP2 | | | | | | PH | | S100A13 |
| 0.63 | CDON | | | | MP2K2 | | | PH | | S100A13 |
| 0.631 | CDON | | | | | | Notch1 | | PTN | |
| 0.631 | CDON | CILP2 | H2A3 | IGFALS | | | Notch1 | | | |
| 0.631 | CDON | | | IGFALS | MP2K2 | NADK | Notch1 | | | |
| 0.632 | CDON | CILP2 | | IGFALS | | NADK | | | | |
| 0.632 | CDON | CILP2 | H2A3 | IGFALS | | NADK | | | | |
| 0.632 | CDON | | | | MP2K2 | NADK | | | PTN | |
| 0.632 | CDON | | H2A3 | | | NADK | | PH | | |
| 0.632 | CDON | | | | | | Notch1 | PH | | S100A13 |
| 0.633 | CDON | | | | | NADK | Notch1 | PH | | |
| 0.633 | CDON | | H2A3 | | | | | | PTN | S100A13 |
| 0.633 | CDON | CILP2 | H2A3 | IGFALS | | | | | | S100A13 |
| 0.633 | CDON | | | IGFALS | | NADK | | | | S100A13 |
| 0.633 | CDON | | | IGFALS | | | Notch1 | | | S100A13 |
| 0.633 | CDON | | H2A3 | | MP2K2 | NADK | Notch1 | | | S100A13 |
| 0.634 | CDON | | H2A3 | | | NADK | | | PTN | |
| 0.634 | CDON | | | | MP2K2 | | Notch1 | | PTN | |
| 0.634 | CDON | CILP2 | | IGFALS | MP2K2 | | Notch1 | | | |
| 0.634 | CDON | | H2A3 | IGFALS | MP2K2 | | Notch1 | | | |
| 0.634 | CDON | CILP2 | | IGFALS | | NADK | Notch1 | | | |
| 0.634 | CDON | CILP2 | H2A3 | | | | | PH | | |
| 0.634 | CDON | | | | MP2K2 | NADK | | PH | | |
| 0.634 | CDON | | H2A3 | IGFALS | | NADK | | | | S100A13 |
| 0.634 | CDON | | | | | NADK | | PH | | S100A13 |
| 0.635 | CDON | | H2A3 | IGFALS | MP2K2 | | | | | S100A13 |
| 0.635 | CDON | CILP2 | | | MP2K2 | NADK | | | | S100A13 |
| 0.636 | CDON | CILP2 | H2A3 | IGFALS | | NADK | Notch1 | | | |
| 0.636 | CDON | | H2A3 | IGFALS | MP2K2 | NADK | Notch1 | | | |
| 0.636 | CDON | | H2A3 | | | | Notch1 | PH | | |
| 0.636 | CDON | CILP2 | H2A3 | | MP2K2 | NADK | | | | S100A13 |
| 0.636 | CDON | CILP2 | H2A3 | | MP2K2 | | Notch1 | | | S100A13 |
| 0.637 | CDON | CILP2 | H2A3 | IGFALS | MP2K2 | | | | | |
| 0.637 | CDON | | H2A3 | | MP2K2 | NADK | | | PTN | |
| 0.637 | CDON | | | | | | | PH | PTN | |
| 0.637 | CDON | | | IGFALS | | | | PH | | |
| 0.637 | CDON | CILP2 | | | MP2K2 | | | PH | | |
| 0.637 | CDON | | H2A3 | | MP2K2 | | | | PTN | S100A13 |
| 0.637 | CDON | | H2A3 | IGFALS | | | Notch1 | | | S100A13 |
| 0.637 | CDON | CILP2 | H2A3 | | | | | PH | | S100A13 |
| 0.637 | CDON | | H2A3 | | | NADK | | PH | | S100A13 |
| 0.638 | CDON | CILP2 | | IGFALS | MP2K2 | NADK | | | | |
| 0.638 | CDON | | H2A3 | | MP2K2 | | | PH | | |
| 0.638 | CDON | | | | | NADK | | | PTN | S100A13 |
| 0.638 | CDON | CILP2 | | IGFALS | | | Notch1 | | | S100A13 |
| 0.638 | CDON | CILP2 | | | | | Notch1 | PH | | S100A13 |
| 0.639 | CDON | | | IGFALS | | | | | PTN | |
| 0.639 | CDON | CILP2 | | | | NADK | | PH | | |
| 0.639 | CDON | | | | MP2K2 | NADK | Notch1 | PH | | |
| 0.639 | CDON | | | IGFALS | MP2K2 | NADK | | | | S100A13 |
| 0.64 | CDON | CILP2 | H2A3 | IGFALS | MP2K2 | NADK | | | | |
| 0.64 | CDON | CILP2 | | IGFALS | MP2K2 | NADK | Notch1 | | | |
| 0.64 | CDON | CILP2 | H2A3 | | | | Notch1 | PH | | |
| 0.64 | CDON | CILP2 | | | MP2K2 | | Notch1 | PH | | |
| 0.64 | CDON | | H2A3 | | | NADK | Notch1 | PH | | |
| 0.64 | CDON | | | | MP2K2 | NADK | | | PTN | S100A13 |
| 0.64 | CDON | | H2A3 | IGFALS | MP2K2 | NADK | | | | S100A13 |
| 0.64 | CDON | CILP2 | | | MP2K2 | NADK | Notch1 | | | S100A13 |
| 0.64 | CDON | | H2A3 | | MP2K2 | | | PH | | S100A13 |
| 0.641 | CDON | | | IGFALS | MP2K2 | | | | PTN | |
| 0.641 | CDON | CILP2 | H2A3 | IGFALS | MP2K2 | | Notch1 | | | |
| 0.641 | CDON | | | | MP2K2 | | | PH | PTN | |
| 0.641 | CDON | CILP2 | H2A3 | | | NADK | | PH | | |
| 0.641 | CDON | | H2A3 | | MP2K2 | NADK | | PH | | |
| 0.641 | CDON | CILP2 | | | | NADK | Notch1 | PH | | |
| 0.641 | CDON | CILP2 | H2A3 | IGFALS | | NADK | | | | S100A13 |
| 0.641 | CDON | CILP2 | H2A3 | IGFALS | | | Notch1 | | | S100A13 |
| 0.641 | CDON | | | IGFALS | MP2K2 | | Notch1 | | | S100A13 |
| 0.641 | CDON | | | IGFALS | | NADK | Notch1 | | | S100A13 |
| 0.641 | CDON | | | | MP2K2 | | Notch1 | PH | | S100A13 |
| 0.642 | CDON | CILP2 | | IGFALS | MP2K2 | | | | | S100A13 |
| 0.642 | CDON | | H2A3 | | | NADK | | | PTN | S100A13 |
| 0.642 | CDON | CILP2 | | IGFALS | | NADK | | | | S100A13 |
| 0.642 | CDON | | | | MP2K2 | NADK | | PH | | S100A13 |
| 0.643 | CDON | | H2A3 | | | | Notch1 | | PTN | |
| 0.643 | CDON | | | IGFALS | MP2K2 | | | PH | | |
| 0.643 | CDON | | | IGFALS | | | Notch1 | PH | | |
| 0.643 | CDON | | H2A3 | IGFALS | | NADK | Notch1 | | | S100A13 |
| 0.643 | CDON | CILP2 | H2A3 | | MP2K2 | NADK | Notch1 | | | S100A13 |
| 0.643 | CDON | | | | | | | PH | PTN | S100A13 |
| 0.643 | CDON | | H2A3 | | | | Notch1 | PH | | S100A13 |
| 0.644 | CDON | | | | | NADK | Notch1 | | PTN | |
| 0.644 | CDON | CILP2 | H2A3 | IGFALS | MP2K2 | NADK | Notch1 | | | |
| 0.644 | CDON | | H2A3 | | MP2K2 | NADK | | | PTN | S100A13 |
| 0.644 | CDON | | | IGFALS | | | | PH | | S100A13 |
| 0.644 | CDON | CILP2 | | | MP2K2 | | | PH | | S100A13 |
| 0.645 | CDON | | | | MP2K2 | NADK | Notch1 | | PTN | |
| 0.645 | CDON | CILP2 | H2A3 | | | NADK | Notch1 | PH | | |
| 0.645 | CDON | CILP2 | H2A3 | IGFALS | MP2K2 | | | | | S100A13 |
| 0.645 | CDON | | H2A3 | IGFALS | MP2K2 | | Notch1 | | | S100A13 |
| 0.645 | CDON | | | | | NADK | Notch1 | PH | | S100A13 |
| 0.646 | CDON | | H2A3 | | MP2K2 | | Notch1 | | PTN | |
| 0.646 | CDON | | H2A3 | | MP2K2 | | Notch1 | PH | | |
| 0.646 | CDON | | | | | | Notch1 | | PTN | S100A13 |
| 0.646 | CDON | | | | MP2K2 | | | PH | PTN | S100A13 |
| 0.646 | CDON | CILP2 | | | | NADK | | PH | | S100A13 |
| 0.646 | CDON | CILP2 | H2A3 | | | NADK | | PH | | S100A13 |
| 0.646 | CDON | | H2A3 | | MP2K2 | NADK | | PH | | S100A13 |
| 0.646 | CDON | CILP2 | H2A3 | | | | Notch1 | PH | | S100A13 |
| 0.647 | CDON | | H2A3 | IGFALS | | | | | PTN | |
| 0.647 | CDON | CILP2 | | IGFALS | | | | PH | | |
| 0.647 | CDON | | H2A3 | IGFALS | | | | PH | | |
| 0.647 | CDON | | | IGFALS | | NADK | | PH | | |
| 0.647 | CDON | CILP2 | | IGFALS | MP2K2 | | Notch1 | | | S100A13 |
| 0.647 | CDON | CILP2 | | IGFALS | | NADK | Notch1 | | | S100A13 |
| 0.647 | CDON | CILP2 | H2A3 | IGFALS | | NADK | Notch1 | | | S100A13 |
| 0.647 | CDON | | | IGFALS | MP2K2 | NADK | Notch1 | | | S100A13 |
| 0.648 | CDON | CILP2 | | | | | | | PTN | |
| 0.648 | CDON | | H2A3 | IGFALS | MP2K2 | | | | PTN | |
| 0.648 | CDON | | H2A3 | | | | | PH | PTN | |
| 0.648 | CDON | CILP2 | | | MP2K2 | NADK | | PH | | |
| 0.648 | CDON | | | IGFALS | MP2K2 | | Notch1 | PH | | |
| 0.648 | CDON | | H2A3 | | MP2K2 | NADK | Notch1 | PH | | |
| 0.648 | CDON | | | | MP2K2 | | Notch1 | | PTN | S100A13 |
| 0.649 | CDON | | | IGFALS | | NADK | | | PTN | |
| 0.649 | CDON | | | IGFALS | MP2K2 | NADK | | | PTN | |
| 0.649 | CDON | | H2A3 | | | NADK | Notch1 | | PTN | |
| 0.649 | CDON | CILP2 | H2A3 | | MP2K2 | | | PH | | |
| 0.649 | CDON | | | | | NADK | | PH | PTN | |
| 0.649 | CDON | CILP2 | | IGFALS | | | Notch1 | PH | | |
| 0.649 | CDON | CILP2 | | | MP2K2 | NADK | Notch1 | PH | | |
| 0.649 | CDON | | | IGFALS | | | | | PTN | S100A13 |
| 0.649 | CDON | | H2A3 | IGFALS | MP2K2 | NADK | Notch1 | | | S100A13 |
| 0.649 | CDON | CILP2 | | | MP2K2 | | Notch1 | PH | | S100A13 |
| 0.649 | CDON | | H2A3 | | | NADK | Notch1 | PH | | S100A13 |
| 0.65 | CDON | | H2A3 | IGFALS | | NADK | | PH | | |
| 0.65 | CDON | | | IGFALS | MP2K2 | NADK | | PH | | |
| 0.65 | CDON | | | IGFALS | | NADK | Notch1 | PH | | |
| 0.651 | CDON | CILP2 | H2A3 | | | | | | PTN | |
| 0.651 | CDON | | H2A3 | | MP2K2 | NADK | Notch1 | | PTN | |
| 0.651 | CDON | | | | MP2K2 | NADK | | PH | PTN | |
| 0.651 | CDON | | | | | | Notch1 | PH | PTN | |
| 0.651 | CDON | | | IGFALS | MP2K2 | | | | PTN | S100A13 |
| 0.651 | CDON | CILP2 | | IGFALS | MP2K2 | NADK | | | | S100A13 |
| 0.651 | CDON | CILP2 | H2A3 | IGFALS | MP2K2 | NADK | | | | S100A13 |
| 0.651 | CDON | | H2A3 | IGFALS | | | | PH | | S100A13 |
| 0.651 | CDON | CILP2 | H2A3 | | MP2K2 | | | PH | | S100A13 |
| 0.651 | CDON | CILP2 | | | | NADK | Notch1 | PH | | S100A13 |
| 0.651 | CDON | | | | MP2K2 | NADK | Notch1 | PH | | S100A13 |
| 0.652 | CDON | | H2A3 | IGFALS | | NADK | | | PTN | |
| 0.652 | CDON | | H2A3 | IGFALS | MP2K2 | NADK | | | PTN | |
| 0.652 | CDON | CILP2 | H2A3 | | MP2K2 | NADK | | PH | | |
| 0.652 | CDON | CILP2 | H2A3 | IGFALS | MP2K2 | | Notch1 | | | S100A13 |
| 0.652 | CDON | | H2A3 | | | | | PH | PTN | S100A13 |
| 0.652 | CDON | CILP2 | | IGFALS | | | | PH | | S100A13 |
| 0.652 | CDON | | | IGFALS | MP2K2 | | | PH | | S100A13 |
| 0.652 | CDON | | H2A3 | | MP2K2 | | Notch1 | PH | | S100A13 |
| 0.653 | CDON | CILP2 | | | MP2K2 | | | | PTN | |
| 0.653 | CDON | | | IGFALS | | | Notch1 | | PTN | |
| 0.653 | CDON | | H2A3 | | MP2K2 | | | PH | PTN | |
| 0.653 | CDON | | H2A3 | IGFALS | | | Notch1 | PH | | |
| 0.653 | CDON | | | | MP2K2 | | Notch1 | PH | PTN | |
| 0.653 | CDON | CILP2 | H2A3 | | MP2K2 | | Notch1 | PH | | |
| 0.653 | CDON | CILP2 | | | | | | | PTN | S100A13 |
| 0.653 | CDON | | | IGFALS | | | Notch1 | PH | | S100A13 |
| 0.654 | CDON | | | IGFALS | MP2K2 | | Notch1 | | PTN | |
| 0.654 | CDON | CILP2 | H2A3 | IGFALS | | | | PH | | |
| 0.654 | CDON | CILP2 | | IGFALS | MP2K2 | | | PH | | |
| 0.654 | CDON | | H2A3 | IGFALS | MP2K2 | | | PH | | |
| 0.654 | CDON | | H2A3 | | | NADK | | PH | PTN | |
| 0.654 | CDON | | | IGFALS | MP2K2 | NADK | Notch1 | PH | | |
| 0.654 | CDON | | H2A3 | IGFALS | | | | | PTN | S100A13 |
| 0.654 | CDON | | H2A3 | | | | Notch1 | | PTN | S100A13 |
| 0.654 | CDON | CILP2 | H2A3 | | | NADK | Notch1 | PH | | S100A13 |
| 0.655 | CDON | | H2A3 | IGFALS | MP2K2 | NADK | | PH | | |
| 0.655 | CDON | CILP2 | | IGFALS | MP2K2 | | Notch1 | PH | | |
| 0.655 | CDON | | H2A3 | IGFALS | | NADK | Notch1 | PH | | |
| 0.655 | CDON | CILP2 | H2A3 | | MP2K2 | NADK | Notch1 | PH | | |
| 0.655 | CDON | CILP2 | H2A3 | | | | | | PTN | S100A13 |
| 0.655 | CDON | CILP2 | | IGFALS | MP2K2 | NADK | Notch1 | | | S100A13 |
| 0.655 | CDON | | | | | NADK | | PH | PTN | S100A13 |
| 0.655 | CDON | | | IGFALS | | NADK | | PH | | S100A13 |
| 0.656 | CDON | CILP2 | | | | | Notch1 | | PTN | |
| 0.656 | CDON | CILP2 | | IGFALS | | NADK | | PH | | |
| 0.656 | CDON | CILP2 | H2A3 | IGFALS | | | Notch1 | PH | | |
| 0.656 | CDON | CILP2 | | IGFALS | | NADK | Notch1 | PH | | |
| 0.656 | CDON | | H2A3 | IGFALS | MP2K2 | | | | PTN | S100A13 |
| 0.656 | CDON | | H2A3 | | MP2K2 | | Notch1 | | PTN | S100A13 |
| 0.656 | CDON | CILP2 | H2A3 | IGFALS | MP2K2 | NADK | Notch1 | | | S100A13 |
| 0.656 | CDON | CILP2 | H2A3 | IGFALS | | | | PH | | S100A13 |
| 0.656 | CDON | | H2A3 | | MP2K2 | | | PH | PTN | S100A13 |
| 0.656 | CDON | CILP2 | | | MP2K2 | NADK | | PH | | S100A13 |
| 0.656 | CDON | | H2A3 | | MP2K2 | NADK | Notch1 | PH | | S100A13 |
| 0.657 | CDON | CILP2 | H2A3 | | MP2K2 | | | | PTN | |
| 0.657 | CDON | | | IGFALS | | | | PH | PTN | |
| 0.657 | CDON | CILP2 | H2A3 | IGFALS | | NADK | | PH | | |
| 0.657 | CDON | | H2A3 | | MP2K2 | NADK | | PH | PTN | |
| 0.657 | CDON | | | | | NADK | Notch1 | | PTN | S100A13 |
| 0.657 | CDON | | H2A3 | IGFALS | | NADK | | PH | | S100A13 |
| 0.657 | CDON | | | | MP2K2 | NADK | | PH | PTN | S100A13 |
| 0.657 | CDON | CILP2 | H2A3 | | MP2K2 | NADK | | PH | | S100A13 |
| 0.657 | CDON | CILP2 | | IGFALS | | | Notch1 | PH | | S100A13 |
| 0.657 | CDON | CILP2 | H2A3 | | MP2K2 | | Notch1 | PH | | S100A13 |
| 0.658 | CDON | | | IGFALS | MP2K2 | | | PH | PTN | |
| 0.658 | CDON | CILP2 | | | MP2K2 | | | | PTN | S100A13 |
| 0.658 | CDON | | | IGFALS | | NADK | | | PTN | S100A13 |
| 0.658 | CDON | | | | MP2K2 | NADK | Notch1 | | PTN | S100A13 |
| 0.658 | CDON | | H2A3 | IGFALS | MP2K2 | | | PH | | S100A13 |
| 0.659 | CDON | CILP2 | | | | NADK | | | PTN | |
| 0.659 | CDON | CILP2 | H2A3 | | | NADK | | | PTN | |
| 0.659 | CDON | | H2A3 | IGFALS | MP2K2 | | Notch1 | PH | | |
| 0.659 | CDON | CILP2 | H2A3 | IGFALS | | NADK | Notch1 | PH | | |
| 0.659 | CDON | | H2A3 | IGFALS | MP2K2 | NADK | Notch1 | PH | | |
| 0.659 | CDON | | H2A3 | IGFALS | | NADK | | | PTN | S100A13 |
| 0.659 | CDON | | | IGFALS | MP2K2 | NADK | | | PTN | S100A13 |
| 0.659 | CDON | | H2A3 | | | NADK | | PH | PTN | S100A13 |
| 0.659 | CDON | | | IGFALS | MP2K2 | NADK | | PH | | S100A13 |
| 0.659 | CDON | | H2A3 | IGFALS | | | Notch1 | PH | | S100A13 |
| 0.659 | CDON | | | IGFALS | MP2K2 | | Notch1 | PH | | S100A13 |
| 0.659 | CDON | CILP2 | | | MP2K2 | NADK | Notch1 | PH | | S100A13 |
| 0.66 | CDON | | H2A3 | IGFALS | | | Notch1 | | PTN | |
| 0.66 | CDON | CILP2 | | | MP2K2 | | Notch1 | | PTN | |
| 0.66 | CDON | | | IGFALS | | NADK | Notch1 | | PTN | |
| 0.66 | CDON | | | IGFALS | MP2K2 | NADK | Notch1 | | PTN | |
| 0.66 | CDON | CILP2 | | IGFALS | MP2K2 | NADK | | PH | | |
| 0.66 | CDON | | H2A3 | IGFALS | MP2K2 | NADK | | | PTN | S100A13 |
| 0.66 | CDON | | H2A3 | | | NADK | Notch1 | | PTN | S100A13 |
| 0.661 | CDON | CILP2 | H2A3 | | | | Notch1 | | PTN | |
| 0.661 | CDON | | H2A3 | IGFALS | MP2K2 | | Notch1 | | PTN | |
| 0.661 | CDON | | | | | NADK | Notch1 | PH | PTN | |
| 0.661 | CDON | CILP2 | | IGFALS | MP2K2 | NADK | Notch1 | PH | | |
| 0.661 | CDON | CILP2 | H2A3 | | MP2K2 | | | | PTN | S100A13 |
| 0.661 | CDON | | H2A3 | | MP2K2 | NADK | Notch1 | | PTN | S100A13 |
| 0.661 | CDON | CILP2 | | IGFALS | MP2K2 | | | PH | | S100A13 |
| 0.661 | CDON | | H2A3 | | MP2K2 | NADK | | PH | PTN | S100A13 |
| 0.661 | CDON | | | | | | Notch1 | PH | PTN | S100A13 |
| 0.662 | CDON | CILP2 | H2A3 | IGFALS | MP2K2 | | | PH | | |
| 0.662 | CDON | | | | MP2K2 | NADK | Notch1 | PH | PTN | |
| 0.662 | CDON | CILP2 | | | | | Notch1 | | PTN | S100A13 |
| 0.662 | CDON | CILP2 | | IGFALS | | NADK | | PH | | S100A13 |
| 0.662 | CDON | CILP2 | H2A3 | IGFALS | | NADK | | PH | | S100A13 |
| 0.662 | CDON | | H2A3 | IGFALS | MP2K2 | NADK | | PH | | S100A13 |
| 0.662 | CDON | CILP2 | H2A3 | IGFALS | | | Notch1 | PH | | S100A13 |
| 0.662 | CDON | | | | MP2K2 | | Notch1 | PH | PTN | S100A13 |
| 0.662 | CDON | | | IGFALS | | NADK | Notch1 | PH | | S100A13 |
| 0.662 | CDON | CILP2 | H2A3 | | MP2K2 | NADK | Notch1 | PH | | S100A13 |
| 0.663 | CDON | CILP2 | | | MP2K2 | NADK | | | PTN | |
| 0.663 | CDON | CILP2 | H2A3 | | MP2K2 | NADK | | | PTN | |
| 0.663 | CDON | CILP2 | | | | | | PH | PTN | |
| 0.663 | CDON | CILP2 | H2A3 | IGFALS | MP2K2 | NADK | | PH | | |
| 0.663 | CDON | CILP2 | H2A3 | | | NADK | | | PTN | S100A13 |
| 0.663 | CDON | | | IGFALS | | | | PH | PTN | S100A13 |
| 0.664 | CDON | CILP2 | | IGFALS | | | | | PTN | |
| 0.664 | CDON | | H2A3 | IGFALS | | NADK | Notch1 | | PTN | |
| 0.664 | CDON | | H2A3 | IGFALS | MP2K2 | NADK | Notch1 | | PTN | |
| 0.664 | CDON | | H2A3 | | | | Notch1 | PH | PTN | |
| 0.664 | CDON | CILP2 | H2A3 | IGFALS | MP2K2 | | Notch1 | PH | | |
| 0.664 | CDON | | | IGFALS | | | Notch1 | | PTN | S100A13 |
| 0.664 | CDON | | H2A3 | IGFALS | | NADK | Notch1 | PH | | S100A13 |
| 0.665 | CDON | CILP2 | H2A3 | | MP2K2 | | Notch1 | | PTN | |
| 0.665 | CDON | | H2A3 | IGFALS | | | | PH | PTN | |
| 0.665 | CDON | | | IGFALS | | NADK | | PH | PTN | |
| 0.665 | CDON | | | IGFALS | MP2K2 | NADK | | PH | PTN | |
| 0.665 | CDON | CILP2 | H2A3 | IGFALS | MP2K2 | NADK | Notch1 | PH | | |
| 0.665 | CDON | CILP2 | | | | NADK | | | PTN | S100A13 |
| 0.665 | CDON | | | IGFALS | MP2K2 | | | PH | PTN | S100A13 |
| 0.665 | CDON | CILP2 | H2A3 | IGFALS | MP2K2 | | | PH | | S100A13 |
| 0.665 | CDON | CILP2 | | IGFALS | MP2K2 | | Notch1 | PH | | S100A13 |
| 0.665 | CDON | | | IGFALS | MP2K2 | NADK | Notch1 | PH | | S100A13 |
| 0.666 | CDON | CILP2 | H2A3 | IGFALS | | | | | PTN | |
| 0.666 | CDON | CILP2 | | IGFALS | MP2K2 | | | | PTN | |
| 0.666 | CDON | CILP2 | | | | NADK | Notch1 | | PTN | |
| 0.666 | CDON | | H2A3 | | MP2K2 | | Notch1 | PH | PTN | |
| 0.666 | CDON | CILP2 | | | MP2K2 | | Notch1 | | PTN | S100A13 |
| 0.666 | CDON | | | IGFALS | MP2K2 | | Notch1 | | PTN | S100A13 |
| 0.666 | CDON | CILP2 | | | | | | PH | PTN | S100A13 |
| 0.666 | CDON | | H2A3 | IGFALS | MP2K2 | | Notch1 | PH | | S100A13 |
| 0.666 | CDON | CILP2 | | IGFALS | | NADK | Notch1 | PH | | S100A13 |
| 0.667 | CDON | CILP2 | H2A3 | | | NADK | Notch1 | | PTN | |
| 0.667 | CDON | CILP2 | H2A3 | | | | | PH | PTN | |
| 0.667 | CDON | | H2A3 | IGFALS | MP2K2 | | | PH | PTN | |
| 0.667 | CDON | | H2A3 | | | NADK | Notch1 | PH | PTN | |
| 0.667 | CDON | CILP2 | H2A3 | | | | Notch1 | | PTN | S100A13 |
| 0.667 | CDON | CILP2 | H2A3 | IGFALS | | NADK | Notch1 | PH | | S100A13 |
| 0.668 | CDON | CILP2 | H2A3 | IGFALS | MP2K2 | | | | PTN | |
| 0.668 | CDON | CILP2 | | | MP2K2 | NADK | Notch1 | | PTN | |
| 0.668 | CDON | CILP2 | | | MP2K2 | | | PH | PTN | |
| 0.668 | CDON | | H2A3 | IGFALS | | NADK | | PH | PTN | |
| 0.668 | CDON | | | IGFALS | | | Notch1 | PH | PTN | |
| 0.668 | CDON | CILP2 | | | MP2K2 | NADK | | | PTN | S100A13 |
| 0.668 | CDON | CILP2 | H2A3 | | MP2K2 | NADK | | | PTN | S100A13 |
| 0.668 | CDON | | H2A3 | IGFALS | | | Notch1 | | PTN | S100A13 |
| 0.668 | CDON | CILP2 | | IGFALS | MP2K2 | NADK | | PH | | S100A13 |
| 0.668 | CDON | | H2A3 | IGFALS | MP2K2 | NADK | Notch1 | PH | | S100A13 |
| 0.669 | CDON | | H2A3 | IGFALS | MP2K2 | NADK | | PH | PTN | |
| 0.669 | CDON | | | IGFALS | MP2K2 | | Notch1 | PH | PTN | |
| 0.669 | CDON | | H2A3 | | MP2K2 | NADK | Notch1 | PH | PTN | |
| 0.669 | CDON | CILP2 | | IGFALS | | | | | PTN | S100A13 |
| 0.669 | CDON | | H2A3 | IGFALS | | | | PH | PTN | S100A13 |
| 0.669 | CDON | CILP2 | H2A3 | IGFALS | MP2K2 | NADK | | PH | | S100A13 |
| 0.669 | CDON | CILP2 | H2A3 | IGFALS | MP2K2 | | Notch1 | PH | | S100A13 |
| 0.67 | CDON | CILP2 | | IGFALS | | | Notch1 | | PTN | |
| 0.67 | CDON | CILP2 | H2A3 | | MP2K2 | NADK | Notch1 | | PTN | |
| 0.67 | CDON | CILP2 | | | | | Notch1 | PH | PTN | |
| 0.67 | CDON | CILP2 | H2A3 | IGFALS | | | | | PTN | S100A13 |
| 0.67 | CDON | | H2A3 | IGFALS | MP2K2 | | Notch1 | | PTN | S100A13 |
| 0.67 | CDON | CILP2 | H2A3 | | | | | PH | PTN | S100A13 |
| 0.67 | CDON | | H2A3 | | | | Notch1 | PH | PTN | S100A13 |
| 0.67 | CDON | | | | | NADK | Notch1 | PH | PTN | S100A13 |
| 0.67 | CDON | | | | MP2K2 | NADK | Notch1 | PH | PTN | S100A13 |
| 0.671 | CDON | CILP2 | H2A3 | IGFALS | | NADK | | | PTN | |
| 0.671 | CDON | CILP2 | H2A3 | | MP2K2 | | Notch1 | | PTN | S100A13 |
| 0.671 | CDON | | | IGFALS | | NADK | Notch1 | | PTN | S100A13 |
| 0.671 | CDON | CILP2 | | | MP2K2 | | | PH | PTN | S100A13 |
| 0.671 | CDON | | H2A3 | IGFALS | MP2K2 | | | PH | PTN | S100A13 |
| 0.671 | CDON | CILP2 | | IGFALS | MP2K2 | NADK | Notch1 | PH | | S100A13 |
| 0.672 | CDON | CILP2 | | IGFALS | | NADK | | | PTN | |
| 0.672 | CDON | CILP2 | H2A3 | IGFALS | MP2K2 | NADK | | | PTN | |
| 0.672 | CDON | CILP2 | | IGFALS | MP2K2 | | Notch1 | | PTN | |
| 0.672 | CDON | CILP2 | H2A3 | | MP2K2 | | | PH | PTN | |
| 0.672 | CDON | CILP2 | | | | NADK | | PH | PTN | |
| 0.672 | CDON | CILP2 | H2A3 | | | NADK | | PH | PTN | |
| 0.672 | CDON | CILP2 | | IGFALS | MP2K2 | | | | PTN | S100A13 |
| 0.672 | CDON | | | IGFALS | MP2K2 | NADK | Notch1 | | PTN | S100A13 |
| 0.672 | CDON | | | IGFALS | | NADK | | PH | PTN | S100A13 |
| 0.672 | CDON | | | IGFALS | MP2K2 | NADK | | PH | PTN | S100A13 |
| 0.672 | CDON | | H2A3 | | MP2K2 | | Notch1 | PH | PTN | S100A13 |
| 0.673 | CDON | CILP2 | | IGFALS | MP2K2 | NADK | | | PTN | |
| 0.673 | CDON | CILP2 | H2A3 | IGFALS | | | Notch1 | | PTN | |
| 0.673 | CDON | CILP2 | | | MP2K2 | | Notch1 | PH | PTN | |
| 0.673 | CDON | CILP2 | | | | NADK | Notch1 | | PTN | S100A13 |
| 0.673 | CDON | CILP2 | H2A3 | | | NADK | Notch1 | | PTN | S100A13 |
| 0.673 | CDON | | H2A3 | IGFALS | | NADK | Notch1 | | PTN | S100A13 |
| 0.673 | CDON | | H2A3 | IGFALS | MP2K2 | NADK | Notch1 | | PTN | S100A13 |
| 0.673 | CDON | CILP2 | H2A3 | IGFALS | MP2K2 | NADK | Notch1 | PH | | S100A13 |
| 0.674 | CDON | | | IGFALS | | NADK | Notch1 | PH | PTN | |
| 0.674 | CDON | | | IGFALS | MP2K2 | NADK | Notch1 | PH | PTN | |
| 0.674 | CDON | CILP2 | H2A3 | IGFALS | MP2K2 | | | | PTN | S100A13 |
| 0.674 | CDON | | H2A3 | IGFALS | | NADK | | PH | PTN | S100A13 |
| 0.674 | CDON | CILP2 | | | | | Notch1 | PH | PTN | S100A13 |
| 0.674 | CDON | | H2A3 | | | NADK | Notch1 | PH | PTN | S100A13 |
| 0.675 | CDON | CILP2 | H2A3 | IGFALS | MP2K2 | | Notch1 | | PTN | |
| 0.675 | CDON | CILP2 | | | MP2K2 | NADK | | PH | PTN | |
| 0.675 | CDON | CILP2 | H2A3 | | MP2K2 | | | PH | PTN | S100A13 |
| 0.675 | CDON | | H2A3 | IGFALS | MP2K2 | NADK | | PH | PTN | S100A13 |
| 0.675 | CDON | | H2A3 | | MP2K2 | NADK | Notch1 | PH | PTN | S100A13 |
| 0.676 | CDON | CILP2 | | IGFALS | | | | PH | PTN | |
| 0.676 | CDON | CILP2 | H2A3 | | MP2K2 | NADK | | PH | PTN | |
| 0.676 | CDON | CILP2 | H2A3 | | | | Notch1 | PH | PTN | |
| 0.676 | CDON | CILP2 | H2A3 | IGFALS | | NADK | | | PTN | S100A13 |
| 0.676 | CDON | CILP2 | | | MP2K2 | NADK | Notch1 | | PTN | S100A13 |
| 0.676 | CDON | CILP2 | H2A3 | | MP2K2 | NADK | Notch1 | | PTN | S100A13 |
| 0.676 | CDON | CILP2 | | | | NADK | | PH | PTN | S100A13 |
| 0.676 | CDON | CILP2 | H2A3 | | | NADK | | PH | PTN | S100A13 |
| 0.676 | CDON | | | IGFALS | | | Notch1 | PH | PTN | S100A13 |
| 0.677 | CDON | CILP2 | | IGFALS | | NADK | Notch1 | | PTN | |
| 0.677 | CDON | CILP2 | H2A3 | IGFALS | | NADK | Notch1 | | PTN | |
| 0.677 | CDON | | H2A3 | IGFALS | | | Notch1 | PH | PTN | |
| 0.677 | CDON | | H2A3 | IGFALS | MP2K2 | | Notch1 | PH | PTN | |
| 0.677 | CDON | CILP2 | | IGFALS | | NADK | | | PTN | S100A13 |
| 0.677 | CDON | CILP2 | | IGFALS | | | Notch1 | | PTN | S100A13 |
| 0.677 | CDON | CILP2 | | | MP2K2 | | Notch1 | PH | PTN | S100A13 |
| 0.677 | CDON | | | IGFALS | MP2K2 | | Notch1 | PH | PTN | S100A13 |
| 0.678 | CDON | CILP2 | | IGFALS | MP2K2 | NADK | Notch1 | | PTN | |
| 0.678 | CDON | CILP2 | H2A3 | IGFALS | MP2K2 | NADK | Notch1 | | PTN | |
| 0.678 | CDON | CILP2 | | | | NADK | Notch1 | PH | PTN | |
| 0.678 | CDON | | H2A3 | IGFALS | | NADK | Notch1 | PH | PTN | |
| 0.678 | CDON | CILP2 | H2A3 | IGFALS | MP2K2 | NADK | | | PTN | S100A13 |
| 0.679 | CDON | CILP2 | H2A3 | IGFALS | | | | PH | PTN | |
| 0.679 | CDON | CILP2 | | IGFALS | MP2K2 | | | PH | PTN | |
| 0.679 | CDON | CILP2 | H2A3 | | MP2K2 | | Notch1 | PH | PTN | |
| 0.679 | CDON | | H2A3 | IGFALS | MP2K2 | NADK | Notch1 | PH | PTN | |
| 0.679 | CDON | CILP2 | | IGFALS | MP2K2 | NADK | | | PTN | S100A13 |
| 0.679 | CDON | CILP2 | H2A3 | IGFALS | | | Notch1 | | PTN | S100A13 |
| 0.679 | CDON | CILP2 | | IGFALS | MP2K2 | | Notch1 | | PTN | S100A13 |
| 0.679 | CDON | CILP2 | | | MP2K2 | NADK | | PH | PTN | S100A13 |
| 0.679 | CDON | CILP2 | H2A3 | | MP2K2 | NADK | | PH | PTN | S100A13 |
| 0.68 | CDON | CILP2 | H2A3 | | | NADK | Notch1 | PH | PTN | |
| 0.68 | CDON | CILP2 | | | MP2K2 | NADK | Notch1 | PH | PTN | |
| 0.68 | CDON | CILP2 | | IGFALS | | | | PH | PTN | S100A13 |
| 0.68 | CDON | CILP2 | H2A3 | | | | Notch1 | PH | PTN | S100A13 |
| 0.681 | CDON | CILP2 | H2A3 | IGFALS | MP2K2 | | Notch1 | | PTN | S100A13 |
| 0.682 | CDON | CILP2 | H2A3 | IGFALS | MP2K2 | | | PH | PTN | |
| 0.682 | CDON | CILP2 | | IGFALS | | NADK | | PH | PTN | |
| 0.682 | CDON | CILP2 | H2A3 | IGFALS | | NADK | | PH | PTN | |
| 0.682 | CDON | CILP2 | | IGFALS | | | Notch1 | PH | PTN | |
| 0.682 | CDON | CILP2 | H2A3 | | MP2K2 | NADK | Notch1 | PH | PTN | |
| 0.682 | CDON | CILP2 | H2A3 | IGFALS | | | | PH | PTN | S100A13 |
| 0.682 | CDON | | H2A3 | IGFALS | | | Notch1 | PH | PTN | S100A13 |
| 0.682 | CDON | | | IGFALS | | NADK | Notch1 | PH | PTN | S100A13 |
| 0.683 | CDON | CILP2 | | IGFALS | MP2K2 | NADK | | PH | PTN | |
| 0.683 | CDON | CILP2 | | IGFALS | | NADK | Notch1 | | PTN | S100A13 |
| 0.683 | CDON | CILP2 | H2A3 | IGFALS | | NADK | Notch1 | | PTN | S100A13 |
| 0.683 | CDON | CILP2 | | IGFALS | MP2K2 | | | PH | PTN | S100A13 |
| 0.683 | CDON | CILP2 | H2A3 | | MP2K2 | | Notch1 | PH | PTN | S100A13 |
| 0.683 | CDON | | H2A3 | IGFALS | MP2K2 | | Notch1 | PH | PTN | S100A13 |
| 0.683 | CDON | CILP2 | | | | NADK | Notch1 | PH | PTN | S100A13 |
| 0.683 | CDON | | | IGFALS | MP2K2 | NADK | Notch1 | PH | PTN | S100A13 |
| 0.684 | CDON | CILP2 | H2A3 | IGFALS | MP2K2 | NADK | | PH | PTN | |
| 0.684 | CDON | CILP2 | | IGFALS | MP2K2 | | Notch1 | PH | PTN | |
| 0.685 | CDON | CILP2 | | IGFALS | MP2K2 | NADK | Notch1 | | PTN | S100A13 |
| 0.685 | CDON | CILP2 | H2A3 | IGFALS | MP2K2 | NADK | Notch1 | | PTN | S100A13 |
| 0.685 | CDON | CILP2 | H2A3 | IGFALS | MP2K2 | | | PH | PTN | S100A13 |
| 0.685 | CDON | CILP2 | H2A3 | | | NADK | Notch1 | PH | PTN | S100A13 |
| 0.685 | CDON | | H2A3 | IGFALS | | NADK | Notch1 | PH | PTN | S100A13 |
| 0.685 | CDON | CILP2 | | | MP2K2 | NADK | Notch1 | PH | PTN | S100A13 |
| 0.685 | CDON | | H2A3 | IGFALS | MP2K2 | NADK | Notch1 | PH | PTN | S100A13 |
| 0.686 | CDON | CILP2 | H2A3 | IGFALS | | | Notch1 | PH | PTN | |
| 0.686 | CDON | CILP2 | H2A3 | IGFALS | | NADK | | PH | PTN | S100A13 |
| 0.686 | CDON | CILP2 | | IGFALS | | | Notch1 | PH | PTN | S100A13 |
| 0.687 | CDON | CILP2 | | IGFALS | | NADK | Notch1 | PH | PTN | |
| 0.687 | CDON | CILP2 | | IGFALS | | NADK | | PH | PTN | S100A13 |
| 0.687 | CDON | CILP2 | H2A3 | | MP2K2 | NADK | Notch1 | PH | PTN | S100A13 |
| 0.688 | CDON | CILP2 | H2A3 | IGFALS | MP2K2 | | Notch1 | PH | PTN | |
| 0.688 | CDON | CILP2 | H2A3 | IGFALS | | NADK | Notch1 | PH | PTN | |
| 0.688 | CDON | CILP2 | | IGFALS | MP2K2 | NADK | Notch1 | PH | PTN | |
| 0.688 | CDON | CILP2 | H2A3 | IGFALS | MP2K2 | NADK | | PH | PTN | S100A13 |
| 0.688 | CDON | CILP2 | | IGFALS | MP2K2 | | Notch1 | PH | PTN | S100A13 |
| 0.689 | CDON | CILP2 | H2A3 | IGFALS | MP2K2 | NADK | Notch1 | PH | PTN | |
| 0.689 | CDON | CILP2 | | IGFALS | MP2K2 | NADK | | PH | PTN | S100A13 |
| 0.69 | CDON | CILP2 | H2A3 | IGFALS | | | Notch1 | PH | PTN | S100A13 |
| 0.692 | CDON | CILP2 | H2A3 | IGFALS | MP2K2 | | Notch1 | PH | PTN | S100A13 |
| 0.692 | CDON | CILP2 | | IGFALS | | NADK | Notch1 | PH | PTN | S100A13 |
| 0.693 | CDON | CILP2 | H2A3 | IGFALS | | NADK | Notch1 | PH | PTN | S100A13 |
| 0.694 | CDON | CILP2 | | IGFALS | MP2K2 | NADK | Notch1 | PH | PTN | S100A13 |
| *0.694 | CDON | CILP2 | H2A3 | IGFALS | MP2K2 | NADK | Notch1 | PH | PTN | S100A13 |

*Included in Table 44

TABLE 44
CDON Biomarker Panels, Includes CDON, CILP2, IGFALS, MP2K2, NADK,
Notch1, PH, PTN and S100A13 plus additional markers-20 Year Model

| | | | | | | | | | | | | | cal- | | | |
|---|---|---|---|---|---|---|---|---|---|---|---|---|---|---|---|---|
| | | | | | | | | | | | | | granulin | | | |
| AUC | * | CDCP1 | NDST1 | TIM-1 | TBCA | ASB9 | OPG | SVEP1 | PARC | ATS13 | YKL-40 | sRAGE | B | IL-18 | NFL | NPTXR |
| 0.694 | * | | | | | | | | | | | | | | | |
| 0.705 | * | CDCP1 | | | | | | | | | | | | | | |
| 0.698 | * | | NDST1 | | | | | | | | | | | | | |
| 0.699 | * | | | TIM-1 | | | | | | | | | | | | |
| 0.693 | * | | | | TBCA | | | | | | | | | | | |
| 0.7 | * | | | | | ASB9 | | | | | | | | | | |
| 0.695 | * | | | | | | OPG | | | | | | | | | |
| 0.696 | * | | | | | | | SVEP1 | | | | | | | | |
| 0.695 | * | | | | | | | | PARC | | | | | | | |
| 0.695 | * | | | | | | | | | ATS13 | | | | | | |
| 0.696 | * | | | | | | | | | | YKL-40 | | | | | |
| 0.701 | * | | | | | | | | | | | sRAGE | | | | |
| 0.697 | * | | | | | | | | | | | | calgranulin B | | | |
| 0.694 | * | | | | | | | | | | | | | IL-18 | | |
| 0.693 | * | | | | | | | | | | | | | | NFL | |
| 0.706 | * | | | | | | | | | | | | | | | NPTXR |

* Includes CDON, CILP2, IGFALS, MP2K2, NADK, Notch1, PH, PTN and S100A13

TABLE 45
MP2K2 Biomarker Panels--20 Year Model

| AUC | MP2K2 | CDON | CILP2 | H2A3 | IGFALS | NADK | Notch1 | PH | PTN | S100A13 |
|---|---|---|---|---|---|---|---|---|---|---|
| 0.579 | MP2K2 | | | | | | | | | S100A13 |
| 0.585 | MP2K2 | | | H2A3 | | | | | | |
| 0.587 | MP2K2 | CDON | | | | | | | | |
| 0.588 | MP2K2 | | | | | NADK | | | | |
| 0.588 | MP2K2 | | | | | | Notch1 | | | |
| 0.594 | MP2K2 | | | H2A3 | | NADK | | | | |
| 0.594 | MP2K2 | | | H2A3 | | | | | | S100A13 |
| 0.598 | MP2K2 | | CILP2 | | | | | | | |
| 0.598 | MP2K2 | CDON | | | | | Notch1 | | | |
| 0.6 | MP2K2 | CDON | | | | | | | | S100A13 |
| 0.602 | MP2K2 | | | | | NADK | | | | S100A13 |
| 0.603 | MP2K2 | CDON | | | | NADK | | | | |
| 0.603 | MP2K2 | | | | | NADK | Notch1 | | | |
| 0.604 | MP2K2 | CDON | | H2A3 | | | | | | |
| 0.605 | MP2K2 | | | | | | | | PTN | |
| 0.606 | MP2K2 | | | H2A3 | | NADK | | | | S100A13 |
| 0.607 | MP2K2 | | | | IGFALS | | | | | |
| 0.607 | MP2K2 | | CILP2 | | | | Notch1 | | | |
| 0.607 | MP2K2 | | | H2A3 | | | Notch1 | | | |
| 0.608 | MP2K2 | CDON | CILP2 | | | | | | | |
| 0.608 | MP2K2 | | | | | | Notch1 | | | S100A13 |
| 0.609 | MP2K2 | CDON | | H2A3 | | NADK | | | | |
| 0.609 | MP2K2 | | | | | | | PH | | |
| 0.61 | MP2K2 | CDON | | | | NADK | Notch1 | | | |
| 0.611 | MP2K2 | | | H2A3 | | NADK | Notch1 | | | |
| 0.612 | MP2K2 | CDON | CILP2 | | | | Notch1 | | | |
| 0.612 | MP2K2 | CDON | | H2A3 | | | | | | S100A13 |
| 0.613 | MP2K2 | | CILP2 | | | | | | | S100A13 |
| 0.614 | MP2K2 | | CILP2 | H2A3 | | | | | | |
| 0.614 | MP2K2 | | | H2A3 | IGFALS | | | | | |
| 0.615 | MP2K2 | CDON | | H2A3 | | | Notch1 | | | |
| 0.615 | MP2K2 | CDON | | | | NADK | | | | S100A13 |
| 0.616 | MP2K2 | CDON | | | IGFALS | | | | | |
| 0.616 | MP2K2 | | CILP2 | | | NADK | | | | |
| 0.616 | MP2K2 | | | | | | | | PTN | S100A13 |
| 0.616 | MP2K2 | | | | | | | PH | | S100A13 |
| 0.617 | MP2K2 | | | H2A3 | | | | | PTN | |
| 0.617 | MP2K2 | | | | IGFALS | NADK | | | | |
| 0.617 | MP2K2 | CDON | | | | | Notch1 | | | S100A13 |
| 0.619 | MP2K2 | CDON | | | | | | | PTN | |
| 0.619 | MP2K2 | | | H2A3 | IGFALS | NADK | | | | |
| 0.619 | MP2K2 | | | | IGFALS | | Notch1 | | | |
| 0.619 | MP2K2 | CDON | | H2A3 | | NADK | Notch1 | | | |
| 0.62 | MP2K2 | | CILP2 | H2A3 | | NADK | | | | |
| 0.62 | MP2K2 | | CILP2 | | | NADK | Notch1 | | | |
| 0.62 | MP2K2 | CDON | CILP2 | | | | | | | S100A13 |
| 0.62 | MP2K2 | CDON | | H2A3 | | NADK | | | | S100A13 |
| 0.621 | MP2K2 | CDON | CILP2 | H2A3 | | | | | | |
| 0.621 | MP2K2 | | CILP2 | H2A3 | | | Notch1 | | | |
| 0.621 | MP2K2 | | | | IGFALS | | | | | S100A13 |
| 0.621 | MP2K2 | | | H2A3 | | | Notch1 | | | S100A13 |
| 0.622 | MP2K2 | CDON | CILP2 | | | NADK | | | | |
| 0.622 | MP2K2 | CDON | | | | | | PH | | |
| 0.622 | MP2K2 | | | | | | Notch1 | PH | | |
| 0.622 | MP2K2 | | CILP2 | H2A3 | | | | | | S100A13 |
| 0.622 | MP2K2 | | | | | NADK | Notch1 | | | S100A13 |
| 0.623 | MP2K2 | | CILP2 | | | | Notch1 | | | S100A13 |
| 0.624 | MP2K2 | CDON | | | IGFALS | | Notch1 | | | |
| 0.625 | MP2K2 | | CILP2 | | IGFALS | | | | | |
| 0.625 | MP2K2 | CDON | | H2A3 | IGFALS | | | | | |
| 0.625 | MP2K2 | | | | | NADK | | | PTN | |
| 0.625 | MP2K2 | CDON | | | IGFALS | NADK | | | | |
| 0.625 | MP2K2 | CDON | CILP2 | | | NADK | Notch1 | | | |
| 0.625 | MP2K2 | | | | IGFALS | NADK | Notch1 | | | |
| 0.625 | MP2K2 | | | H2A3 | | | | | PTN | S100A13 |
| 0.626 | MP2K2 | CDON | CILP2 | H2A3 | | | Notch1 | | | |
| 0.626 | MP2K2 | | CILP2 | H2A3 | | NADK | Notch1 | | | |
| 0.626 | MP2K2 | | | | | NADK | | PH | | |
| 0.626 | MP2K2 | | | H2A3 | IGFALS | | | | | S100A13 |
| 0.627 | MP2K2 | | | H2A3 | | NADK | | | PTN | |
| 0.627 | MP2K2 | CDON | CILP2 | H2A3 | | NADK | | | | |
| 0.627 | MP2K2 | CDON | CILP2 | | | | Notch1 | | | S100A13 |
| 0.627 | MP2K2 | CDON | | H2A3 | | | Notch1 | | | S100A13 |
| 0.627 | MP2K2 | | | H2A3 | | NADK | Notch1 | | | S100A13 |
| 0.628 | MP2K2 | | | H2A3 | IGFALS | | Notch1 | | | |
| 0.628 | MP2K2 | | | H2A3 | | | | PH | | |
| 0.628 | MP2K2 | CDON | | | | | | | PTN | S100A13 |
| 0.628 | MP2K2 | CDON | CILP2 | H2A3 | | | | | | S100A13 |
| 0.628 | MP2K2 | CDON | | | | NADK | Notch1 | | | S100A13 |
| 0.629 | MP2K2 | CDON | CILP2 | | IGFALS | | | | | |
| 0.629 | MP2K2 | CDON | | H2A3 | IGFALS | NADK | | | | |
| 0.629 | MP2K2 | CDON | | | | | Notch1 | PH | | |
| 0.629 | MP2K2 | | | H2A3 | | | | PH | | S100A13 |
| 0.63 | MP2K2 | CDON | | H2A3 | | | | | PTN | |
| 0.63 | MP2K2 | | | | | | Notch1 | | PTN | |
| 0.63 | MP2K2 | CDON | CILP2 | H2A3 | | NADK | Notch1 | | | |
| 0.63 | MP2K2 | | CILP2 | | | | | PH | | |
| 0.63 | MP2K2 | CDON | | | IGFALS | | | | | S100A13 |
| 0.63 | MP2K2 | CDON | | | | | | PH | | S100A13 |
| 0.631 | MP2K2 | | CILP2 | | IGFALS | | Notch1 | | | |
| 0.631 | MP2K2 | CDON | | | IGFALS | NADK | Notch1 | | | |
| 0.631 | MP2K2 | | | H2A3 | IGFALS | NADK | Notch1 | | | |
| 0.631 | MP2K2 | | | | | | | PH | PTN | |
| 0.631 | MP2K2 | | CILP2 | | | NADK | | | | S100A13 |
| 0.632 | MP2K2 | | CILP2 | H2A3 | IGFALS | | | | | |
| 0.632 | MP2K2 | CDON | | | | NADK | | | PTN | |
| 0.632 | MP2K2 | | | H2A3 | | NADK | | PH | | |
| 0.632 | MP2K2 | | CILP2 | H2A3 | | NADK | | | | S100A13 |
| 0.632 | MP2K2 | | | | IGFALS | NADK | | | | S100A13 |
| 0.632 | MP2K2 | | | H2A3 | IGFALS | NADK | | | | S100A13 |
| 0.632 | MP2K2 | | CILP2 | H2A3 | | | Notch1 | | | S100A13 |
| 0.633 | MP2K2 | | | | IGFALS | | | | PTN | |
| 0.633 | MP2K2 | | | | | NADK | | | PTN | S100A13 |
| 0.633 | MP2K2 | CDON | | H2A3 | | NADK | Notch1 | | | S100A13 |
| 0.633 | MP2K2 | | | | | NADK | | PH | | S100A13 |
| 0.634 | MP2K2 | | CILP2 | | IGFALS | NADK | | | | |
| 0.634 | MP2K2 | CDON | | | | | Notch1 | | PTN | |
| 0.634 | MP2K2 | CDON | CILP2 | | IGFALS | | Notch1 | | | |
| 0.634 | MP2K2 | CDON | | H2A3 | IGFALS | | Notch1 | | | |
| 0.634 | MP2K2 | | | | IGFALS | | | PH | | |
| 0.634 | MP2K2 | CDON | | | | NADK | | PH | | |
| 0.634 | MP2K2 | | | | | NADK | Notch1 | PH | | |
| 0.634 | MP2K2 | | | | | | Notch1 | PH | | S100A13 |
| 0.635 | MP2K2 | | CILP2 | | | | Notch1 | PH | | |
| 0.635 | MP2K2 | CDON | | H2A3 | IGFALS | | | | | S100A13 |
| 0.635 | MP2K2 | CDON | CILP2 | | | NADK | | | | S100A13 |
| 0.635 | MP2K2 | | | H2A3 | | NADK | | | PTN | S100A13 |
| 0.636 | MP2K2 | | CILP2 | H2A3 | IGFALS | NADK | | | | |
| 0.636 | MP2K2 | CDON | | H2A3 | IGFALS | NADK | Notch1 | | | |
| 0.636 | MP2K2 | CDON | CILP2 | H2A3 | | NADK | | | | S100A13 |
| 0.636 | MP2K2 | CDON | CILP2 | H2A3 | | | Notch1 | | | S100A13 |
| 0.637 | MP2K2 | CDON | CILP2 | H2A3 | IGFALS | | | | | |
| 0.637 | MP2K2 | CDON | | H2A3 | | NADK | | | PTN | |
| 0.637 | MP2K2 | CDON | CILP2 | | | | | PH | | |
| 0.637 | MP2K2 | CDON | | H2A3 | | | | | PTN | S100A13 |
| 0.637 | MP2K2 | | | | IGFALS | | Notch1 | | | S100A13 |
| 0.637 | MP2K2 | | CILP2 | | | NADK | Notch1 | | | S100A13 |
| 0.637 | MP2K2 | | | | | | | PH | PTN | S100A13 |
| 0.637 | MP2K2 | | | H2A3 | | NADK | | PH | | S100A13 |
| 0.638 | MP2K2 | CDON | CILP2 | | IGFALS | NADK | | | | |
| 0.638 | MP2K2 | | CILP2 | H2A3 | IGFALS | | Notch1 | | | |
| 0.638 | MP2K2 | | CILP2 | | IGFALS | NADK | Notch1 | | | |
| 0.638 | MP2K2 | CDON | | H2A3 | | | | PH | | |
| 0.638 | MP2K2 | | CILP2 | | IGFALS | | | | | S100A13 |
| 0.638 | MP2K2 | | CILP2 | | | | | PH | | S100A13 |
| 0.639 | MP2K2 | | | H2A3 | IGFALS | | | | PTN | |
| 0.639 | MP2K2 | CDON | | | | NADK | Notch1 | PH | | |
| 0.639 | MP2K2 | CDON | | | IGFALS | NADK | | | | S100A13 |
| 0.64 | MP2K2 | CDON | CILP2 | H2A3 | IGFALS | NADK | | | | |
| 0.64 | MP2K2 | CDON | CILP2 | | IGFALS | NADK | Notch1 | | | |
| 0.64 | MP2K2 | CDON | CILP2 | | | | Notch1 | PH | | |
| 0.64 | MP2K2 | CDON | | | | NADK | | | PTN | S100A13 |
| 0.64 | MP2K2 | CDON | | H2A3 | IGFALS | NADK | | | | S100A13 |
| 0.64 | MP2K2 | CDON | CILP2 | | | NADK | Notch1 | | | S100A13 |
| 0.64 | MP2K2 | | CILP2 | H2A3 | | NADK | Notch1 | | | S100A13 |
| 0.64 | MP2K2 | CDON | | H2A3 | | | | PH | | S100A13 |
| 0.641 | MP2K2 | CDON | | | IGFALS | | | | PTN | |
| 0.641 | MP2K2 | CDON | CILP2 | H2A3 | IGFALS | | Notch1 | | | |
| 0.641 | MP2K2 | | CILP2 | H2A3 | IGFALS | NADK | Notch1 | | | |
| 0.641 | MP2K2 | CDON | | | | | | PH | PTN | |
| 0.641 | MP2K2 | CDON | | H2A3 | | NADK | | PH | | |
| 0.641 | MP2K2 | | | H2A3 | | | Notch1 | PH | | |
| 0.641 | MP2K2 | CDON | | | IGFALS | | Notch1 | | | S100A13 |
| 0.641 | MP2K2 | CDON | | | | | Notch1 | PH | | S100A13 |
| 0.642 | MP2K2 | | | H2A3 | | | Notch1 | | PTN | |
| 0.642 | MP2K2 | | CILP2 | | | NADK | | PH | | |
| 0.642 | MP2K2 | CDON | CILP2 | | IGFALS | | | | | S100A13 |
| 0.642 | MP2K2 | | CILP2 | H2A3 | IGFALS | | | | | S100A13 |
| 0.642 | MP2K2 | | | H2A3 | IGFALS | | Notch1 | | | S100A13 |
| 0.642 | MP2K2 | CDON | | | | NADK | | PH | | S100A13 |
| 0.643 | MP2K2 | | | | | NADK | Notch1 | | PTN | |
| 0.643 | MP2K2 | CDON | | | IGFALS | | | PH | | |
| 0.643 | MP2K2 | | | | IGFALS | NADK | | PH | | |
| 0.643 | MP2K2 | | | H2A3 | | NADK | Notch1 | PH | | |
| 0.643 | MP2K2 | CDON | CILP2 | H2A3 | | NADK | Notch1 | | | S100A13 |
| 0.644 | MP2K2 | CDON | CILP2 | H2A3 | IGFALS | NADK | Notch1 | | | |
| 0.644 | MP2K2 | | | H2A3 | | | | PH | PTN | |
| 0.644 | MP2K2 | | CILP2 | H2A3 | | | | PH | | |
| 0.644 | MP2K2 | | | | IGFALS | | Notch1 | PH | | |
| 0.644 | MP2K2 | | | | IGFALS | | | | PTN | S100A13 |
| 0.644 | MP2K2 | CDON | | H2A3 | | NADK | | | PTN | S100A13 |
| 0.644 | MP2K2 | | | | | | Notch1 | | PTN | S100A13 |
| 0.644 | MP2K2 | | | | IGFALS | NADK | Notch1 | | | S100A13 |
| 0.644 | MP2K2 | CDON | CILP2 | | | | | PH | | S100A13 |
| 0.644 | MP2K2 | | | | IGFALS | | | PH | | S100A13 |
| 0.645 | MP2K2 | | | | IGFALS | NADK | | | PTN | |
| 0.645 | MP2K2 | | | H2A3 | IGFALS | NADK | | | PTN | |
| 0.645 | MP2K2 | CDON | | | | NADK | Notch1 | | PTN | |
| 0.645 | MP2K2 | | | H2A3 | IGFALS | | | PH | | |
| 0.645 | MP2K2 | CDON | CILP2 | H2A3 | IGFALS | | | | | S100A13 |
| 0.645 | MP2K2 | | CILP2 | | IGFALS | | Notch1 | | | S100A13 |
| 0.645 | MP2K2 | CDON | | H2A3 | IGFALS | | Notch1 | | | S100A13 |
| 0.645 | MP2K2 | | | H2A3 | IGFALS | NADK | Notch1 | | | S100A13 |
| 0.645 | MP2K2 | | CILP2 | | | | Notch1 | PH | | S100A13 |
| 0.646 | MP2K2 | CDON | | H2A3 | | | Notch1 | | PTN | |
| 0.646 | MP2K2 | | | | | NADK | | PH | PTN | |
| 0.646 | MP2K2 | CDON | | H2A3 | | | Notch1 | PH | | |
| 0.646 | MP2K2 | | CILP2 | | | NADK | Notch1 | PH | | |
| 0.646 | MP2K2 | CDON | | | | | | PH | PTN | S100A13 |
| 0.646 | MP2K2 | CDON | | H2A3 | | NADK | | PH | | S100A13 |
| 0.646 | MP2K2 | | | | | NADK | Notch1 | PH | | S100A13 |
| 0.647 | MP2K2 | | | H2A3 | | NADK | Notch1 | | PTN | |
| 0.647 | MP2K2 | | CILP2 | H2A3 | | NADK | | PH | | |
| 0.647 | MP2K2 | | | H2A3 | IGFALS | NADK | | PH | | |
| 0.647 | MP2K2 | CDON | CILP2 | | IGFALS | | Notch1 | | | S100A13 |
| 0.647 | MP2K2 | CDON | | | IGFALS | NADK | Notch1 | | | S100A13 |
| 0.647 | MP2K2 | | CILP2 | H2A3 | | | | PH | | S100A13 |
| 0.647 | MP2K2 | | | H2A3 | | | Notch1 | PH | | S100A13 |
| 0.648 | MP2K2 | CDON | | H2A3 | IGFALS | | | | PTN | |
| 0.648 | MP2K2 | CDON | CILP2 | | | NADK | | PH | | |
| 0.648 | MP2K2 | CDON | | | IGFALS | | Notch1 | PH | | |
| 0.648 | MP2K2 | CDON | | H2A3 | | NADK | Notch1 | PH | | |
| 0.648 | MP2K2 | | | H2A3 | IGFALS | | | | PTN | S100A13 |
| 0.648 | MP2K2 | | CILP2 | | IGFALS | NADK | | | | S100A13 |
| 0.648 | MP2K2 | | CILP2 | H2A3 | IGFALS | NADK | | | | S100A13 |
| 0.648 | MP2K2 | CDON | | | | | Notch1 | | PTN | S100A13 |
| 0.648 | MP2K2 | | | H2A3 | | | | PH | PTN | S100A13 |
| 0.649 | MP2K2 | CDON | | | IGFALS | NADK | | | PTN | |
| 0.649 | MP2K2 | CDON | CILP2 | H2A3 | | | | PH | | |
| 0.649 | MP2K2 | | CILP2 | | IGFALS | | | PH | | |
| 0.649 | MP2K2 | CDON | CILP2 | | | NADK | Notch1 | PH | | |
| 0.649 | MP2K2 | | | | IGFALS | NADK | Notch1 | PH | | |
| 0.649 | MP2K2 | CDON | | H2A3 | IGFALS | NADK | Notch1 | | | S100A13 |
| 0.649 | MP2K2 | CDON | CILP2 | | | | Notch1 | PH | | S100A13 |
| 0.65 | MP2K2 | | | H2A3 | | NADK | | PH | PTN | |
| 0.65 | MP2K2 | CDON | | | IGFALS | NADK | | PH | | |
| 0.65 | MP2K2 | | | | | | Notch1 | PH | PTN | |
| 0.65 | MP2K2 | | CILP2 | H2A3 | | | Notch1 | PH | | |
| 0.65 | MP2K2 | | CILP2 | H2A3 | IGFALS | | Notch1 | | | S100A13 |
| 0.651 | MP2K2 | CDON | | H2A3 | | NADK | Notch1 | | PTN | |
| 0.651 | MP2K2 | | | | IGFALS | | | PH | PTN | |
| 0.651 | MP2K2 | CDON | | | | NADK | | PH | PTN | |
| 0.651 | MP2K2 | CDON | | | IGFALS | | | | PTN | S100A13 |
| 0.651 | MP2K2 | CDON | CILP2 | | IGFALS | NADK | | | | S100A13 |
| 0.651 | MP2K2 | CDON | CILP2 | H2A3 | IGFALS | NADK | | | | S100A13 |
| 0.651 | MP2K2 | CDON | CILP2 | H2A3 | | | | PH | | S100A13 |
| 0.651 | MP2K2 | | | H2A3 | IGFALS | | | PH | | S100A13 |
| 0.651 | MP2K2 | | | | | NADK | | PH | PTN | S100A13 |
| 0.651 | MP2K2 | CDON | | | | NADK | Notch1 | PH | | S100A13 |
| 0.652 | MP2K2 | | CILP2 | | | | | | PTN | |
| 0.652 | MP2K2 | CDON | | H2A3 | IGFALS | NADK | | | PTN | |
| 0.652 | MP2K2 | | | | IGFALS | | Notch1 | | PTN | |
| 0.652 | MP2K2 | CDON | CILP2 | H2A3 | | NADK | | PH | | |
| 0.652 | MP2K2 | | CILP2 | H2A3 | | NADK | Notch1 | PH | | |
| 0.652 | MP2K2 | CDON | CILP2 | H2A3 | IGFALS | | Notch1 | | | S100A13 |
| 0.652 | MP2K2 | CDON | | | IGFALS | | | PH | | S100A13 |
| 0.652 | MP2K2 | CDON | | H2A3 | | | Notch1 | PH | | S100A13 |
| 0.652 | MP2K2 | | | H2A3 | | NADK | Notch1 | PH | | S100A13 |
| 0.653 | MP2K2 | CDON | CILP2 | | | | | | PTN | |
| 0.653 | MP2K2 | CDON | | H2A3 | | | | PH | PTN | |
| 0.653 | MP2K2 | CDON | | | | | Notch1 | PH | PTN | |
| 0.653 | MP2K2 | CDON | CILP2 | H2A3 | | | Notch1 | PH | | |
| 0.653 | MP2K2 | | CILP2 | | IGFALS | | Notch1 | PH | | |
| 0.653 | MP2K2 | | | H2A3 | | | Notch1 | | PTN | S100A13 |
| 0.653 | MP2K2 | | CILP2 | | IGFALS | NADK | Notch1 | | | S100A13 |
| 0.653 | MP2K2 | | CILP2 | | | NADK | | PH | | S100A13 |
| 0.654 | MP2K2 | | CILP2 | H2A3 | | | | | PTN | |
| 0.654 | MP2K2 | CDON | | | IGFALS | | Notch1 | | PTN | |
| 0.654 | MP2K2 | CDON | CILP2 | | IGFALS | | | PH | | |
| 0.654 | MP2K2 | CDON | | H2A3 | IGFALS | | | PH | | |
| 0.654 | MP2K2 | CDON | | | IGFALS | NADK | Notch1 | PH | | |
| 0.654 | MP2K2 | | | | IGFALS | NADK | | | PTN | S100A13 |
| 0.654 | MP2K2 | | | H2A3 | IGFALS | NADK | | | PTN | S100A13 |
| 0.654 | MP2K2 | | CILP2 | H2A3 | IGFALS | NADK | Notch1 | | | S100A13 |
| 0.654 | MP2K2 | | | H2A3 | | NADK | | PH | PTN | S100A13 |
| 0.654 | MP2K2 | | CILP2 | H2A3 | | NADK | | PH | | S100A13 |
| 0.655 | MP2K2 | CDON | | H2A3 | IGFALS | NADK | | PH | | |
| 0.655 | MP2K2 | CDON | CILP2 | | IGFALS | | Notch1 | PH | | |
| 0.655 | MP2K2 | | | H2A3 | IGFALS | | Notch1 | PH | | |
| 0.655 | MP2K2 | CDON | CILP2 | H2A3 | | NADK | Notch1 | PH | | |
| 0.655 | MP2K2 | | | | | NADK | Notch1 | | PTN | S100A13 |
| 0.655 | MP2K2 | CDON | CILP2 | | IGFALS | NADK | Notch1 | | | S100A13 |
| 0.655 | MP2K2 | | | | IGFALS | NADK | | PH | | S100A13 |
| 0.655 | MP2K2 | | CILP2 | H2A3 | | | Notch1 | PH | | S100A13 |
| 0.656 | MP2K2 | | CILP2 | | IGFALS | NADK | | PH | | |
| 0.656 | MP2K2 | | | H2A3 | IGFALS | NADK | Notch1 | PH | | |
| 0.656 | MP2K2 | CDON | | H2A3 | IGFALS | | | | PTN | S100A13 |
| 0.656 | MP2K2 | CDON | | H2A3 | | | Notch1 | | PTN | S100A13 |
| 0.656 | MP2K2 | CDON | CILP2 | H2A3 | IGFALS | NADK | Notch1 | | | S100A13 |
| 0.656 | MP2K2 | CDON | | H2A3 | | | | PH | PTN | S100A13 |
| 0.656 | MP2K2 | CDON | CILP2 | | | NADK | | PH | | S100A13 |
| 0.656 | MP2K2 | | | H2A3 | IGFALS | NADK | | PH | | S100A13 |
| 0.656 | MP2K2 | | | | IGFALS | | Notch1 | PH | | S100A13 |
| 0.656 | MP2K2 | CDON | | H2A3 | | NADK | Notch1 | PH | | S100A13 |
| 0.657 | MP2K2 | CDON | CILP2 | H2A3 | | | | | PTN | |
| 0.657 | MP2K2 | | CILP2 | H2A3 | IGFALS | | | PH | | |
| 0.657 | MP2K2 | CDON | | H2A3 | | NADK | | PH | PTN | |
| 0.657 | MP2K2 | | CILP2 | | | | | | PTN | S100A13 |
| 0.657 | MP2K2 | | CILP2 | | IGFALS | | | PH | | S100A13 |
| 0.657 | MP2K2 | CDON | | | | NADK | | PH | PTN | S100A13 |
| 0.657 | MP2K2 | CDON | CILP2 | H2A3 | | NADK | | PH | | S100A13 |
| 0.657 | MP2K2 | CDON | CILP2 | H2A3 | | | Notch1 | PH | | S100A13 |
| 0.657 | MP2K2 | | CILP2 | | | NADK | Notch1 | PH | | S100A13 |
| 0.658 | MP2K2 | | | H2A3 | IGFALS | | Notch1 | | PTN | |
| 0.658 | MP2K2 | CDON | | | IGFALS | | | PH | PTN | |
| 0.658 | MP2K2 | CDON | CILP2 | | | | | | PTN | S100A13 |
| 0.658 | MP2K2 | CDON | | | | NADK | Notch1 | | PTN | S100A13 |
| 0.658 | MP2K2 | | | H2A3 | | NADK | Notch1 | | PTN | S100A13 |
| 0.658 | MP2K2 | CDON | | H2A3 | IGFALS | | | PH | | S100A13 |
| 0.659 | MP2K2 | | | | IGFALS | NADK | Notch1 | | PTN | |
| 0.659 | MP2K2 | | | H2A3 | IGFALS | | | PH | PTN | |
| 0.659 | MP2K2 | | CILP2 | H2A3 | IGFALS | NADK | | PH | | |
| 0.659 | MP2K2 | CDON | | H2A3 | IGFALS | | Notch1 | PH | | |
| 0.659 | MP2K2 | | CILP2 | | IGFALS | NADK | Notch1 | PH | | |
| 0.659 | MP2K2 | CDON | | H2A3 | IGFALS | NADK | Notch1 | PH | | |
| 0.659 | MP2K2 | | CILP2 | H2A3 | | | | | PTN | S100A13 |
| 0.659 | MP2K2 | CDON | | | IGFALS | NADK | | | PTN | S100A13 |
| 0.659 | MP2K2 | CDON | | | IGFALS | NADK | | PH | | S100A13 |
| 0.659 | MP2K2 | | | | | | Notch1 | PH | PTN | S100A13 |
| 0.659 | MP2K2 | CDON | | | IGFALS | | Notch1 | PH | | S100A13 |
| 0.659 | MP2K2 | CDON | CILP2 | | | NADK | Notch1 | PH | | S100A13 |
| 0.66 | MP2K2 | CDON | CILP2 | | | | Notch1 | | PTN | |
| 0.66 | MP2K2 | CDON | | | IGFALS | NADK | Notch1 | | PTN | |
| 0.66 | MP2K2 | | | | IGFALS | NADK | | PH | PTN | |
| 0.66 | MP2K2 | CDON | CILP2 | | IGFALS | NADK | | PH | | |
| 0.66 | MP2K2 | | | | | NADK | Notch1 | PH | PTN | |
| 0.66 | MP2K2 | CDON | | H2A3 | IGFALS | NADK | | | PTN | S100A13 |
| 0.66 | MP2K2 | | | | IGFALS | | | PH | PTN | S100A13 |
| 0.661 | MP2K2 | | CILP2 | H2A3 | | NADK | | | PTN | |
| 0.661 | MP2K2 | | CILP2 | | | | Notch1 | | PTN | |
| 0.661 | MP2K2 | CDON | | H2A3 | IGFALS | | Notch1 | | PTN | |
| 0.661 | MP2K2 | CDON | CILP2 | | IGFALS | NADK | Notch1 | PH | | |
| 0.661 | MP2K2 | CDON | CILP2 | H2A3 | | | | | PTN | S100A13 |
| 0.661 | MP2K2 | CDON | | H2A3 | | NADK | Notch1 | | PTN | S100A13 |
| 0.661 | MP2K2 | CDON | CILP2 | | IGFALS | | | PH | | S100A13 |
| 0.661 | MP2K2 | CDON | | H2A3 | | NADK | | PH | PTN | S100A13 |
| 0.661 | MP2K2 | | CILP2 | H2A3 | | NADK | Notch1 | PH | | S100A13 |
| 0.662 | MP2K2 | | | H2A3 | IGFALS | NADK | Notch1 | | PTN | |
| 0.662 | MP2K2 | CDON | CILP2 | H2A3 | IGFALS | | | PH | | |
| 0.662 | MP2K2 | | CILP2 | H2A3 | IGFALS | | Notch1 | PH | | |
| 0.662 | MP2K2 | CDON | | | | NADK | Notch1 | PH | PTN | |
| 0.662 | MP2K2 | | CILP2 | H2A3 | IGFALS | | | PH | | S100A13 |
| 0.662 | MP2K2 | CDON | | H2A3 | IGFALS | NADK | | PH | | S100A13 |
| 0.662 | MP2K2 | CDON | | | | | Notch1 | PH | PTN | S100A13 |
| 0.662 | MP2K2 | CDON | CILP2 | H2A3 | | NADK | Notch1 | PH | | S100A13 |
| 0.663 | MP2K2 | | CILP2 | | | NADK | | | PTN | |
| 0.663 | MP2K2 | CDON | CILP2 | | | NADK | | | PTN | |
| 0.663 | MP2K2 | CDON | CILP2 | H2A3 | | NADK | | | PTN | |
| 0.663 | MP2K2 | | | H2A3 | IGFALS | NADK | | PH | PTN | |
| 0.663 | MP2K2 | CDON | CILP2 | H2A3 | IGFALS | NADK | | PH | | |
| 0.663 | MP2K2 | | CILP2 | H2A3 | IGFALS | NADK | Notch1 | PH | | |
| 0.663 | MP2K2 | | CILP2 | | IGFALS | | Notch1 | PH | | S100A13 |
| 0.663 | MP2K2 | | | H2A3 | IGFALS | | Notch1 | PH | | S100A13 |
| 0.663 | MP2K2 | | | | IGFALS | NADK | Notch1 | PH | | S100A13 |
| 0.664 | MP2K2 | CDON | | H2A3 | IGFALS | NADK | Notch1 | | PTN | |
| 0.664 | MP2K2 | | | H2A3 | | | Notch1 | PH | PTN | |
| 0.664 | MP2K2 | CDON | CILP2 | H2A3 | IGFALS | | Notch1 | PH | | |
| 0.665 | MP2K2 | | CILP2 | H2A3 | | | Notch1 | | PTN | |
| 0.665 | MP2K2 | CDON | CILP2 | H2A3 | | | Notch1 | | PTN | |
| 0.665 | MP2K2 | CDON | | | IGFALS | NADK | | PH | PTN | |
| 0.665 | MP2K2 | CDON | CILP2 | H2A3 | IGFALS | NADK | Notch1 | PH | | |
| 0.665 | MP2K2 | | | | IGFALS | | Notch1 | | PTN | S100A13 |
| 0.665 | MP2K2 | CDON | | | IGFALS | | | PH | PTN | S100A13 |
| 0.665 | MP2K2 | | | H2A3 | IGFALS | | | PH | PTN | S100A13 |
| 0.665 | MP2K2 | CDON | CILP2 | H2A3 | IGFALS | | | PH | | S100A13 |
| 0.665 | MP2K2 | CDON | CILP2 | | IGFALS | | Notch1 | PH | | S100A13 |
| 0.665 | MP2K2 | CDON | | | IGFALS | NADK | Notch1 | PH | | S100A13 |
| 0.666 | MP2K2 | CDON | CILP2 | | IGFALS | | | | PTN | |
| 0.666 | MP2K2 | CDON | | H2A3 | | | Notch1 | PH | PTN | |
| 0.666 | MP2K2 | | CILP2 | H2A3 | | NADK | | | PTN | S100A13 |
| 0.666 | MP2K2 | CDON | CILP2 | | | | Notch1 | | PTN | S100A13 |
| 0.666 | MP2K2 | CDON | | | IGFALS | | Notch1 | | PTN | S100A13 |
| 0.666 | MP2K2 | | CILP2 | | IGFALS | NADK | | PH | | S100A13 |
| 0.666 | MP2K2 | CDON | | H2A3 | IGFALS | | Notch1 | PH | | S100A13 |
| 0.666 | MP2K2 | | | H2A3 | IGFALS | NADK | Notch1 | PH | | S100A13 |
| 0.667 | MP2K2 | | CILP2 | | IGFALS | | | | PTN | |
| 0.667 | MP2K2 | | CILP2 | H2A3 | IGFALS | | | | PTN | |
| 0.667 | MP2K2 | | CILP2 | | | | | PH | PTN | |
| 0.667 | MP2K2 | CDON | | H2A3 | IGFALS | | | PH | PTN | |
| 0.667 | MP2K2 | | | | IGFALS | | Notch1 | PH | PTN | |
| 0.667 | MP2K2 | | | H2A3 | | NADK | Notch1 | PH | PTN | |
| 0.667 | MP2K2 | | CILP2 | | | | Notch1 | | PTN | S100A13 |
| 0.667 | MP2K2 | | CILP2 | H2A3 | IGFALS | NADK | | PH | | S100A13 |
| 0.668 | MP2K2 | CDON | CILP2 | H2A3 | IGFALS | | | | PTN | |
| 0.668 | MP2K2 | CDON | CILP2 | | | NADK | Notch1 | | PTN | |
| 0.668 | MP2K2 | CDON | CILP2 | | | | | PH | PTN | |
| 0.668 | MP2K2 | | CILP2 | | | NADK | | | PTN | S100A13 |
| 0.668 | MP2K2 | CDON | CILP2 | | | NADK | | | PTN | S100A13 |
| 0.668 | MP2K2 | CDON | CILP2 | H2A3 | | NADK | | | PTN | S100A13 |
| 0.668 | MP2K2 | | | H2A3 | IGFALS | | Notch1 | | PTN | S100A13 |
| 0.668 | MP2K2 | CDON | CILP2 | | IGFALS | NADK | | PH | | S100A13 |
| 0.668 | MP2K2 | | CILP2 | H2A3 | IGFALS | | Notch1 | PH | | S100A13 |
| 0.668 | MP2K2 | CDON | | H2A3 | IGFALS | NADK | Notch1 | PH | | S100A13 |
| 0.669 | MP2K2 | | CILP2 | | | NADK | Notch1 | | PTN | |
| 0.669 | MP2K2 | CDON | | H2A3 | IGFALS | NADK | | PH | PTN | |
| 0.669 | MP2K2 | CDON | | | IGFALS | | Notch1 | PH | PTN | |
| 0.669 | MP2K2 | CDON | | H2A3 | | NADK | Notch1 | PH | PTN | |
| 0.669 | MP2K2 | | | | IGFALS | NADK | | PH | PTN | S100A13 |
| 0.669 | MP2K2 | CDON | CILP2 | H2A3 | IGFALS | NADK | | PH | | S100A13 |
| 0.669 | MP2K2 | CDON | CILP2 | H2A3 | IGFALS | | Notch1 | PH | | S100A13 |
| 0.669 | MP2K2 | | | | | NADK | Notch1 | PH | PTN | S100A13 |
| 0.67 | MP2K2 | | CILP2 | H2A3 | | NADK | Notch1 | | PTN | |
| 0.67 | MP2K2 | CDON | CILP2 | H2A3 | | NADK | Notch1 | | PTN | |
| 0.67 | MP2K2 | | CILP2 | H2A3 | | | | PH | PTN | |
| 0.67 | MP2K2 | CDON | | H2A3 | IGFALS | | Notch1 | | PTN | S100A13 |
| 0.67 | MP2K2 | | CILP2 | | | | | PH | PTN | S100A13 |
| 0.67 | MP2K2 | | | H2A3 | IGFALS | NADK | | PH | PTN | S100A13 |
| 0.67 | MP2K2 | | | H2A3 | | | Notch1 | PH | PTN | S100A13 |
| 0.67 | MP2K2 | CDON | | | | NADK | Notch1 | PH | PTN | S100A13 |
| 0.67 | MP2K2 | | CILP2 | | IGFALS | NADK | Notch1 | PH | | S100A13 |
| 0.671 | MP2K2 | | CILP2 | H2A3 | IGFALS | NADK | | | PTN | |
| 0.671 | MP2K2 | | CILP2 | H2A3 | | | Notch1 | | PTN | S100A13 |
| 0.671 | MP2K2 | CDON | CILP2 | H2A3 | | | Notch1 | | PTN | S100A13 |
| 0.671 | MP2K2 | | | | IGFALS | NADK | Notch1 | | PTN | S100A13 |
| 0.671 | MP2K2 | CDON | CILP2 | | | | | PH | PTN | S100A13 |
| 0.671 | MP2K2 | CDON | | H2A3 | IGFALS | | | PH | PTN | S100A13 |
| 0.671 | MP2K2 | CDON | CILP2 | | IGFALS | NADK | Notch1 | PH | | S100A13 |
| 0.672 | MP2K2 | CDON | CILP2 | H2A3 | IGFALS | NADK | | | PTN | |
| 0.672 | MP2K2 | CDON | CILP2 | | IGFALS | | Notch1 | | PTN | |
| 0.672 | MP2K2 | CDON | CILP2 | H2A3 | | | | PH | PTN | |
| 0.672 | MP2K2 | CDON | CILP2 | | IGFALS | | | | PTN | S100A13 |
| 0.672 | MP2K2 | CDON | | | IGFALS | NADK | Notch1 | | PTN | S100A13 |
| 0.672 | MP2K2 | | | H2A3 | IGFALS | NADK | Notch1 | | PTN | S100A13 |
| 0.672 | MP2K2 | CDON | | | IGFALS | NADK | | PH | PTN | S100A13 |
| 0.672 | MP2K2 | CDON | | H2A3 | | | Notch1 | PH | PTN | S100A13 |
| 0.672 | MP2K2 | | CILP2 | H2A3 | IGFALS | NADK | Notch1 | PH | | S100A13 |
| 0.673 | MP2K2 | CDON | CILP2 | | IGFALS | NADK | | | PTN | |
| 0.673 | MP2K2 | CDON | CILP2 | | | | Notch1 | PH | PTN | |
| 0.673 | MP2K2 | | | | IGFALS | NADK | Notch1 | PH | PTN | |
| 0.673 | MP2K2 | | CILP2 | | IGFALS | | | | PTN | S100A13 |
| 0.673 | MP2K2 | | CILP2 | H2A3 | IGFALS | | | | PTN | S100A13 |
| 0.673 | MP2K2 | CDON | | H2A3 | IGFALS | NADK | Notch1 | | PTN | S100A13 |
| 0.673 | MP2K2 | | CILP2 | H2A3 | | | | PH | PTN | S100A13 |
| 0.673 | MP2K2 | | | H2A3 | | NADK | Notch1 | PH | PTN | S100A13 |
| 0.673 | MP2K2 | CDON | CILP2 | H2A3 | IGFALS | NADK | Notch1 | PH | | S100A13 |
| 0.674 | MP2K2 | | CILP2 | | IGFALS | NADK | | | PTN | |
| 0.674 | MP2K2 | | CILP2 | | IGFALS | | Notch1 | | PTN | |
| 0.674 | MP2K2 | | CILP2 | H2A3 | | NADK | | PH | PTN | |
| 0.674 | MP2K2 | | CILP2 | | | | Notch1 | PH | PTN | |
| 0.674 | MP2K2 | CDON | | | IGFALS | NADK | Notch1 | PH | PTN | |
| 0.674 | MP2K2 | CDON | CILP2 | H2A3 | IGFALS | | | | PTN | S100A13 |
| 0.675 | MP2K2 | | CILP2 | H2A3 | IGFALS | | Notch1 | | PTN | |
| 0.675 | MP2K2 | CDON | CILP2 | H2A3 | IGFALS | | Notch1 | | PTN | |
| 0.675 | MP2K2 | | CILP2 | | | NADK | | PH | PTN | |
| 0.675 | MP2K2 | CDON | CILP2 | | | NADK | | PH | PTN | |
| 0.675 | MP2K2 | CDON | CILP2 | H2A3 | | | | PH | PTN | S100A13 |
| 0.675 | MP2K2 | CDON | | H2A3 | IGFALS | NADK | | PH | PTN | S100A13 |
| 0.675 | MP2K2 | CDON | | H2A3 | | NADK | Notch1 | PH | PTN | S100A13 |
| 0.676 | MP2K2 | CDON | CILP2 | H2A3 | | NADK | | PH | PTN | |
| 0.676 | MP2K2 | | | H2A3 | IGFALS | | Notch1 | PH | PTN | |
| 0.676 | MP2K2 | | CILP2 | | | NADK | Notch1 | | PTN | S100A13 |
| 0.676 | MP2K2 | CDON | CILP2 | | | NADK | Notch1 | | PTN | S100A13 |
| 0.676 | MP2K2 | CDON | CILP2 | H2A3 | | NADK | Notch1 | | PTN | S100A13 |
| 0.676 | MP2K2 | | | | IGFALS | | Notch1 | PH | PTN | S100A13 |
| 0.677 | MP2K2 | CDON | | H2A3 | IGFALS | | Notch1 | PH | PTN | |
| 0.677 | MP2K2 | | | H2A3 | IGFALS | NADK | Notch1 | PH | PTN | |
| 0.677 | MP2K2 | | CILP2 | H2A3 | | NADK | Notch1 | | PTN | S100A13 |
| 0.677 | MP2K2 | CDON | CILP2 | | | | Notch1 | PH | PTN | S100A13 |
| 0.677 | MP2K2 | CDON | | | IGFALS | | Notch1 | PH | PTN | S100A13 |
| 0.678 | MP2K2 | CDON | CILP2 | | IGFALS | NADK | Notch1 | | PTN | |
| 0.678 | MP2K2 | CDON | CILP2 | H2A3 | IGFALS | NADK | Notch1 | | PTN | |
| 0.678 | MP2K2 | | CILP2 | | IGFALS | | | PH | PTN | |
| 0.678 | MP2K2 | | CILP2 | H2A3 | IGFALS | NADK | | | PTN | S100A13 |
| 0.678 | MP2K2 | CDON | CILP2 | H2A3 | IGFALS | NADK | | | PTN | S100A13 |
| 0.678 | MP2K2 | | CILP2 | | | | Notch1 | PH | PTN | S100A13 |
| 0.679 | MP2K2 | | CILP2 | | IGFALS | NADK | Notch1 | | PTN | |
| 0.679 | MP2K2 | | CILP2 | H2A3 | IGFALS | NADK | Notch1 | | PTN | |
| 0.679 | MP2K2 | CDON | CILP2 | | IGFALS | | | PH | PTN | |
| 0.679 | MP2K2 | CDON | CILP2 | H2A3 | | | Notch1 | PH | PTN | |
| 0.679 | MP2K2 | CDON | | H2A3 | IGFALS | NADK | Notch1 | PH | PTN | |
| 0.679 | MP2K2 | CDON | CILP2 | | IGFALS | NADK | | | PTN | S100A13 |
| 0.679 | MP2K2 | CDON | CILP2 | | IGFALS | | Notch1 | | PTN | S100A13 |
| 0.679 | MP2K2 | CDON | CILP2 | | | NADK | | PH | PTN | S100A13 |
| 0.679 | MP2K2 | | CILP2 | H2A3 | | NADK | | PH | PTN | S100A13 |
| 0.679 | MP2K2 | CDON | CILP2 | H2A3 | | NADK | | PH | PTN | S100A13 |
| 0.68 | MP2K2 | | CILP2 | H2A3 | IGFALS | | | PH | PTN | |
| 0.68 | MP2K2 | | CILP2 | H2A3 | | | Notch1 | PH | PTN | |
| 0.68 | MP2K2 | CDON | CILP2 | | | NADK | Notch1 | PH | PTN | |
| 0.68 | MP2K2 | | CILP2 | | IGFALS | NADK | | | PTN | S100A13 |
| 0.68 | MP2K2 | | CILP2 | | IGFALS | | Notch1 | | PTN | S100A13 |
| 0.68 | MP2K2 | | CILP2 | | | NADK | | PH | PTN | S100A13 |
| 0.681 | MP2K2 | | CILP2 | | | NADK | Notch1 | PH | PTN | |
| 0.681 | MP2K2 | CDON | CILP2 | H2A3 | IGFALS | | Notch1 | | PTN | S100A13 |
| 0.682 | MP2K2 | CDON | CILP2 | H2A3 | IGFALS | | | PH | PTN | |
| 0.682 | MP2K2 | CDON | CILP2 | H2A3 | | NADK | Notch1 | PH | PTN | |
| 0.682 | MP2K2 | | CILP2 | H2A3 | IGFALS | | Notch1 | | PTN | S100A13 |
| 0.682 | MP2K2 | | | H2A3 | IGFALS | | Notch1 | PH | PTN | S100A13 |
| 0.682 | MP2K2 | | | | IGFALS | NADK | Notch1 | PH | PTN | S100A13 |
| 0.683 | MP2K2 | CDON | CILP2 | | IGFALS | NADK | | PH | PTN | |
| 0.683 | MP2K2 | | CILP2 | H2A3 | IGFALS | NADK | | PH | PTN | |
| 0.683 | MP2K2 | | CILP2 | H2A3 | | NADK | Notch1 | PH | PTN | |
| 0.683 | MP2K2 | | CILP2 | | IGFALS | | | PH | PTN | S100A13 |
| 0.683 | MP2K2 | CDON | CILP2 | | IGFALS | | | PH | PTN | S100A13 |
| 0.683 | MP2K2 | CDON | CILP2 | H2A3 | | | Notch1 | PH | PTN | S100A13 |
| 0.683 | MP2K2 | CDON | | H2A3 | IGFALS | | Notch1 | PH | PTN | S100A13 |
| 0.683 | MP2K2 | CDON | | | IGFALS | NADK | Notch1 | PH | PTN | S100A13 |
| 0.684 | MP2K2 | | CILP2 | | IGFALS | NADK | | PH | PTN | |
| 0.684 | MP2K2 | CDON | CILP2 | H2A3 | IGFALS | NADK | | PH | PTN | |
| 0.684 | MP2K2 | CDON | CILP2 | | IGFALS | | Notch1 | PH | PTN | |
| 0.684 | MP2K2 | | CILP2 | H2A3 | IGFALS | | | PH | PTN | S100A13 |
| 0.684 | MP2K2 | | CILP2 | H2A3 | | | Notch1 | PH | PTN | S100A13 |
| 0.685 | MP2K2 | | CILP2 | | IGFALS | | Notch1 | PH | PTN | |
| 0.685 | MP2K2 | CDON | CILP2 | | IGFALS | NADK | Notch1 | | PTN | S100A13 |
| 0.685 | MP2K2 | CDON | CILP2 | H2A3 | IGFALS | NADK | Notch1 | | PTN | S100A13 |
| 0.685 | MP2K2 | CDON | CILP2 | H2A3 | IGFALS | | | PH | PTN | S100A13 |
| 0.685 | MP2K2 | CDON | CILP2 | | | NADK | Notch1 | PH | PTN | S100A13 |
| 0.685 | MP2K2 | | | H2A3 | IGFALS | NADK | Notch1 | PH | PTN | S100A13 |
| 0.685 | MP2K2 | CDON | | H2A3 | IGFALS | NADK | Notch1 | PH | PTN | S100A13 |
| 0.686 | MP2K2 | | CILP2 | H2A3 | IGFALS | NADK | Notch1 | | PTN | S100A13 |
| 0.686 | MP2K2 | | CILP2 | | | NADK | Notch1 | PH | PTN | S100A13 |
| 0.687 | MP2K2 | | CILP2 | | IGFALS | NADK | Notch1 | | PTN | S100A13 |
| 0.687 | MP2K2 | CDON | CILP2 | H2A3 | | NADK | Notch1 | PH | PTN | S100A13 |
| 0.688 | MP2K2 | | CILP2 | H2A3 | IGFALS | | Notch1 | PH | PTN | |
| 0.688 | MP2K2 | CDON | CILP2 | H2A3 | IGFALS | | Notch1 | PH | PTN | |
| 0.688 | MP2K2 | CDON | CILP2 | | IGFALS | NADK | Notch1 | PH | PTN | |
| 0.688 | MP2K2 | | CILP2 | H2A3 | IGFALS | NADK | | PH | PTN | S100A13 |
| 0.688 | MP2K2 | CDON | CILP2 | H2A3 | IGFALS | NADK | | PH | PTN | S100A13 |
| 0.688 | MP2K2 | CDON | CILP2 | | IGFALS | | Notch1 | PH | PTN | S100A13 |
| 0.688 | MP2K2 | | CILP2 | H2A3 | | NADK | Notch1 | PH | PTN | S100A13 |
| 0.689 | MP2K2 | | CILP2 | | IGFALS | NADK | Notch1 | PH | PTN | |
| 0.689 | MP2K2 | CDON | CILP2 | H2A3 | IGFALS | NADK | Notch1 | PH | PTN | |
| 0.689 | MP2K2 | | CILP2 | | IGFALS | NADK | | PH | PTN | S100A13 |
| 0.689 | MP2K2 | CDON | CILP2 | | IGFALS | NADK | | PH | PTN | S100A13 |
| 0.69 | MP2K2 | | CILP2 | H2A3 | IGFALS | NADK | Notch1 | PH | PTN | |
| 0.69 | MP2K2 | | CILP2 | | IGFALS | | Notch1 | PH | PTN | S100A13 |
| 0.692 | MP2K2 | CDON | CILP2 | H2A3 | IGFALS | | Notch1 | PH | PTN | S100A13 |
| 0.693 | MP2K2 | | CILP2 | H2A3 | IGFALS | | Notch1 | PH | PTN | S100A13 |
| 0.694 | MP2K2 | CDON | CILP2 | | IGFALS | NADK | Notch1 | PH | PTN | S100A13 |
| 0.694 | MP2K2 | CDON | CILP2 | H2A3 | IGFALS | NADK | Notch1 | PH | PTN | S100A13 |
| 0.695 | MP2K2 | | CILP2 | | IGFALS | NADK | Notch1 | PH | PTN | S100A13 |
| *0.696 | MP2K2 | | CILP2 | H2A3 | IGFALS | NADK | Notch1 | PH | PTN | S100A13 |

*Included in Table 38

TABLE 46
H2A3 Biomarker Panels-20 Year Model

| AUC | H2A3 | CDON | CILP2 | IGFALS | MP2K2 | NADK | Notch1 | PH | PTN | S100A13 |
|---|---|---|---|---|---|---|---|---|---|---|
| 0.572 | H2A3 | | | | | | | | | S100A13 |
| 0.574 | H2A3 | | | | | NADK | | | | |
| 0.583 | H2A3 | CDON | | | | | | | | |
| 0.585 | H2A3 | | | | MP2K2 | | | | | |
| 0.588 | H2A3 | | CILP2 | | | | | | | |
| 0.589 | H2A3 | | | | | | Notch1 | | | |
| 0.591 | H2A3 | | | | | NADK | | | | S100A13 |
| 0.594 | H2A3 | CDON | | | | NADK | | | | |
| 0.594 | H2A3 | | | | MP2K2 | NADK | | | | |
| 0.594 | H2A3 | | | | MP2K2 | | | | | S100A13 |
| 0.595 | H2A3 | CDON | | | | | | | | S100A13 |
| 0.599 | H2A3 | CDON | | | | | Notch1 | | | |
| 0.599 | H2A3 | | | | | NADK | Notch1 | | | |
| 0.599 | H2A3 | | CILP2 | | | | | | | S100A13 |
| 0.6 | H2A3 | CDON | CILP2 | | | | | | | |
| 0.602 | H2A3 | | | IGFALS | | | | | | |
| 0.602 | H2A3 | | CILP2 | | | NADK | | | | |
| 0.602 | H2A3 | | CILP2 | | | | Notch1 | | | |
| 0.604 | H2A3 | CDON | | | MP2K2 | | | | | |
| 0.606 | H2A3 | | | | MP2K2 | NADK | | | | S100A13 |
| 0.606 | H2A3 | | | | | | Notch1 | | | S100A13 |
| 0.607 | H2A3 | | | | MP2K2 | | Notch1 | | | |
| 0.607 | H2A3 | CDON | | | | NADK | Notch1 | | | |
| 0.608 | H2A3 | CDON | CILP2 | | | | Notch1 | | | |
| 0.608 | H2A3 | CDON | | | | NADK | | | | S100A13 |
| 0.609 | H2A3 | CDON | | | MP2K2 | NADK | | | | |
| 0.609 | H2A3 | CDON | CILP2 | | | | | | | S100A13 |
| 0.611 | H2A3 | | | | | | | | PTN | |
| 0.611 | H2A3 | CDON | CILP2 | | | NADK | | | | |
| 0.611 | H2A3 | | | | MP2K2 | NADK | Notch1 | | | |
| 0.611 | H2A3 | | | | | | | PH | | |
| 0.612 | H2A3 | | | IGFALS | | NADK | | | | |
| 0.612 | H2A3 | | CILP2 | | | NADK | Notch1 | | | |
| 0.612 | H2A3 | CDON | | | MP2K2 | | | | | S100A13 |
| 0.613 | H2A3 | | | | | | | PH | | S100A13 |
| 0.614 | H2A3 | | CILP2 | | MP2K2 | | | | | |
| 0.614 | H2A3 | | | IGFALS | MP2K2 | | | | | |
| 0.614 | H2A3 | | CILP2 | | | NADK | | | | S100A13 |
| 0.615 | H2A3 | CDON | | | MP2K2 | | Notch1 | | | |
| 0.615 | H2A3 | | | IGFALS | | | | | | S100A13 |
| 0.615 | H2A3 | CDON | | | | | Notch1 | | | S100A13 |
| 0.615 | H2A3 | | CILP2 | | | | Notch1 | | | S100A13 |
| 0.616 | H2A3 | CDON | | IGFALS | | | | | | |
| 0.617 | H2A3 | | | | MP2K2 | | | | PTN | |
| 0.617 | H2A3 | CDON | CILP2 | | | NADK | Notch1 | | | |
| 0.617 | H2A3 | | | | | NADK | Notch1 | | | S100A13 |
| 0.619 | H2A3 | | CILP2 | IGFALS | | | | | | |
| 0.619 | H2A3 | | | IGFALS | MP2K2 | NADK | | | | |
| 0.619 | H2A3 | | | IGFALS | | | Notch1 | | | |
| 0.619 | H2A3 | CDON | | | MP2K2 | NADK | Notch1 | | | |
| 0.619 | H2A3 | | | | | | | | PTN | S100A13 |
| 0.62 | H2A3 | | CILP2 | | MP2K2 | NADK | | | | |
| 0.62 | H2A3 | | | | | NADK | | PH | | |
| 0.62 | H2A3 | CDON | | | MP2K2 | NADK | | | | S100A13 |
| 0.621 | H2A3 | CDON | CILP2 | | MP2K2 | | | | | |
| 0.621 | H2A3 | | CILP2 | | MP2K2 | | Notch1 | | | |
| 0.621 | H2A3 | CDON | CILP2 | | | NADK | | | | S100A13 |
| 0.621 | H2A3 | CDON | CILP2 | | | | Notch1 | | | S100A13 |
| 0.621 | H2A3 | | | | MP2K2 | | Notch1 | | | S100A13 |
| 0.622 | H2A3 | CDON | | IGFALS | | NADK | | | | |
| 0.622 | H2A3 | | CILP2 | | MP2K2 | | | | | S100A13 |
| 0.624 | H2A3 | | | | | NADK | | | PTN | |
| 0.624 | H2A3 | CDON | | | | NADK | Notch1 | | | S100A13 |
| 0.625 | H2A3 | CDON | | | | | | | PTN | |
| 0.625 | H2A3 | CDON | | IGFALS | MP2K2 | | | | | |
| 0.625 | H2A3 | CDON | | IGFALS | | | Notch1 | | | |
| 0.625 | H2A3 | | | IGFALS | | NADK | Notch1 | | | |
| 0.625 | H2A3 | CDON | | | | | | PH | | |
| 0.625 | H2A3 | | | | MP2K2 | | | | PTN | S100A13 |
| 0.625 | H2A3 | | | | | NADK | | PH | | S100A13 |
| 0.626 | H2A3 | CDON | CILP2 | IGFALS | | | | | | |
| 0.626 | H2A3 | CDON | CILP2 | | MP2K2 | | Notch1 | | | |
| 0.626 | H2A3 | | CILP2 | | MP2K2 | NADK | Notch1 | | | |
| 0.626 | H2A3 | CDON | | IGFALS | | | | | | S100A13 |
| 0.626 | H2A3 | | | IGFALS | MP2K2 | | | | | S100A13 |
| 0.626 | H2A3 | | | IGFALS | | NADK | | | | S100A13 |
| 0.627 | H2A3 | | | | MP2K2 | NADK | | | PTN | |
| 0.627 | H2A3 | CDON | CILP2 | | MP2K2 | NADK | | | | |
| 0.627 | H2A3 | | CILP2 | | | | | PH | | |
| 0.627 | H2A3 | CDON | | | MP2K2 | | Notch1 | | | S100A13 |
| 0.627 | H2A3 | | CILP2 | | | NADK | Notch1 | | | S100A13 |
| 0.627 | H2A3 | | | | MP2K2 | NADK | Notch1 | | | S100A13 |
| 0.628 | H2A3 | | CILP2 | IGFALS | | NADK | | | | |
| 0.628 | H2A3 | | CILP2 | IGFALS | | | Notch1 | | | |
| 0.628 | H2A3 | | | IGFALS | MP2K2 | | Notch1 | | | |
| 0.628 | H2A3 | | | | MP2K2 | | | PH | | |
| 0.628 | H2A3 | | CILP2 | IGFALS | | | | | | S100A13 |
| 0.628 | H2A3 | CDON | CILP2 | | MP2K2 | | | | | S100A13 |
| 0.629 | H2A3 | CDON | | IGFALS | MP2K2 | NADK | | | | |
| 0.629 | H2A3 | CDON | | IGFALS | | NADK | Notch1 | | | |
| 0.629 | H2A3 | CDON | | | | | | PH | | S100A13 |
| 0.629 | H2A3 | | | | MP2K2 | | | PH | | S100A13 |
| 0.63 | H2A3 | CDON | | | MP2K2 | | | | PTN | |
| 0.63 | H2A3 | CDON | CILP2 | | MP2K2 | NADK | Notch1 | | | |
| 0.63 | H2A3 | | | | | | Notch1 | PH | | |
| 0.63 | H2A3 | CDON | CILP2 | | | NADK | Notch1 | | | S100A13 |
| 0.63 | H2A3 | | CILP2 | | | | | PH | | S100A13 |
| 0.631 | H2A3 | CDON | CILP2 | IGFALS | | | Notch1 | | | |
| 0.631 | H2A3 | | | IGFALS | MP2K2 | NADK | Notch1 | | | |
| 0.631 | H2A3 | | | | | NADK | | | PTN | S100A13 |
| 0.632 | H2A3 | | CILP2 | IGFALS | MP2K2 | | | | | |
| 0.632 | H2A3 | CDON | CILP2 | IGFALS | | NADK | | | | |
| 0.632 | H2A3 | CDON | | | | NADK | | PH | | |
| 0.632 | H2A3 | | | | MP2K2 | NADK | | PH | | |
| 0.632 | H2A3 | | CILP2 | | MP2K2 | NADK | | | | S100A13 |
| 0.632 | H2A3 | | | IGFALS | MP2K2 | NADK | | | | S100A13 |
| 0.632 | H2A3 | | | IGFALS | | | Notch1 | | | S100A13 |
| 0.632 | H2A3 | | CILP2 | | MP2K2 | | Notch1 | | | S100A13 |
| 0.633 | H2A3 | CDON | | | | | | | PTN | S100A13 |
| 0.633 | H2A3 | CDON | CILP2 | IGFALS | | | | | | S100A13 |
| 0.633 | H2A3 | CDON | | | MP2K2 | NADK | Notch1 | | | S100A13 |
| 0.634 | H2A3 | CDON | | | | NADK | | | PTN | |
| 0.634 | H2A3 | CDON | | IGFALS | MP2K2 | | Notch1 | | | |
| 0.634 | H2A3 | | CILP2 | IGFALS | | NADK | Notch1 | | | |
| 0.634 | H2A3 | CDON | CILP2 | | | | | PH | | |
| 0.634 | H2A3 | | CILP2 | | | NADK | | PH | | |
| 0.634 | H2A3 | CDON | | IGFALS | | NADK | | | | S100A13 |
| 0.635 | H2A3 | | | | | NADK | Notch1 | PH | | |
| 0.635 | H2A3 | CDON | | IGFALS | MP2K2 | | | | | S100A13 |
| 0.635 | H2A3 | | | | MP2K2 | NADK | | | PTN | S100A13 |
| 0.636 | H2A3 | | CILP2 | IGFALS | MP2K2 | NADK | | | | |
| 0.636 | H2A3 | CDON | CILP2 | IGFALS | | NADK | Notch1 | | | |
| 0.636 | H2A3 | CDON | | IGFALS | MP2K2 | NADK | Notch1 | | | |
| 0.636 | H2A3 | CDON | | | | | Notch1 | PH | | |
| 0.636 | H2A3 | | CILP2 | | | | Notch1 | PH | | |
| 0.636 | H2A3 | CDON | CILP2 | | MP2K2 | NADK | | | | S100A13 |
| 0.636 | H2A3 | CDON | CILP2 | | MP2K2 | | Notch1 | | | S100A13 |
| 0.637 | H2A3 | CDON | CILP2 | IGFALS | MP2K2 | | | | | |
| 0.637 | H2A3 | CDON | | | MP2K2 | NADK | | | PTN | |
| 0.637 | H2A3 | CDON | | | MP2K2 | | | | PTN | S100A13 |
| 0.637 | H2A3 | CDON | | IGFALS | | | Notch1 | | | S100A13 |
| 0.637 | H2A3 | CDON | CILP2 | | | | | PH | | S100A13 |
| 0.637 | H2A3 | CDON | | | | NADK | | PH | | S100A13 |
| 0.637 | H2A3 | | | | MP2K2 | NADK | | PH | | S100A13 |
| 0.637 | H2A3 | | | | | | Notch1 | PH | | S100A13 |
| 0.638 | H2A3 | | | IGFALS | | | | | PTN | |
| 0.638 | H2A3 | | CILP2 | IGFALS | MP2K2 | | Notch1 | | | |
| 0.638 | H2A3 | | | | | | | PH | PTN | |
| 0.638 | H2A3 | | | IGFALS | | | | PH | | |
| 0.638 | H2A3 | CDON | | | MP2K2 | | | PH | | |
| 0.638 | H2A3 | | CILP2 | IGFALS | | NADK | | | | S100A13 |
| 0.639 | H2A3 | | | IGFALS | MP2K2 | | | | PTN | |
| 0.639 | H2A3 | | CILP2 | IGFALS | | | Notch1 | | | S100A13 |
| 0.639 | H2A3 | | | IGFALS | | NADK | Notch1 | | | S100A13 |
| 0.64 | H2A3 | CDON | CILP2 | IGFALS | MP2K2 | NADK | | | | |
| 0.64 | H2A3 | | | | | | Notch1 | | PTN | |
| 0.64 | H2A3 | CDON | CILP2 | | | | Notch1 | PH | | |
| 0.64 | H2A3 | CDON | | | | NADK | Notch1 | PH | | |
| 0.64 | H2A3 | CDON | | IGFALS | MP2K2 | NADK | | | | S100A13 |
| 0.64 | H2A3 | | CILP2 | | MP2K2 | NADK | Notch1 | | | S100A13 |
| 0.64 | H2A3 | CDON | | | MP2K2 | | | PH | | S100A13 |
| 0.641 | H2A3 | CDON | CILP2 | IGFALS | MP2K2 | | Notch1 | | | |
| 0.641 | H2A3 | | CILP2 | IGFALS | MP2K2 | NADK | Notch1 | | | |
| 0.641 | H2A3 | CDON | CILP2 | | | NADK | | PH | | |
| 0.641 | H2A3 | CDON | | | MP2K2 | NADK | | PH | | |
| 0.641 | H2A3 | | | | MP2K2 | | Notch1 | PH | | |
| 0.641 | H2A3 | CDON | CILP2 | IGFALS | | NADK | | | | S100A13 |
| 0.641 | H2A3 | CDON | CILP2 | IGFALS | | | Notch1 | | | S100A13 |
| 0.641 | H2A3 | | CILP2 | | | NADK | | PH | | S100A13 |
| 0.642 | H2A3 | | | | MP2K2 | | Notch1 | | PTN | |
| 0.642 | H2A3 | | | IGFALS | | NADK | | PH | | |
| 0.642 | H2A3 | | CILP2 | | | NADK | Notch1 | PH | | |
| 0.642 | H2A3 | | CILP2 | IGFALS | MP2K2 | | | | | S100A13 |
| 0.642 | H2A3 | CDON | | | | NADK | | | PTN | S100A13 |
| 0.642 | H2A3 | | | IGFALS | MP2K2 | | Notch1 | | | S100A13 |
| 0.642 | H2A3 | | | | | | | PH | PTN | S100A13 |
| 0.642 | H2A3 | | CILP2 | | | | Notch1 | PH | | S100A13 |
| 0.643 | H2A3 | CDON | | | | | Notch1 | | PTN | |
| 0.643 | H2A3 | | | | MP2K2 | NADK | Notch1 | PH | | |
| 0.643 | H2A3 | CDON | | IGFALS | | NADK | Notch1 | | | S100A13 |
| 0.643 | H2A3 | CDON | CILP2 | | MP2K2 | NADK | Notch1 | | | S100A13 |
| 0.643 | H2A3 | | | IGFALS | | | | PH | | S100A13 |
| 0.643 | H2A3 | CDON | | | | | Notch1 | PH | | S100A13 |
| 0.644 | H2A3 | CDON | CILP2 | IGFALS | MP2K2 | NADK | Notch1 | | | |
| 0.644 | H2A3 | | | | MP2K2 | | | PH | PTN | |
| 0.644 | H2A3 | | CILP2 | | MP2K2 | | | PH | | |
| 0.644 | H2A3 | CDON | | | MP2K2 | NADK | | | PTN | S100A13 |
| 0.644 | H2A3 | | | | | NADK | Notch1 | PH | | S100A13 |
| 0.645 | H2A3 | | | IGFALS | | NADK | | | PTN | |
| 0.645 | H2A3 | | | IGFALS | MP2K2 | NADK | | | PTN | |
| 0.645 | H2A3 | | | IGFALS | MP2K2 | | | PH | | |
| 0.645 | H2A3 | CDON | CILP2 | | | NADK | Notch1 | PH | | |
| 0.645 | H2A3 | CDON | CILP2 | IGFALS | MP2K2 | | | | | S100A13 |
| 0.645 | H2A3 | CDON | | IGFALS | MP2K2 | | Notch1 | | | S100A13 |
| 0.645 | H2A3 | | | IGFALS | MP2K2 | NADK | Notch1 | | | S100A13 |
| 0.646 | H2A3 | CDON | | | MP2K2 | | Notch1 | | PTN | |
| 0.646 | H2A3 | | | | | NADK | Notch1 | | PTN | |
| 0.646 | H2A3 | | | | | NADK | | PH | PTN | |
| 0.646 | H2A3 | CDON | | | MP2K2 | | Notch1 | PH | | |
| 0.646 | H2A3 | | | IGFALS | | | | | PTN | S100A13 |
| 0.646 | H2A3 | | CILP2 | IGFALS | | NADK | Notch1 | | | S100A13 |
| 0.646 | H2A3 | CDON | CILP2 | | | NADK | | PH | | S100A13 |
| 0.646 | H2A3 | CDON | | | MP2K2 | NADK | | PH | | S100A13 |
| 0.646 | H2A3 | CDON | CILP2 | | | | Notch1 | PH | | S100A13 |
| 0.647 | H2A3 | | CILP2 | | | | | | PTN | |
| 0.647 | H2A3 | CDON | | IGFALS | | | | | PTN | |
| 0.647 | H2A3 | | | | MP2K2 | NADK | Notch1 | | PTN | |
| 0.647 | H2A3 | CDON | | IGFALS | | | | PH | | |
| 0.647 | H2A3 | | CILP2 | | MP2K2 | NADK | | PH | | |
| 0.647 | H2A3 | | | IGFALS | MP2K2 | NADK | | PH | | |
| 0.647 | H2A3 | CDON | CILP2 | IGFALS | | NADK | Notch1 | | | S100A13 |
| 0.647 | H2A3 | | CILP2 | | MP2K2 | | | PH | | S100A13 |
| 0.647 | H2A3 | | | | MP2K2 | | Notch1 | PH | | S100A13 |
| 0.648 | H2A3 | CDON | | IGFALS | MP2K2 | | | | PTN | |
| 0.648 | H2A3 | CDON | | | | | | PH | PTN | |
| 0.648 | H2A3 | CDON | | | MP2K2 | NADK | Notch1 | PH | | |
| 0.648 | H2A3 | | | IGFALS | MP2K2 | | | | PTN | S100A13 |
| 0.648 | H2A3 | | CILP2 | IGFALS | MP2K2 | NADK | | | | S100A13 |
| 0.648 | H2A3 | | | | MP2K2 | | | PH | PTN | S100A13 |
| 0.649 | H2A3 | CDON | | | | NADK | Notch1 | | PTN | |
| 0.649 | H2A3 | | CILP2 | IGFALS | | | | PH | | |
| 0.649 | H2A3 | CDON | CILP2 | | MP2K2 | | | PH | | |
| 0.649 | H2A3 | | | IGFALS | | | Notch1 | PH | | |
| 0.649 | H2A3 | CDON | | IGFALS | MP2K2 | NADK | Notch1 | | | S100A13 |
| 0.649 | H2A3 | CDON | | | | NADK | Notch1 | PH | | S100A13 |
| 0.65 | H2A3 | CDON | | IGFALS | | NADK | | PH | | |
| 0.65 | H2A3 | | | | MP2K2 | NADK | | PH | PTN | |
| 0.65 | H2A3 | | CILP2 | | MP2K2 | | Notch1 | PH | | |
| 0.65 | H2A3 | | CILP2 | IGFALS | MP2K2 | | Notch1 | | | S100A13 |
| 0.65 | H2A3 | | | IGFALS | | NADK | | PH | | S100A13 |
| 0.651 | H2A3 | CDON | CILP2 | | | | | | PTN | |
| 0.651 | H2A3 | CDON | | | MP2K2 | NADK | Notch1 | | PTN | |
| 0.651 | H2A3 | | | IGFALS | | NADK | Notch1 | PH | | |
| 0.651 | H2A3 | CDON | CILP2 | IGFALS | MP2K2 | NADK | | | | S100A13 |
| 0.651 | H2A3 | | | | | | Notch1 | | PTN | S100A13 |
| 0.651 | H2A3 | CDON | | IGFALS | | | | PH | | S100A13 |
| 0.651 | H2A3 | CDON | CILP2 | | MP2K2 | | | PH | | S100A13 |
| 0.651 | H2A3 | | | IGFALS | MP2K2 | | | PH | | S100A13 |
| 0.651 | H2A3 | | | | | NADK | | PH | PTN | S100A13 |
| 0.651 | H2A3 | | CILP2 | | | NADK | Notch1 | PH | | S100A13 |
| 0.652 | H2A3 | CDON | | IGFALS | | NADK | | | PTN | |
| 0.652 | H2A3 | CDON | | IGFALS | MP2K2 | NADK | | | PTN | |
| 0.652 | H2A3 | CDON | CILP2 | | MP2K2 | NADK | | PH | | |
| 0.652 | H2A3 | | CILP2 | | MP2K2 | NADK | Notch1 | PH | | |
| 0.652 | H2A3 | CDON | CILP2 | IGFALS | MP2K2 | | Notch1 | | | S100A13 |
| 0.652 | H2A3 | CDON | | | | | | PH | PTN | S100A13 |
| 0.652 | H2A3 | | CILP2 | IGFALS | | | | PH | | S100A13 |
| 0.652 | H2A3 | CDON | | | MP2K2 | | Notch1 | PH | | S100A13 |
| 0.652 | H2A3 | | | | MP2K2 | NADK | Notch1 | PH | | S100A13 |
| 0.653 | H2A3 | CDON | | | MP2K2 | | | PH | PTN | |
| 0.653 | H2A3 | | CILP2 | IGFALS | | NADK | | PH | | |
| 0.653 | H2A3 | CDON | | IGFALS | | | Notch1 | PH | | |
| 0.653 | H2A3 | CDON | CILP2 | | MP2K2 | | Notch1 | PH | | |
| 0.653 | H2A3 | | CILP2 | | | | | | PTN | S100A13 |
| 0.653 | H2A3 | | | IGFALS | | NADK | | | PTN | S100A13 |
| 0.653 | H2A3 | | | | MP2K2 | | Notch1 | | PTN | S100A13 |
| 0.654 | H2A3 | | CILP2 | | MP2K2 | | | | PTN | |
| 0.654 | H2A3 | CDON | CILP2 | IGFALS | | | | PH | | |
| 0.654 | H2A3 | CDON | | IGFALS | MP2K2 | | | PH | | |
| 0.654 | H2A3 | CDON | | | | NADK | | PH | PTN | |
| 0.654 | H2A3 | | CILP2 | IGFALS | | | Notch1 | PH | | |
| 0.654 | H2A3 | CDON | | IGFALS | | | | | PTN | S100A13 |
| 0.654 | H2A3 | | | IGFALS | MP2K2 | NADK | | | PTN | S100A13 |
| 0.654 | H2A3 | CDON | | | | | Notch1 | | PTN | S100A13 |
| 0.654 | H2A3 | | CILP2 | IGFALS | MP2K2 | NADK | Notch1 | | | S100A13 |
| 0.654 | H2A3 | | | | MP2K2 | NADK | | PH | PTN | S100A13 |
| 0.654 | H2A3 | | CILP2 | | MP2K2 | NADK | | PH | | S100A13 |
| 0.654 | H2A3 | CDON | CILP2 | | | NADK | Notch1 | PH | | S100A13 |
| 0.655 | H2A3 | CDON | | IGFALS | MP2K2 | NADK | | PH | | |
| 0.655 | H2A3 | | | IGFALS | MP2K2 | | Notch1 | PH | | |
| 0.655 | H2A3 | CDON | | IGFALS | | NADK | Notch1 | PH | | |
| 0.655 | H2A3 | CDON | CILP2 | | MP2K2 | NADK | Notch1 | PH | | |
| 0.655 | H2A3 | CDON | CILP2 | | | | | | PTN | S100A13 |
| 0.655 | H2A3 | | CILP2 | | MP2K2 | | Notch1 | PH | | S100A13 |
| 0.656 | H2A3 | | CILP2 | | | NADK | | | PTN | |
| 0.656 | H2A3 | CDON | CILP2 | IGFALS | | | Notch1 | PH | | |
| 0.656 | H2A3 | | | IGFALS | MP2K2 | NADK | Notch1 | PH | | |
| 0.656 | H2A3 | CDON | | IGFALS | MP2K2 | | | | PTN | S100A13 |
| 0.656 | H2A3 | CDON | | | MP2K2 | | Notch1 | | PTN | S100A13 |
| 0.656 | H2A3 | CDON | CILP2 | IGFALS | MP2K2 | NADK | Notch1 | | | S100A13 |
| 0.656 | H2A3 | CDON | CILP2 | IGFALS | | | | PH | | S100A13 |
| 0.656 | H2A3 | CDON | | | MP2K2 | | | PH | PTN | S100A13 |
| 0.656 | H2A3 | | | IGFALS | MP2K2 | NADK | | PH | | S100A13 |
| 0.656 | H2A3 | | | IGFALS | | | Notch1 | PH | | S100A13 |
| 0.656 | H2A3 | CDON | | | MP2K2 | NADK | Notch1 | PH | | S100A13 |
| 0.657 | H2A3 | CDON | CILP2 | | MP2K2 | | | | PTN | |
| 0.657 | H2A3 | | | IGFALS | | | | PH | PTN | |
| 0.657 | H2A3 | | CILP2 | IGFALS | MP2K2 | | | PH | | |
| 0.657 | H2A3 | CDON | CILP2 | IGFALS | | NADK | | PH | | |
| 0.657 | H2A3 | CDON | | | MP2K2 | NADK | | PH | PTN | |
| 0.657 | H2A3 | | CILP2 | IGFALS | | NADK | Notch1 | PH | | |
| 0.657 | H2A3 | | | | | NADK | Notch1 | | PTN | S100A13 |
| 0.657 | H2A3 | CDON | | IGFALS | | NADK | | PH | | S100A13 |
| 0.657 | H2A3 | CDON | CILP2 | | MP2K2 | NADK | | PH | | S100A13 |
| 0.657 | H2A3 | CDON | CILP2 | | MP2K2 | | Notch1 | PH | | S100A13 |
| 0.658 | H2A3 | | | IGFALS | | | Notch1 | | PTN | |
| 0.658 | H2A3 | | | IGFALS | MP2K2 | | Notch1 | | PTN | |
| 0.658 | H2A3 | | | | MP2K2 | NADK | Notch1 | | PTN | S100A13 |
| 0.658 | H2A3 | CDON | | IGFALS | MP2K2 | | | PH | | S100A13 |
| 0.659 | H2A3 | CDON | CILP2 | | | NADK | | | PTN | |
| 0.659 | H2A3 | | | IGFALS | MP2K2 | | | PH | PTN | |
| 0.659 | H2A3 | | CILP2 | IGFALS | MP2K2 | NADK | | PH | | |
| 0.659 | H2A3 | CDON | | IGFALS | MP2K2 | | Notch1 | PH | | |
| 0.659 | H2A3 | CDON | CILP2 | IGFALS | | NADK | Notch1 | PH | | |
| 0.659 | H2A3 | CDON | | IGFALS | MP2K2 | NADK | Notch1 | PH | | |
| 0.659 | H2A3 | | CILP2 | | MP2K2 | | | | PTN | S100A13 |
| 0.659 | H2A3 | CDON | | IGFALS | | NADK | | | PTN | S100A13 |
| 0.659 | H2A3 | CDON | | | | NADK | | PH | PTN | S100A13 |
| 0.659 | H2A3 | | CILP2 | IGFALS | | NADK | | PH | | S100A13 |
| 0.659 | H2A3 | CDON | | IGFALS | | | Notch1 | PH | | S100A13 |
| 0.66 | H2A3 | CDON | | IGFALS | | | Notch1 | | PTN | |
| 0.66 | H2A3 | CDON | | IGFALS | MP2K2 | NADK | | | PTN | S100A13 |
| 0.66 | H2A3 | CDON | | | | NADK | Notch1 | | PTN | S100A13 |
| 0.66 | H2A3 | | CILP2 | IGFALS | | | Notch1 | PH | | S100A13 |
| 0.661 | H2A3 | | CILP2 | | MP2K2 | NADK | | | PTN | |
| 0.661 | H2A3 | | CILP2 | | | | Notch1 | | PTN | |
| 0.661 | H2A3 | CDON | CILP2 | | | | Notch1 | | PTN | |
| 0.661 | H2A3 | CDON | | IGFALS | MP2K2 | | Notch1 | | PTN | |
| 0.661 | H2A3 | CDON | CILP2 | | MP2K2 | | | | PTN | S100A13 |
| 0.661 | H2A3 | CDON | | | MP2K2 | NADK | Notch1 | | PTN | S100A13 |
| 0.661 | H2A3 | CDON | | | MP2K2 | NADK | | PH | PTN | S100A13 |
| 0.661 | H2A3 | | | IGFALS | | NADK | Notch1 | PH | | S100A13 |
| 0.661 | H2A3 | | CILP2 | | MP2K2 | NADK | Notch1 | PH | | S100A13 |
| 0.662 | H2A3 | | | IGFALS | | NADK | Notch1 | | PTN | |
| 0.662 | H2A3 | | | IGFALS | MP2K2 | NADK | Notch1 | | PTN | |
| 0.662 | H2A3 | CDON | CILP2 | IGFALS | MP2K2 | | | PH | | |
| 0.662 | H2A3 | | | IGFALS | | NADK | | PH | PTN | |
| 0.662 | H2A3 | | | | | | Notch1 | PH | PTN | |
| 0.662 | H2A3 | | CILP2 | IGFALS | MP2K2 | | Notch1 | PH | | |
| 0.662 | H2A3 | | CILP2 | | | NADK | | | PTN | S100A13 |
| 0.662 | H2A3 | | CILP2 | IGFALS | MP2K2 | | | PH | | S100A13 |
| 0.662 | H2A3 | CDON | CILP2 | IGFALS | | NADK | | PH | | S100A13 |
| 0.662 | H2A3 | CDON | | IGFALS | MP2K2 | NADK | | PH | | S100A13 |
| 0.662 | H2A3 | CDON | CILP2 | IGFALS | | | Notch1 | PH | | S100A13 |
| 0.662 | H2A3 | CDON | CILP2 | | MP2K2 | NADK | Notch1 | PH | | S100A13 |
| 0.663 | H2A3 | CDON | CILP2 | | MP2K2 | NADK | | | PTN | |
| 0.663 | H2A3 | | | IGFALS | MP2K2 | NADK | | PH | PTN | |
| 0.663 | H2A3 | CDON | CILP2 | IGFALS | MP2K2 | NADK | | PH | | |
| 0.663 | H2A3 | | CILP2 | IGFALS | MP2K2 | NADK | Notch1 | PH | | |
| 0.663 | H2A3 | CDON | CILP2 | | | NADK | | | PTN | S100A13 |
| 0.663 | H2A3 | | | IGFALS | | | | PH | PTN | S100A13 |
| 0.663 | H2A3 | | | IGFALS | MP2K2 | | Notch1 | PH | | S100A13 |
| 0.664 | H2A3 | | CILP2 | IGFALS | | | | | PTN | |
| 0.664 | H2A3 | CDON | | IGFALS | | NADK | Notch1 | | PTN | |
| 0.664 | H2A3 | CDON | | IGFALS | MP2K2 | NADK | Notch1 | | PTN | |
| 0.664 | H2A3 | | CILP2 | | | | | PH | PTN | |
| 0.664 | H2A3 | CDON | | | | | Notch1 | PH | PTN | |
| 0.664 | H2A3 | | | | MP2K2 | | Notch1 | PH | PTN | |
| 0.664 | H2A3 | CDON | CILP2 | IGFALS | MP2K2 | | Notch1 | PH | | |
| 0.664 | H2A3 | CDON | | IGFALS | | NADK | Notch1 | PH | | S100A13 |
| 0.665 | H2A3 | | CILP2 | | MP2K2 | | Notch1 | | PTN | |
| 0.665 | H2A3 | CDON | CILP2 | | MP2K2 | | Notch1 | | PTN | |
| 0.665 | H2A3 | CDON | | IGFALS | | | | PH | PTN | |
| 0.665 | H2A3 | CDON | CILP2 | IGFALS | MP2K2 | NADK | Notch1 | PH | | |
| 0.665 | H2A3 | | | IGFALS | MP2K2 | | | PH | PTN | S100A13 |
| 0.665 | H2A3 | CDON | CILP2 | IGFALS | MP2K2 | | | PH | | S100A13 |
| 0.665 | H2A3 | | CILP2 | IGFALS | | NADK | Notch1 | PH | | S100A13 |
| 0.666 | H2A3 | CDON | CILP2 | IGFALS | | | | | PTN | |
| 0.666 | H2A3 | CDON | | | MP2K2 | | Notch1 | PH | PTN | |
| 0.666 | H2A3 | | | | | NADK | Notch1 | PH | PTN | |
| 0.666 | H2A3 | | CILP2 | | MP2K2 | NADK | | | PTN | S100A13 |
| 0.666 | H2A3 | CDON | | IGFALS | MP2K2 | | Notch1 | PH | | S100A13 |
| 0.666 | H2A3 | | | IGFALS | MP2K2 | NADK | Notch1 | PH | | S100A13 |
| 0.667 | H2A3 | | CILP2 | IGFALS | MP2K2 | | | | PTN | |
| 0.667 | H2A3 | | CILP2 | | | NADK | Notch1 | | PTN | |
| 0.667 | H2A3 | CDON | CILP2 | | | NADK | Notch1 | | PTN | |
| 0.667 | H2A3 | CDON | CILP2 | | | | | PH | PTN | |
| 0.667 | H2A3 | CDON | | IGFALS | MP2K2 | | | PH | PTN | |
| 0.667 | H2A3 | CDON | | | | NADK | Notch1 | PH | PTN | |
| 0.667 | H2A3 | | | | MP2K2 | NADK | Notch1 | PH | PTN | |
| 0.667 | H2A3 | | CILP2 | | | | Notch1 | | PTN | S100A13 |
| 0.667 | H2A3 | CDON | CILP2 | | | | Notch1 | | PTN | S100A13 |
| 0.667 | H2A3 | | | IGFALS | | | Notch1 | | PTN | S100A13 |
| 0.667 | H2A3 | | CILP2 | IGFALS | MP2K2 | NADK | | PH | | S100A13 |
| 0.667 | H2A3 | CDON | CILP2 | IGFALS | | NADK | Notch1 | PH | | S100A13 |
| 0.668 | H2A3 | CDON | CILP2 | IGFALS | MP2K2 | | | | PTN | |
| 0.668 | H2A3 | CDON | | IGFALS | | NADK | | PH | PTN | |
| 0.668 | H2A3 | CDON | CILP2 | | MP2K2 | NADK | | | PTN | S100A13 |
| 0.668 | H2A3 | CDON | | IGFALS | | | Notch1 | | PTN | S100A13 |
| 0.668 | H2A3 | | | IGFALS | MP2K2 | | Notch1 | | PTN | S100A13 |
| 0.668 | H2A3 | | CILP2 | | | | | PH | PTN | S100A13 |
| 0.668 | H2A3 | | | | | | Notch1 | PH | PTN | S100A13 |
| 0.668 | H2A3 | | CILP2 | IGFALS | MP2K2 | | Notch1 | PH | | S100A13 |
| 0.668 | H2A3 | CDON | | IGFALS | MP2K2 | NADK | Notch1 | PH | | S100A13 |
| 0.669 | H2A3 | CDON | | IGFALS | MP2K2 | NADK | | PH | PTN | |
| 0.669 | H2A3 | CDON | | | MP2K2 | NADK | Notch1 | PH | PTN | |
| 0.669 | H2A3 | | CILP2 | IGFALS | | | | | PTN | S100A13 |
| 0.669 | H2A3 | CDON | | IGFALS | | | | PH | PTN | S100A13 |
| 0.669 | H2A3 | | | IGFALS | | NADK | | PH | PTN | S100A13 |
| 0.669 | H2A3 | CDON | CILP2 | IGFALS | MP2K2 | NADK | | PH | | S100A13 |
| 0.669 | H2A3 | CDON | CILP2 | IGFALS | MP2K2 | | Notch1 | PH | | S100A13 |
| 0.67 | H2A3 | | CILP2 | IGFALS | | NADK | | | PTN | |
| 0.67 | H2A3 | | CILP2 | | MP2K2 | NADK | Notch1 | | PTN | |
| 0.67 | H2A3 | CDON | CILP2 | | MP2K2 | NADK | Notch1 | | PTN | |
| 0.67 | H2A3 | | CILP2 | | MP2K2 | | | PH | PTN | |
| 0.67 | H2A3 | | CILP2 | | | NADK | | PH | PTN | |
| 0.67 | H2A3 | CDON | CILP2 | IGFALS | | | | | PTN | S100A13 |
| 0.67 | H2A3 | CDON | | IGFALS | MP2K2 | | Notch1 | | PTN | S100A13 |
| 0.67 | H2A3 | CDON | CILP2 | | | | | PH | PTN | S100A13 |
| 0.67 | H2A3 | | | IGFALS | MP2K2 | NADK | | PH | PTN | S100A13 |
| 0.67 | H2A3 | CDON | | | | | Notch1 | PH | PTN | S100A13 |
| 0.67 | H2A3 | | | | MP2K2 | | Notch1 | PH | PTN | S100A13 |
| 0.671 | H2A3 | CDON | CILP2 | IGFALS | | NADK | | | PTN | |
| 0.671 | H2A3 | | CILP2 | IGFALS | MP2K2 | NADK | | | PTN | |
| 0.671 | H2A3 | | CILP2 | | MP2K2 | | Notch1 | | PTN | S100A13 |
| 0.671 | H2A3 | CDON | CILP2 | | MP2K2 | | Notch1 | | PTN | S100A13 |
| 0.671 | H2A3 | CDON | | IGFALS | MP2K2 | | | PH | PTN | S100A13 |
| 0.672 | H2A3 | CDON | CILP2 | IGFALS | MP2K2 | NADK | | | PTN | |
| 0.672 | H2A3 | CDON | CILP2 | | MP2K2 | | | PH | PTN | |
| 0.672 | H2A3 | CDON | CILP2 | | | NADK | | PH | PTN | |
| 0.672 | H2A3 | | | IGFALS | | NADK | Notch1 | | PTN | S100A13 |
| 0.672 | H2A3 | | | IGFALS | MP2K2 | NADK | Notch1 | | PTN | S100A13 |
| 0.672 | H2A3 | CDON | | | MP2K2 | | Notch1 | PH | PTN | S100A13 |
| 0.672 | H2A3 | | | | | NADK | Notch1 | PH | PTN | S100A13 |
| 0.672 | H2A3 | | CILP2 | IGFALS | MP2K2 | NADK | Notch1 | PH | | S100A13 |
| 0.673 | H2A3 | CDON | CILP2 | IGFALS | | | Notch1 | | PTN | |
| 0.673 | H2A3 | | CILP2 | IGFALS | MP2K2 | | | | PTN | S100A13 |
| 0.673 | H2A3 | CDON | CILP2 | | | NADK | Notch1 | | PTN | S100A13 |
| 0.673 | H2A3 | CDON | | IGFALS | | NADK | Notch1 | | PTN | S100A13 |
| 0.673 | H2A3 | CDON | | IGFALS | MP2K2 | NADK | Notch1 | | PTN | S100A13 |
| 0.673 | H2A3 | | CILP2 | | MP2K2 | | | PH | PTN | S100A13 |
| 0.673 | H2A3 | | | | MP2K2 | NADK | Notch1 | PH | PTN | S100A13 |
| 0.673 | H2A3 | CDON | CILP2 | IGFALS | MP2K2 | NADK | Notch1 | PH | | S100A13 |
| 0.674 | H2A3 | | CILP2 | IGFALS | | | Notch1 | | PTN | |
| 0.674 | H2A3 | | CILP2 | | MP2K2 | NADK | | PH | PTN | |
| 0.674 | H2A3 | CDON | CILP2 | IGFALS | MP2K2 | | | | PTN | S100A13 |
| 0.674 | H2A3 | | CILP2 | | | NADK | Notch1 | | PTN | S100A13 |
| 0.674 | H2A3 | | CILP2 | | | NADK | | PH | PTN | S100A13 |
| 0.674 | H2A3 | CDON | | IGFALS | | NADK | | PH | PTN | S100A13 |
| 0.674 | H2A3 | CDON | | | | NADK | Notch1 | PH | PTN | S100A13 |
| 0.675 | H2A3 | | CILP2 | IGFALS | MP2K2 | | Notch1 | | PTN | |
| 0.675 | H2A3 | CDON | CILP2 | IGFALS | MP2K2 | | Notch1 | | PTN | |
| 0.675 | H2A3 | | | IGFALS | | | Notch1 | PH | PTN | |
| 0.675 | H2A3 | | CILP2 | IGFALS | | NADK | | | PTN | S100A13 |
| 0.675 | H2A3 | CDON | CILP2 | | MP2K2 | | | PH | PTN | S100A13 |
| 0.675 | H2A3 | CDON | | IGFALS | MP2K2 | NADK | | PH | PTN | S100A13 |
| 0.675 | H2A3 | CDON | | | MP2K2 | NADK | Notch1 | PH | PTN | S100A13 |
| 0.676 | H2A3 | CDON | CILP2 | | MP2K2 | NADK | | PH | PTN | |
| 0.676 | H2A3 | | CILP2 | | | | Notch1 | PH | PTN | |
| 0.676 | H2A3 | CDON | CILP2 | | | | Notch1 | PH | PTN | |
| 0.676 | H2A3 | | | IGFALS | MP2K2 | | Notch1 | PH | PTN | |
| 0.676 | H2A3 | CDON | CILP2 | IGFALS | | NADK | | | PTN | S100A13 |
| 0.676 | H2A3 | CDON | CILP2 | | MP2K2 | NADK | Notch1 | | PTN | S100A13 |
| 0.676 | H2A3 | CDON | CILP2 | | | NADK | | PH | PTN | S100A13 |
| 0.677 | H2A3 | CDON | CILP2 | IGFALS | | NADK | Notch1 | | PTN | |
| 0.677 | H2A3 | | CILP2 | IGFALS | | | | PH | PTN | |
| 0.677 | H2A3 | CDON | | IGFALS | | | Notch1 | PH | PTN | |
| 0.677 | H2A3 | CDON | | IGFALS | MP2K2 | | Notch1 | PH | PTN | |
| 0.677 | H2A3 | | | IGFALS | | NADK | Notch1 | PH | PTN | |
| 0.677 | H2A3 | | | IGFALS | MP2K2 | NADK | Notch1 | PH | PTN | |
| 0.677 | H2A3 | | CILP2 | | MP2K2 | NADK | Notch1 | | PTN | S100A13 |
| 0.678 | H2A3 | | CILP2 | IGFALS | | NADK | Notch1 | | PTN | |
| 0.678 | H2A3 | CDON | CILP2 | IGFALS | MP2K2 | NADK | Notch1 | | PTN | |
| 0.678 | H2A3 | CDON | | IGFALS | | NADK | Notch1 | PH | PTN | |
| 0.678 | H2A3 | | CILP2 | IGFALS | MP2K2 | NADK | | | PTN | S100A13 |
| 0.678 | H2A3 | CDON | CILP2 | IGFALS | MP2K2 | NADK | | | PTN | S100A13 |
| 0.679 | H2A3 | | CILP2 | IGFALS | MP2K2 | NADK | Notch1 | | PTN | |
| 0.679 | H2A3 | CDON | CILP2 | IGFALS | | | | PH | PTN | |
| 0.679 | H2A3 | CDON | CILP2 | | MP2K2 | | Notch1 | PH | PTN | |
| 0.679 | H2A3 | CDON | | IGFALS | MP2K2 | NADK | Notch1 | PH | PTN | |
| 0.679 | H2A3 | | CILP2 | IGFALS | | | Notch1 | | PTN | S100A13 |
| 0.679 | H2A3 | CDON | CILP2 | IGFALS | | | Notch1 | | PTN | S100A13 |
| 0.679 | H2A3 | | CILP2 | | MP2K2 | NADK | | PH | PTN | S100A13 |
| 0.679 | H2A3 | CDON | CILP2 | | MP2K2 | NADK | | PH | PTN | S100A13 |
| 0.68 | H2A3 | | CILP2 | IGFALS | MP2K2 | | | PH | PTN | |
| 0.68 | H2A3 | | CILP2 | | MP2K2 | | Notch1 | PH | PTN | |
| 0.68 | H2A3 | | CILP2 | | | NADK | Notch1 | PH | PTN | |
| 0.68 | H2A3 | CDON | CILP2 | | | NADK | Notch1 | PH | PTN | |
| 0.68 | H2A3 | | CILP2 | | | | Notch1 | PH | PTN | S100A13 |
| 0.68 | H2A3 | CDON | CILP2 | | | | Notch1 | PH | PTN | S100A13 |
| 0.681 | H2A3 | | CILP2 | IGFALS | | NADK | | PH | PTN | |
| 0.681 | H2A3 | CDON | CILP2 | IGFALS | MP2K2 | | Notch1 | | PTN | S100A13 |
| 0.681 | H2A3 | | CILP2 | IGFALS | | | | PH | PTN | S100A13 |
| 0.681 | H2A3 | | | IGFALS | | | Notch1 | PH | PTN | S100A13 |
| 0.682 | H2A3 | CDON | CILP2 | IGFALS | MP2K2 | | | PH | PTN | |
| 0.682 | H2A3 | CDON | CILP2 | IGFALS | | NADK | | PH | PTN | |
| 0.682 | H2A3 | CDON | CILP2 | | MP2K2 | NADK | Notch1 | PH | PTN | |
| 0.682 | H2A3 | | CILP2 | IGFALS | MP2K2 | | Notch1 | | PTN | S100A13 |
| 0.682 | H2A3 | CDON | CILP2 | IGFALS | | | | PH | PTN | S100A13 |
| 0.682 | H2A3 | CDON | | IGFALS | | | Notch1 | PH | PTN | S100A13 |
| 0.682 | H2A3 | | | IGFALS | MP2K2 | | Notch1 | PH | PTN | S100A13 |
| 0.683 | H2A3 | | CILP2 | IGFALS | MP2K2 | NADK | | PH | PTN | |
| 0.683 | H2A3 | | CILP2 | | MP2K2 | NADK | Notch1 | PH | PTN | |
| 0.683 | H2A3 | CDON | CILP2 | IGFALS | | NADK | Notch1 | | PTN | S100A13 |
| 0.683 | H2A3 | CDON | CILP2 | | MP2K2 | | Notch1 | PH | PTN | S100A13 |
| 0.683 | H2A3 | CDON | | IGFALS | MP2K2 | | Notch1 | PH | PTN | S100A13 |
| 0.684 | H2A3 | CDON | CILP2 | IGFALS | MP2K2 | NADK | | PH | PTN | |
| 0.684 | H2A3 | | CILP2 | IGFALS | | NADK | Notch1 | | PTN | S100A13 |
| 0.684 | H2A3 | | CILP2 | IGFALS | MP2K2 | | | PH | PTN | S100A13 |
| 0.684 | H2A3 | | CILP2 | | MP2K2 | | Notch1 | PH | PTN | S100A13 |
| 0.685 | H2A3 | CDON | CILP2 | IGFALS | MP2K2 | NADK | Notch1 | | PTN | S100A13 |
| 0.685 | H2A3 | CDON | CILP2 | IGFALS | MP2K2 | | | PH | PTN | S100A13 |
| 0.685 | H2A3 | | CILP2 | | | NADK | Notch1 | PH | PTN | S100A13 |
| 0.685 | H2A3 | CDON | CILP2 | | | NADK | Notch1 | PH | PTN | S100A13 |
| 0.685 | H2A3 | | | IGFALS | | NADK | Notch1 | PH | PTN | S100A13 |
| 0.685 | H2A3 | CDON | | IGFALS | | NADK | Notch1 | PH | PTN | S100A13 |
| 0.685 | H2A3 | | | IGFALS | MP2K2 | NADK | Notch1 | PH | PTN | S100A13 |
| 0.685 | H2A3 | CDON | | IGFALS | MP2K2 | NADK | Notch1 | PH | PTN | S100A13 |
| 0.686 | H2A3 | | CILP2 | IGFALS | | | Notch1 | PH | PTN | |
| 0.686 | H2A3 | CDON | CILP2 | IGFALS | | | Notch1 | PH | PTN | |
| 0.686 | H2A3 | | CILP2 | IGFALS | MP2K2 | NADK | Notch1 | | PTN | S100A13 |
| 0.686 | H2A3 | | CILP2 | IGFALS | | NADK | | PH | PTN | S100A13 |
| 0.686 | H2A3 | CDON | CILP2 | IGFALS | | NADK | | PH | PTN | S100A13 |
| 0.687 | H2A3 | CDON | CILP2 | | MP2K2 | NADK | Notch1 | PH | PTN | S100A13 |
| 0.688 | H2A3 | | CILP2 | IGFALS | MP2K2 | | Notch1 | PH | PTN | |
| 0.688 | H2A3 | CDON | CILP2 | IGFALS | MP2K2 | | Notch1 | PH | PTN | |
| 0.688 | H2A3 | CDON | CILP2 | IGFALS | | NADK | Notch1 | PH | PTN | |
| 0.688 | H2A3 | | CILP2 | IGFALS | MP2K2 | NADK | | PH | PTN | S100A13 |
| 0.688 | H2A3 | CDON | CILP2 | IGFALS | MP2K2 | NADK | | PH | PTN | S100A13 |
| 0.688 | H2A3 | | CILP2 | | MP2K2 | NADK | Notch1 | PH | PTN | S100A13 |
| 0.689 | H2A3 | | CILP2 | IGFALS | | NADK | Notch1 | PH | PTN | |
| 0.689 | H2A3 | CDON | CILP2 | IGFALS | MP2K2 | NADK | Notch1 | PH | PTN | |
| 0.69 | H2A3 | | CILP2 | IGFALS | MP2K2 | NADK | Notch1 | PH | PTN | |
| 0.69 | H2A3 | CDON | CILP2 | IGFALS | | | Notch1 | PH | PTN | S100A13 |
| 0.691 | H2A3 | | CILP2 | IGFALS | | | Notch1 | PH | PTN | S100A13 |
| 0.692 | H2A3 | CDON | CILP2 | IGFALS | MP2K2 | | Notch1 | PH | PTN | S100A13 |
| 0.693 | H2A3 | | CILP2 | IGFALS | MP2K2 | | Notch1 | PH | PTN | S100A13 |
| 0.693 | H2A3 | CDON | CILP2 | IGFALS | | NADK | Notch1 | PH | PTN | S100A13 |
| 0.694 | H2A3 | | CILP2 | IGFALS | | NADK | Notch1 | PH | PTN | S100A13 |
| 0.694 | H2A3 | CDON | CILP2 | IGFALS | MP2K2 | NADK | Notch1 | PH | PTN | S100A13 |
| *0.696 | H2A3 | | CILP2 | IGFALS | MP2K2 | NADK | Notch1 | PH | PTN | S100A13 |

*Included in Table 38

TABLE 47
IGFALS Biomarker Panels-20 Year Model

| AUC | IGFALS | CDON | CILP2 | H2A3 | MP2K2 | NADK | Notch1 | PH | PTN | S100A13 |
|---|---|---|---|---|---|---|---|---|---|---|
| 0.602 | IGFALS | | | H2A3 | | | | | | |
| 0.607 | IGFALS | CDON | | | | | | | | |
| 0.607 | IGFALS | | | | MP2K2 | | | | | |
| 0.609 | IGFALS | | | | | | | | | S100A13 |
| 0.61 | IGFALS | | | | | | Notch1 | | | |
| 0.611 | IGFALS | | | | | NADK | | | | |
| 0.612 | IGFALS | | | H2A3 | | NADK | | | | |
| 0.613 | IGFALS | | CILP2 | | | | | | | |
| 0.614 | IGFALS | | | H2A3 | MP2K2 | | | | | |
| 0.615 | IGFALS | | | H2A3 | | | | | | S100A13 |
| 0.616 | IGFALS | CDON | | H2A3 | | | | | | |
| 0.616 | IGFALS | CDON | | | MP2K2 | | | | | |
| 0.616 | IGFALS | CDON | | | | | Notch1 | | | |
| 0.617 | IGFALS | | | | MP2K2 | NADK | | | | |
| 0.619 | IGFALS | | CILP2 | H2A3 | | | | | | |
| 0.619 | IGFALS | | | H2A3 | MP2K2 | NADK | | | | |
| 0.619 | IGFALS | | | H2A3 | | | Notch1 | | | |
| 0.619 | IGFALS | | | | MP2K2 | | Notch1 | | | |
| 0.62 | IGFALS | CDON | CILP2 | | | | | | | |
| 0.62 | IGFALS | CDON | | | | NADK | | | | |
| 0.621 | IGFALS | | CILP2 | | | | Notch1 | | | |
| 0.621 | IGFALS | | | | | NADK | Notch1 | | | |
| 0.621 | IGFALS | CDON | | | | | | | | S100A13 |
| 0.621 | IGFALS | | | | MP2K2 | | | | | S100A13 |
| 0.622 | IGFALS | CDON | | H2A3 | | NADK | | | | |
| 0.624 | IGFALS | CDON | CILP2 | | | | Notch1 | | | |
| 0.624 | IGFALS | CDON | | | MP2K2 | | Notch1 | | | |
| 0.625 | IGFALS | | CILP2 | | MP2K2 | | | | | |
| 0.625 | IGFALS | CDON | | H2A3 | MP2K2 | | | | | |
| 0.625 | IGFALS | CDON | | | MP2K2 | NADK | | | | |
| 0.625 | IGFALS | CDON | | H2A3 | | | Notch1 | | | |
| 0.625 | IGFALS | | | H2A3 | | NADK | Notch1 | | | |
| 0.625 | IGFALS | | | | MP2K2 | NADK | Notch1 | | | |
| 0.625 | IGFALS | | CILP2 | | | | | | | S100A13 |
| 0.626 | IGFALS | CDON | CILP2 | H2A3 | | | | | | |
| 0.626 | IGFALS | CDON | | | | NADK | Notch1 | | | |
| 0.626 | IGFALS | CDON | | H2A3 | | | | | | S100A13 |
| 0.626 | IGFALS | | | H2A3 | MP2K2 | | | | | S100A13 |
| 0.626 | IGFALS | | | | | NADK | | | | S100A13 |
| 0.626 | IGFALS | | | H2A3 | | NADK | | | | S100A13 |
| 0.627 | IGFALS | | | | | | | PH | | |
| 0.628 | IGFALS | | CILP2 | | | NADK | | | | |
| 0.628 | IGFALS | | CILP2 | H2A3 | | NADK | | | | |
| 0.628 | IGFALS | | CILP2 | H2A3 | | | Notch1 | | | |
| 0.628 | IGFALS | | | H2A3 | MP2K2 | | Notch1 | | | |
| 0.628 | IGFALS | | CILP2 | H2A3 | | | | | | S100A13 |
| 0.628 | IGFALS | | | | | | Notch1 | | | S100A13 |
| 0.629 | IGFALS | CDON | CILP2 | | MP2K2 | | | | | |
| 0.629 | IGFALS | CDON | | H2A3 | MP2K2 | NADK | | | | |
| 0.629 | IGFALS | CDON | | H2A3 | | NADK | Notch1 | | | |
| 0.63 | IGFALS | CDON | CILP2 | | | | | | | S100A13 |
| 0.63 | IGFALS | CDON | | | MP2K2 | | | | | S100A13 |
| 0.631 | IGFALS | CDON | CILP2 | H2A3 | | | Notch1 | | | |
| 0.631 | IGFALS | | CILP2 | | MP2K2 | | Notch1 | | | |
| 0.631 | IGFALS | CDON | | | MP2K2 | NADK | Notch1 | | | |
| 0.631 | IGFALS | | | H2A3 | MP2K2 | NADK | Notch1 | | | |
| 0.632 | IGFALS | | | | | | | | PTN | |
| 0.632 | IGFALS | | CILP2 | H2A3 | MP2K2 | | | | | |
| 0.632 | IGFALS | CDON | CILP2 | | | NADK | | | | |
| 0.632 | IGFALS | CDON | CILP2 | H2A3 | | NADK | | | | |
| 0.632 | IGFALS | | CILP2 | | | NADK | Notch1 | | | |
| 0.632 | IGFALS | | | | MP2K2 | NADK | | | | S100A13 |
| 0.632 | IGFALS | | | H2A3 | MP2K2 | NADK | | | | S100A13 |
| 0.632 | IGFALS | | | H2A3 | | | Notch1 | | | S100A13 |
| 0.633 | IGFALS | | | | MP2K2 | | | | PTN | |
| 0.633 | IGFALS | CDON | CILP2 | H2A3 | | | | | | S100A13 |
| 0.633 | IGFALS | CDON | | | | NADK | | | | S100A13 |
| 0.633 | IGFALS | CDON | | | | | Notch1 | | | S100A13 |
| 0.634 | IGFALS | | CILP2 | | MP2K2 | NADK | | | | |
| 0.634 | IGFALS | CDON | CILP2 | | MP2K2 | | Notch1 | | | |
| 0.634 | IGFALS | CDON | | H2A3 | MP2K2 | | Notch1 | | | |
| 0.634 | IGFALS | CDON | CILP2 | | | NADK | Notch1 | | | |
| 0.634 | IGFALS | | CILP2 | H2A3 | | NADK | Notch1 | | | |
| 0.634 | IGFALS | | | | MP2K2 | | | PH | | |
| 0.634 | IGFALS | CDON | | H2A3 | | NADK | | | | S100A13 |
| 0.635 | IGFALS | CDON | | H2A3 | MP2K2 | | | | | S100A13 |
| 0.635 | IGFALS | | CILP2 | | | | Notch1 | | | S100A13 |
| 0.635 | IGFALS | | | | | | | PH | | S100A13 |
| 0.636 | IGFALS | | CILP2 | H2A3 | MP2K2 | NADK | | | | |
| 0.636 | IGFALS | CDON | CILP2 | H2A3 | | NADK | Notch1 | | | |
| 0.636 | IGFALS | CDON | | H2A3 | MP2K2 | NADK | Notch1 | | | |
| 0.637 | IGFALS | CDON | CILP2 | H2A3 | MP2K2 | | | | | |
| 0.637 | IGFALS | CDON | | | | | | PH | | |
| 0.637 | IGFALS | CDON | | H2A3 | | | Notch1 | | | S100A13 |
| 0.637 | IGFALS | | | | MP2K2 | | Notch1 | | | S100A13 |
| 0.638 | IGFALS | | | H2A3 | | | | | PTN | |
| 0.638 | IGFALS | CDON | CILP2 | | MP2K2 | NADK | | | | |
| 0.638 | IGFALS | | CILP2 | H2A3 | MP2K2 | | Notch1 | | | |
| 0.638 | IGFALS | | CILP2 | | MP2K2 | NADK | Notch1 | | | |
| 0.638 | IGFALS | | | H2A3 | | | | PH | | |
| 0.638 | IGFALS | | CILP2 | | MP2K2 | | | | | S100A13 |
| 0.638 | IGFALS | | CILP2 | H2A3 | | NADK | | | | S100A13 |
| 0.638 | IGFALS | CDON | CILP2 | | | | Notch1 | | | S100A13 |
| 0.638 | IGFALS | | | | | NADK | Notch1 | | | S100A13 |
| 0.639 | IGFALS | CDON | | | | | | | PTN | |
| 0.639 | IGFALS | | | H2A3 | MP2K2 | | | | PTN | |
| 0.639 | IGFALS | | | | | | Notch1 | PH | | |
| 0.639 | IGFALS | | CILP2 | | | NADK | | | | S100A13 |
| 0.639 | IGFALS | CDON | | | MP2K2 | NADK | | | | S100A13 |
| 0.639 | IGFALS | | CILP2 | H2A3 | | | Notch1 | | | S100A13 |
| 0.639 | IGFALS | | | H2A3 | | NADK | Notch1 | | | S100A13 |
| 0.64 | IGFALS | CDON | CILP2 | H2A3 | MP2K2 | NADK | | | | |
| 0.64 | IGFALS | CDON | CILP2 | | MP2K2 | NADK | Notch1 | | | |
| 0.64 | IGFALS | | | | | NADK | | PH | | |
| 0.64 | IGFALS | CDON | | H2A3 | MP2K2 | NADK | | | | S100A13 |
| 0.641 | IGFALS | CDON | | | MP2K2 | | | | PTN | |
| 0.641 | IGFALS | CDON | CILP2 | H2A3 | MP2K2 | | Notch1 | | | |
| 0.641 | IGFALS | | CILP2 | H2A3 | MP2K2 | NADK | Notch1 | | | |
| 0.641 | IGFALS | CDON | CILP2 | H2A3 | | NADK | | | | S100A13 |
| 0.641 | IGFALS | CDON | CILP2 | H2A3 | | | Notch1 | | | S100A13 |
| 0.641 | IGFALS | CDON | | | MP2K2 | | Notch1 | | | S100A13 |
| 0.641 | IGFALS | CDON | | | | NADK | Notch1 | | | S100A13 |
| 0.642 | IGFALS | | CILP2 | | | | | PH | | |
| 0.642 | IGFALS | | | H2A3 | | NADK | | PH | | |
| 0.642 | IGFALS | | | | | | | | PTN | S100A13 |
| 0.642 | IGFALS | CDON | CILP2 | | MP2K2 | | | | | S100A13 |
| 0.642 | IGFALS | | CILP2 | H2A3 | MP2K2 | | | | | S100A13 |
| 0.642 | IGFALS | CDON | CILP2 | | | NADK | | | | S100A13 |
| 0.642 | IGFALS | | | H2A3 | MP2K2 | | Notch1 | | | S100A13 |
| 0.643 | IGFALS | CDON | | | MP2K2 | | | PH | | |
| 0.643 | IGFALS | | | | MP2K2 | NADK | | PH | | |
| 0.643 | IGFALS | CDON | | | | | Notch1 | PH | | |
| 0.643 | IGFALS | CDON | | H2A3 | | NADK | Notch1 | | | S100A13 |
| 0.643 | IGFALS | | | H2A3 | | | | PH | | S100A13 |
| 0.644 | IGFALS | CDON | CILP2 | H2A3 | MP2K2 | NADK | Notch1 | | | |
| 0.644 | IGFALS | | | | MP2K2 | | Notch1 | PH | | |
| 0.644 | IGFALS | | | | MP2K2 | | | | PTN | S100A13 |
| 0.644 | IGFALS | | | | MP2K2 | NADK | Notch1 | | | S100A13 |
| 0.644 | IGFALS | CDON | | | | | | PH | | S100A13 |
| 0.644 | IGFALS | | | | MP2K2 | | | PH | | S100A13 |
| 0.645 | IGFALS | | | | | NADK | | | PTN | |
| 0.645 | IGFALS | | | H2A3 | | NADK | | | PTN | |
| 0.645 | IGFALS | | | | MP2K2 | NADK | | | PTN | |
| 0.645 | IGFALS | | | H2A3 | MP2K2 | NADK | | | PTN | |
| 0.645 | IGFALS | | | H2A3 | MP2K2 | | | PH | | |
| 0.645 | IGFALS | CDON | CILP2 | H2A3 | MP2K2 | | | | | S100A13 |
| 0.645 | IGFALS | | CILP2 | | MP2K2 | | Notch1 | | | S100A13 |
| 0.645 | IGFALS | CDON | | H2A3 | MP2K2 | | Notch1 | | | S100A13 |
| 0.645 | IGFALS | | | H2A3 | MP2K2 | NADK | Notch1 | | | S100A13 |
| 0.646 | IGFALS | | CILP2 | | | | Notch1 | PH | | |
| 0.646 | IGFALS | | | H2A3 | | | | | PTN | S100A13 |
| 0.646 | IGFALS | | CILP2 | | | NADK | Notch1 | | | S100A13 |
| 0.646 | IGFALS | | CILP2 | H2A3 | | NADK | Notch1 | | | S100A13 |
| 0.647 | IGFALS | CDON | | H2A3 | | | | | PTN | |
| 0.647 | IGFALS | CDON | CILP2 | | | | | PH | | |
| 0.647 | IGFALS | CDON | | H2A3 | | | | PH | | |
| 0.647 | IGFALS | CDON | | | | NADK | | PH | | |
| 0.647 | IGFALS | | | H2A3 | MP2K2 | NADK | | PH | | |
| 0.647 | IGFALS | | | | | NADK | Notch1 | PH | | |
| 0.647 | IGFALS | CDON | CILP2 | | MP2K2 | | Notch1 | | | S100A13 |
| 0.647 | IGFALS | CDON | CILP2 | | | NADK | Notch1 | | | S100A13 |
| 0.647 | IGFALS | CDON | CILP2 | H2A3 | | NADK | Notch1 | | | S100A13 |
| 0.647 | IGFALS | CDON | | | MP2K2 | NADK | Notch1 | | | S100A13 |
| 0.647 | IGFALS | | CILP2 | | | | | PH | | S100A13 |
| 0.648 | IGFALS | CDON | | H2A3 | MP2K2 | | | | PTN | |
| 0.648 | IGFALS | CDON | | | MP2K2 | | Notch1 | PH | | |
| 0.648 | IGFALS | | | H2A3 | MP2K2 | | | | PTN | S100A13 |
| 0.648 | IGFALS | | CILP2 | | MP2K2 | NADK | | | | S100A13 |
| 0.648 | IGFALS | | CILP2 | H2A3 | MP2K2 | NADK | | | | S100A13 |
| 0.649 | IGFALS | CDON | | | | NADK | | | PTN | |
| 0.649 | IGFALS | CDON | | | MP2K2 | NADK | | | PTN | |
| 0.649 | IGFALS | | | | | | | PH | PTN | |
| 0.649 | IGFALS | | CILP2 | H2A3 | | | | PH | | |
| 0.649 | IGFALS | | CILP2 | | MP2K2 | | | PH | | |
| 0.649 | IGFALS | CDON | CILP2 | | | | Notch1 | PH | | |
| 0.649 | IGFALS | | | H2A3 | | | Notch1 | PH | | |
| 0.649 | IGFALS | | | | MP2K2 | NADK | Notch1 | PH | | |
| 0.649 | IGFALS | CDON | | | | | | | PTN | S100A13 |
| 0.649 | IGFALS | CDON | | H2A3 | MP2K2 | NADK | Notch1 | | | S100A13 |
| 0.649 | IGFALS | | | | | NADK | | PH | | S100A13 |
| 0.649 | IGFALS | | | | | | Notch1 | PH | | S100A13 |
| 0.65 | IGFALS | CDON | | H2A3 | | NADK | | PH | | |
| 0.65 | IGFALS | CDON | | | MP2K2 | NADK | | PH | | |
| 0.65 | IGFALS | CDON | | | | NADK | Notch1 | PH | | |
| 0.65 | IGFALS | | CILP2 | H2A3 | MP2K2 | | Notch1 | | | S100A13 |
| 0.65 | IGFALS | | | H2A3 | | NADK | | PH | | S100A13 |
| 0.651 | IGFALS | | | | MP2K2 | | | PH | PTN | |
| 0.651 | IGFALS | | | H2A3 | | NADK | Notch1 | PH | | |
| 0.651 | IGFALS | CDON | | | MP2K2 | | | | PTN | S100A13 |
| 0.651 | IGFALS | CDON | CILP2 | | MP2K2 | NADK | | | | S100A13 |
| 0.651 | IGFALS | CDON | CILP2 | H2A3 | MP2K2 | NADK | | | | S100A13 |
| 0.651 | IGFALS | CDON | | H2A3 | | | | PH | | S100A13 |
| 0.651 | IGFALS | | | H2A3 | MP2K2 | | | PH | | S100A13 |
| 0.652 | IGFALS | CDON | | H2A3 | | NADK | | | PTN | |
| 0.652 | IGFALS | CDON | | H2A3 | MP2K2 | NADK | | | PTN | |
| 0.652 | IGFALS | | | | | | Notch1 | | PTN | |
| 0.652 | IGFALS | | | | MP2K2 | | Notch1 | | PTN | |
| 0.652 | IGFALS | | CILP2 | | | NADK | | PH | | |
| 0.652 | IGFALS | CDON | CILP2 | H2A3 | MP2K2 | | Notch1 | | | S100A13 |
| 0.652 | IGFALS | CDON | CILP2 | | | | | PH | | S100A13 |
| 0.652 | IGFALS | | CILP2 | H2A3 | | | | PH | | S100A13 |
| 0.652 | IGFALS | CDON | | | MP2K2 | | | PH | | S100A13 |
| 0.653 | IGFALS | CDON | | | | | Notch1 | | PTN | |
| 0.653 | IGFALS | | CILP2 | H2A3 | | NADK | | PH | | |
| 0.653 | IGFALS | CDON | | H2A3 | | | Notch1 | PH | | |
| 0.653 | IGFALS | | CILP2 | | MP2K2 | | Notch1 | PH | | |
| 0.653 | IGFALS | | | H2A3 | | NADK | | | PTN | S100A13 |
| 0.653 | IGFALS | | CILP2 | | MP2K2 | NADK | Notch1 | | | S100A13 |
| 0.653 | IGFALS | CDON | | | | | Notch1 | PH | | S100A13 |
| 0.654 | IGFALS | CDON | | | MP2K2 | | Notch1 | | PTN | |
| 0.654 | IGFALS | CDON | CILP2 | H2A3 | | | | PH | | |
| 0.654 | IGFALS | CDON | CILP2 | | MP2K2 | | | PH | | |
| 0.654 | IGFALS | CDON | | H2A3 | MP2K2 | | | PH | | |
| 0.654 | IGFALS | | CILP2 | H2A3 | | | Notch1 | PH | | |
| 0.654 | IGFALS | | CILP2 | | | NADK | Notch1 | PH | | |
| 0.654 | IGFALS | CDON | | | MP2K2 | NADK | Notch1 | PH | | |
| 0.654 | IGFALS | CDON | | H2A3 | | | | | PTN | S100A13 |
| 0.654 | IGFALS | | | | | NADK | | | PTN | S100A13 |
| 0.654 | IGFALS | | | | MP2K2 | NADK | | | PTN | S100A13 |
| 0.654 | IGFALS | | | H2A3 | MP2K2 | NADK | | | PTN | S100A13 |
| 0.654 | IGFALS | | CILP2 | H2A3 | MP2K2 | NADK | Notch1 | | | S100A13 |
| 0.654 | IGFALS | | CILP2 | | | | Notch1 | PH | | S100A13 |
| 0.655 | IGFALS | CDON | | H2A3 | MP2K2 | NADK | | PH | | |
| 0.655 | IGFALS | CDON | CILP2 | | MP2K2 | | Notch1 | PH | | |
| 0.655 | IGFALS | | | H2A3 | MP2K2 | | Notch1 | PH | | |
| 0.655 | IGFALS | CDON | | H2A3 | | NADK | Notch1 | PH | | |
| 0.655 | IGFALS | CDON | CILP2 | | MP2K2 | NADK | Notch1 | | | S100A13 |
| 0.655 | IGFALS | CDON | | | | NADK | | PH | | S100A13 |
| 0.655 | IGFALS | | | | MP2K2 | NADK | | PH | | S100A13 |
| 0.656 | IGFALS | CDON | CILP2 | | | NADK | | PH | | |
| 0.656 | IGFALS | | CILP2 | | MP2K2 | NADK | | PH | | |
| 0.656 | IGFALS | CDON | CILP2 | H2A3 | | | Notch1 | PH | | |
| 0.656 | IGFALS | CDON | CILP2 | | | NADK | Notch1 | PH | | |
| 0.656 | IGFALS | | | H2A3 | MP2K2 | NADK | Notch1 | PH | | |
| 0.656 | IGFALS | CDON | | H2A3 | MP2K2 | | | | PTN | S100A13 |
| 0.656 | IGFALS | CDON | CILP2 | H2A3 | MP2K2 | NADK | Notch1 | | | S100A13 |
| 0.656 | IGFALS | CDON | CILP2 | H2A3 | | | | PH | | S100A13 |
| 0.656 | IGFALS | | | H2A3 | MP2K2 | NADK | | PH | | S100A13 |
| 0.656 | IGFALS | | | H2A3 | | | Notch1 | PH | | S100A13 |
| 0.656 | IGFALS | | | | MP2K2 | | Notch1 | PH | | S100A13 |
| 0.657 | IGFALS | CDON | | | | | | PH | PTN | |
| 0.657 | IGFALS | | | H2A3 | | | | PH | PTN | |
| 0.657 | IGFALS | | CILP2 | H2A3 | MP2K2 | | | PH | | |
| 0.657 | IGFALS | CDON | CILP2 | H2A3 | | NADK | | PH | | |
| 0.657 | IGFALS | | CILP2 | H2A3 | | NADK | Notch1 | PH | | |
| 0.657 | IGFALS | | | | | | | PH | PTN | S100A13 |
| 0.657 | IGFALS | | CILP2 | | MP2K2 | | | PH | | S100A13 |
| 0.657 | IGFALS | CDON | | H2A3 | | NADK | | PH | | S100A13 |
| 0.657 | IGFALS | CDON | CILP2 | | | | Notch1 | PH | | S100A13 |
| 0.658 | IGFALS | | | H2A3 | | | Notch1 | | PTN | |
| 0.658 | IGFALS | | | H2A3 | MP2K2 | | Notch1 | | PTN | |
| 0.658 | IGFALS | CDON | | | MP2K2 | | | PH | PTN | |
| 0.658 | IGFALS | CDON | | | | NADK | | | PTN | S100A13 |
| 0.658 | IGFALS | CDON | | H2A3 | MP2K2 | | | PH | | S100A13 |
| 0.658 | IGFALS | | | | | NADK | Notch1 | PH | | S100A13 |
| 0.659 | IGFALS | | | | MP2K2 | NADK | Notch1 | | PTN | |
| 0.659 | IGFALS | | | H2A3 | MP2K2 | | | PH | PTN | |
| 0.659 | IGFALS | | | | | NADK | | PH | PTN | |
| 0.659 | IGFALS | | CILP2 | H2A3 | MP2K2 | NADK | | PH | | |
| 0.659 | IGFALS | CDON | | H2A3 | MP2K2 | | Notch1 | PH | | |
| 0.659 | IGFALS | CDON | CILP2 | H2A3 | | NADK | Notch1 | PH | | |
| 0.659 | IGFALS | | CILP2 | | MP2K2 | NADK | Notch1 | PH | | |
| 0.659 | IGFALS | CDON | | H2A3 | MP2K2 | NADK | Notch1 | PH | | |
| 0.659 | IGFALS | CDON | | H2A3 | | NADK | | | PTN | S100A13 |
| 0.659 | IGFALS | CDON | | | MP2K2 | NADK | | | PTN | S100A13 |
| 0.659 | IGFALS | | CILP2 | H2A3 | | NADK | | PH | | S100A13 |
| 0.659 | IGFALS | CDON | | | MP2K2 | NADK | | PH | | S100A13 |
| 0.659 | IGFALS | CDON | | H2A3 | | | Notch1 | PH | | S100A13 |
| 0.659 | IGFALS | CDON | | | MP2K2 | | Notch1 | PH | | S100A13 |
| 0.66 | IGFALS | CDON | | H2A3 | | | Notch1 | | PTN | |
| 0.66 | IGFALS | | | | | NADK | Notch1 | | PTN | |
| 0.66 | IGFALS | CDON | | | | NADK | Notch1 | | PTN | |
| 0.66 | IGFALS | CDON | | | MP2K2 | NADK | Notch1 | | PTN | |
| 0.66 | IGFALS | | | | MP2K2 | NADK | | PH | PTN | |
| 0.66 | IGFALS | CDON | CILP2 | | MP2K2 | NADK | | PH | | |
| 0.66 | IGFALS | CDON | | H2A3 | MP2K2 | NADK | | | PTN | S100A13 |
| 0.66 | IGFALS | | | | MP2K2 | | | PH | PTN | S100A13 |
| 0.66 | IGFALS | | CILP2 | | | NADK | | PH | | S100A13 |
| 0.66 | IGFALS | | CILP2 | H2A3 | | | Notch1 | PH | | S100A13 |
| 0.661 | IGFALS | CDON | | H2A3 | MP2K2 | | Notch1 | | PTN | |
| 0.661 | IGFALS | CDON | CILP2 | | MP2K2 | NADK | Notch1 | PH | | |
| 0.661 | IGFALS | CDON | CILP2 | | MP2K2 | | | PH | | S100A13 |
| 0.661 | IGFALS | | | H2A3 | | NADK | Notch1 | PH | | S100A13 |
| 0.662 | IGFALS | | | H2A3 | | NADK | Notch1 | | PTN | |
| 0.662 | IGFALS | | | H2A3 | MP2K2 | NADK | Notch1 | | PTN | |
| 0.662 | IGFALS | CDON | CILP2 | H2A3 | MP2K2 | | | PH | | |
| 0.662 | IGFALS | | | H2A3 | | NADK | | PH | PTN | |
| 0.662 | IGFALS | | CILP2 | H2A3 | MP2K2 | | Notch1 | PH | | |
| 0.662 | IGFALS | | CILP2 | H2A3 | MP2K2 | | | PH | | S100A13 |
| 0.662 | IGFALS | CDON | CILP2 | | | NADK | | PH | | S100A13 |
| 0.662 | IGFALS | CDON | CILP2 | H2A3 | | NADK | | PH | | S100A13 |
| 0.662 | IGFALS | CDON | | H2A3 | MP2K2 | NADK | | PH | | S100A13 |
| 0.662 | IGFALS | CDON | CILP2 | H2A3 | | | Notch1 | PH | | S100A13 |
| 0.662 | IGFALS | CDON | | | | NADK | Notch1 | PH | | S100A13 |
| 0.663 | IGFALS | | | H2A3 | MP2K2 | NADK | | PH | PTN | |
| 0.663 | IGFALS | CDON | CILP2 | H2A3 | MP2K2 | NADK | | PH | | |
| 0.663 | IGFALS | | CILP2 | H2A3 | MP2K2 | NADK | Notch1 | PH | | |
| 0.663 | IGFALS | CDON | | | | | | PH | PTN | S100A13 |
| 0.663 | IGFALS | | | H2A3 | | | | PH | PTN | S100A13 |
| 0.663 | IGFALS | | CILP2 | | MP2K2 | | Notch1 | PH | | S100A13 |
| 0.663 | IGFALS | | | H2A3 | MP2K2 | | Notch1 | PH | | S100A13 |
| 0.663 | IGFALS | | | | MP2K2 | NADK | Notch1 | PH | | S100A13 |
| 0.664 | IGFALS | CDON | CILP2 | | | | | | PTN | |
| 0.664 | IGFALS | | CILP2 | H2A3 | | | | | PTN | |
| 0.664 | IGFALS | CDON | | H2A3 | | NADK | Notch1 | | PTN | |
| 0.664 | IGFALS | CDON | | H2A3 | MP2K2 | NADK | Notch1 | | PTN | |
| 0.664 | IGFALS | CDON | CILP2 | H2A3 | MP2K2 | | Notch1 | PH | | |
| 0.664 | IGFALS | | | | | | Notch1 | | PTN | S100A13 |
| 0.664 | IGFALS | CDON | | | | | Notch1 | | PTN | S100A13 |
| 0.664 | IGFALS | | CILP2 | | | NADK | Notch1 | PH | | S100A13 |
| 0.664 | IGFALS | CDON | | H2A3 | | NADK | Notch1 | PH | | S100A13 |
| 0.665 | IGFALS | | CILP2 | | | | | | PTN | |
| 0.665 | IGFALS | CDON | | H2A3 | | | | PH | PTN | |
| 0.665 | IGFALS | CDON | | | | NADK | | PH | PTN | |
| 0.665 | IGFALS | CDON | | | MP2K2 | NADK | | PH | PTN | |
| 0.665 | IGFALS | CDON | CILP2 | H2A3 | MP2K2 | NADK | Notch1 | PH | | |
| 0.665 | IGFALS | | | | MP2K2 | | Notch1 | | PTN | S100A13 |
| 0.665 | IGFALS | CDON | | | MP2K2 | | | PH | PTN | S100A13 |
| 0.665 | IGFALS | | | H2A3 | MP2K2 | | | PH | PTN | S100A13 |
| 0.665 | IGFALS | CDON | CILP2 | H2A3 | MP2K2 | | | PH | | S100A13 |
| 0.665 | IGFALS | CDON | CILP2 | | MP2K2 | | Notch1 | PH | | S100A13 |
| 0.665 | IGFALS | | CILP2 | H2A3 | | NADK | Notch1 | PH | | S100A13 |
| 0.665 | IGFALS | CDON | | | MP2K2 | NADK | Notch1 | PH | | S100A13 |
| 0.666 | IGFALS | CDON | CILP2 | H2A3 | | | | | PTN | |
| 0.666 | IGFALS | CDON | CILP2 | | MP2K2 | | | | PTN | |
| 0.666 | IGFALS | CDON | | | MP2K2 | | Notch1 | | PTN | S100A13 |
| 0.666 | IGFALS | | CILP2 | | MP2K2 | NADK | | PH | | S100A13 |
| 0.666 | IGFALS | CDON | | H2A3 | MP2K2 | | Notch1 | PH | | S100A13 |
| 0.666 | IGFALS | CDON | CILP2 | | | NADK | Notch1 | PH | | S100A13 |
| 0.666 | IGFALS | | | H2A3 | MP2K2 | NADK | Notch1 | PH | | S100A13 |
| 0.667 | IGFALS | | CILP2 | | MP2K2 | | | | PTN | |
| 0.667 | IGFALS | | CILP2 | H2A3 | MP2K2 | | | | PTN | |
| 0.667 | IGFALS | CDON | | H2A3 | MP2K2 | | | PH | PTN | |
| 0.667 | IGFALS | | | | | | Notch1 | PH | PTN | |
| 0.667 | IGFALS | | | | MP2K2 | | Notch1 | PH | PTN | |
| 0.667 | IGFALS | | | H2A3 | | | Notch1 | | PTN | S100A13 |
| 0.667 | IGFALS | | CILP2 | H2A3 | MP2K2 | NADK | | PH | | S100A13 |
| 0.667 | IGFALS | CDON | CILP2 | H2A3 | | NADK | Notch1 | PH | | S100A13 |
| 0.668 | IGFALS | CDON | CILP2 | H2A3 | MP2K2 | | | | PTN | |
| 0.668 | IGFALS | CDON | | H2A3 | | NADK | | PH | PTN | |
| 0.668 | IGFALS | CDON | | | | | Notch1 | PH | PTN | |
| 0.668 | IGFALS | CDON | | H2A3 | | | Notch1 | | PTN | S100A13 |
| 0.668 | IGFALS | | | H2A3 | MP2K2 | | Notch1 | | PTN | S100A13 |
| 0.668 | IGFALS | | | | | NADK | | PH | PTN | S100A13 |
| 0.668 | IGFALS | CDON | CILP2 | | MP2K2 | NADK | | PH | | S100A13 |
| 0.668 | IGFALS | | CILP2 | H2A3 | MP2K2 | | Notch1 | PH | | S100A13 |
| 0.668 | IGFALS | CDON | | H2A3 | MP2K2 | NADK | Notch1 | PH | | S100A13 |
| 0.669 | IGFALS | CDON | | H2A3 | MP2K2 | NADK | | PH | PTN | |
| 0.669 | IGFALS | CDON | | | MP2K2 | | Notch1 | PH | PTN | |
| 0.669 | IGFALS | CDON | CILP2 | | | | | | PTN | S100A13 |
| 0.669 | IGFALS | | CILP2 | H2A3 | | | | | PTN | S100A13 |
| 0.669 | IGFALS | CDON | | H2A3 | | | | PH | PTN | S100A13 |
| 0.669 | IGFALS | | | H2A3 | | NADK | | PH | PTN | S100A13 |
| 0.669 | IGFALS | | | | MP2K2 | NADK | | PH | PTN | S100A13 |
| 0.669 | IGFALS | CDON | CILP2 | H2A3 | MP2K2 | NADK | | PH | | S100A13 |
| 0.669 | IGFALS | CDON | CILP2 | H2A3 | MP2K2 | | Notch1 | PH | | S100A13 |
| 0.67 | IGFALS | | CILP2 | H2A3 | | NADK | | | PTN | |
| 0.67 | IGFALS | CDON | CILP2 | | | | Notch1 | | PTN | |
| 0.67 | IGFALS | | CILP2 | | | | | | PTN | S100A13 |
| 0.67 | IGFALS | CDON | CILP2 | H2A3 | | | | | PTN | S100A13 |
| 0.67 | IGFALS | CDON | | H2A3 | MP2K2 | | Notch1 | | PTN | S100A13 |
| 0.67 | IGFALS | | | H2A3 | MP2K2 | NADK | | PH | PTN | S100A13 |
| 0.67 | IGFALS | | CILP2 | | MP2K2 | NADK | Notch1 | PH | | S100A13 |
| 0.671 | IGFALS | CDON | CILP2 | H2A3 | | NADK | | | PTN | |
| 0.671 | IGFALS | | CILP2 | H2A3 | MP2K2 | NADK | | | PTN | |
| 0.671 | IGFALS | | | | | NADK | Notch1 | | PTN | S100A13 |
| 0.671 | IGFALS | CDON | | | | NADK | Notch1 | | PTN | S100A13 |
| 0.671 | IGFALS | | | | MP2K2 | NADK | Notch1 | | PTN | S100A13 |
| 0.671 | IGFALS | CDON | | H2A3 | MP2K2 | | | PH | PTN | S100A13 |
| 0.671 | IGFALS | CDON | CILP2 | | MP2K2 | NADK | Notch1 | PH | | S100A13 |
| 0.672 | IGFALS | CDON | CILP2 | | | NADK | | | PTN | |
| 0.672 | IGFALS | CDON | CILP2 | H2A3 | MP2K2 | NADK | | | PTN | |
| 0.672 | IGFALS | | CILP2 | | | | Notch1 | | PTN | |
| 0.672 | IGFALS | CDON | CILP2 | | MP2K2 | | Notch1 | | PTN | |
| 0.672 | IGFALS | CDON | CILP2 | | MP2K2 | | | | PTN | S100A13 |
| 0.672 | IGFALS | | | H2A3 | | NADK | Notch1 | | PTN | S100A13 |
| 0.672 | IGFALS | CDON | | | MP2K2 | NADK | Notch1 | | PTN | S100A13 |
| 0.672 | IGFALS | | | H2A3 | MP2K2 | NADK | Notch1 | | PTN | S100A13 |
| 0.672 | IGFALS | CDON | | | | NADK | | PH | PTN | S100A13 |
| 0.672 | IGFALS | CDON | | | MP2K2 | NADK | | PH | PTN | S100A13 |
| 0.672 | IGFALS | | CILP2 | H2A3 | MP2K2 | NADK | Notch1 | PH | | S100A13 |
| 0.673 | IGFALS | | CILP2 | | | NADK | | | PTN | |
| 0.673 | IGFALS | CDON | CILP2 | | MP2K2 | NADK | | | PTN | |
| 0.673 | IGFALS | CDON | CILP2 | H2A3 | | | Notch1 | | PTN | |
| 0.673 | IGFALS | | | | | NADK | Notch1 | PH | PTN | |
| 0.673 | IGFALS | | | | MP2K2 | NADK | Notch1 | PH | PTN | |
| 0.673 | IGFALS | | CILP2 | | MP2K2 | | | | PTN | S100A13 |
| 0.673 | IGFALS | | CILP2 | H2A3 | MP2K2 | | | | PTN | S100A13 |
| 0.673 | IGFALS | CDON | | H2A3 | | NADK | Notch1 | | PTN | S100A13 |
| 0.673 | IGFALS | CDON | | H2A3 | MP2K2 | NADK | Notch1 | | PTN | S100A13 |
| 0.673 | IGFALS | CDON | CILP2 | H2A3 | MP2K2 | NADK | Notch1 | PH | | S100A13 |
| 0.674 | IGFALS | | CILP2 | | MP2K2 | NADK | | | PTN | |
| 0.674 | IGFALS | | CILP2 | H2A3 | | | Notch1 | | PTN | |
| 0.674 | IGFALS | | CILP2 | | MP2K2 | | Notch1 | | PTN | |
| 0.674 | IGFALS | CDON | | | | NADK | Notch1 | PH | PTN | |
| 0.674 | IGFALS | CDON | | | MP2K2 | NADK | Notch1 | PH | PTN | |
| 0.674 | IGFALS | CDON | CILP2 | H2A3 | MP2K2 | | | | PTN | S100A13 |
| 0.674 | IGFALS | CDON | | H2A3 | | NADK | | PH | PTN | S100A13 |
| 0.675 | IGFALS | | CILP2 | H2A3 | MP2K2 | | Notch1 | | PTN | |
| 0.675 | IGFALS | CDON | CILP2 | H2A3 | MP2K2 | | Notch1 | | PTN | |
| 0.675 | IGFALS | | | H2A3 | | | Notch1 | PH | PTN | |
| 0.675 | IGFALS | | CILP2 | H2A3 | | NADK | | | PTN | S100A13 |
| 0.675 | IGFALS | CDON | | H2A3 | MP2K2 | NADK | | PH | PTN | S100A13 |
| 0.675 | IGFALS | | | | | | Notch1 | PH | PTN | S100A13 |
| 0.676 | IGFALS | | CILP2 | | | | | PH | PTN | |
| 0.676 | IGFALS | CDON | CILP2 | | | | | PH | PTN | |
| 0.676 | IGFALS | | | H2A3 | MP2K2 | | Notch1 | PH | PTN | |
| 0.676 | IGFALS | CDON | CILP2 | H2A3 | | NADK | | | PTN | S100A13 |
| 0.676 | IGFALS | CDON | | | | | Notch1 | PH | PTN | S100A13 |
| 0.676 | IGFALS | | | | MP2K2 | | Notch1 | PH | PTN | S100A13 |
| 0.677 | IGFALS | CDON | CILP2 | | | NADK | Notch1 | | PTN | |
| 0.677 | IGFALS | CDON | CILP2 | H2A3 | | NADK | Notch1 | | PTN | |
| 0.677 | IGFALS | | CILP2 | H2A3 | | | | PH | PTN | |
| 0.677 | IGFALS | CDON | | H2A3 | | | Notch1 | PH | PTN | |
| 0.677 | IGFALS | CDON | | H2A3 | MP2K2 | | Notch1 | PH | PTN | |
| 0.677 | IGFALS | | | H2A3 | | NADK | Notch1 | PH | PTN | |
| 0.677 | IGFALS | | | H2A3 | MP2K2 | NADK | Notch1 | PH | PTN | |
| 0.677 | IGFALS | CDON | CILP2 | | | NADK | | | PTN | S100A13 |
| 0.677 | IGFALS | CDON | CILP2 | | | | Notch1 | | PTN | S100A13 |
| 0.677 | IGFALS | CDON | | | MP2K2 | | Notch1 | PH | PTN | S100A13 |
| 0.678 | IGFALS | | CILP2 | H2A3 | | NADK | Notch1 | | PTN | |
| 0.678 | IGFALS | CDON | CILP2 | | MP2K2 | NADK | Notch1 | | PTN | |
| 0.678 | IGFALS | CDON | CILP2 | H2A3 | MP2K2 | NADK | Notch1 | | PTN | |
| 0.678 | IGFALS | | CILP2 | | MP2K2 | | | PH | PTN | |
| 0.678 | IGFALS | CDON | | H2A3 | | NADK | Notch1 | PH | PTN | |
| 0.678 | IGFALS | | CILP2 | | | NADK | | | PTN | S100A13 |
| 0.678 | IGFALS | | CILP2 | H2A3 | MP2K2 | NADK | | | PTN | S100A13 |
| 0.678 | IGFALS | CDON | CILP2 | H2A3 | MP2K2 | NADK | | | PTN | S100A13 |
| 0.678 | IGFALS | | CILP2 | | | | Notch1 | | PTN | S100A13 |
| 0.679 | IGFALS | | CILP2 | | | NADK | Notch1 | | PTN | |
| 0.679 | IGFALS | | CILP2 | | MP2K2 | NADK | Notch1 | | PTN | |
| 0.679 | IGFALS | | CILP2 | H2A3 | MP2K2 | NADK | Notch1 | | PTN | |
| 0.679 | IGFALS | CDON | CILP2 | H2A3 | | | | PH | PTN | |
| 0.679 | IGFALS | CDON | CILP2 | | MP2K2 | | | PH | PTN | |
| 0.679 | IGFALS | CDON | | H2A3 | MP2K2 | NADK | Notch1 | PH | PTN | |
| 0.679 | IGFALS | CDON | CILP2 | | MP2K2 | NADK | | | PTN | S100A13 |
| 0.679 | IGFALS | | CILP2 | H2A3 | | | Notch1 | | PTN | S100A13 |
| 0.679 | IGFALS | CDON | CILP2 | H2A3 | | | Notch1 | | PTN | S100A13 |
| 0.679 | IGFALS | CDON | CILP2 | | MP2K2 | | Notch1 | | PTN | S100A13 |
| 0.68 | IGFALS | | CILP2 | H2A3 | MP2K2 | | | PH | PTN | |
| 0.68 | IGFALS | | CILP2 | | MP2K2 | NADK | | | PTN | S100A13 |
| 0.68 | IGFALS | | CILP2 | | MP2K2 | | Notch1 | | PTN | S100A13 |
| 0.68 | IGFALS | | CILP2 | | | | | PH | PTN | S100A13 |
| 0.68 | IGFALS | CDON | CILP2 | | | | | PH | PTN | S100A13 |
| 0.681 | IGFALS | | CILP2 | H2A3 | | NADK | | PH | PTN | |
| 0.681 | IGFALS | CDON | CILP2 | H2A3 | MP2K2 | | Notch1 | | PTN | S100A13 |
| 0.681 | IGFALS | | CILP2 | H2A3 | | | | PH | PTN | S100A13 |
| 0.681 | IGFALS | | | H2A3 | | | Notch1 | PH | PTN | S100A13 |
| 0.682 | IGFALS | CDON | CILP2 | H2A3 | MP2K2 | | | PH | PTN | |
| 0.682 | IGFALS | | CILP2 | | | NADK | | PH | PTN | |
| 0.682 | IGFALS | CDON | CILP2 | | | NADK | | PH | PTN | |
| 0.682 | IGFALS | CDON | CILP2 | H2A3 | | NADK | | PH | PTN | |
| 0.682 | IGFALS | CDON | CILP2 | | | | Notch1 | PH | PTN | |
| 0.682 | IGFALS | | CILP2 | H2A3 | MP2K2 | | Notch1 | | PTN | S100A13 |
| 0.682 | IGFALS | CDON | CILP2 | H2A3 | | | | PH | PTN | S100A13 |
| 0.682 | IGFALS | CDON | | H2A3 | | | Notch1 | PH | PTN | S100A13 |
| 0.682 | IGFALS | | | H2A3 | MP2K2 | | Notch1 | PH | PTN | S100A13 |
| 0.682 | IGFALS | | | | | NADK | Notch1 | PH | PTN | S100A13 |
| 0.682 | IGFALS | CDON | | | | NADK | Notch1 | PH | PTN | S100A13 |
| 0.682 | IGFALS | | | | MP2K2 | NADK | Notch1 | PH | PTN | S100A13 |
| 0.683 | IGFALS | CDON | CILP2 | | MP2K2 | NADK | | PH | PTN | |
| 0.683 | IGFALS | | CILP2 | H2A3 | MP2K2 | NADK | | PH | PTN | |
| 0.683 | IGFALS | | CILP2 | | | | Notch1 | PH | PTN | |
| 0.683 | IGFALS | CDON | CILP2 | | | NADK | Notch1 | | PTN | S100A13 |
| 0.683 | IGFALS | CDON | CILP2 | H2A3 | | NADK | Notch1 | | PTN | S100A13 |
| 0.683 | IGFALS | | CILP2 | | MP2K2 | | | PH | PTN | S100A13 |
| 0.683 | IGFALS | CDON | CILP2 | | MP2K2 | | | PH | PTN | S100A13 |
| 0.683 | IGFALS | CDON | | H2A3 | MP2K2 | | Notch1 | PH | PTN | S100A13 |
| 0.683 | IGFALS | CDON | | | MP2K2 | NADK | Notch1 | PH | PTN | S100A13 |
| 0.684 | IGFALS | | CILP2 | | MP2K2 | NADK | | PH | PTN | |
| 0.684 | IGFALS | CDON | CILP2 | H2A3 | MP2K2 | NADK | | PH | PTN | |
| 0.684 | IGFALS | CDON | CILP2 | | MP2K2 | | Notch1 | PH | PTN | |
| 0.684 | IGFALS | | CILP2 | H2A3 | | NADK | Notch1 | | PTN | S100A13 |
| 0.684 | IGFALS | | CILP2 | H2A3 | MP2K2 | | | PH | PTN | S100A13 |
| 0.685 | IGFALS | | CILP2 | | MP2K2 | | Notch1 | PH | PTN | |
| 0.685 | IGFALS | | CILP2 | | | NADK | Notch1 | | PTN | S100A13 |
| 0.685 | IGFALS | CDON | CILP2 | | MP2K2 | NADK | Notch1 | | PTN | S100A13 |
| 0.685 | IGFALS | CDON | CILP2 | H2A3 | MP2K2 | NADK | Notch1 | | PTN | S100A13 |
| 0.685 | IGFALS | CDON | CILP2 | H2A3 | MP2K2 | | | PH | PTN | S100A13 |
| 0.685 | IGFALS | | | H2A3 | | NADK | Notch1 | PH | PTN | S100A13 |
| 0.685 | IGFALS | CDON | | H2A3 | | NADK | Notch1 | PH | PTN | S100A13 |
| 0.685 | IGFALS | | | H2A3 | MP2K2 | NADK | Notch1 | PH | PTN | S100A13 |
| 0.685 | IGFALS | CDON | | H2A3 | MP2K2 | NADK | Notch1 | PH | PTN | S100A13 |
| 0.686 | IGFALS | | CILP2 | H2A3 | | | Notch1 | PH | PTN | |
| 0.686 | IGFALS | CDON | CILP2 | H2A3 | | | Notch1 | PH | PTN | |
| 0.686 | IGFALS | | CILP2 | H2A3 | MP2K2 | NADK | Notch1 | | PTN | S100A13 |
| 0.686 | IGFALS | | CILP2 | H2A3 | | NADK | | PH | PTN | S100A13 |
| 0.686 | IGFALS | CDON | CILP2 | H2A3 | | NADK | | PH | PTN | S100A13 |
| 0.686 | IGFALS | CDON | CILP2 | | | | Notch1 | PH | PTN | S100A13 |
| 0.687 | IGFALS | CDON | CILP2 | | | NADK | Notch1 | PH | PTN | |
| 0.687 | IGFALS | | CILP2 | | MP2K2 | NADK | Notch1 | | PTN | S100A13 |
| 0.687 | IGFALS | | CILP2 | | | NADK | | PH | PTN | S100A13 |
| 0.687 | IGFALS | CDON | CILP2 | | | NADK | | PH | PTN | S100A13 |
| 0.687 | IGFALS | | CILP2 | | | | Notch1 | PH | PTN | S100A13 |
| 0.688 | IGFALS | | CILP2 | H2A3 | MP2K2 | | Notch1 | PH | PTN | |
| 0.688 | IGFALS | CDON | CILP2 | H2A3 | MP2K2 | | Notch1 | PH | PTN | |
| 0.688 | IGFALS | | CILP2 | | | NADK | Notch1 | PH | PTN | |
| 0.688 | IGFALS | CDON | CILP2 | H2A3 | | NADK | Notch1 | PH | PTN | |
| 0.688 | IGFALS | CDON | CILP2 | | MP2K2 | NADK | Notch1 | PH | PTN | |
| 0.688 | IGFALS | | CILP2 | H2A3 | MP2K2 | NADK | | PH | PTN | S100A13 |
| 0.688 | IGFALS | CDON | CILP2 | H2A3 | MP2K2 | NADK | | PH | PTN | S100A13 |
| 0.688 | IGFALS | CDON | CILP2 | | MP2K2 | | Notch1 | PH | PTN | S100A13 |
| 0.689 | IGFALS | | CILP2 | H2A3 | | NADK | Notch1 | PH | PTN | |
| 0.689 | IGFALS | | CILP2 | | MP2K2 | NADK | Notch1 | PH | PTN | |
| 0.689 | IGFALS | CDON | CILP2 | H2A3 | MP2K2 | NADK | Notch1 | PH | PTN | |
| 0.689 | IGFALS | | CILP2 | | MP2K2 | NADK | | PH | PTN | S100A13 |
| 0.689 | IGFALS | CDON | CILP2 | | MP2K2 | NADK | | PH | PTN | S100A13 |
| 0.69 | IGFALS | | CILP2 | H2A3 | MP2K2 | NADK | Notch1 | PH | PTN | |
| 0.69 | IGFALS | CDON | CILP2 | H2A3 | | | Notch1 | PH | PTN | S100A13 |
| 0.69 | IGFALS | | CILP2 | | MP2K2 | | Notch1 | PH | PTN | S100A13 |
| 0.691 | IGFALS | | CILP2 | H2A3 | | | Notch1 | PH | PTN | S100A13 |
| 0.692 | IGFALS | CDON | CILP2 | H2A3 | MP2K2 | | Notch1 | PH | PTN | S100A13 |
| 0.692 | IGFALS | CDON | CILP2 | | | NADK | Notch1 | PH | PTN | S100A13 |
| 0.693 | IGFALS | | CILP2 | H2A3 | MP2K2 | | Notch1 | PH | PTN | S100A13 |
| 0.693 | IGFALS | CDON | CILP2 | H2A3 | | NADK | Notch1 | PH | PTN | S100A13 |
| 0.694 | IGFALS | | CILP2 | | | NADK | Notch1 | PH | PTN | S100A13 |
| 0.694 | IGFALS | | CILP2 | H2A3 | | NADK | Notch1 | PH | PTN | S100A13 |
| 0.694 | IGFALS | CDON | CILP2 | | MP2K2 | NADK | Notch1 | PH | PTN | S100A13 |
| 0.694 | IGFALS | CDON | CILP2 | H2A3 | MP2K2 | NADK | Notch1 | PH | PTN | S100A13 |
| 0.695 | IGFALS | | CILP2 | | MP2K2 | NADK | Notch1 | PH | PTN | S100A13 |
| *0.696 | IGFALS | | CILP2 | H2A3 | MP2K2 | NADK | Notch1 | PH | PTN | S100A13 |

*Included in Table 38

TABLE 48
S100A13 Biomarker Panels-20 Year Model

| AUC | S100A13 | CDON | CILP2 | H2A3 | IGFALS | MP2K2 | NADK | Notch1 | PH | PTN |
|---|---|---|---|---|---|---|---|---|---|---|
| 0.572 | S100A13 | | | H2A3 | | | | | | |
| 0.579 | S100A13 | | | | | MP2K2 | | | | |
| 0.583 | S100A13 | CDON | | | | | | | | |
| 0.587 | S100A13 | | | | | | NADK | | | |
| 0.591 | S100A13 | | CILP2 | | | | | | | |
| 0.591 | S100A13 | | | H2A3 | | | NADK | | | |
| 0.594 | S100A13 | | | H2A3 | | MP2K2 | | | | |
| 0.594 | S100A13 | | | | | | | Notch1 | | |
| 0.595 | S100A13 | CDON | | H2A3 | | | | | | |
| 0.597 | S100A13 | | | | | | | | PH | |
| 0.599 | S100A13 | | CILP2 | H2A3 | | | | | | |
| 0.6 | S100A13 | CDON | | | | MP2K2 | | | | |
| 0.601 | S100A13 | CDON | CILP2 | | | | | | | |
| 0.602 | S100A13 | | | | | MP2K2 | NADK | | | |
| 0.605 | S100A13 | CDON | | | | | NADK | | | |
| 0.605 | S100A13 | CDON | | | | | | Notch1 | | |
| 0.606 | S100A13 | | | H2A3 | | MP2K2 | NADK | | | |
| 0.606 | S100A13 | | | H2A3 | | | | Notch1 | | |
| 0.607 | S100A13 | | CILP2 | | | | | Notch1 | | |
| 0.608 | S100A13 | CDON | | H2A3 | | | NADK | | | |
| 0.608 | S100A13 | | | | | MP2K2 | | Notch1 | | |
| 0.609 | S100A13 | CDON | CILP2 | H2A3 | | | | | | |
| 0.609 | S100A13 | | | | IGFALS | | | | | |
| 0.61 | S100A13 | | | | | | | | | PTN |
| 0.612 | S100A13 | CDON | | H2A3 | | MP2K2 | | | | |
| 0.613 | S100A13 | | CILP2 | | | MP2K2 | | | | |
| 0.613 | S100A13 | CDON | CILP2 | | | | | Notch1 | | |
| 0.613 | S100A13 | | | | | | NADK | Notch1 | | |
| 0.613 | S100A13 | | | H2A3 | | | | | PH | |
| 0.614 | S100A13 | | CILP2 | | | | NADK | | | |
| 0.614 | S100A13 | | CILP2 | H2A3 | | | NADK | | | |
| 0.615 | S100A13 | | | H2A3 | IGFALS | | | | | |
| 0.615 | S100A13 | CDON | | | | MP2K2 | NADK | | | |
| 0.615 | S100A13 | CDON | | H2A3 | | | | Notch1 | | |
| 0.615 | S100A13 | | CILP2 | H2A3 | | | | Notch1 | | |
| 0.616 | S100A13 | | | | | MP2K2 | | | | PTN |
| 0.616 | S100A13 | | | | | MP2K2 | | | PH | |
| 0.617 | S100A13 | CDON | | | | MP2K2 | | Notch1 | | |
| 0.617 | S100A13 | | | H2A3 | | | NADK | Notch1 | | |
| 0.617 | S100A13 | CDON | | | | | | | PH | |
| 0.619 | S100A13 | | | H2A3 | | | | | | PTN |
| 0.62 | S100A13 | CDON | CILP2 | | | MP2K2 | | | | |
| 0.62 | S100A13 | CDON | | H2A3 | | MP2K2 | NADK | | | |
| 0.621 | S100A13 | CDON | | | IGFALS | | | | | |
| 0.621 | S100A13 | | | | IGFALS | MP2K2 | | | | |
| 0.621 | S100A13 | CDON | CILP2 | | | | NADK | | | |
| 0.621 | S100A13 | CDON | CILP2 | H2A3 | | | NADK | | | |
| 0.621 | S100A13 | CDON | CILP2 | H2A3 | | | | Notch1 | | |
| 0.621 | S100A13 | | | H2A3 | | MP2K2 | | Notch1 | | |
| 0.621 | S100A13 | CDON | | | | | NADK | Notch1 | | |
| 0.621 | S100A13 | | CILP2 | | | | | | PH | |
| 0.622 | S100A13 | | CILP2 | H2A3 | | MP2K2 | | | | |
| 0.622 | S100A13 | | | | | MP2K2 | NADK | Notch1 | | |
| 0.622 | S100A13 | | | | | | NADK | | PH | |
| 0.623 | S100A13 | | CILP2 | | | MP2K2 | | Notch1 | | |
| 0.624 | S100A13 | CDON | | | | | | | | PTN |
| 0.624 | S100A13 | CDON | | H2A3 | | | NADK | Notch1 | | |
| 0.624 | S100A13 | | | | | | | Notch1 | PH | |
| 0.625 | S100A13 | | CILP2 | | IGFALS | | | | | |
| 0.625 | S100A13 | | | H2A3 | | MP2K2 | | | | PTN |
| 0.625 | S100A13 | | CILP2 | | | | NADK | Notch1 | | |
| 0.625 | S100A13 | | | H2A3 | | | NADK | | PH | |
| 0.626 | S100A13 | CDON | | H2A3 | IGFALS | | | | | |
| 0.626 | S100A13 | | | H2A3 | IGFALS | MP2K2 | | | | |
| 0.626 | S100A13 | | | | IGFALS | | NADK | | | |
| 0.626 | S100A13 | | | H2A3 | IGFALS | | NADK | | | |
| 0.627 | S100A13 | CDON | CILP2 | | | MP2K2 | | Notch1 | | |
| 0.627 | S100A13 | CDON | | H2A3 | | MP2K2 | | Notch1 | | |
| 0.627 | S100A13 | | CILP2 | H2A3 | | | NADK | Notch1 | | |
| 0.627 | S100A13 | | | H2A3 | | MP2K2 | NADK | Notch1 | | |
| 0.628 | S100A13 | | CILP2 | H2A3 | IGFALS | | | | | |
| 0.628 | S100A13 | CDON | | | | MP2K2 | | | | PTN |
| 0.628 | S100A13 | CDON | CILP2 | H2A3 | | MP2K2 | | | | |
| 0.628 | S100A13 | | | | IGFALS | | | Notch1 | | |
| 0.628 | S100A13 | CDON | | | | MP2K2 | NADK | Notch1 | | |
| 0.629 | S100A13 | CDON | CILP2 | | | | NADK | Notch1 | | |
| 0.629 | S100A13 | CDON | | H2A3 | | | | | PH | |
| 0.629 | S100A13 | | | H2A3 | | MP2K2 | | | PH | |
| 0.63 | S100A13 | CDON | CILP2 | | IGFALS | | | | | |
| 0.63 | S100A13 | CDON | | | IGFALS | MP2K2 | | | | |
| 0.63 | S100A13 | | | | | | NADK | | | PTN |
| 0.63 | S100A13 | CDON | CILP2 | H2A3 | | | NADK | Notch1 | | |
| 0.63 | S100A13 | CDON | CILP2 | | | | | | PH | |
| 0.63 | S100A13 | | CILP2 | H2A3 | | | | | PH | |
| 0.63 | S100A13 | CDON | | | | MP2K2 | | | PH | |
| 0.631 | S100A13 | | | H2A3 | | | NADK | | | PTN |
| 0.631 | S100A13 | | CILP2 | | | MP2K2 | NADK | | | |
| 0.632 | S100A13 | | CILP2 | H2A3 | | MP2K2 | NADK | | | |
| 0.632 | S100A13 | | | | IGFALS | MP2K2 | NADK | | | |
| 0.632 | S100A13 | | | H2A3 | IGFALS | MP2K2 | NADK | | | |
| 0.632 | S100A13 | | | H2A3 | IGFALS | | | Notch1 | | |
| 0.632 | S100A13 | | CILP2 | H2A3 | | MP2K2 | | Notch1 | | |
| 0.632 | S100A13 | | | | | | | | PH | PTN |
| 0.632 | S100A13 | CDON | | | | | | Notch1 | PH | |
| 0.633 | S100A13 | CDON | | H2A3 | | | | | | PTN |
| 0.633 | S100A13 | CDON | CILP2 | H2A3 | IGFALS | | | | | |
| 0.633 | S100A13 | CDON | | | IGFALS | | NADK | | | |
| 0.633 | S100A13 | | | | | MP2K2 | NADK | | | PTN |
| 0.633 | S100A13 | CDON | | | IGFALS | | | Notch1 | | |
| 0.633 | S100A13 | CDON | | H2A3 | | MP2K2 | NADK | Notch1 | | |
| 0.633 | S100A13 | | | | | MP2K2 | NADK | | PH | |
| 0.633 | S100A13 | | CILP2 | | | | | Notch1 | PH | |
| 0.634 | S100A13 | CDON | | H2A3 | IGFALS | | NADK | | | |
| 0.634 | S100A13 | CDON | | | | | NADK | | PH | |
| 0.634 | S100A13 | | | | | MP2K2 | | Notch1 | PH | |
| 0.635 | S100A13 | CDON | | H2A3 | IGFALS | MP2K2 | | | | |
| 0.635 | S100A13 | CDON | CILP2 | | | MP2K2 | NADK | | | |
| 0.635 | S100A13 | | | H2A3 | | MP2K2 | NADK | | | PTN |
| 0.635 | S100A13 | | CILP2 | | IGFALS | | | Notch1 | | |
| 0.635 | S100A13 | | | | IGFALS | | | | PH | |
| 0.636 | S100A13 | CDON | CILP2 | H2A3 | | MP2K2 | NADK | | | |
| 0.636 | S100A13 | CDON | CILP2 | H2A3 | | MP2K2 | | Notch1 | | |
| 0.637 | S100A13 | CDON | | H2A3 | | MP2K2 | | | | PTN |
| 0.637 | S100A13 | CDON | | H2A3 | IGFALS | | | Notch1 | | |
| 0.637 | S100A13 | | | | IGFALS | MP2K2 | | Notch1 | | |
| 0.637 | S100A13 | | CILP2 | | | MP2K2 | NADK | Notch1 | | |
| 0.637 | S100A13 | CDON | CILP2 | H2A3 | | | | | PH | |
| 0.637 | S100A13 | | | | | MP2K2 | | | PH | PTN |
| 0.637 | S100A13 | CDON | | H2A3 | | | NADK | | PH | |
| 0.637 | S100A13 | | | H2A3 | | MP2K2 | NADK | | PH | |
| 0.637 | S100A13 | | | H2A3 | | | | Notch1 | PH | |
| 0.638 | S100A13 | | CILP2 | | IGFALS | MP2K2 | | | | |
| 0.638 | S100A13 | CDON | | | | | NADK | | | PTN |
| 0.638 | S100A13 | | CILP2 | H2A3 | IGFALS | | NADK | | | |
| 0.638 | S100A13 | CDON | CILP2 | | IGFALS | | | Notch1 | | |
| 0.638 | S100A13 | | | | IGFALS | | NADK | Notch1 | | |
| 0.638 | S100A13 | | CILP2 | | | MP2K2 | | | PH | |
| 0.638 | S100A13 | CDON | CILP2 | | | | | Notch1 | PH | |
| 0.639 | S100A13 | | CILP2 | | IGFALS | | NADK | | | |
| 0.639 | S100A13 | CDON | | | IGFALS | MP2K2 | NADK | | | |
| 0.639 | S100A13 | | CILP2 | H2A3 | IGFALS | | | Notch1 | | |
| 0.639 | S100A13 | | | H2A3 | IGFALS | | NADK | Notch1 | | |
| 0.64 | S100A13 | CDON | | | | MP2K2 | NADK | | | PTN |
| 0.64 | S100A13 | CDON | | H2A3 | IGFALS | MP2K2 | NADK | | | |
| 0.64 | S100A13 | CDON | CILP2 | | | MP2K2 | NADK | Notch1 | | |
| 0.64 | S100A13 | | CILP2 | H2A3 | | MP2K2 | NADK | Notch1 | | |
| 0.64 | S100A13 | CDON | | H2A3 | | MP2K2 | | | PH | |
| 0.64 | S100A13 | | | | | | NADK | Notch1 | PH | |
| 0.641 | S100A13 | CDON | CILP2 | H2A3 | IGFALS | | NADK | | | |
| 0.641 | S100A13 | CDON | CILP2 | H2A3 | IGFALS | | | Notch1 | | |
| 0.641 | S100A13 | CDON | | | IGFALS | MP2K2 | | Notch1 | | |
| 0.641 | S100A13 | CDON | | | IGFALS | | NADK | Notch1 | | |
| 0.641 | S100A13 | | CILP2 | | | | NADK | | PH | |
| 0.641 | S100A13 | | CILP2 | H2A3 | | | NADK | | PH | |
| 0.641 | S100A13 | CDON | | | | MP2K2 | | Notch1 | PH | |
| 0.642 | S100A13 | | | | IGFALS | | | | | PTN |
| 0.642 | S100A13 | CDON | CILP2 | | IGFALS | MP2K2 | | | | |
| 0.642 | S100A13 | | CILP2 | H2A3 | IGFALS | MP2K2 | | | | |
| 0.642 | S100A13 | CDON | | H2A3 | | | NADK | | | PTN |
| 0.642 | S100A13 | CDON | CILP2 | | IGFALS | | NADK | | | |
| 0.642 | S100A13 | | | | | | | Notch1 | | PTN |
| 0.642 | S100A13 | | | H2A3 | IGFALS | MP2K2 | | Notch1 | | |
| 0.642 | S100A13 | | | H2A3 | | | | | PH | PTN |
| 0.642 | S100A13 | CDON | | | | MP2K2 | NADK | | PH | |
| 0.642 | S100A13 | | CILP2 | H2A3 | | | | Notch1 | PH | |
| 0.643 | S100A13 | CDON | | H2A3 | IGFALS | | NADK | Notch1 | | |
| 0.643 | S100A13 | CDON | CILP2 | H2A3 | | MP2K2 | NADK | Notch1 | | |
| 0.643 | S100A13 | CDON | | | | | | | PH | PTN |
| 0.643 | S100A13 | | | H2A3 | IGFALS | | | | PH | |
| 0.643 | S100A13 | CDON | | H2A3 | | | | Notch1 | PH | |
| 0.644 | S100A13 | | | | IGFALS | MP2K2 | | | | PTN |
| 0.644 | S100A13 | CDON | | H2A3 | | MP2K2 | NADK | | | PTN |
| 0.644 | S100A13 | | | | | MP2K2 | | Notch1 | | PTN |
| 0.644 | S100A13 | | | | IGFALS | MP2K2 | NADK | Notch1 | | |
| 0.644 | S100A13 | CDON | | | IGFALS | | | | PH | |
| 0.644 | S100A13 | CDON | CILP2 | | | MP2K2 | | | PH | |
| 0.644 | S100A13 | | | | IGFALS | MP2K2 | | | PH | |
| 0.644 | S100A13 | | | H2A3 | | | NADK | Notch1 | PH | |
| 0.645 | S100A13 | CDON | CILP2 | H2A3 | IGFALS | MP2K2 | | | | |
| 0.645 | S100A13 | | CILP2 | | IGFALS | MP2K2 | | Notch1 | | |
| 0.645 | S100A13 | CDON | | H2A3 | IGFALS | MP2K2 | | Notch1 | | |
| 0.645 | S100A13 | | | H2A3 | IGFALS | MP2K2 | NADK | Notch1 | | |
| 0.645 | S100A13 | | CILP2 | | | MP2K2 | | Notch1 | PH | |
| 0.645 | S100A13 | CDON | | | | | NADK | Notch1 | PH | |
| 0.646 | S100A13 | | | H2A3 | IGFALS | | | | | PTN |
| 0.646 | S100A13 | CDON | | | | | | Notch1 | | PTN |
| 0.646 | S100A13 | | CILP2 | | IGFALS | | NADK | Notch1 | | |
| 0.646 | S100A13 | | CILP2 | H2A3 | IGFALS | | NADK | Notch1 | | |
| 0.646 | S100A13 | CDON | | | | MP2K2 | | | PH | PTN |
| 0.646 | S100A13 | CDON | CILP2 | | | | NADK | | PH | |
| 0.646 | S100A13 | CDON | CILP2 | H2A3 | | | NADK | | PH | |
| 0.646 | S100A13 | CDON | | H2A3 | | MP2K2 | NADK | | PH | |
| 0.646 | S100A13 | CDON | CILP2 | H2A3 | | | | Notch1 | PH | |
| 0.646 | S100A13 | | | | | MP2K2 | NADK | Notch1 | PH | |
| 0.647 | S100A13 | CDON | CILP2 | | IGFALS | MP2K2 | | Notch1 | | |
| 0.647 | S100A13 | CDON | CILP2 | | IGFALS | | NADK | Notch1 | | |
| 0.647 | S100A13 | CDON | CILP2 | H2A3 | IGFALS | | NADK | Notch1 | | |
| 0.647 | S100A13 | CDON | | | IGFALS | MP2K2 | NADK | Notch1 | | |
| 0.647 | S100A13 | | CILP2 | | IGFALS | | | | PH | |
| 0.647 | S100A13 | | CILP2 | H2A3 | | MP2K2 | | | PH | |
| 0.647 | S100A13 | | | H2A3 | | MP2K2 | | Notch1 | PH | |
| 0.648 | S100A13 | | | H2A3 | IGFALS | MP2K2 | | | | PTN |
| 0.648 | S100A13 | | CILP2 | | IGFALS | MP2K2 | NADK | | | |
| 0.648 | S100A13 | | CILP2 | H2A3 | IGFALS | MP2K2 | NADK | | | |
| 0.648 | S100A13 | CDON | | | | MP2K2 | | Notch1 | | PTN |
| 0.648 | S100A13 | | | H2A3 | | MP2K2 | | | PH | PTN |
| 0.648 | S100A13 | | | | | | NADK | | PH | PTN |
| 0.648 | S100A13 | | CILP2 | | | | NADK | Notch1 | PH | |
| 0.649 | S100A13 | CDON | | | IGFALS | | | | | PTN |
| 0.649 | S100A13 | CDON | | H2A3 | IGFALS | MP2K2 | NADK | Notch1 | | |
| 0.649 | S100A13 | | | | IGFALS | | NADK | | PH | |
| 0.649 | S100A13 | | | | IGFALS | | | Notch1 | PH | |
| 0.649 | S100A13 | CDON | CILP2 | | | MP2K2 | | Notch1 | PH | |
| 0.649 | S100A13 | CDON | | H2A3 | | | NADK | Notch1 | PH | |
| 0.65 | S100A13 | | CILP2 | H2A3 | IGFALS | MP2K2 | | Notch1 | | |
| 0.65 | S100A13 | | | H2A3 | IGFALS | | NADK | | PH | |
| 0.651 | S100A13 | | CILP2 | | | | | | | PTN |
| 0.651 | S100A13 | CDON | | | IGFALS | MP2K2 | | | | PTN |
| 0.651 | S100A13 | CDON | CILP2 | | IGFALS | MP2K2 | NADK | | | |
| 0.651 | S100A13 | CDON | CILP2 | H2A3 | IGFALS | MP2K2 | NADK | | | |
| 0.651 | S100A13 | | | H2A3 | | | | Notch1 | | PTN |
| 0.651 | S100A13 | CDON | | H2A3 | IGFALS | | | | PH | |
| 0.651 | S100A13 | CDON | CILP2 | H2A3 | | MP2K2 | | | PH | |
| 0.651 | S100A13 | | | H2A3 | IGFALS | MP2K2 | | | PH | |
| 0.651 | S100A13 | | | H2A3 | | | NADK | | PH | PTN |
| 0.651 | S100A13 | | | | | MP2K2 | NADK | | PH | PTN |
| 0.651 | S100A13 | CDON | CILP2 | | | | NADK | Notch1 | PH | |
| 0.651 | S100A13 | | CILP2 | H2A3 | | | NADK | Notch1 | PH | |
| 0.651 | S100A13 | CDON | | | | MP2K2 | NADK | Notch1 | PH | |
| 0.652 | S100A13 | CDON | CILP2 | H2A3 | IGFALS | MP2K2 | | Notch1 | | |
| 0.652 | S100A13 | CDON | | H2A3 | | | | | PH | PTN |
| 0.652 | S100A13 | CDON | CILP2 | | IGFALS | | | | PH | |
| 0.652 | S100A13 | | CILP2 | H2A3 | IGFALS | | | | PH | |
| 0.652 | S100A13 | CDON | | | IGFALS | MP2K2 | | | PH | |
| 0.652 | S100A13 | CDON | | H2A3 | | MP2K2 | | Notch1 | PH | |
| 0.652 | S100A13 | | | H2A3 | | MP2K2 | NADK | Notch1 | PH | |
| 0.653 | S100A13 | CDON | CILP2 | | | | | | | PTN |
| 0.653 | S100A13 | | CILP2 | H2A3 | | | | | | PTN |
| 0.653 | S100A13 | | | H2A3 | IGFALS | | NADK | | | PTN |
| 0.653 | S100A13 | | | H2A3 | | MP2K2 | | Notch1 | | PTN |
| 0.653 | S100A13 | | CILP2 | | IGFALS | MP2K2 | NADK | Notch1 | | |
| 0.653 | S100A13 | | CILP2 | | | MP2K2 | NADK | | PH | |
| 0.653 | S100A13 | CDON | | | IGFALS | | | Notch1 | PH | |
| 0.654 | S100A13 | CDON | | H2A3 | IGFALS | | | | | PTN |
| 0.654 | S100A13 | | | | IGFALS | | NADK | | | PTN |
| 0.654 | S100A13 | | | | IGFALS | MP2K2 | NADK | | | PTN |
| 0.654 | S100A13 | | | H2A3 | IGFALS | MP2K2 | NADK | | | PTN |
| 0.654 | S100A13 | CDON | | H2A3 | | | | Notch1 | | PTN |
| 0.654 | S100A13 | | CILP2 | H2A3 | IGFALS | MP2K2 | NADK | Notch1 | | |
| 0.654 | S100A13 | | | H2A3 | | MP2K2 | NADK | | PH | PTN |
| 0.654 | S100A13 | | CILP2 | H2A3 | | MP2K2 | NADK | | PH | |
| 0.654 | S100A13 | | CILP2 | | IGFALS | | | Notch1 | PH | |
| 0.654 | S100A13 | CDON | CILP2 | H2A3 | | | NADK | Notch1 | PH | |
| 0.655 | S100A13 | CDON | CILP2 | H2A3 | | | | | | PTN |
| 0.655 | S100A13 | | | | | | NADK | Notch1 | | PTN |
| 0.655 | S100A13 | | | | | MP2K2 | NADK | Notch1 | | PTN |
| 0.655 | S100A13 | CDON | CILP2 | | IGFALS | MP2K2 | NADK | Notch1 | | |
| 0.655 | S100A13 | CDON | | | | | NADK | | PH | PTN |
| 0.655 | S100A13 | CDON | | | IGFALS | | NADK | | PH | |
| 0.655 | S100A13 | | | | IGFALS | MP2K2 | NADK | | PH | |
| 0.655 | S100A13 | | CILP2 | H2A3 | | MP2K2 | | Notch1 | PH | |
| 0.656 | S100A13 | CDON | | H2A3 | IGFALS | MP2K2 | | | | PTN |
| 0.656 | S100A13 | CDON | | H2A3 | | MP2K2 | | Notch1 | | PTN |
| 0.656 | S100A13 | CDON | CILP2 | H2A3 | IGFALS | MP2K2 | NADK | Notch1 | | |
| 0.656 | S100A13 | CDON | CILP2 | H2A3 | IGFALS | | | | PH | |
| 0.656 | S100A13 | CDON | | H2A3 | | MP2K2 | | | PH | PTN |
| 0.656 | S100A13 | CDON | CILP2 | | | MP2K2 | NADK | | PH | |
| 0.656 | S100A13 | | | H2A3 | IGFALS | MP2K2 | NADK | | PH | |
| 0.656 | S100A13 | | | H2A3 | IGFALS | | | Notch1 | PH | |
| 0.656 | S100A13 | | | | IGFALS | MP2K2 | | Notch1 | PH | |
| 0.656 | S100A13 | CDON | | H2A3 | | MP2K2 | NADK | Notch1 | PH | |
| 0.657 | S100A13 | | CILP2 | | | MP2K2 | | | | PTN |
| 0.657 | S100A13 | CDON | | | | | NADK | Notch1 | | PTN |
| 0.657 | S100A13 | | | H2A3 | | | NADK | Notch1 | | PTN |
| 0.657 | S100A13 | | | | IGFALS | | | | PH | PTN |
| 0.657 | S100A13 | | CILP2 | | IGFALS | MP2K2 | | | PH | |
| 0.657 | S100A13 | CDON | | H2A3 | IGFALS | | NADK | | PH | |
| 0.657 | S100A13 | CDON | | | | MP2K2 | NADK | | PH | PTN |
| 0.657 | S100A13 | CDON | CILP2 | H2A3 | | MP2K2 | NADK | | PH | |
| 0.657 | S100A13 | CDON | CILP2 | | IGFALS | | | Notch1 | PH | |
| 0.657 | S100A13 | CDON | CILP2 | H2A3 | | MP2K2 | | Notch1 | PH | |
| 0.657 | S100A13 | | CILP2 | | | MP2K2 | NADK | Notch1 | PH | |
| 0.658 | S100A13 | CDON | CILP2 | | | MP2K2 | | | | PTN |
| 0.658 | S100A13 | CDON | | | IGFALS | | NADK | | | PTN |
| 0.658 | S100A13 | CDON | | | | MP2K2 | NADK | Notch1 | | PTN |
| 0.658 | S100A13 | | | H2A3 | | MP2K2 | NADK | Notch1 | | PTN |
| 0.658 | S100A13 | CDON | | H2A3 | IGFALS | MP2K2 | | | PH | |
| 0.658 | S100A13 | | | | | | | Notch1 | PH | PTN |
| 0.658 | S100A13 | | | | IGFALS | | NADK | Notch1 | PH | |
| 0.659 | S100A13 | | CILP2 | H2A3 | | MP2K2 | | | | PTN |
| 0.659 | S100A13 | CDON | | H2A3 | IGFALS | | NADK | | | PTN |
| 0.659 | S100A13 | CDON | | | IGFALS | MP2K2 | NADK | | | PTN |
| 0.659 | S100A13 | CDON | | H2A3 | | | NADK | | PH | PTN |
| 0.659 | S100A13 | | CILP2 | H2A3 | IGFALS | | NADK | | PH | |
| 0.659 | S100A13 | CDON | | | IGFALS | MP2K2 | NADK | | PH | |
| 0.659 | S100A13 | CDON | | H2A3 | IGFALS | | | Notch1 | PH | |
| 0.659 | S100A13 | | | | | MP2K2 | | Notch1 | PH | PTN |
| 0.659 | S100A13 | CDON | | | IGFALS | MP2K2 | | Notch1 | PH | |
| 0.659 | S100A13 | CDON | CILP2 | | | MP2K2 | NADK | Notch1 | PH | |
| 0.66 | S100A13 | CDON | | H2A3 | IGFALS | MP2K2 | NADK | | | PTN |
| 0.66 | S100A13 | CDON | | H2A3 | | | NADK | Notch1 | | PTN |
| 0.66 | S100A13 | | | | IGFALS | MP2K2 | | | PH | PTN |
| 0.66 | S100A13 | | CILP2 | | IGFALS | | NADK | | PH | |
| 0.66 | S100A13 | | CILP2 | H2A3 | IGFALS | | | Notch1 | PH | |
| 0.661 | S100A13 | CDON | CILP2 | H2A3 | | MP2K2 | | | | PTN |
| 0.661 | S100A13 | CDON | | H2A3 | | MP2K2 | NADK | Notch1 | | PTN |
| 0.661 | S100A13 | CDON | CILP2 | | IGFALS | MP2K2 | | | PH | |
| 0.661 | S100A13 | CDON | | H2A3 | | MP2K2 | NADK | | PH | PTN |
| 0.661 | S100A13 | CDON | | | | | | Notch1 | PH | PTN |
| 0.661 | S100A13 | | | H2A3 | IGFALS | | NADK | Notch1 | PH | |
| 0.661 | S100A13 | | CILP2 | H2A3 | | MP2K2 | NADK | Notch1 | PH | |
| 0.662 | S100A13 | | CILP2 | H2A3 | | | NADK | | | PTN |
| 0.662 | S100A13 | CDON | CILP2 | | | | | Notch1 | | PTN |
| 0.662 | S100A13 | | CILP2 | H2A3 | IGFALS | MP2K2 | | | PH | |
| 0.662 | S100A13 | CDON | CILP2 | | IGFALS | | NADK | | PH | |
| 0.662 | S100A13 | CDON | CILP2 | H2A3 | IGFALS | | NADK | | PH | |
| 0.662 | S100A13 | CDON | | H2A3 | IGFALS | MP2K2 | NADK | | PH | |
| 0.662 | S100A13 | CDON | CILP2 | H2A3 | IGFALS | | | Notch1 | PH | |
| 0.662 | S100A13 | CDON | | | | MP2K2 | | Notch1 | PH | PTN |
| 0.662 | S100A13 | CDON | | | IGFALS | | NADK | Notch1 | PH | |
| 0.662 | S100A13 | CDON | CILP2 | H2A3 | | MP2K2 | NADK | Notch1 | PH | |
| 0.663 | S100A13 | CDON | CILP2 | H2A3 | | | NADK | | | PTN |
| 0.663 | S100A13 | | CILP2 | | | | | Notch1 | | PTN |
| 0.663 | S100A13 | CDON | | | IGFALS | | | | PH | PTN |
| 0.663 | S100A13 | | | H2A3 | IGFALS | | | | PH | PTN |
| 0.663 | S100A13 | | CILP2 | | IGFALS | MP2K2 | | Notch1 | PH | |
| 0.663 | S100A13 | | | H2A3 | IGFALS | MP2K2 | | Notch1 | PH | |
| 0.663 | S100A13 | | | | IGFALS | MP2K2 | NADK | Notch1 | PH | |
| 0.664 | S100A13 | | CILP2 | | | | NADK | | | PTN |
| 0.664 | S100A13 | | | | IGFALS | | | Notch1 | | PTN |
| 0.664 | S100A13 | CDON | | | IGFALS | | | Notch1 | | PTN |
| 0.664 | S100A13 | | CILP2 | | IGFALS | | NADK | Notch1 | PH | |
| 0.664 | S100A13 | CDON | | H2A3 | IGFALS | | NADK | Notch1 | PH | |
| 0.665 | S100A13 | CDON | CILP2 | | | | NADK | | | PTN |
| 0.665 | S100A13 | | | | IGFALS | MP2K2 | | Notch1 | | PTN |
| 0.665 | S100A13 | | CILP2 | | | | | | PH | PTN |
| 0.665 | S100A13 | CDON | | | IGFALS | MP2K2 | | | PH | PTN |
| 0.665 | S100A13 | | | H2A3 | IGFALS | MP2K2 | | | PH | PTN |
| 0.665 | S100A13 | CDON | CILP2 | H2A3 | IGFALS | MP2K2 | | | PH | |
| 0.665 | S100A13 | CDON | CILP2 | | IGFALS | MP2K2 | | Notch1 | PH | |
| 0.665 | S100A13 | | CILP2 | H2A3 | IGFALS | | NADK | Notch1 | PH | |
| 0.665 | S100A13 | CDON | | | IGFALS | MP2K2 | NADK | Notch1 | PH | |
| 0.666 | S100A13 | | CILP2 | H2A3 | | MP2K2 | NADK | | | PTN |
| 0.666 | S100A13 | CDON | CILP2 | | | MP2K2 | | Notch1 | | PTN |
| 0.666 | S100A13 | CDON | | | IGFALS | MP2K2 | | Notch1 | | PTN |
| 0.666 | S100A13 | CDON | CILP2 | | | | | | PH | PTN |
| 0.666 | S100A13 | | CILP2 | | IGFALS | MP2K2 | NADK | | PH | |
| 0.666 | S100A13 | CDON | | H2A3 | IGFALS | MP2K2 | | Notch1 | PH | |
| 0.666 | S100A13 | CDON | CILP2 | | IGFALS | | NADK | Notch1 | PH | |
| 0.666 | S100A13 | | | H2A3 | IGFALS | MP2K2 | NADK | Notch1 | PH | |
| 0.667 | S100A13 | | CILP2 | H2A3 | | | | Notch1 | | PTN |
| 0.667 | S100A13 | CDON | CILP2 | H2A3 | | | | Notch1 | | PTN |
| 0.667 | S100A13 | | | H2A3 | IGFALS | | | Notch1 | | PTN |
| 0.667 | S100A13 | | CILP2 | | | MP2K2 | | Notch1 | | PTN |
| 0.667 | S100A13 | | CILP2 | H2A3 | IGFALS | MP2K2 | NADK | | PH | |
| 0.667 | S100A13 | CDON | CILP2 | H2A3 | IGFALS | | NADK | Notch1 | PH | |
| 0.668 | S100A13 | | CILP2 | | | MP2K2 | NADK | | | PTN |
| 0.668 | S100A13 | CDON | CILP2 | | | MP2K2 | NADK | | | PTN |
| 0.668 | S100A13 | CDON | CILP2 | H2A3 | | MP2K2 | NADK | | | PTN |
| 0.668 | S100A13 | CDON | | H2A3 | IGFALS | | | Notch1 | | PTN |
| 0.668 | S100A13 | | | H2A3 | IGFALS | MP2K2 | | Notch1 | | PTN |
| 0.668 | S100A13 | | CILP2 | H2A3 | | | | | PH | PTN |
| 0.668 | S100A13 | | | | IGFALS | | NADK | | PH | PTN |
| 0.668 | S100A13 | CDON | CILP2 | | IGFALS | MP2K2 | NADK | | PH | |
| 0.668 | S100A13 | | | H2A3 | | | | Notch1 | PH | PTN |
| 0.668 | S100A13 | | CILP2 | H2A3 | IGFALS | MP2K2 | | Notch1 | PH | |
| 0.668 | S100A13 | | | | | | NADK | Notch1 | PH | PTN |
| 0.668 | S100A13 | CDON | | H2A3 | IGFALS | MP2K2 | NADK | Notch1 | PH | |
| 0.669 | S100A13 | CDON | CILP2 | | IGFALS | | | | | PTN |
| 0.669 | S100A13 | | CILP2 | H2A3 | IGFALS | | | | | PTN |
| 0.669 | S100A13 | CDON | | H2A3 | IGFALS | | | | PH | PTN |
| 0.669 | S100A13 | | | H2A3 | IGFALS | | NADK | | PH | PTN |
| 0.669 | S100A13 | | | | IGFALS | MP2K2 | NADK | | PH | PTN |
| 0.669 | S100A13 | CDON | CILP2 | H2A3 | IGFALS | MP2K2 | NADK | | PH | |
| 0.669 | S100A13 | CDON | CILP2 | H2A3 | IGFALS | MP2K2 | | Notch1 | PH | |
| 0.669 | S100A13 | | | | | MP2K2 | NADK | Notch1 | PH | PTN |
| 0.67 | S100A13 | | CILP2 | | IGFALS | | | | | PTN |
| 0.67 | S100A13 | CDON | CILP2 | H2A3 | IGFALS | | | | | PTN |
| 0.67 | S100A13 | CDON | | H2A3 | IGFALS | MP2K2 | | Notch1 | | PTN |
| 0.67 | S100A13 | CDON | CILP2 | H2A3 | | | | | PH | PTN |
| 0.67 | S100A13 | | CILP2 | | | MP2K2 | | | PH | PTN |
| 0.67 | S100A13 | | | H2A3 | IGFALS | MP2K2 | NADK | | PH | PTN |
| 0.67 | S100A13 | CDON | | H2A3 | | | | Notch1 | PH | PTN |
| 0.67 | S100A13 | | | H2A3 | | MP2K2 | | Notch1 | PH | PTN |
| 0.67 | S100A13 | CDON | | | | | NADK | Notch1 | PH | PTN |
| 0.67 | S100A13 | CDON | | | | MP2K2 | NADK | Notch1 | PH | PTN |
| 0.67 | S100A13 | | CILP2 | | IGFALS | MP2K2 | NADK | Notch1 | PH | |
| 0.671 | S100A13 | | CILP2 | H2A3 | | MP2K2 | | Notch1 | | PTN |
| 0.671 | S100A13 | CDON | CILP2 | H2A3 | | MP2K2 | | Notch1 | | PTN |
| 0.671 | S100A13 | | | | IGFALS | | NADK | Notch1 | | PTN |
| 0.671 | S100A13 | CDON | | | IGFALS | | NADK | Notch1 | | PTN |
| 0.671 | S100A13 | | | | IGFALS | MP2K2 | NADK | Notch1 | | PTN |
| 0.671 | S100A13 | CDON | CILP2 | | | MP2K2 | | | PH | PTN |
| 0.671 | S100A13 | CDON | | H2A3 | IGFALS | MP2K2 | | | PH | PTN |
| 0.671 | S100A13 | CDON | CILP2 | | IGFALS | MP2K2 | NADK | Notch1 | PH | |
| 0.672 | S100A13 | CDON | CILP2 | | IGFALS | MP2K2 | | | | PTN |
| 0.672 | S100A13 | | | H2A3 | IGFALS | | NADK | Notch1 | | PTN |
| 0.672 | S100A13 | CDON | | | IGFALS | MP2K2 | NADK | Notch1 | | PTN |
| 0.672 | S100A13 | | | H2A3 | IGFALS | MP2K2 | NADK | Notch1 | | PTN |
| 0.672 | S100A13 | CDON | | | IGFALS | | NADK | | PH | PTN |
| 0.672 | S100A13 | CDON | | | IGFALS | MP2K2 | NADK | | PH | PTN |
| 0.672 | S100A13 | CDON | | H2A3 | | MP2K2 | | Notch1 | PH | PTN |
| 0.672 | S100A13 | | | H2A3 | | | NADK | Notch1 | PH | PTN |
| 0.672 | S100A13 | | CILP2 | H2A3 | IGFALS | MP2K2 | NADK | Notch1 | PH | |
| 0.673 | S100A13 | | CILP2 | | IGFALS | MP2K2 | | | | PTN |
| 0.673 | S100A13 | | CILP2 | H2A3 | IGFALS | MP2K2 | | | | PTN |
| 0.673 | S100A13 | CDON | CILP2 | | | | NADK | Notch1 | | PTN |
| 0.673 | S100A13 | CDON | CILP2 | H2A3 | | | NADK | Notch1 | | PTN |
| 0.673 | S100A13 | CDON | | H2A3 | IGFALS | | NADK | Notch1 | | PTN |
| 0.673 | S100A13 | CDON | | H2A3 | IGFALS | MP2K2 | NADK | Notch1 | | PTN |
| 0.673 | S100A13 | | CILP2 | H2A3 | | MP2K2 | | | PH | PTN |
| 0.673 | S100A13 | | | H2A3 | | MP2K2 | NADK | Notch1 | PH | PTN |
| 0.673 | S100A13 | CDON | CILP2 | H2A3 | IGFALS | MP2K2 | NADK | Notch1 | PH | |
| 0.674 | S100A13 | CDON | CILP2 | H2A3 | IGFALS | MP2K2 | | | | PTN |
| 0.674 | S100A13 | | CILP2 | | | | NADK | Notch1 | | PTN |
| 0.674 | S100A13 | | CILP2 | H2A3 | | | NADK | Notch1 | | PTN |
| 0.674 | S100A13 | | CILP2 | H2A3 | | | NADK | | PH | PTN |
| 0.674 | S100A13 | CDON | | H2A3 | IGFALS | | NADK | | PH | PTN |
| 0.674 | S100A13 | | CILP2 | | | | | Notch1 | PH | PTN |
| 0.674 | S100A13 | CDON | CILP2 | | | | | Notch1 | PH | PTN |
| 0.674 | S100A13 | CDON | | H2A3 | | | NADK | Notch1 | PH | PTN |
| 0.675 | S100A13 | | CILP2 | H2A3 | IGFALS | | NADK | | | PTN |
| 0.675 | S100A13 | CDON | CILP2 | H2A3 | | MP2K2 | | | PH | PTN |
| 0.675 | S100A13 | CDON | | H2A3 | IGFALS | MP2K2 | NADK | | PH | PTN |
| 0.675 | S100A13 | | | | IGFALS | | | Notch1 | PH | PTN |
| 0.675 | S100A13 | CDON | | H2A3 | | MP2K2 | NADK | Notch1 | PH | PTN |
| 0.676 | S100A13 | CDON | CILP2 | H2A3 | IGFALS | | NADK | | | PTN |
| 0.676 | S100A13 | | CILP2 | | | MP2K2 | NADK | Notch1 | | PTN |
| 0.676 | S100A13 | CDON | CILP2 | | | MP2K2 | NADK | Notch1 | | PTN |
| 0.676 | S100A13 | CDON | CILP2 | H2A3 | | MP2K2 | NADK | Notch1 | | PTN |
| 0.676 | S100A13 | | CILP2 | | | | NADK | | PH | PTN |
| 0.676 | S100A13 | CDON | CILP2 | | | | NADK | | PH | PTN |
| 0.676 | S100A13 | CDON | CILP2 | H2A3 | | | NADK | | PH | PTN |
| 0.676 | S100A13 | CDON | | | IGFALS | | | Notch1 | PH | PTN |
| 0.676 | S100A13 | | | | IGFALS | MP2K2 | | Notch1 | PH | PTN |
| 0.677 | S100A13 | CDON | CILP2 | | IGFALS | | NADK | | | PTN |
| 0.677 | S100A13 | CDON | CILP2 | | IGFALS | | | Notch1 | | PTN |
| 0.677 | S100A13 | | CILP2 | H2A3 | | MP2K2 | NADK | Notch1 | | PTN |
| 0.677 | S100A13 | CDON | CILP2 | | | MP2K2 | | Notch1 | PH | PTN |
| 0.677 | S100A13 | CDON | | | IGFALS | MP2K2 | | Notch1 | PH | PTN |
| 0.678 | S100A13 | | CILP2 | | IGFALS | | NADK | | | PTN |
| 0.678 | S100A13 | | CILP2 | H2A3 | IGFALS | MP2K2 | NADK | | | PTN |
| 0.678 | S100A13 | CDON | CILP2 | H2A3 | IGFALS | MP2K2 | NADK | | | PTN |
| 0.678 | S100A13 | | CILP2 | | IGFALS | | | Notch1 | | PTN |
| 0.678 | S100A13 | | CILP2 | | | MP2K2 | | Notch1 | PH | PTN |
| 0.679 | S100A13 | CDON | CILP2 | | IGFALS | MP2K2 | NADK | | | PTN |
| 0.679 | S100A13 | | CILP2 | H2A3 | IGFALS | | | Notch1 | | PTN |
| 0.679 | S100A13 | CDON | CILP2 | H2A3 | IGFALS | | | Notch1 | | PTN |
| 0.679 | S100A13 | CDON | CILP2 | | IGFALS | MP2K2 | | Notch1 | | PTN |
| 0.679 | S100A13 | CDON | CILP2 | | | MP2K2 | NADK | | PH | PTN |
| 0.679 | S100A13 | | CILP2 | H2A3 | | MP2K2 | NADK | | PH | PTN |
| 0.679 | S100A13 | CDON | CILP2 | H2A3 | | MP2K2 | NADK | | PH | PTN |
| 0.68 | S100A13 | | CILP2 | | IGFALS | MP2K2 | NADK | | | PTN |
| 0.68 | S100A13 | | CILP2 | | IGFALS | MP2K2 | | Notch1 | | PTN |
| 0.68 | S100A13 | | CILP2 | | IGFALS | | | | PH | PTN |
| 0.68 | S100A13 | CDON | CILP2 | | IGFALS | | | | PH | PTN |
| 0.68 | S100A13 | | CILP2 | | | MP2K2 | NADK | | PH | PTN |
| 0.68 | S100A13 | | CILP2 | H2A3 | | | | Notch1 | PH | PTN |
| 0.68 | S100A13 | CDON | CILP2 | H2A3 | | | | Notch1 | PH | PTN |
| 0.681 | S100A13 | CDON | CILP2 | H2A3 | IGFALS | MP2K2 | | Notch1 | | PTN |
| 0.681 | S100A13 | | CILP2 | H2A3 | IGFALS | | | | PH | PTN |
| 0.681 | S100A13 | | | H2A3 | IGFALS | | | Notch1 | PH | PTN |
| 0.682 | S100A13 | | CILP2 | H2A3 | IGFALS | MP2K2 | | Notch1 | | PTN |
| 0.682 | S100A13 | CDON | CILP2 | H2A3 | IGFALS | | | | PH | PTN |
| 0.682 | S100A13 | CDON | | H2A3 | IGFALS | | | Notch1 | PH | PTN |
| 0.682 | S100A13 | | | H2A3 | IGFALS | MP2K2 | | Notch1 | PH | PTN |
| 0.682 | S100A13 | | | | IGFALS | | NADK | Notch1 | PH | PTN |
| 0.682 | S100A13 | CDON | | | IGFALS | | NADK | Notch1 | PH | PTN |
| 0.682 | S100A13 | | | | IGFALS | MP2K2 | NADK | Notch1 | PH | PTN |
| 0.683 | S100A13 | CDON | CILP2 | | IGFALS | | NADK | Notch1 | | PTN |
| 0.683 | S100A13 | CDON | CILP2 | H2A3 | IGFALS | | NADK | Notch1 | | PTN |
| 0.683 | S100A13 | | CILP2 | | IGFALS | MP2K2 | | | PH | PTN |
| 0.683 | S100A13 | CDON | CILP2 | | IGFALS | MP2K2 | | | PH | PTN |
| 0.683 | S100A13 | CDON | CILP2 | H2A3 | | MP2K2 | | Notch1 | PH | PTN |
| 0.683 | S100A13 | CDON | | H2A3 | IGFALS | MP2K2 | | Notch1 | PH | PTN |
| 0.683 | S100A13 | CDON | CILP2 | | | | NADK | Notch1 | PH | PTN |
| 0.683 | S100A13 | CDON | | | IGFALS | MP2K2 | NADK | Notch1 | PH | PTN |
| 0.684 | S100A13 | | CILP2 | H2A3 | IGFALS | | NADK | Notch1 | | PTN |
| 0.684 | S100A13 | | CILP2 | H2A3 | IGFALS | MP2K2 | | | PH | PTN |
| 0.684 | S100A13 | | CILP2 | H2A3 | | MP2K2 | | Notch1 | PH | PTN |
| 0.684 | S100A13 | | CILP2 | | | | NADK | Notch1 | PH | PTN |
| 0.685 | S100A13 | | CILP2 | | IGFALS | | NADK | Notch1 | | PTN |
| 0.685 | S100A13 | CDON | CILP2 | | IGFALS | MP2K2 | NADK | Notch1 | | PTN |
| 0.685 | S100A13 | CDON | CILP2 | H2A3 | IGFALS | MP2K2 | NADK | Notch1 | | PTN |
| 0.685 | S100A13 | CDON | CILP2 | H2A3 | IGFALS | MP2K2 | | | PH | PTN |
| 0.685 | S100A13 | | CILP2 | H2A3 | | | NADK | Notch1 | PH | PTN |
| 0.685 | S100A13 | CDON | CILP2 | H2A3 | | | NADK | Notch1 | PH | PTN |
| 0.685 | S100A13 | | | H2A3 | IGFALS | | NADK | Notch1 | PH | PTN |
| 0.685 | S100A13 | CDON | | H2A3 | IGFALS | | NADK | Notch1 | PH | PTN |
| 0.685 | S100A13 | CDON | CILP2 | | | MP2K2 | NADK | Notch1 | PH | PTN |
| 0.685 | S100A13 | | | H2A3 | IGFALS | MP2K2 | NADK | Notch1 | PH | PTN |
| 0.685 | S100A13 | CDON | | H2A3 | IGFALS | MP2K2 | NADK | Notch1 | PH | PTN |
| 0.686 | S100A13 | | CILP2 | H2A3 | IGFALS | MP2K2 | NADK | Notch1 | | PTN |
| 0.686 | S100A13 | | CILP2 | H2A3 | IGFALS | | NADK | | PH | PTN |
| 0.686 | S100A13 | CDON | CILP2 | H2A3 | IGFALS | | NADK | | PH | PTN |
| 0.686 | S100A13 | CDON | CILP2 | | IGFALS | | | Notch1 | PH | PTN |
| 0.686 | S100A13 | | CILP2 | | | MP2K2 | NADK | Notch1 | PH | PTN |
| 0.687 | S100A13 | | CILP2 | | IGFALS | MP2K2 | NADK | Notch1 | | PTN |
| 0.687 | S100A13 | | CILP2 | | IGFALS | | NADK | | PH | PTN |
| 0.687 | S100A13 | CDON | CILP2 | | IGFALS | | NADK | | PH | PTN |
| 0.687 | S100A13 | | CILP2 | | IGFALS | | | Notch1 | PH | PTN |
| 0.687 | S100A13 | CDON | CILP2 | H2A3 | | MP2K2 | NADK | Notch1 | PH | PTN |
| 0.688 | S100A13 | | CILP2 | H2A3 | IGFALS | MP2K2 | NADK | | PH | PTN |
| 0.688 | S100A13 | CDON | CILP2 | H2A3 | IGFALS | MP2K2 | NADK | | PH | PTN |
| 0.688 | S100A13 | CDON | CILP2 | | IGFALS | MP2K2 | | Notch1 | PH | PTN |
| 0.688 | S100A13 | | CILP2 | H2A3 | | MP2K2 | NADK | Notch1 | PH | PTN |
| 0.689 | S100A13 | | CILP2 | | IGFALS | MP2K2 | NADK | | PH | PTN |
| 0.689 | S100A13 | CDON | CILP2 | | IGFALS | MP2K2 | NADK | | PH | PTN |
| 0.69 | S100A13 | CDON | CILP2 | H2A3 | IGFALS | | | Notch1 | PH | PTN |
| 0.69 | S100A13 | | CILP2 | | IGFALS | MP2K2 | | Notch1 | PH | PTN |
| 0.691 | S100A13 | | CILP2 | H2A3 | IGFALS | | | Notch1 | PH | PTN |
| 0.692 | S100A13 | CDON | CILP2 | H2A3 | IGFALS | MP2K2 | | Notch1 | PH | PTN |
| 0.692 | S100A13 | CDON | CILP2 | | IGFALS | | NADK | Notch1 | PH | PTN |
| 0.693 | S100A13 | | CILP2 | H2A3 | IGFALS | MP2K2 | | Notch1 | PH | PTN |
| 0.693 | S100A13 | CDON | CILP2 | H2A3 | IGFALS | | NADK | Notch1 | PH | PTN |
| 0.694 | S100A13 | | CILP2 | | IGFALS | | NADK | Notch1 | PH | PTN |
| 0.694 | S100A13 | | CILP2 | H2A3 | IGFALS | | NADK | Notch1 | PH | PTN |
| 0.694 | S100A13 | CDON | CILP2 | | IGFALS | MP2K2 | NADK | Notch1 | PH | PTN |
| 0.694 | S100A13 | CDON | CILP2 | H2A3 | IGFALS | MP2K2 | NADK | Notch1 | PH | PTN |
| 0.695 | S100A13 | | CILP2 | | IGFALS | MP2K2 | NADK | Notch1 | PH | PTN |
| *0.696 | S100A13 | | CILP2 | H2A3 | IGFALS | MP2K2 | NADK | Notch1 | PH | PTN |

*Included in Table 38

TABLE 49
Univariate performance for each
marker from Table 6 alone-20 year

| Target | AUC |
|---|---|
| CDCP1 | 0.626 |
| PTN | 0.598 |
| TIM-1 | 0.595 |
| NDST1 | 0.594 |
| IGFALS | 0.592 |
| PH | 0.589 |
| ASB9 | 0.588 |
| OPG | 0.586 |
| SVEP1 | 0.576 |
| YKL-40 | 0.574 |
| ATS13 | 0.574 |
| Notch 1 | 0.572 |
| NADK | 0.572 |
| CILP2 | 0.572 |
| NPTXR | 0.567 |
| PARC | 0.567 |
| CDON | 0.566 |
| MP2K2 | 0.565 |
| IL-18 | 0.561 |
| calgranulin B | 0.561 |
| sRAGE | 0.56 |
| TBCA | 0.556 |
| H2A3 | 0.553 |
| S100A13 | 0.551 |
| NFL | 0.541 |

TABLE 50
Performance for combinations of markers from Table 6-5 year model

| AUC | NADK | H2A3 | PTN | MP2K2 | CDON | PH | Notch1 | IGFALS | S100A13 | CILP2 |
|---|---|---|---|---|---|---|---|---|---|---|
| 0.575852 | NADK | H2A3 | | | | | | | | |
| 0.626623 | NADK | | PTN | | | | | | | |
| 0.594948 | NADK | | | MP2K2 | | | | | | |
| 0.61957 | NADK | | | | CDON | | | | | |
| 0.597273 | NADK | | | | | PH | | | | |
| 0.57669 | NADK | | | | | | Notch1 | | | |
| 0.583553 | NADK | | | | | | | IGFALS | | |
| 0.57057 | NADK | | | | | | | | S100A13 | |
| 0.591329 | NADK | | | | | | | | | CILP2 |
| 0.629241 | | H2A3 | PTN | | | | | | | |
| 0.599524 | | H2A3 | | MP2K2 | | | | | | |
| 0.622765 | | H2A3 | | | CDON | | | | | |
| 0.59674 | | H2A3 | | | | PH | | | | |
| 0.575768 | | H2A3 | | | | | Notch1 | | | |
| 0.58466 | | H2A3 | | | | | | IGFALS | | |
| 0.564721 | | H2A3 | | | | | | | S100A13 | |
| 0.588647 | | H2A3 | | | | | | | | CILP2 |
| 0.623954 | | | PTN | MP2K2 | | | | | | |
| 0.652178 | | | PTN | | CDON | | | | | |
| 0.624166 | | | PTN | | | PH | | | | |
| 0.633046 | | | PTN | | | | Notch1 | | | |
| 0.623635 | | | PTN | | | | | IGFALS | | |
| 0.621062 | | | PTN | | | | | | S100A13 | |
| 0.655352 | | | PTN | | | | | | | CILP2 |
| 0.628802 | | | | MP2K2 | CDON | | | | | |
| 0.604081 | | | | MP2K2 | | PH | | | | |
| 0.587324 | | | | MP2K2 | | | Notch1 | | | |
| 0.596001 | | | | MP2K2 | | | | IGFALS | | |
| 0.576375 | | | | MP2K2 | | | | | S100A13 | |
| 0.611607 | | | | MP2K2 | | | | | | CILP2 |
| 0.621375 | | | | | CDON | PH | | | | |
| 0.610122 | | | | | CDON | | Notch1 | | | |
| 0.614156 | | | | | CDON | | | IGFALS | | |
| 0.609691 | | | | | CDON | | | | S100A13 | |
| 0.615357 | | | | | CDON | | | | | CILP2 |
| 0.58407 | | | | | | PH | Notch1 | | | |
| 0.588718 | | | | | | PH | | IGFALS | | |
| 0.572949 | | | | | | PH | | | S100A13 | |
| 0.595561 | | | | | | PH | | | | CILP2 |
| 0.581485 | | | | | | | Notch1 | IGFALS | | |
| 0.551075 | | | | | | | Notch1 | | S100A13 | |
| 0.573785 | | | | | | | Notch1 | | | CILP2 |
| 0.574145 | | | | | | | | IGFALS | S100A13 | |
| 0.590608 | | | | | | | | IGFALS | | CILP2 |
| 0.574881 | | | | | | | | | S100A13 | CILP2 |
| 0.635119 | NADK | H2A3 | PTN | | | | | | | |
| 0.605335 | NADK | H2A3 | | MP2K2 | | | | | | |
| 0.628764 | NADK | H2A3 | | | CDON | | | | | |
| 0.606326 | NADK | H2A3 | | | | PH | | | | |
| 0.587993 | NADK | H2A3 | | | | | Notch1 | | | |
| 0.591764 | NADK | H2A3 | | | | | | IGFALS | | |
| 0.575995 | NADK | H2A3 | | | | | | | S100A13 | |
| 0.598986 | NADK | H2A3 | | | | | | | | CILP2 |
| 0.635027 | NADK | | PTN | MP2K2 | | | | | | |
| 0.659603 | NADK | | PTN | | CDON | | | | | |
| 0.635816 | NADK | | PTN | | | PH | | | | |
| 0.642654 | NADK | | PTN | | | | Notch1 | | | |
| 0.633492 | NADK | | PTN | | | | | IGFALS | | |
| 0.632316 | NADK | | PTN | | | | | | S100A13 | |
| 0.663054 | NADK | | PTN | | | | | | | CILP2 |
| 0.636219 | NADK | | | MP2K2 | CDON | | | | | |
| 0.619703 | NADK | | | MP2K2 | | PH | | | | |
| 0.602535 | NADK | | | MP2K2 | | | Notch1 | | | |
| 0.608506 | NADK | | | MP2K2 | | | | IGFALS | | |
| 0.595946 | NADK | | | MP2K2 | | | | | S100A13 | |
| 0.620875 | NADK | | | MP2K2 | | | | | | CILP2 |
| 0.630901 | NADK | | | | CDON | PH | | | | |
| 0.620843 | NADK | | | | CDON | | Notch1 | | | |
| 0.622869 | NADK | | | | CDON | | | IGFALS | | |
| 0.620324 | NADK | | | | CDON | | | | S100A13 | |
| 0.623823 | NADK | | | | CDON | | | | | CILP2 |
| 0.604277 | NADK | | | | | PH | Notch1 | | | |
| 0.604933 | NADK | | | | | PH | | IGFALS | | |
| 0.598095 | NADK | | | | | PH | | | S100A13 | |
| 0.60924 | NADK | | | | | PH | | | | CILP2 |
| 0.593884 | NADK | | | | | | Notch1 | IGFALS | | |
| 0.575962 | NADK | | | | | | Notch1 | | S100A13 | |
| 0.591494 | NADK | | | | | | Notch1 | | | CILP2 |
| 0.585659 | NADK | | | | | | | IGFALS | S100A13 | |
| 0.602184 | NADK | | | | | | | IGFALS | | CILP2 |
| 0.591736 | NADK | | | | | | | | S100A13 | CILP2 |
| 0.637939 | | H2A3 | PTN | MP2K2 | | | | | | |
| 0.658091 | | H2A3 | PTN | | CDON | | | | | |
| 0.638818 | | H2A3 | PTN | | | PH | | | | |
| 0.643547 | | H2A3 | PTN | | | | Notch1 | | | |
| 0.633778 | | H2A3 | PTN | | | | | IGFALS | | |
| 0.634809 | | H2A3 | PTN | | | | | | S100A13 | |
| 0.658681 | | H2A3 | PTN | | | | | | | CILP2 |
| 0.640555 | | H2A3 | | MP2K2 | CDON | | | | | |
| 0.622552 | | H2A3 | | MP2K2 | | PH | | | | |
| 0.608771 | | H2A3 | | MP2K2 | | | Notch1 | | | |
| 0.611193 | | H2A3 | | MP2K2 | | | | IGFALS | | |
| 0.600763 | | H2A3 | | MP2K2 | | | | | S100A13 | |
| 0.620505 | | H2A3 | | MP2K2 | | | | | | CILP2 |
| 0.634009 | | H2A3 | | | CDON | PH | | | | |
| 0.62335 | | H2A3 | | | CDON | | Notch1 | | | |
| 0.624283 | | H2A3 | | | CDON | | | IGFALS | | |
| 0.623389 | | H2A3 | | | CDON | | | | S100A13 | |
| 0.626049 | | H2A3 | | | CDON | | | | | CILP2 |
| 0.604392 | | H2A3 | | | | PH | Notch1 | | | |
| 0.603401 | | H2A3 | | | | PH | | IGFALS | | |
| 0.59741 | | H2A3 | | | | PH | | | S100A13 | |
| 0.60808 | | H2A3 | | | | PH | | | | CILP2 |
| 0.5929 | | H2A3 | | | | | Notch1 | IGFALS | | |
| 0.575849 | | H2A3 | | | | | Notch1 | | S100A13 | |
| 0.588356 | | H2A3 | | | | | Notch1 | | | CILP2 |
| 0.587448 | | H2A3 | | | | | | IGFALS | S100A13 | |
| 0.599673 | | H2A3 | | | | | | IGFALS | | CILP2 |
| 0.589123 | | H2A3 | | | | | | | S100A13 | CILP2 |
| 0.656883 | | | PTN | MP2K2 | CDON | | | | | |
| 0.633127 | | | PTN | MP2K2 | | PH | | | | |
| 0.638116 | | | PTN | MP2K2 | | | Notch1 | | | |
| 0.632325 | | | PTN | MP2K2 | | | | IGFALS | | |
| 0.628765 | | | PTN | MP2K2 | | | | | S100A13 | |
| 0.662224 | | | PTN | MP2K2 | | | | | | CILP2 |
| 0.656231 | | | PTN | | CDON | PH | | | | |
| 0.656144 | | | PTN | | CDON | | Notch1 | | | |
| 0.65551 | | | PTN | | CDON | | | IGFALS | | |
| 0.655379 | | | PTN | | CDON | | | | S100A13 | |
| 0.668615 | | | PTN | | CDON | | | | | CILP2 |
| 0.639871 | | | PTN | | | PH | Notch1 | | | |
| 0.631451 | | | PTN | | | PH | | IGFALS | | |
| 0.630592 | | | PTN | | | PH | | | S100A13 | |
| 0.657318 | | | PTN | | | PH | | | | CILP2 |
| 0.638308 | | | PTN | | | | Notch1 | IGFALS | | |
| 0.637031 | | | PTN | | | | Notch1 | | S100A13 | |
| 0.6579 | | | PTN | | | | Notch1 | | | CILP2 |
| 0.629622 | | | PTN | | | | | IGFALS | S100A13 | |
| 0.658753 | | | PTN | | | | | IGFALS | | CILP2 |
| 0.658112 | | | PTN | | | | | | S100A13 | CILP2 |
| 0.639074 | | | | MP2K2 | CDON | PH | | | | |
| 0.630032 | | | | MP2K2 | CDON | | Notch1 | | | |
| 0.632613 | | | | MP2K2 | CDON | | | IGFALS | | |
| 0.629931 | | | | MP2K2 | CDON | | | | S100A13 | |
| 0.635457 | | | | MP2K2 | CDON | | | | | CILP2 |
| 0.612276 | | | | MP2K2 | | PH | Notch1 | | | |
| 0.61621 | | | | MP2K2 | | PH | | IGFALS | | |
| 0.604946 | | | | MP2K2 | | PH | | | S100A13 | |
| 0.626045 | | | | MP2K2 | | PH | | | | CILP2 |
| 0.601964 | | | | MP2K2 | | | Notch1 | IGFALS | | |
| 0.5886 | | | | MP2K2 | | | Notch1 | | S100A13 | |
| 0.611417 | | | | MP2K2 | | | Notch1 | | | CILP2 |
| 0.597683 | | | | MP2K2 | | | | IGFALS | S100A13 | |
| 0.620161 | | | | MP2K2 | | | | IGFALS | | CILP2 |
| 0.611975 | | | | MP2K2 | | | | | S100A13 | CILP2 |
| 0.622648 | | | | | CDON | PH | Notch1 | | | |
| 0.625384 | | | | | CDON | PH | | IGFALS | | |
| 0.622313 | | | | | CDON | PH | | | S100A13 | |
| 0.624652 | | | | | CDON | PH | | | | CILP2 |
| 0.615375 | | | | | CDON | | Notch1 | IGFALS | | |
| 0.611581 | | | | | CDON | | Notch1 | | S100A13 | |
| 0.615454 | | | | | CDON | | Notch1 | | | CILP2 |
| 0.615293 | | | | | CDON | | | IGFALS | S100A13 | |
| 0.619826 | | | | | CDON | | | IGFALS | | CILP2 |
| 0.615999 | | | | | CDON | | | | S100A13 | CILP2 |
| 0.598435 | | | | | | PH | Notch1 | IGFALS | | |
| 0.584826 | | | | | | PH | Notch1 | | S100A13 | |
| 0.594334 | | | | | | PH | Notch1 | | | CILP2 |
| 0.590475 | | | | | | PH | | IGFALS | S100A13 | |
| 0.604587 | | | | | | PH | | IGFALS | | CILP2 |
| 0.595677 | | | | | | PH | | | S100A13 | CILP2 |
| 0.581818 | | | | | | | Notch1 | IGFALS | S100A13 | |
| 0.590616 | | | | | | | Notch1 | IGFALS | | CILP2 |
| 0.574394 | | | | | | | Notch1 | | S100A13 | CILP2 |
| 0.591323 | | | | | | | | IGFALS | S100A13 | CILP2 |
| 0.642577 | NADK | H2A3 | PTN | MP2K2 | | | | | | |
| 0.662869 | NADK | H2A3 | PTN | | CDON | | | | | |
| 0.644605 | NADK | H2A3 | PTN | | | PH | | | | |
| 0.648595 | NADK | H2A3 | PTN | | | | Notch1 | | | |
| 0.63901 | NADK | H2A3 | PTN | | | | | IGFALS | | |
| 0.639837 | NADK | H2A3 | PTN | | | | | | S100A13 | |
| 0.664515 | NADK | H2A3 | PTN | | | | | | | CILP2 |
| 0.644363 | NADK | H2A3 | | MP2K2 | CDON | | | | | |
| 0.627408 | NADK | H2A3 | | MP2K2 | | PH | | | | |
| 0.614182 | NADK | H2A3 | | MP2K2 | | | Notch1 | | | |
| 0.615229 | NADK | H2A3 | | MP2K2 | | | | IGFALS | | |
| 0.605675 | NADK | H2A3 | | MP2K2 | | | | | S100A13 | |
| 0.625607 | NADK | H2A3 | | MP2K2 | | | | | | CILP2 |
| 0.639904 | NADK | H2A3 | | | CDON | PH | | | | |
| 0.629635 | NADK | H2A3 | | | CDON | | Notch1 | | | |
| 0.629767 | NADK | H2A3 | | | CDON | | | IGFALS | | |
| 0.628847 | NADK | H2A3 | | | CDON | | | | S100A13 | |
| 0.631329 | NADK | H2A3 | | | CDON | | | | | CILP2 |
| 0.613771 | NADK | H2A3 | | | | PH | Notch1 | | | |
| 0.611781 | NADK | H2A3 | | | | PH | | IGFALS | | |
| 0.606269 | NADK | H2A3 | | | | PH | | | S100A13 | |
| 0.616532 | NADK | H2A3 | | | | PH | | | | CILP2 |
| 0.600469 | NADK | H2A3 | | | | | Notch1 | IGFALS | | |
| 0.586837 | NADK | H2A3 | | | | | Notch1 | | S100A13 | |
| 0.599737 | NADK | H2A3 | | | | | Notch1 | | | CILP2 |
| 0.592941 | NADK | H2A3 | | | | | | IGFALS | S100A13 | |
| 0.607606 | NADK | H2A3 | | | | | | IGFALS | | CILP2 |
| 0.599392 | NADK | H2A3 | | | | | | | S100A13 | CILP2 |
| 0.663918 | NADK | | PTN | MP2K2 | CDON | | | | | |
| 0.644429 | NADK | | PTN | MP2K2 | | PH | | | | |
| 0.647356 | NADK | | PTN | MP2K2 | | | Notch1 | | | |
| 0.641238 | NADK | | PTN | MP2K2 | | | | IGFALS | | |
| 0.63876 | NADK | | PTN | MP2K2 | | | | | S100A13 | |
| 0.669371 | NADK | | PTN | MP2K2 | | | | | | CILP2 |
| 0.663071 | NADK | | PTN | | CDON | PH | | | | |
| 0.662973 | NADK | | PTN | | CDON | | Notch1 | | | |
| 0.66249 | NADK | | PTN | | CDON | | | IGFALS | | |
| 0.66188 | NADK | | PTN | | CDON | | | | S100A13 | |
| 0.674956 | NADK | | PTN | | CDON | | | | | CILP2 |
| 0.649185 | NADK | | PTN | | | PH | Notch1 | | | |
| 0.641115 | NADK | | PTN | | | PH | | IGFALS | | |
| 0.641399 | NADK | | PTN | | | PH | | | S100A13 | |
| 0.664893 | NADK | | PTN | | | PH | | | | CILP2 |
| 0.64648 | NADK | | PTN | | | | Notch1 | IGFALS | | |
| 0.645336 | NADK | | PTN | | | | Notch1 | | S100A13 | |
| 0.665215 | NADK | | PTN | | | | Notch1 | | | CILP2 |
| 0.638202 | NADK | | PTN | | | | | IGFALS | S100A13 | |
| 0.665353 | NADK | | PTN | | | | | IGFALS | | CILP2 |
| 0.665161 | NADK | | PTN | | | | | | S100A13 | CILP2 |
| 0.646136 | NADK | | | MP2K2 | CDON | PH | | | | |
| 0.636765 | NADK | | | MP2K2 | CDON | | Notch1 | | | |
| 0.638974 | NADK | | | MP2K2 | CDON | | | IGFALS | | |
| 0.636481 | NADK | | | MP2K2 | CDON | | | | S100A13 | |
| 0.641204 | NADK | | | MP2K2 | CDON | | | | | CILP2 |
| 0.62474 | NADK | | | MP2K2 | | PH | Notch1 | | | |
| 0.627012 | NADK | | | MP2K2 | | PH | | IGFALS | | |
| 0.619918 | NADK | | | MP2K2 | | PH | | | S100A13 | |
| 0.634278 | NADK | | | MP2K2 | | PH | | | | CILP2 |
| 0.613033 | NADK | | | MP2K2 | | | Notch1 | IGFALS | | |
| 0.602425 | NADK | | | MP2K2 | | | Notch1 | | S100A13 | |
| 0.621302 | NADK | | | MP2K2 | | | Notch1 | | | CILP2 |
| 0.609612 | NADK | | | MP2K2 | | | | IGFALS | S100A13 | |
| 0.627022 | NADK | | | MP2K2 | | | | IGFALS | | CILP2 |
| 0.621052 | NADK | | | MP2K2 | | | | | S100A13 | CILP2 |
| 0.631849 | NADK | | | | CDON | PH | Notch1 | | | |
| 0.633869 | NADK | | | | CDON | PH | | IGFALS | | |
| 0.631507 | NADK | | | | CDON | PH | | | S100A13 | |
| 0.63262 | NADK | | | | CDON | PH | | | | CILP2 |
| 0.624055 | NADK | | | | CDON | | Notch1 | IGFALS | | |
| 0.621325 | NADK | | | | CDON | | Notch1 | | S100A13 | |
| 0.624081 | NADK | | | | CDON | | Notch1 | | | CILP2 |
| 0.623707 | NADK | | | | CDON | | | IGFALS | S100A13 | |
| 0.627186 | NADK | | | | CDON | | | IGFALS | | CILP2 |
| 0.624034 | NADK | | | | CDON | | | | S100A13 | CILP2 |
| 0.612176 | NADK | | | | | PH | Notch1 | IGFALS | | |
| 0.604534 | NADK | | | | | PH | Notch1 | | S100A13 | |
| 0.609728 | NADK | | | | | PH | Notch1 | | | CILP2 |
| 0.606123 | NADK | | | | | PH | | IGFALS | S100A13 | |
| 0.615505 | NADK | | | | | PH | | IGFALS | | CILP2 |
| 0.609349 | NADK | | | | | PH | | | S100A13 | CILP2 |
| 0.593823 | NADK | | | | | | Notch1 | IGFALS | S100A13 | |
| 0.603846 | NADK | | | | | | Notch1 | IGFALS | | CILP2 |
| 0.591652 | NADK | | | | | | Notch1 | | S100A13 | CILP2 |
| 0.602821 | NADK | | | | | | | IGFALS | S100A13 | CILP2 |
| 0.662677 | | H2A3 | PTN | MP2K2 | CDON | | | | | |
| 0.646603 | | H2A3 | PTN | MP2K2 | | PH | | | | |
| 0.648668 | | H2A3 | PTN | MP2K2 | | | Notch1 | | | |
| 0.642302 | | H2A3 | PTN | MP2K2 | | | | IGFALS | | |
| 0.642 | | H2A3 | PTN | MP2K2 | | | | | S100A13 | |
| 0.665181 | | H2A3 | PTN | MP2K2 | | | | | | CILP2 |
| 0.662424 | | H2A3 | PTN | | CDON | PH | | | | |
| 0.662453 | | H2A3 | PTN | | CDON | | Notch1 | | | |
| 0.659707 | | H2A3 | PTN | | CDON | | | IGFALS | | |
| 0.66076 | | H2A3 | PTN | | CDON | | | | S100A13 | |
| 0.67169 | | H2A3 | PTN | | CDON | | | | | CILP2 |
| 0.650192 | | H2A3 | PTN | | | PH | Notch1 | | | |
| 0.641528 | | H2A3 | PTN | | | PH | | IGFALS | | |
| 0.644462 | | H2A3 | PTN | | | PH | | | S100A13 | |
| 0.661461 | | H2A3 | PTN | | | PH | | | | CILP2 |
| 0.646161 | | H2A3 | PTN | | | | Notch1 | IGFALS | | |
| 0.646436 | | H2A3 | PTN | | | | Notch1 | | S100A13 | |
| 0.661254 | | H2A3 | PTN | | | | Notch1 | | | CILP2 |
| 0.638655 | | H2A3 | PTN | | | | | IGFALS | S100A13 | |
| 0.661377 | | H2A3 | PTN | | | | | IGFALS | | CILP2 |
| 0.661552 | | H2A3 | PTN | | | | | | S100A13 | CILP2 |
| 0.649427 | | H2A3 | | MP2K2 | CDON | PH | | | | |
| 0.640783 | | H2A3 | | MP2K2 | CDON | | Notch1 | | | |
| 0.641549 | | H2A3 | | MP2K2 | CDON | | | IGFALS | | |
| 0.640799 | | H2A3 | | MP2K2 | CDON | | | | S100A13 | |
| 0.644277 | | H2A3 | | MP2K2 | CDON | | | | | CILP2 |
| 0.628294 | | H2A3 | | MP2K2 | | PH | Notch1 | | | |
| 0.628674 | | H2A3 | | MP2K2 | | PH | | IGFALS | | |
| 0.623156 | | H2A3 | | MP2K2 | | PH | | | S100A13 | |
| 0.63466 | | H2A3 | | MP2K2 | | PH | | | | CILP2 |
| 0.61841 | | H2A3 | | MP2K2 | | | Notch1 | IGFALS | | |
| 0.609309 | | H2A3 | | MP2K2 | | | Notch1 | | S100A13 | |
| 0.621244 | | H2A3 | | MP2K2 | | | Notch1 | | | CILP2 |
| 0.612466 | | H2A3 | | MP2K2 | | | | IGFALS | S100A13 | |
| 0.627196 | | H2A3 | | MP2K2 | | | | IGFALS | | CILP2 |
| 0.620908 | | H2A3 | | MP2K2 | | | | | S100A13 | CILP2 |
| 0.634657 | | H2A3 | | | CDON | PH | Notch1 | | | |
| 0.635049 | | H2A3 | | | CDON | PH | | IGFALS | | |
| 0.634371 | | H2A3 | | | CDON | PH | | | S100A13 | |
| 0.635766 | | H2A3 | | | CDON | PH | | | | CILP2 |
| 0.625769 | | H2A3 | | | CDON | | Notch1 | IGFALS | | |
| 0.623614 | | H2A3 | | | CDON | | Notch1 | | S100A13 | |
| 0.626117 | | H2A3 | | | CDON | | Notch1 | | | CILP2 |
| 0.625056 | | H2A3 | | | CDON | | | IGFALS | S100A13 | |
| 0.628104 | | H2A3 | | | CDON | | | IGFALS | | CILP2 |
| 0.626569 | | H2A3 | | | CDON | | | | S100A13 | CILP2 |
| 0.610807 | | H2A3 | | | | PH | Notch1 | IGFALS | | |
| 0.604194 | | H2A3 | | | | PH | Notch1 | | S100A13 | |
| 0.608499 | | H2A3 | | | | PH | Notch1 | | | CILP2 |
| 0.604565 | | H2A3 | | | | PH | | IGFALS | S100A13 | |
| 0.613268 | | H2A3 | | | | PH | | IGFALS | | CILP2 |
| 0.607793 | | H2A3 | | | | PH | | | S100A13 | CILP2 |
| 0.593333 | | H2A3 | | | | | Notch1 | IGFALS | S100A13 | |
| 0.600587 | | H2A3 | | | | | Notch1 | IGFALS | | CILP2 |
| 0.588725 | | H2A3 | | | | | Notch1 | | S100A13 | CILP2 |
| 0.600525 | | H2A3 | | | | | | IGFALS | S100A13 | CILP2 |
| 0.661039 | | | PTN | MP2K2 | CDON | PH | | | | |
| 0.660081 | | | PTN | MP2K2 | CDON | | Notch1 | | | |
| 0.660048 | | | PTN | MP2K2 | CDON | | | IGFALS | | |
| 0.659472 | | | PTN | MP2K2 | CDON | | | | S100A13 | |
| 0.672235 | | | PTN | MP2K2 | CDON | | | | | CILP2 |
| 0.645417 | | | PTN | MP2K2 | | PH | Notch1 | | | |
| 0.639999 | | | PTN | MP2K2 | | PH | | IGFALS | | |
| 0.6376 | | | PTN | MP2K2 | | PH | | | S100A13 | |
| 0.664461 | | | PTN | MP2K2 | | PH | | | | CILP2 |
| 0.643333 | | | PTN | MP2K2 | | | Notch1 | IGFALS | | |
| 0.641073 | | | PTN | MP2K2 | | | Notch1 | | S100A13 | |
| 0.663171 | | | PTN | MP2K2 | | | Notch1 | | | CILP2 |
| 0.636419 | | | PTN | MP2K2 | | | | IGFALS | S100A13 | |
| 0.665983 | | | PTN | MP2K2 | | | | IGFALS | | CILP2 |
| 0.663951 | | | PTN | MP2K2 | | | | | S100A13 | CILP2 |
| 0.659969 | | | PTN | | CDON | PH | Notch1 | | | |
| 0.659387 | | | PTN | | CDON | PH | | IGFALS | | |
| 0.659622 | | | PTN | | CDON | PH | | | S100A13 | |
| 0.670051 | | | PTN | | CDON | PH | | | | CILP2 |
| 0.659204 | | | PTN | | CDON | | Notch1 | IGFALS | | |
| 0.659249 | | | PTN | | CDON | | Notch1 | | S100A13 | |
| 0.66936 | | | PTN | | CDON | | Notch1 | | | CILP2 |
| 0.6588 | | | PTN | | CDON | | | IGFALS | S100A13 | |
| 0.670735 | | | PTN | | CDON | | | IGFALS | | CILP2 |
| 0.670585 | | | PTN | | CDON | | | | S100A13 | CILP2 |
| 0.644094 | | | PTN | | | PH | Notch1 | IGFALS | | |
| 0.643929 | | | PTN | | | PH | Notch1 | | S100A13 | |
| 0.659534 | | | PTN | | | PH | Notch1 | | | CILP2 |
| 0.6374 | | | PTN | | | PH | | IGFALS | S100A13 | |
| 0.66079 | | | PTN | | | PH | | IGFALS | | CILP2 |
| 0.660343 | | | PTN | | | PH | | | S100A13 | CILP2 |
| 0.641622 | | | PTN | | | | Notch1 | IGFALS | S100A13 | |
| 0.660758 | | | PTN | | | | Notch1 | IGFALS | | CILP2 |
| 0.660829 | | | PTN | | | | Notch1 | | S100A13 | CILP2 |
| 0.662013 | | | PTN | | | | | IGFALS | S100A13 | CILP2 |
| 0.640112 | | | | MP2K2 | CDON | PH | Notch1 | | | |
| 0.641651 | | | | MP2K2 | CDON | PH | | IGFALS | | |
| 0.639524 | | | | MP2K2 | CDON | PH | | | S100A13 | |
| 0.643296 | | | | MP2K2 | CDON | PH | | | | CILP2 |
| 0.633224 | | | | MP2K2 | CDON | | Notch1 | IGFALS | | |
| 0.630956 | | | | MP2K2 | CDON | | Notch1 | | S100A13 | |
| 0.635454 | | | | MP2K2 | CDON | | Notch1 | | | CILP2 |
| 0.633598 | | | | MP2K2 | CDON | | | IGFALS | S100A13 | |
| 0.638521 | | | | MP2K2 | CDON | | | IGFALS | | CILP2 |
| 0.635764 | | | | MP2K2 | CDON | | | | S100A13 | CILP2 |
| 0.621313 | | | | MP2K2 | | PH | Notch1 | IGFALS | | |
| 0.612718 | | | | MP2K2 | | PH | Notch1 | | S100A13 | |
| 0.62573 | | | | MP2K2 | | PH | Notch1 | | | CILP2 |
| 0.61711 | | | | MP2K2 | | PH | | IGFALS | S100A13 | |
| 0.6325 | | | | MP2K2 | | PH | | IGFALS | | CILP2 |
| 0.62605 | | | | MP2K2 | | PH | | | S100A13 | CILP2 |
| 0.602895 | | | | MP2K2 | | | Notch1 | IGFALS | S100A13 | |
| 0.620228 | | | | MP2K2 | | | Notch1 | IGFALS | | CILP2 |
| 0.61178 | | | | MP2K2 | | | Notch1 | | S100A13 | CILP2 |
| 0.620678 | | | | MP2K2 | | | | IGFALS | S100A13 | CILP2 |
| 0.626082 | | | | | CDON | PH | Notch1 | IGFALS | | |
| 0.623366 | | | | | CDON | PH | Notch1 | | S100A13 | |
| 0.624915 | | | | | CDON | PH | Notch1 | | | CILP2 |
| 0.626233 | | | | | CDON | PH | | IGFALS | S100A13 | |
| 0.628562 | | | | | CDON | PH | | IGFALS | | CILP2 |
| 0.625232 | | | | | CDON | PH | | | S100A13 | CILP2 |
| 0.616172 | | | | | CDON | | Notch1 | IGFALS | S100A13 | |
| 0.620093 | | | | | CDON | | Notch1 | IGFALS | | CILP2 |
| 0.61632 | | | | | CDON | | Notch1 | | S100A13 | CILP2 |
| 0.620522 | | | | | CDON | | | IGFALS | S100A13 | CILP2 |
| 0.598893 | | | | | | PH | Notch1 | IGFALS | S100A13 | |
| 0.605227 | | | | | | PH | Notch1 | IGFALS | | CILP2 |
| 0.594459 | | | | | | PH | Notch1 | | S100A13 | CILP2 |
| 0.60483 | | | | | | PH | | IGFALS | S100A13 | CILP2 |
| 0.591298 | | | | | | | Notch1 | IGFALS | S100A13 | CILP2 |
| 0.667407 | NADK | H2A3 | PTN | MP2K2 | CDON | | | | | |
| 0.651718 | NADK | H2A3 | PTN | MP2K2 | | PH | | | | |
| 0.653071 | NADK | H2A3 | PTN | MP2K2 | | | Notch1 | | | |
| 0.646574 | NADK | H2A3 | PTN | MP2K2 | | | | IGFALS | | |
| 0.645908 | NADK | H2A3 | PTN | MP2K2 | | | | | S100A13 | |
| 0.670617 | NADK | H2A3 | PTN | MP2K2 | | | | | | CILP2 |
| 0.666822 | NADK | H2A3 | PTN | | CDON | PH | | | | |
| 0.666639 | NADK | H2A3 | PTN | | CDON | | Notch1 | | | |
| 0.664152 | NADK | H2A3 | PTN | | CDON | | | IGFALS | | |
| 0.664777 | NADK | H2A3 | PTN | | CDON | | | | S100A13 | |
| 0.676379 | NADK | H2A3 | PTN | | CDON | | | | | CILP2 |
| 0.654844 | NADK | H2A3 | PTN | | | PH | Notch1 | | | |
| 0.647035 | NADK | H2A3 | PTN | | | PH | | IGFALS | | |
| 0.649463 | NADK | H2A3 | PTN | | | PH | | | S100A13 | |
| 0.666808 | NADK | H2A3 | PTN | | | PH | | | | CILP2 |
| 0.650789 | NADK | H2A3 | PTN | | | | Notch1 | IGFALS | | |
| 0.650505 | NADK | H2A3 | PTN | | | | Notch1 | | S100A13 | |
| 0.66699 | NADK | H2A3 | PTN | | | | Notch1 | | | CILP2 |
| 0.643534 | NADK | H2A3 | PTN | | | | | IGFALS | S100A13 | |
| 0.666562 | NADK | H2A3 | PTN | | | | | IGFALS | | CILP2 |
| 0.666809 | NADK | H2A3 | PTN | | | | | | S100A13 | CILP2 |
| 0.653395 | NADK | H2A3 | | MP2K2 | CDON | PH | | | | |
| 0.644813 | NADK | H2A3 | | MP2K2 | CDON | | Notch1 | | | |
| 0.645112 | NADK | H2A3 | | MP2K2 | CDON | | | IGFALS | | |
| 0.644362 | NADK | H2A3 | | MP2K2 | CDON | | | | S100A13 | |
| 0.647794 | NADK | H2A3 | | MP2K2 | CDON | | | | | CILP2 |
| 0.633232 | NADK | H2A3 | | MP2K2 | | PH | Notch1 | | | |
| 0.632403 | NADK | H2A3 | | MP2K2 | | PH | | IGFALS | | |
| 0.62745 | NADK | H2A3 | | MP2K2 | | PH | | | S100A13 | |
| 0.639318 | NADK | H2A3 | | MP2K2 | | PH | | | | CILP2 |
| 0.622391 | NADK | H2A3 | | MP2K2 | | | Notch1 | IGFALS | | |
| 0.61424 | NADK | H2A3 | | MP2K2 | | | Notch1 | | S100A13 | |
| 0.626552 | NADK | H2A3 | | MP2K2 | | | Notch1 | | | CILP2 |
| 0.615973 | NADK | H2A3 | | MP2K2 | | | | IGFALS | S100A13 | |
| 0.631078 | NADK | H2A3 | | MP2K2 | | | | IGFALS | | CILP2 |
| 0.625967 | NADK | H2A3 | | MP2K2 | | | | | S100A13 | CILP2 |
| 0.640476 | NADK | H2A3 | | | CDON | PH | Notch1 | | | |
| 0.640323 | NADK | H2A3 | | | CDON | PH | | IGFALS | | |
| 0.63967 | NADK | H2A3 | | | CDON | PH | | | S100A13 | |
| 0.640615 | NADK | H2A3 | | | CDON | PH | | | | CILP2 |
| 0.631264 | NADK | H2A3 | | | CDON | | Notch1 | IGFALS | | |
| 0.629598 | NADK | H2A3 | | | CDON | | Notch1 | | S100A13 | |
| 0.631724 | NADK | H2A3 | | | CDON | | Notch1 | | | CILP2 |
| 0.630074 | NADK | H2A3 | | | CDON | | | IGFALS | S100A13 | |
| 0.633297 | NADK | H2A3 | | | CDON | | | IGFALS | | CILP2 |
| 0.631447 | NADK | H2A3 | | | CDON | | | | S100A13 | CILP2 |
| 0.618333 | NADK | H2A3 | | | | PH | Notch1 | IGFALS | | |
| 0.613417 | NADK | H2A3 | | | | PH | Notch1 | | S100A13 | |
| 0.617907 | NADK | H2A3 | | | | PH | Notch1 | | | CILP2 |
| 0.612429 | NADK | H2A3 | | | | PH | | IGFALS | S100A13 | |
| 0.620566 | NADK | H2A3 | | | | PH | | IGFALS | | CILP2 |
| 0.616451 | NADK | H2A3 | | | | PH | | | S100A13 | CILP2 |
| 0.600411 | NADK | H2A3 | | | | | Notch1 | IGFALS | S100A13 | |
| 0.60926 | NADK | H2A3 | | | | | Notch1 | IGFALS | | CILP2 |
| 0.599834 | NADK | H2A3 | | | | | Notch1 | | S100A13 | CILP2 |
| 0.608084 | NADK | H2A3 | | | | | | IGFALS | S100A13 | CILP2 |
| 0.667442 | NADK | | PTN | MP2K2 | CDON | PH | | | | |
| 0.666638 | NADK | | PTN | MP2K2 | CDON | | Notch1 | | | |
| 0.666367 | NADK | | PTN | MP2K2 | CDON | | | IGFALS | | |
| 0.665597 | NADK | | PTN | MP2K2 | CDON | | | | S100A13 | |
| 0.678117 | NADK | | PTN | MP2K2 | CDON | | | | | CILP2 |
| 0.654073 | NADK | | PTN | MP2K2 | | PH | Notch1 | | | |
| 0.648784 | NADK | | PTN | MP2K2 | | PH | | IGFALS | | |
| 0.648078 | NADK | | PTN | MP2K2 | | PH | | | S100A13 | |
| 0.671138 | NADK | | PTN | MP2K2 | | PH | | | | CILP2 |
| 0.651107 | NADK | | PTN | MP2K2 | | | Notch1 | IGFALS | | |
| 0.6491 | NADK | | PTN | MP2K2 | | | Notch1 | | S100A13 | |
| 0.670033 | NADK | | PTN | MP2K2 | | | Notch1 | | | CILP2 |
| 0.644749 | NADK | | PTN | MP2K2 | | | | IGFALS | S100A13 | |
| 0.671762 | NADK | | PTN | MP2K2 | | | | IGFALS | | CILP2 |
| 0.670625 | NADK | | PTN | MP2K2 | | | | | S100A13 | CILP2 |
| 0.666153 | NADK | | PTN | | CDON | PH | Notch1 | | | |
| 0.665608 | NADK | | PTN | | CDON | PH | | IGFALS | | |
| 0.665843 | NADK | | PTN | | CDON | PH | | | S100A13 | |
| 0.675935 | NADK | | PTN | | CDON | PH | | | | CILP2 |
| 0.665234 | NADK | | PTN | | CDON | | Notch1 | IGFALS | | |
| 0.665384 | NADK | | PTN | | CDON | | Notch1 | | S100A13 | |
| 0.675425 | NADK | | PTN | | CDON | | Notch1 | | | CILP2 |
| 0.664735 | NADK | | PTN | | CDON | | | IGFALS | S100A13 | |
| 0.6766 | NADK | | PTN | | CDON | | | IGFALS | | CILP2 |
| 0.67624 | NADK | | PTN | | CDON | | | | S100A13 | CILP2 |
| 0.652204 | NADK | | PTN | | | PH | Notch1 | IGFALS | | |
| 0.652147 | NADK | | PTN | | | PH | Notch1 | | S100A13 | |
| 0.666825 | NADK | | PTN | | | PH | Notch1 | | | CILP2 |
| 0.64584 | NADK | | PTN | | | PH | | IGFALS | S100A13 | |
| 0.667524 | NADK | | PTN | | | PH | | IGFALS | | CILP2 |
| 0.667049 | NADK | | PTN | | | PH | | | S100A13 | CILP2 |
| 0.648987 | NADK | | PTN | | | | Notch1 | IGFALS | S100A13 | |
| 0.667252 | NADK | | PTN | | | | Notch1 | IGFALS | | CILP2 |
| 0.667463 | NADK | | PTN | | | | Notch1 | | S100A13 | CILP2 |
| 0.667686 | NADK | | PTN | | | | | IGFALS | S100A13 | CILP2 |
| 0.64673 | NADK | | | MP2K2 | CDON | PH | Notch1 | | | |
| 0.64822 | NADK | | | MP2K2 | CDON | PH | | IGFALS | | |
| 0.646644 | NADK | | | MP2K2 | CDON | PH | | | S100A13 | |
| 0.648308 | NADK | | | MP2K2 | CDON | PH | | | | CILP2 |
| 0.639351 | NADK | | | MP2K2 | CDON | | Notch1 | IGFALS | | |
| 0.63693 | NADK | | | MP2K2 | CDON | | Notch1 | | S100A13 | |
| 0.641234 | NADK | | | MP2K2 | CDON | | Notch1 | | | CILP2 |
| 0.639746 | NADK | | | MP2K2 | CDON | | | IGFALS | S100A13 | |
| 0.64381 | NADK | | | MP2K2 | CDON | | | IGFALS | | CILP2 |
| 0.641042 | NADK | | | MP2K2 | CDON | | | | S100A13 | CILP2 |
| 0.630159 | NADK | | | MP2K2 | | PH | Notch1 | IGFALS | | |
| 0.624753 | NADK | | | MP2K2 | | PH | Notch1 | | S100A13 | |
| 0.634781 | NADK | | | MP2K2 | | PH | Notch1 | | | CILP2 |
| 0.627702 | NADK | | | MP2K2 | | PH | | IGFALS | S100A13 | |
| 0.639032 | NADK | | | MP2K2 | | PH | | IGFALS | | CILP2 |
| 0.634343 | NADK | | | MP2K2 | | PH | | | S100A13 | CILP2 |
| 0.613145 | NADK | | | MP2K2 | | | Notch1 | IGFALS | S100A13 | |
| 0.627612 | NADK | | | MP2K2 | | | Notch1 | IGFALS | | CILP2 |
| 0.621401 | NADK | | | MP2K2 | | | Notch1 | | S100A13 | CILP2 |
| 0.62734 | NADK | | | MP2K2 | | | | IGFALS | S100A13 | CILP2 |
| 0.634744 | NADK | | | | CDON | PH | Notch1 | IGFALS | | |
| 0.632327 | NADK | | | | CDON | PH | Notch1 | | S100A13 | |
| 0.633005 | NADK | | | | CDON | PH | Notch1 | | | CILP2 |
| 0.634556 | NADK | | | | CDON | PH | | IGFALS | S100A13 | |
| 0.635597 | NADK | | | | CDON | PH | | IGFALS | | CILP2 |
| 0.632859 | NADK | | | | CDON | PH | | | S100A13 | CILP2 |
| 0.624654 | NADK | | | | CDON | | Notch1 | IGFALS | S100A13 | |
| 0.627574 | NADK | | | | CDON | | Notch1 | IGFALS | | CILP2 |
| 0.624429 | NADK | | | | CDON | | Notch1 | | S100A13 | CILP2 |
| 0.627548 | NADK | | | | CDON | | | IGFALS | S100A13 | CILP2 |
| 0.612458 | NADK | | | | | PH | Notch1 | IGFALS | S100A13 | |
| 0.617426 | NADK | | | | | PH | Notch1 | IGFALS | | CILP2 |
| 0.60965 | NADK | | | | | PH | Notch1 | | S100A13 | CILP2 |
| 0.615578 | NADK | | | | | PH | | IGFALS | S100A13 | CILP2 |
| 0.604077 | NADK | | | | | | Notch1 | IGFALS | S100A13 | CILP2 |
| 0.666796 | | H2A3 | PTN | MP2K2 | CDON | PH | | | | |
| 0.666127 | | H2A3 | PTN | MP2K2 | CDON | | Notch1 | | | |
| 0.664343 | | H2A3 | PTN | MP2K2 | CDON | | | IGFALS | | |
| 0.664953 | | H2A3 | PTN | MP2K2 | CDON | | | | S100A13 | |
| 0.675311 | | H2A3 | PTN | MP2K2 | CDON | | | | | CILP2 |
| 0.654717 | | H2A3 | PTN | MP2K2 | | PH | Notch1 | | | |
| 0.649667 | | H2A3 | PTN | MP2K2 | | PH | | IGFALS | | |
| 0.650856 | | H2A3 | PTN | MP2K2 | | PH | | | S100A13 | |
| 0.668047 | | H2A3 | PTN | MP2K2 | | PH | | | | CILP2 |
| 0.651222 | | H2A3 | PTN | MP2K2 | | | Notch1 | IGFALS | | |
| 0.650724 | | H2A3 | PTN | MP2K2 | | | Notch1 | | S100A13 | |
| 0.666589 | | H2A3 | PTN | MP2K2 | | | Notch1 | | | CILP2 |
| 0.645856 | | H2A3 | PTN | MP2K2 | | | | IGFALS | S100A13 | |
| 0.667902 | | H2A3 | PTN | MP2K2 | | | | IGFALS | | CILP2 |
| 0.667109 | | H2A3 | PTN | MP2K2 | | | | | S100A13 | CILP2 |
| 0.665889 | | H2A3 | PTN | | CDON | PH | Notch1 | | | |
| 0.663424 | | H2A3 | PTN | | CDON | PH | | IGFALS | | |
| 0.665427 | | H2A3 | PTN | | CDON | PH | | | S100A13 | |
| 0.673066 | | H2A3 | PTN | | CDON | PH | | | | CILP2 |
| 0.663795 | | H2A3 | PTN | | CDON | | Notch1 | IGFALS | | |
| 0.664418 | | H2A3 | PTN | | CDON | | Notch1 | | S100A13 | |
| 0.67265 | | H2A3 | PTN | | CDON | | Notch1 | | | CILP2 |
| 0.662374 | | H2A3 | PTN | | CDON | | | IGFALS | S100A13 | |
| 0.673103 | | H2A3 | PTN | | CDON | | | IGFALS | | CILP2 |
| 0.673463 | | H2A3 | PTN | | CDON | | | | S100A13 | CILP2 |
| 0.652156 | | H2A3 | PTN | | | PH | Notch1 | IGFALS | | |
| 0.653107 | | H2A3 | PTN | | | PH | Notch1 | | S100A13 | |
| 0.663913 | | H2A3 | PTN | | | PH | Notch1 | | | CILP2 |
| 0.646477 | | H2A3 | PTN | | | PH | | IGFALS | S100A13 | |
| 0.664073 | | H2A3 | PTN | | | PH | | IGFALS | | CILP2 |
| 0.664529 | | H2A3 | PTN | | | PH | | | S100A13 | CILP2 |
| 0.648883 | | H2A3 | PTN | | | | Notch1 | IGFALS | S100A13 | |
| 0.663154 | | H2A3 | PTN | | | | Notch1 | IGFALS | | CILP2 |
| 0.663822 | | H2A3 | PTN | | | | Notch1 | | S100A13 | CILP2 |
| 0.664378 | | H2A3 | PTN | | | | | IGFALS | S100A13 | CILP2 |
| 0.649924 | | H2A3 | | MP2K2 | CDON | PH | Notch1 | | | |
| 0.650424 | | H2A3 | | MP2K2 | CDON | PH | | IGFALS | | |
| 0.6494 | | H2A3 | | MP2K2 | CDON | PH | | | S100A13 | |
| 0.651678 | | H2A3 | | MP2K2 | CDON | PH | | | | CILP2 |
| 0.642178 | | H2A3 | | MP2K2 | CDON | | Notch1 | IGFALS | | |
| 0.641313 | | H2A3 | | MP2K2 | CDON | | Notch1 | | S100A13 | |
| 0.644376 | | H2A3 | | MP2K2 | CDON | | Notch1 | | | CILP2 |
| 0.642272 | | H2A3 | | MP2K2 | CDON | | | IGFALS | S100A13 | |
| 0.645808 | | H2A3 | | MP2K2 | CDON | | | IGFALS | | CILP2 |
| 0.644472 | | H2A3 | | MP2K2 | CDON | | | | S100A13 | CILP2 |
| 0.633568 | | H2A3 | | MP2K2 | | PH | Notch1 | IGFALS | | |
| 0.628408 | | H2A3 | | MP2K2 | | PH | Notch1 | | S100A13 | |
| 0.635783 | | H2A3 | | MP2K2 | | PH | Notch1 | | | CILP2 |
| 0.629319 | | H2A3 | | MP2K2 | | PH | | IGFALS | S100A13 | |
| 0.639463 | | H2A3 | | MP2K2 | | PH | | IGFALS | | CILP2 |
| 0.634801 | | H2A3 | | MP2K2 | | PH | | | S100A13 | CILP2 |
| 0.618799 | | H2A3 | | MP2K2 | | | Notch1 | IGFALS | S100A13 | |
| 0.628532 | | H2A3 | | MP2K2 | | | Notch1 | IGFALS | | CILP2 |
| 0.621587 | | H2A3 | | MP2K2 | | | Notch1 | | S100A13 | CILP2 |
| 0.627705 | | H2A3 | | MP2K2 | | | | IGFALS | S100A13 | CILP2 |
| 0.636014 | | H2A3 | | | CDON | PH | Notch1 | IGFALS | | |
| 0.635095 | | H2A3 | | | CDON | PH | Notch1 | | S100A13 | |
| 0.635759 | | H2A3 | | | CDON | PH | Notch1 | | | CILP2 |
| 0.635308 | | H2A3 | | | CDON | PH | | IGFALS | S100A13 | |
| 0.636944 | | H2A3 | | | CDON | PH | | IGFALS | | CILP2 |
| 0.635868 | | H2A3 | | | CDON | PH | | | S100A13 | CILP2 |
| 0.625978 | | H2A3 | | | CDON | | Notch1 | IGFALS | S100A13 | |
| 0.628466 | | H2A3 | | | CDON | | Notch1 | IGFALS | | CILP2 |
| 0.626522 | | H2A3 | | | CDON | | Notch1 | | S100A13 | CILP2 |
| 0.628447 | | H2A3 | | | CDON | | | IGFALS | S100A13 | CILP2 |
| 0.611324 | | H2A3 | | | | PH | Notch1 | IGFALS | S100A13 | |
| 0.615486 | | H2A3 | | | | PH | Notch1 | IGFALS | | CILP2 |
| 0.60849 | | H2A3 | | | | PH | Notch1 | | S100A13 | CILP2 |
| 0.613427 | | H2A3 | | | | PH | | IGFALS | S100A13 | CILP2 |
| 0.600971 | | H2A3 | | | | | Notch1 | IGFALS | S100A13 | CILP2 |
| 0.663813 | | | PTN | MP2K2 | CDON | PH | Notch1 | | | |
| 0.663867 | | | PTN | MP2K2 | CDON | PH | | IGFALS | | |
| 0.663908 | | | PTN | MP2K2 | CDON | PH | | | S100A13 | |
| 0.673563 | | | PTN | MP2K2 | CDON | PH | | | | CILP2 |
| 0.662726 | | | PTN | MP2K2 | CDON | | Notch1 | IGFALS | | |
| 0.662708 | | | PTN | MP2K2 | CDON | | Notch1 | | S100A13 | |
| 0.672726 | | | PTN | MP2K2 | CDON | | Notch1 | | | CILP2 |
| 0.662857 | | | PTN | MP2K2 | CDON | | | IGFALS | S100A13 | |
| 0.674625 | | | PTN | MP2K2 | CDON | | | IGFALS | | CILP2 |
| 0.673731 | | | PTN | MP2K2 | CDON | | | | S100A13 | CILP2 |
| 0.649349 | | | PTN | MP2K2 | | PH | Notch1 | IGFALS | | |
| 0.647868 | | | PTN | MP2K2 | | PH | Notch1 | | S100A13 | |
| 0.665079 | | | PTN | MP2K2 | | PH | Notch1 | | | CILP2 |
| 0.64405 | | | PTN | MP2K2 | | PH | | IGFALS | S100A13 | |
| 0.667965 | | | PTN | MP2K2 | | PH | | IGFALS | | CILP2 |
| 0.66609 | | | PTN | MP2K2 | | PH | | | S100A13 | CILP2 |
| 0.64579 | | | PTN | MP2K2 | | | Notch1 | IGFALS | S100A13 | |
| 0.666283 | | | PTN | MP2K2 | | | Notch1 | IGFALS | | CILP2 |
| 0.665378 | | | PTN | MP2K2 | | | Notch1 | | S100A13 | CILP2 |
| 0.667724 | | | PTN | MP2K2 | | | | IGFALS | S100A13 | CILP2 |
| 0.66246 | | | PTN | | CDON | PH | Notch1 | IGFALS | | |
| 0.662951 | | | PTN | | CDON | PH | Notch1 | | S100A13 | |
| 0.670559 | | | PTN | | CDON | PH | Notch1 | | | CILP2 |
| 0.662911 | | | PTN | | CDON | PH | | IGFALS | S100A13 | |
| 0.671954 | | | PTN | | CDON | PH | | IGFALS | | CILP2 |
| 0.672359 | | | PTN | | CDON | PH | | | S100A13 | CILP2 |
| 0.66181 | | | PTN | | CDON | | Notch1 | IGFALS | S100A13 | |
| 0.671156 | | | PTN | | CDON | | Notch1 | IGFALS | | CILP2 |
| 0.671525 | | | PTN | | CDON | | Notch1 | | S100A13 | CILP2 |
| 0.672701 | | | PTN | | CDON | | | IGFALS | S100A13 | CILP2 |
| 0.647513 | | | PTN | | | PH | Notch1 | IGFALS | S100A13 | |
| 0.662396 | | | PTN | | | PH | Notch1 | IGFALS | | CILP2 |
| 0.662468 | | | PTN | | | PH | Notch1 | | S100A13 | CILP2 |
| 0.66388 | | | PTN | | | PH | | IGFALS | S100A13 | CILP2 |
| 0.663488 | | | PTN | | | | Notch1 | IGFALS | S100A13 | CILP2 |
| 0.642633 | | | | MP2K2 | CDON | PH | Notch1 | IGFALS | | |
| 0.640566 | | | | MP2K2 | CDON | PH | Notch1 | | S100A13 | |
| 0.643487 | | | | MP2K2 | CDON | PH | Notch1 | | | CILP2 |
| 0.642324 | | | | MP2K2 | CDON | PH | | IGFALS | S100A13 | |
| 0.645725 | | | | MP2K2 | CDON | PH | | IGFALS | | CILP2 |
| 0.643327 | | | | MP2K2 | CDON | PH | | | S100A13 | CILP2 |
| 0.634214 | | | | MP2K2 | CDON | | Notch1 | IGFALS | S100A13 | |
| 0.638506 | | | | MP2K2 | CDON | | Notch1 | IGFALS | | CILP2 |
| 0.635863 | | | | MP2K2 | CDON | | Notch1 | | S100A13 | CILP2 |
| 0.638993 | | | | MP2K2 | CDON | | | IGFALS | S100A13 | CILP2 |
| 0.621755 | | | | MP2K2 | | PH | Notch1 | IGFALS | S100A13 | |
| 0.632799 | | | | MP2K2 | | PH | Notch1 | IGFALS | | CILP2 |
| 0.625827 | | | | MP2K2 | | PH | Notch1 | | S100A13 | CILP2 |
| 0.632641 | | | | MP2K2 | | PH | | IGFALS | S100A13 | CILP2 |
| 0.620534 | | | | MP2K2 | | | Notch1 | IGFALS | S100A13 | CILP2 |
| 0.627074 | | | | | CDON | PH | Notch1 | IGFALS | S100A13 | |
| 0.628658 | | | | | CDON | PH | Notch1 | IGFALS | | CILP2 |
| 0.625479 | | | | | CDON | PH | Notch1 | | S100A13 | CILP2 |
| 0.628869 | | | | | CDON | PH | | IGFALS | S100A13 | CILP2 |
| 0.620802 | | | | | CDON | | Notch1 | IGFALS | S100A13 | CILP2 |
| 0.605437 | | | | | | PH | Notch1 | IGFALS | S100A13 | CILP2 |
| 0.671041 | NADK | H2A3 | PTN | MP2K2 | CDON | PH | | | | |
| 0.669933 | NADK | H2A3 | PTN | MP2K2 | CDON | | Notch1 | | | |
| 0.668535 | NADK | H2A3 | PTN | MP2K2 | CDON | | | IGFALS | | |
| 0.668687 | NADK | H2A3 | PTN | MP2K2 | CDON | | | | S100A13 | |
| 0.680032 | NADK | H2A3 | PTN | MP2K2 | CDON | | | | | CILP2 |
| 0.659359 | NADK | H2A3 | PTN | MP2K2 | | PH | Notch1 | | | |
| 0.654003 | NADK | H2A3 | PTN | MP2K2 | | PH | | IGFALS | | |
| 0.654771 | NADK | H2A3 | PTN | MP2K2 | | PH | | | S100A13 | |
| 0.672993 | NADK | H2A3 | PTN | MP2K2 | | PH | | | | CILP2 |
| 0.655032 | NADK | H2A3 | PTN | MP2K2 | | | Notch1 | IGFALS | | |
| 0.654584 | NADK | H2A3 | PTN | MP2K2 | | | Notch1 | | S100A13 | |
| 0.671836 | NADK | H2A3 | PTN | MP2K2 | | | Notch1 | | | CILP2 |
| 0.649447 | NADK | H2A3 | PTN | MP2K2 | | | | IGFALS | S100A13 | |
| 0.672693 | NADK | H2A3 | PTN | MP2K2 | | | | IGFALS | | CILP2 |
| 0.671884 | NADK | H2A3 | PTN | MP2K2 | | | | | S100A13 | CILP2 |
| 0.670129 | NADK | H2A3 | PTN | | CDON | PH | Notch1 | | | |
| 0.667665 | NADK | H2A3 | PTN | | CDON | PH | | IGFALS | | |
| 0.669076 | NADK | H2A3 | PTN | | CDON | PH | | | S100A13 | |
| 0.677779 | NADK | H2A3 | PTN | | CDON | PH | | | | CILP2 |
| 0.667597 | NADK | H2A3 | PTN | | CDON | | Notch1 | IGFALS | | |
| 0.668206 | NADK | H2A3 | PTN | | CDON | | Notch1 | | S100A13 | |
| 0.677299 | NADK | H2A3 | PTN | | CDON | | Notch1 | | | CILP2 |
| 0.666019 | NADK | H2A3 | PTN | | CDON | | | IGFALS | S100A13 | |
| 0.67753 | NADK | H2A3 | PTN | | CDON | | | IGFALS | | CILP2 |
| 0.677519 | NADK | H2A3 | PTN | | CDON | | | | S100A13 | CILP2 |
| 0.656771 | NADK | H2A3 | PTN | | | PH | Notch1 | IGFALS | | |
| 0.657301 | NADK | H2A3 | PTN | | | PH | Notch1 | | S100A13 | |
| 0.668934 | NADK | H2A3 | PTN | | | PH | Notch1 | | | CILP2 |
| 0.651529 | NADK | H2A3 | PTN | | | PH | | IGFALS | S100A13 | |
| 0.668885 | NADK | H2A3 | PTN | | | PH | | IGFALS | | CILP2 |
| 0.669203 | NADK | H2A3 | PTN | | | PH | | | S100A13 | CILP2 |
| 0.652658 | NADK | H2A3 | PTN | | | | Notch1 | IGFALS | S100A13 | |
| 0.668387 | NADK | H2A3 | PTN | | | | Notch1 | IGFALS | | CILP2 |
| 0.66899 | NADK | H2A3 | PTN | | | | Notch1 | | S100A13 | CILP2 |
| 0.669085 | NADK | H2A3 | PTN | | | | | IGFALS | S100A13 | CILP2 |
| 0.65373 | NADK | H2A3 | | MP2K2 | CDON | PH | Notch1 | | | |
| 0.65387 | NADK | H2A3 | | MP2K2 | CDON | PH | | IGFALS | | |
| 0.653109 | NADK | H2A3 | | MP2K2 | CDON | PH | | | S100A13 | |
| 0.655108 | NADK | H2A3 | | MP2K2 | CDON | PH | | | | CILP2 |
| 0.645695 | NADK | H2A3 | | MP2K2 | CDON | | Notch1 | IGFALS | | |
| 0.644872 | NADK | H2A3 | | MP2K2 | CDON | | Notch1 | | S100A13 | |
| 0.647947 | NADK | H2A3 | | MP2K2 | CDON | | Notch1 | | | CILP2 |
| 0.645638 | NADK | H2A3 | | MP2K2 | CDON | | | IGFALS | S100A13 | |
| 0.649114 | NADK | H2A3 | | MP2K2 | CDON | | | IGFALS | | CILP2 |
| 0.647796 | NADK | H2A3 | | MP2K2 | CDON | | | | S100A13 | CILP2 |
| 0.637379 | NADK | H2A3 | | MP2K2 | | PH | Notch1 | IGFALS | | |
| 0.632974 | NADK | H2A3 | | MP2K2 | | PH | Notch1 | | S100A13 | |
| 0.640493 | NADK | H2A3 | | MP2K2 | | PH | Notch1 | | | CILP2 |
| 0.632816 | NADK | H2A3 | | MP2K2 | | PH | | IGFALS | S100A13 | |
| 0.643338 | NADK | H2A3 | | MP2K2 | | PH | | IGFALS | | CILP2 |
| 0.639318 | NADK | H2A3 | | MP2K2 | | PH | | | S100A13 | CILP2 |
| 0.622506 | NADK | H2A3 | | MP2K2 | | | Notch1 | IGFALS | S100A13 | |
| 0.6323 | NADK | H2A3 | | MP2K2 | | | Notch1 | IGFALS | | CILP2 |
| 0.626743 | NADK | H2A3 | | MP2K2 | | | Notch1 | | S100A13 | CILP2 |
| 0.631274 | NADK | H2A3 | | MP2K2 | | | | IGFALS | S100A13 | CILP2 |
| 0.641333 | NADK | H2A3 | | | CDON | PH | Notch1 | IGFALS | | |
| 0.640385 | NADK | H2A3 | | | CDON | PH | Notch1 | | S100A13 | |
| 0.641216 | NADK | H2A3 | | | CDON | PH | Notch1 | | | CILP2 |
| 0.640165 | NADK | H2A3 | | | CDON | PH | | IGFALS | S100A13 | |
| 0.641869 | NADK | H2A3 | | | CDON | PH | | IGFALS | | CILP2 |
| 0.640574 | NADK | H2A3 | | | CDON | PH | | | S100A13 | CILP2 |
| 0.630997 | NADK | H2A3 | | | CDON | | Notch1 | IGFALS | S100A13 | |
| 0.633493 | NADK | H2A3 | | | CDON | | Notch1 | IGFALS | | CILP2 |
| 0.631537 | NADK | H2A3 | | | CDON | | Notch1 | | S100A13 | CILP2 |
| 0.633484 | NADK | H2A3 | | | CDON | | | IGFALS | S100A13 | CILP2 |
| 0.61825 | NADK | H2A3 | | | | PH | Notch1 | IGFALS | S100A13 | |
| 0.622952 | NADK | H2A3 | | | | PH | Notch1 | IGFALS | | CILP2 |
| 0.617856 | NADK | H2A3 | | | | PH | Notch1 | | S100A13 | CILP2 |
| 0.620694 | NADK | H2A3 | | | | PH | | IGFALS | S100A13 | CILP2 |
| 0.609666 | NADK | H2A3 | | | | | Notch1 | IGFALS | S100A13 | CILP2 |
| 0.669721 | NADK | | PTN | MP2K2 | CDON | PH | Notch1 | | | |
| 0.669909 | NADK | | PTN | MP2K2 | CDON | PH | | IGFALS | | |
| 0.669312 | NADK | | PTN | MP2K2 | CDON | PH | | | S100A13 | |
| 0.679439 | NADK | | PTN | MP2K2 | CDON | PH | | | | CILP2 |
| 0.668752 | NADK | | PTN | MP2K2 | CDON | | Notch1 | IGFALS | | |
| 0.668325 | NADK | | PTN | MP2K2 | CDON | | Notch1 | | S100A13 | |
| 0.678414 | NADK | | PTN | MP2K2 | CDON | | Notch1 | | | CILP2 |
| 0.6684 | NADK | | PTN | MP2K2 | CDON | | | IGFALS | S100A13 | |
| 0.680073 | NADK | | PTN | MP2K2 | CDON | | | IGFALS | | CILP2 |
| 0.679152 | NADK | | PTN | MP2K2 | CDON | | | | S100A13 | CILP2 |
| 0.657036 | NADK | | PTN | MP2K2 | | PH | Notch1 | IGFALS | | |
| 0.656233 | NADK | | PTN | MP2K2 | | PH | Notch1 | | S100A13 | |
| 0.672126 | NADK | | PTN | MP2K2 | | PH | Notch1 | | | CILP2 |
| 0.652275 | NADK | | PTN | MP2K2 | | PH | | IGFALS | S100A13 | |
| 0.67383 | NADK | | PTN | MP2K2 | | PH | | IGFALS | | CILP2 |
| 0.672734 | NADK | | PTN | MP2K2 | | PH | | | S100A13 | CILP2 |
| 0.653101 | NADK | | PTN | MP2K2 | | | Notch1 | IGFALS | S100A13 | |
| 0.672303 | NADK | | PTN | MP2K2 | | | Notch1 | IGFALS | | CILP2 |
| 0.671704 | NADK | | PTN | MP2K2 | | | Notch1 | | S100A13 | CILP2 |
| 0.673186 | NADK | | PTN | MP2K2 | | | | IGFALS | S100A13 | CILP2 |
| 0.668239 | NADK | | PTN | | CDON | PH | Notch1 | IGFALS | | |
| 0.668652 | NADK | | PTN | | CDON | PH | Notch1 | | S100A13 | |
| 0.676427 | NADK | | PTN | | CDON | PH | Notch1 | | | CILP2 |
| 0.66849 | NADK | | PTN | | CDON | PH | | IGFALS | S100A13 | |
| 0.677747 | NADK | | PTN | | CDON | PH | | IGFALS | | CILP2 |
| 0.677706 | NADK | | PTN | | CDON | PH | | | S100A13 | CILP2 |
| 0.667407 | NADK | | PTN | | CDON | | Notch1 | IGFALS | S100A13 | |
| 0.677114 | NADK | | PTN | | CDON | | Notch1 | IGFALS | | CILP2 |
| 0.677051 | NADK | | PTN | | CDON | | Notch1 | | S100A13 | CILP2 |
| 0.678072 | NADK | | PTN | | CDON | | | IGFALS | S100A13 | CILP2 |
| 0.654881 | NADK | | PTN | | | PH | Notch1 | IGFALS | S100A13 | |
| 0.669039 | NADK | | PTN | | | PH | Notch1 | IGFALS | | CILP2 |
| 0.669012 | NADK | | PTN | | | PH | Notch1 | | S100A13 | CILP2 |
| 0.669815 | NADK | | PTN | | | PH | | IGFALS | S100A13 | CILP2 |
| 0.669341 | NADK | | PTN | | | | Notch1 | IGFALS | S100A13 | CILP2 |
| 0.648754 | NADK | | | MP2K2 | CDON | PH | Notch1 | IGFALS | | |
| 0.646824 | NADK | | | MP2K2 | CDON | PH | Notch1 | | S100A13 | |
| 0.648439 | NADK | | | MP2K2 | CDON | PH | Notch1 | | | CILP2 |
| 0.648602 | NADK | | | MP2K2 | CDON | PH | | IGFALS | S100A13 | |
| 0.651027 | NADK | | | MP2K2 | CDON | PH | | IGFALS | | CILP2 |
| 0.648227 | NADK | | | MP2K2 | CDON | PH | | | S100A13 | CILP2 |
| 0.639984 | NADK | | | MP2K2 | CDON | | Notch1 | IGFALS | S100A13 | |
| 0.643786 | NADK | | | MP2K2 | CDON | | Notch1 | IGFALS | | CILP2 |
| 0.641147 | NADK | | | MP2K2 | CDON | | Notch1 | | S100A13 | CILP2 |
| 0.644167 | NADK | | | MP2K2 | CDON | | | IGFALS | S100A13 | CILP2 |
| 0.630459 | NADK | | | MP2K2 | | PH | Notch1 | IGFALS | S100A13 | |
| 0.639899 | NADK | | | MP2K2 | | PH | Notch1 | IGFALS | | CILP2 |
| 0.634743 | NADK | | | MP2K2 | | PH | Notch1 | | S100A13 | CILP2 |
| 0.639067 | NADK | | | MP2K2 | | PH | | IGFALS | S100A13 | CILP2 |
| 0.627569 | NADK | | | MP2K2 | | | Notch1 | IGFALS | S100A13 | CILP2 |
| 0.634945 | NADK | | | | CDON | PH | Notch1 | IGFALS | S100A13 | |
| 0.636005 | NADK | | | | CDON | PH | Notch1 | IGFALS | | CILP2 |
| 0.633276 | NADK | | | | CDON | PH | Notch1 | | S100A13 | CILP2 |
| 0.635872 | NADK | | | | CDON | PH | | IGFALS | S100A13 | CILP2 |
| 0.62782 | NADK | | | | CDON | | Notch1 | IGFALS | S100A13 | CILP2 |
| 0.617452 | NADK | | | | | PH | Notch1 | IGFALS | S100A13 | CILP2 |
| 0.66927 | | H2A3 | PTN | MP2K2 | CDON | PH | Notch1 | | | |
| 0.66793 | | H2A3 | PTN | MP2K2 | CDON | PH | | IGFALS | | |
| 0.668793 | | H2A3 | PTN | MP2K2 | CDON | PH | | | S100A13 | |
| 0.676683 | | H2A3 | PTN | MP2K2 | CDON | PH | | | | CILP2 |
| 0.667321 | | H2A3 | PTN | MP2K2 | CDON | | Notch1 | IGFALS | | |
| 0.667592 | | H2A3 | PTN | MP2K2 | CDON | | Notch1 | | S100A13 | |
| 0.675799 | | H2A3 | PTN | MP2K2 | CDON | | Notch1 | | | CILP2 |
| 0.666487 | | H2A3 | PTN | MP2K2 | CDON | | | IGFALS | S100A13 | |
| 0.676957 | | H2A3 | PTN | MP2K2 | CDON | | | IGFALS | | CILP2 |
| 0.67633 | | H2A3 | PTN | MP2K2 | CDON | | | | S100A13 | CILP2 |
| 0.657006 | | H2A3 | PTN | MP2K2 | | PH | Notch1 | IGFALS | | |
| 0.657131 | | H2A3 | PTN | MP2K2 | | PH | Notch1 | | S100A13 | |
| 0.669009 | | H2A3 | PTN | MP2K2 | | PH | Notch1 | | | CILP2 |
| 0.653381 | | H2A3 | PTN | MP2K2 | | PH | | IGFALS | S100A13 | |
| 0.670658 | | H2A3 | PTN | MP2K2 | | PH | | IGFALS | | CILP2 |
| 0.670082 | | H2A3 | PTN | MP2K2 | | PH | | | S100A13 | CILP2 |
| 0.653455 | | H2A3 | PTN | MP2K2 | | | Notch1 | IGFALS | S100A13 | |
| 0.66863 | | H2A3 | PTN | MP2K2 | | | Notch1 | IGFALS | | CILP2 |
| 0.668303 | | H2A3 | PTN | MP2K2 | | | Notch1 | | S100A13 | CILP2 |
| 0.669703 | | H2A3 | PTN | MP2K2 | | | | IGFALS | S100A13 | CILP2 |
| 0.667126 | | H2A3 | PTN | | CDON | PH | Notch1 | IGFALS | | |
| 0.66816 | | H2A3 | PTN | | CDON | PH | Notch1 | | S100A13 | |
| 0.67422 | | H2A3 | PTN | | CDON | PH | Notch1 | | | CILP2 |
| 0.666557 | | H2A3 | PTN | | CDON | PH | | IGFALS | S100A13 | |
| 0.6747 | | H2A3 | PTN | | CDON | PH | | IGFALS | | CILP2 |
| 0.675355 | | H2A3 | PTN | | CDON | PH | | | S100A13 | CILP2 |
| 0.665838 | | H2A3 | PTN | | CDON | | Notch1 | IGFALS | S100A13 | |
| 0.673967 | | H2A3 | PTN | | CDON | | Notch1 | IGFALS | | CILP2 |
| 0.674496 | | H2A3 | PTN | | CDON | | Notch1 | | S100A13 | CILP2 |
| 0.674737 | | H2A3 | PTN | | CDON | | | IGFALS | S100A13 | CILP2 |
| 0.655037 | | H2A3 | PTN | | | PH | Notch1 | IGFALS | S100A13 | |
| 0.665625 | | H2A3 | PTN | | | PH | Notch1 | IGFALS | | CILP2 |
| 0.666144 | | H2A3 | PTN | | | PH | Notch1 | | S100A13 | CILP2 |
| 0.666936 | | H2A3 | PTN | | | PH | | IGFALS | S100A13 | CILP2 |
| 0.665931 | | H2A3 | PTN | | | | Notch1 | IGFALS | S100A13 | CILP2 |
| 0.651006 | | H2A3 | | MP2K2 | CDON | PH | Notch1 | IGFALS | | |
| 0.649928 | | H2A3 | | MP2K2 | CDON | PH | Notch1 | | S100A13 | |
| 0.651714 | | H2A3 | | MP2K2 | CDON | PH | Notch1 | | | CILP2 |
| 0.650823 | | H2A3 | | MP2K2 | CDON | PH | | IGFALS | S100A13 | |
| 0.653071 | | H2A3 | | MP2K2 | CDON | PH | | IGFALS | | CILP2 |
| 0.651386 | | H2A3 | | MP2K2 | CDON | PH | | | S100A13 | CILP2 |
| 0.642834 | | H2A3 | | MP2K2 | CDON | | Notch1 | IGFALS | S100A13 | |
| 0.645856 | | H2A3 | | MP2K2 | CDON | | Notch1 | IGFALS | | CILP2 |
| 0.644582 | | H2A3 | | MP2K2 | CDON | | Notch1 | | S100A13 | CILP2 |
| 0.645948 | | H2A3 | | MP2K2 | CDON | | | IGFALS | S100A13 | CILP2 |
| 0.633781 | | H2A3 | | MP2K2 | | PH | Notch1 | IGFALS | S100A13 | |
| 0.640678 | | H2A3 | | MP2K2 | | PH | Notch1 | IGFALS | | CILP2 |
| 0.635965 | | H2A3 | | MP2K2 | | PH | Notch1 | | S100A13 | CILP2 |
| 0.639665 | | H2A3 | | MP2K2 | | PH | | IGFALS | S100A13 | CILP2 |
| 0.628658 | | H2A3 | | MP2K2 | | | Notch1 | IGFALS | S100A13 | CILP2 |
| 0.6363 | | H2A3 | | | CDON | PH | Notch1 | IGFALS | S100A13 | |
| 0.637266 | | H2A3 | | | CDON | PH | Notch1 | IGFALS | | CILP2 |
| 0.636122 | | H2A3 | | | CDON | PH | Notch1 | | S100A13 | CILP2 |
| 0.637051 | | H2A3 | | | CDON | PH | | IGFALS | S100A13 | CILP2 |
| 0.62868 | | H2A3 | | | CDON | | Notch1 | IGFALS | S100A13 | CILP2 |
| 0.615535 | | H2A3 | | | | PH | Notch1 | IGFALS | S100A13 | CILP2 |
| 0.666179 | | | PTN | MP2K2 | CDON | PH | Notch1 | IGFALS | | |
| 0.666004 | | | PTN | MP2K2 | CDON | PH | Notch1 | | S100A13 | |
| 0.673994 | | | PTN | MP2K2 | CDON | PH | Notch1 | | | CILP2 |
| 0.666539 | | | PTN | MP2K2 | CDON | PH | | IGFALS | S100A13 | |
| 0.675824 | | | PTN | MP2K2 | CDON | PH | | IGFALS | | CILP2 |
| 0.67539 | | | PTN | MP2K2 | CDON | PH | | | S100A13 | CILP2 |
| 0.665258 | | | PTN | MP2K2 | CDON | | Notch1 | IGFALS | S100A13 | |
| 0.674817 | | | PTN | MP2K2 | CDON | | Notch1 | IGFALS | | CILP2 |
| 0.674234 | | | PTN | MP2K2 | CDON | | Notch1 | | S100A13 | CILP2 |
| 0.676323 | | | PTN | MP2K2 | CDON | | | IGFALS | S100A13 | CILP2 |
| 0.651602 | | | PTN | MP2K2 | | PH | Notch1 | IGFALS | S100A13 | |
| 0.668459 | | | PTN | MP2K2 | | PH | Notch1 | IGFALS | | CILP2 |
| 0.66687 | | | PTN | MP2K2 | | PH | Notch1 | | S100A13 | CILP2 |
| 0.66997 | | | PTN | MP2K2 | | PH | | IGFALS | S100A13 | CILP2 |
| 0.668321 | | | PTN | MP2K2 | | | Notch1 | IGFALS | S100A13 | CILP2 |
| 0.665279 | | | PTN | | CDON | PH | Notch1 | IGFALS | S100A13 | |
| 0.672691 | | | PTN | | CDON | PH | Notch1 | IGFALS | | CILP2 |
| 0.673094 | | | PTN | | CDON | PH | Notch1 | | S100A13 | CILP2 |
| 0.674536 | | | PTN | | CDON | PH | | IGFALS | S100A13 | CILP2 |
| 0.673485 | | | PTN | | CDON | | Notch1 | IGFALS | S100A13 | CILP2 |
| 0.665099 | | | PTN | | | PH | Notch1 | IGFALS | S100A13 | CILP2 |
| 0.64304 | | | | MP2K2 | CDON | PH | Notch1 | IGFALS | S100A13 | |
| 0.645815 | | | | MP2K2 | CDON | PH | Notch1 | IGFALS | | CILP2 |
| 0.643483 | | | | MP2K2 | CDON | PH | Notch1 | | S100A13 | CILP2 |
| 0.645933 | | | | MP2K2 | CDON | PH | | IGFALS | S100A13 | CILP2 |
| 0.63897 | | | | MP2K2 | CDON | | Notch1 | IGFALS | S100A13 | CILP2 |
| 0.632858 | | | | MP2K2 | | PH | Notch1 | IGFALS | S100A13 | CILP2 |
| 0.628975 | | | | | CDON | PH | Notch1 | IGFALS | S100A13 | CILP2 |
| 0.673254 | NADK | H2A3 | PTN | MP2K2 | CDON | PH | Notch1 | | | |
| 0.67228 | NADK | H2A3 | PTN | MP2K2 | CDON | PH | | IGFALS | | |
| 0.672661 | NADK | H2A3 | PTN | MP2K2 | CDON | PH | | | S100A13 | |
| 0.681207 | NADK | H2A3 | PTN | MP2K2 | CDON | PH | | | | CILP2 |
| 0.6709 | NADK | H2A3 | PTN | MP2K2 | CDON | | Notch1 | IGFALS | | |
| 0.671153 | NADK | H2A3 | PTN | MP2K2 | CDON | | Notch1 | | S100A13 | |
| 0.680449 | NADK | H2A3 | PTN | MP2K2 | CDON | | Notch1 | | | CILP2 |
| 0.670142 | NADK | H2A3 | PTN | MP2K2 | CDON | | | IGFALS | S100A13 | |
| 0.681348 | NADK | H2A3 | PTN | MP2K2 | CDON | | | IGFALS | | CILP2 |
| 0.680495 | NADK | H2A3 | PTN | MP2K2 | CDON | | | | S100A13 | CILP2 |
| 0.661301 | NADK | H2A3 | PTN | MP2K2 | | PH | Notch1 | IGFALS | | |
| 0.661215 | NADK | H2A3 | PTN | MP2K2 | | PH | Notch1 | | S100A13 | |
| 0.673971 | NADK | H2A3 | PTN | MP2K2 | | PH | Notch1 | | | CILP2 |
| 0.656924 | NADK | H2A3 | PTN | MP2K2 | | PH | | IGFALS | S100A13 | |
| 0.674985 | NADK | H2A3 | PTN | MP2K2 | | PH | | IGFALS | | CILP2 |
| 0.674462 | NADK | H2A3 | PTN | MP2K2 | | PH | | | S100A13 | CILP2 |
| 0.656791 | NADK | H2A3 | PTN | MP2K2 | | | Notch1 | IGFALS | S100A13 | |
| 0.673266 | NADK | H2A3 | PTN | MP2K2 | | | Notch1 | IGFALS | | CILP2 |
| 0.673057 | NADK | H2A3 | PTN | MP2K2 | | | Notch1 | | S100A13 | CILP2 |
| 0.674188 | NADK | H2A3 | PTN | MP2K2 | | | | IGFALS | S100A13 | CILP2 |
| 0.670973 | NADK | H2A3 | PTN | | CDON | PH | Notch1 | IGFALS | | |
| 0.671866 | NADK | H2A3 | PTN | | CDON | PH | Notch1 | | S100A13 | |
| 0.678563 | NADK | H2A3 | PTN | | CDON | PH | Notch1 | | | CILP2 |
| 0.670181 | NADK | H2A3 | PTN | | CDON | PH | | IGFALS | S100A13 | |
| 0.679051 | NADK | H2A3 | PTN | | CDON | PH | | IGFALS | | CILP2 |
| 0.679376 | NADK | H2A3 | PTN | | CDON | PH | | | S100A13 | CILP2 |
| 0.669449 | NADK | H2A3 | PTN | | CDON | | Notch1 | IGFALS | S100A13 | |
| 0.678479 | NADK | H2A3 | PTN | | CDON | | Notch1 | IGFALS | | CILP2 |
| 0.678581 | NADK | H2A3 | PTN | | CDON | | Notch1 | | S100A13 | CILP2 |
| 0.678875 | NADK | H2A3 | PTN | | CDON | | | IGFALS | S100A13 | CILP2 |
| 0.659112 | NADK | H2A3 | PTN | | | PH | Notch1 | IGFALS | S100A13 | |
| 0.670567 | NADK | H2A3 | PTN | | | PH | Notch1 | IGFALS | | CILP2 |
| 0.670986 | NADK | H2A3 | PTN | | | PH | Notch1 | | S100A13 | CILP2 |
| 0.671266 | NADK | H2A3 | PTN | | | PH | | IGFALS | S100A13 | CILP2 |
| 0.670685 | NADK | H2A3 | PTN | | | | Notch1 | IGFALS | S100A13 | CILP2 |
| 0.654782 | NADK | H2A3 | | MP2K2 | CDON | PH | Notch1 | IGFALS | | |
| 0.653544 | NADK | H2A3 | | MP2K2 | CDON | PH | Notch1 | | S100A13 | |
| 0.655199 | NADK | H2A3 | | MP2K2 | CDON | PH | Notch1 | | | CILP2 |
| 0.654216 | NADK | H2A3 | | MP2K2 | CDON | PH | | IGFALS | S100A13 | |
| 0.656599 | NADK | H2A3 | | MP2K2 | CDON | PH | | IGFALS | | CILP2 |
| 0.654876 | NADK | H2A3 | | MP2K2 | CDON | PH | | | S100A13 | CILP2 |
| 0.646178 | NADK | H2A3 | | MP2K2 | CDON | | Notch1 | IGFALS | S100A13 | |
| 0.649266 | NADK | H2A3 | | MP2K2 | CDON | | Notch1 | IGFALS | | CILP2 |
| 0.647911 | NADK | H2A3 | | MP2K2 | CDON | | Notch1 | | S100A13 | CILP2 |
| 0.649251 | NADK | H2A3 | | MP2K2 | CDON | | | IGFALS | S100A13 | CILP2 |
| 0.637543 | NADK | H2A3 | | MP2K2 | | PH | Notch1 | IGFALS | S100A13 | |
| 0.644474 | NADK | H2A3 | | MP2K2 | | PH | Notch1 | IGFALS | | CILP2 |
| 0.640413 | NADK | H2A3 | | MP2K2 | | PH | Notch1 | | S100A13 | CILP2 |
| 0.643481 | NADK | H2A3 | | MP2K2 | | PH | | IGFALS | S100A13 | CILP2 |
| 0.632358 | NADK | H2A3 | | MP2K2 | | | Notch1 | IGFALS | S100A13 | CILP2 |
| 0.641338 | NADK | H2A3 | | | CDON | PH | Notch1 | IGFALS | S100A13 | |
| 0.642599 | NADK | H2A3 | | | CDON | PH | Notch1 | IGFALS | | CILP2 |
| 0.640962 | NADK | H2A3 | | | CDON | PH | Notch1 | | S100A13 | CILP2 |
| 0.641885 | NADK | H2A3 | | | CDON | PH | | IGFALS | S100A13 | CILP2 |
| 0.633757 | NADK | H2A3 | | | CDON | | Notch1 | IGFALS | S100A13 | CILP2 |
| 0.622976 | NADK | H2A3 | | | | PH | Notch1 | IGFALS | S100A13 | CILP2 |
| 0.671864 | NADK | | PTN | MP2K2 | CDON | PH | Notch1 | IGFALS | | |
| 0.671599 | NADK | | PTN | MP2K2 | CDON | PH | Notch1 | | S100A13 | |
| 0.679665 | NADK | | PTN | MP2K2 | CDON | PH | Notch1 | | | CILP2 |
| 0.671913 | NADK | | PTN | MP2K2 | CDON | PH | | IGFALS | S100A13 | |
| 0.681485 | NADK | | PTN | MP2K2 | CDON | PH | | IGFALS | | CILP2 |
| 0.680779 | NADK | | PTN | MP2K2 | CDON | PH | | | S100A13 | CILP2 |
| 0.670678 | NADK | | PTN | MP2K2 | CDON | | Notch1 | IGFALS | S100A13 | |
| 0.680317 | NADK | | PTN | MP2K2 | CDON | | Notch1 | IGFALS | | CILP2 |
| 0.67969 | NADK | | PTN | MP2K2 | CDON | | Notch1 | | S100A13 | CILP2 |
| 0.68124 | NADK | | PTN | MP2K2 | CDON | | | IGFALS | S100A13 | CILP2 |
| 0.658933 | NADK | | PTN | MP2K2 | | PH | Notch1 | IGFALS | S100A13 | |
| 0.67441 | NADK | | PTN | MP2K2 | | PH | Notch1 | IGFALS | | CILP2 |
| 0.67373 | NADK | | PTN | MP2K2 | | PH | Notch1 | | S100A13 | CILP2 |
| 0.675349 | NADK | | PTN | MP2K2 | | PH | | IGFALS | S100A13 | CILP2 |
| 0.673869 | NADK | | PTN | MP2K2 | | | Notch1 | IGFALS | S100A13 | CILP2 |
| 0.670493 | NADK | | PTN | | CDON | PH | Notch1 | IGFALS | S100A13 | |
| 0.678178 | NADK | | PTN | | CDON | PH | Notch1 | IGFALS | | CILP2 |
| 0.678257 | NADK | | PTN | | CDON | PH | Notch1 | | S100A13 | CILP2 |
| 0.679582 | NADK | | PTN | | CDON | PH | | IGFALS | S100A13 | CILP2 |
| 0.678602 | NADK | | PTN | | CDON | | Notch1 | IGFALS | S100A13 | CILP2 |
| 0.670995 | NADK | | PTN | | | PH | Notch1 | IGFALS | S100A13 | CILP2 |
| 0.649008 | NADK | | | MP2K2 | CDON | PH | Notch | IGFALS | S100A13 | |
| 0.65098 | NADK | | | MP2K2 | CDON | PH | Notch1 | IGFALS | | CILP2 |
| 0.64834 | NADK | | | MP2K2 | CDON | PH | Notch1 | | S100A13 | CILP2 |
| 0.651106 | NADK | | | MP2K2 | CDON | PH | | IGFALS | S100A13 | CILP2 |
| 0.644182 | NADK | | | MP2K2 | CDON | | Notch1 | IGFALS | S100A13 | CILP2 |
| 0.639866 | NADK | | | MP2K2 | | PH | Notch1 | IGFALS | S100A13 | CILP2 |
| 0.636111 | NADK | | | | CDON | PH | Notch1 | IGFALS | S100A13 | CILP2 |
| 0.670428 | | H2A3 | PTN | MP2K2 | CDON | PH | Notch1 | IGFALS | | |
| 0.67094 | | H2A3 | PTN | MP2K2 | CDON | PH | Notch1 | | S100A13 | |
| 0.677463 | | H2A3 | PTN | MP2K2 | CDON | PH | Notch1 | | | CILP2 |
| 0.670114 | | H2A3 | PTN | MP2K2 | CDON | PH | | IGFALS | S100A13 | |
| 0.67833 | | H2A3 | PTN | MP2K2 | CDON | PH | | IGFALS | | CILP2 |
| 0.678338 | | H2A3 | PTN | MP2K2 | CDON | PH | | | S100A13 | CILP2 |
| 0.668921 | | H2A3 | PTN | MP2K2 | CDON | | Notch1 | IGFALS | S100A13 | |
| 0.677215 | | H2A3 | PTN | MP2K2 | CDON | | Notch1 | IGFALS | | CILP2 |
| 0.677263 | | H2A3 | PTN | MP2K2 | CDON | | Notch1 | | S100A13 | CILP2 |
| 0.678003 | | H2A3 | PTN | MP2K2 | CDON | | | IGFALS | S100A13 | CILP2 |
| 0.65927 | | H2A3 | PTN | MP2K2 | | PH | Notch1 | IGFALS | S100A13 | |
| 0.670961 | | H2A3 | PTN | MP2K2 | | PH | Notch1 | IGFALS | | CILP2 |
| 0.670556 | | H2A3 | PTN | MP2K2 | | PH | Notch1 | | S100A13 | CILP2 |
| 0.672643 | | H2A3 | PTN | MP2K2 | | PH | | IGFALS | S100A13 | CILP2 |
| 0.670535 | | H2A3 | PTN | MP2K2 | | | Notch1 | IGFALS | S100A13 | CILP2 |
| 0.669293 | | H2A3 | PTN | | CDON | PH | Notch1 | IGFALS | S100A13 | |
| 0.675447 | | H2A3 | PTN | | CDON | PH | Notch1 | IGFALS | | CILP2 |
| 0.676283 | | H2A3 | PTN | | CDON | PH | Notch1 | | S100A13 | CILP2 |
| 0.676711 | | H2A3 | PTN | | CDON | PH | | IGFALS | S100A13 | CILP2 |
| 0.675897 | | H2A3 | PTN | | CDON | | Notch1 | IGFALS | S100A13 | CILP2 |
| 0.667956 | | H2A3 | PTN | | | PH | Notch1 | IGFALS | S100A13 | CILP2 |
| 0.651219 | | H2A3 | | MP2K2 | CDON | PH | Notch1 | IGFALS | S100A13 | |
| 0.65302 | | H2A3 | | MP2K2 | CDON | PH | Notch1 | IGFALS | | CILP2 |
| 0.65182 | | H2A3 | | MP2K2 | CDON | PH | Notch1 | | S100A13 | CILP2 |
| 0.652972 | | H2A3 | | MP2K2 | CDON | PH | | IGFALS | S100A13 | CILP2 |
| 0.646263 | | H2A3 | | MP2K2 | CDON | | Notch1 | IGFALS | S100A13 | CILP2 |
| 0.640834 | | H2A3 | | MP2K2 | | PH | Notch1 | IGFALS | S100A13 | CILP2 |
| 0.637355 | | H2A3 | | | CDON | PH | Notch1 | IGFALS | S100A13 | CILP2 |
| 0.668314 | | | PTN | MP2K2 | CDON | PH | Notch1 | IGFALS | S100A13 | |
| 0.676031 | | | PTN | MP2K2 | CDON | PH | Notch1 | IGFALS | | CILP2 |
| 0.675662 | | | PTN | MP2K2 | CDON | PH | Notch1 | | S100A13 | CILP2 |
| 0.677753 | | | PTN | MP2K2 | CDON | PH | | IGFALS | S100A13 | CILP2 |
| 0.676463 | | | PTN | MP2K2 | CDON | | Notch1 | IGFALS | S100A13 | CILP2 |
| 0.670012 | | | PTN | MP2K2 | | PH | Notch1 | IGFALS | S100A13 | CILP2 |
| 0.674826 | | | PTN | | CDON | PH | Notch1 | IGFALS | S100A13 | CILP2 |
| 0.645954 | | | | MP2K2 | CDON | PH | Notch1 | IGFALS | S100A13 | CILP2 |
| 0.674224 | NADK | H2A3 | PTN | MP2K2 | CDON | PH | Notch1 | IGFALS | | |
| 0.674542 | NADK | H2A3 | PTN | MP2K2 | CDON | PH | Notch1 | | S100A13 | |
| 0.68172 | NADK | H2A3 | PTN | MP2K2 | CDON | PH | Notch1 | | | CILP2 |
| 0.673855 | NADK | H2A3 | PTN | MP2K2 | CDON | PH | | IGFALS | S100A13 | |
| 0.682618 | NADK | H2A3 | PTN | MP2K2 | CDON | PH | | IGFALS | | CILP2 |
| 0.682271 | NADK | H2A3 | PTN | MP2K2 | CDON | PH | | | S100A13 | CILP2 |
| 0.672439 | NADK | H2A3 | PTN | MP2K2 | CDON | | Notch1 | IGFALS | S100A13 | |
| 0.681421 | NADK | H2A3 | PTN | MP2K2 | CDON | | Notch1 | IGFALS | | CILP2 |
| 0.681123 | NADK | H2A3 | PTN | MP2K2 | CDON | | Notch1 | | S100A13 | CILP2 |
| 0.682011 | NADK | H2A3 | PTN | MP2K2 | CDON | | | IGFALS | S100A13 | CILP2 |
| 0.662772 | NADK | H2A3 | PTN | MP2K2 | | PH | Notch1 | IGFALS | S100A13 | |
| 0.675447 | NADK | H2A3 | PTN | MP2K2 | | PH | Notch1 | IGFALS | | CILP2 |
| 0.675195 | NADK | H2A3 | PTN | MP2K2 | | PH | Notch1 | | S100A13 | CILP2 |
| 0.676554 | NADK | H2A3 | PTN | MP2K2 | | PH | | IGFALS | S100A13 | CILP2 |
| 0.674744 | NADK | H2A3 | PTN | MP2K2 | | | Notch1 | IGFALS | S100A13 | CILP2 |
| 0.672693 | NADK | H2A3 | PTN | | CDON | PH | Notch1 | IGFALS | S100A13 | |
| 0.679678 | NADK | H2A3 | PTN | | CDON | PH | Notch1 | IGFALS | | CILP2 |
| 0.679855 | NADK | H2A3 | PTN | | CDON | PH | Notch1 | | S100A13 | CILP2 |
| 0.68048 | NADK | H2A3 | PTN | | CDON | PH | | IGFALS | S100A13 | CILP2 |
| 0.679836 | NADK | H2A3 | PTN | | CDON | | Notch1 | IGFALS | S100A13 | CILP2 |
| 0.672556 | NADK | H2A3 | PTN | | | PH | Notch1 | IGFALS | S100A13 | CILP2 |
| 0.654752 | NADK | H2A3 | | MP2K2 | CDON | PH | Notch1 | IGFALS | S100A13 | |
| 0.656586 | NADK | H2A3 | | MP2K2 | CDON | PH | Notch1 | IGFALS | | CILP2 |
| 0.655103 | NADK | H2A3 | | MP2K2 | CDON | PH | Notch1 | | S100A13 | CILP2 |
| 0.656393 | NADK | H2A3 | | MP2K2 | CDON | PH | | IGFALS | S100A13 | CILP2 |
| 0.649305 | NADK | H2A3 | | MP2K2 | CDON | | Notch1 | IGFALS | S100A13 | CILP2 |
| 0.644456 | NADK | H2A3 | | MP2K2 | | PH | Notch1 | IGFALS | S100A13 | CILP2 |
| 0.642445 | NADK | H2A3 | | | CDON | PH | Notch1 | IGFALS | S100A13 | CILP2 |
| 0.67357 | NADK | | PTN | MP2K2 | CDON | PH | Notch1 | IGFALS | S100A13 | |
| 0.6816 | NADK | | PTN | MP2K2 | CDON | PH | Notch1 | IGFALS | | CILP2 |
| 0.681039 | NADK | | PTN | MP2K2 | CDON | PH | Notch1 | | S100A13 | CILP2 |
| 0.682489 | NADK | | PTN | MP2K2 | CDON | PH | | IGFALS | S100A13 | CILP2 |
| 0.68157 | NADK | | PTN | MP2K2 | CDON | | Notch1 | IGFALS | S100A13 | CILP2 |
| 0.675657 | NADK | | PTN | MP2K2 | | PH | Notch1 | IGFALS | S100A13 | CILP2 |
| 0.679746 | NADK | | PTN | | CDON | PH | Notch1 | IGFALS | S100A13 | CILP2 |
| 0.651144 | NADK | | | MP2K2 | CDON | PH | Notch1 | IGFALS | S100A13 | CILP2 |
| 0.672061 | | H2A3 | PTN | MP2K2 | CDON | PH | Notch1 | IGFALS | S100A13 | |
| 0.678724 | | H2A3 | PTN | MP2K2 | CDON | PH | Notch1 | IGFALS | | CILP2 |
| 0.678812 | | H2A3 | PTN | MP2K2 | CDON | PH | Notch1 | | S100A13 | CILP2 |
| 0.679952 | | H2A3 | PTN | MP2K2 | CDON | PH | | IGFALS | S100A13 | CILP2 |
| 0.678714 | | H2A3 | PTN | MP2K2 | CDON | | Notch1 | IGFALS | S100A13 | CILP2 |
| 0.672757 | | H2A3 | PTN | MP2K2 | | PH | Notch1 | IGFALS | S100A13 | CILP2 |
| 0.677167 | | H2A3 | PTN | | CDON | PH | Notch1 | IGFALS | S100A13 | CILP2 |
| 0.653138 | | H2A3 | | MP2K2 | CDON | PH | Notch1 | IGFALS | S100A13 | CILP2 |
| 0.677857 | | | PTN | MP2K2 | CDON | PH | Notch1 | IGFALS | S100A13 | CILP2 |
| 0.675594 | NADK | H2A3 | PTN | MP2K2 | CDON | PH | Notch1 | IGFALS | S100A13 | |
| 0.682779 | NADK | H2A3 | PTN | MP2K2 | CDON | PH | Notch1 | IGFALS | | CILP2 |
| 0.682571 | NADK | H2A3 | PTN | MP2K2 | CDON | PH | Notch1 | | S100A13 | CILP2 |
| 0.68363 | NADK | H2A3 | PTN | MP2K2 | CDON | PH | | IGFALS | S100A13 | CILP2 |
| 0.682561 | NADK | H2A3 | PTN | MP2K2 | CDON | | Notch1 | IGFALS | S100A13 | CILP2 |
| 0.676878 | NADK | H2A3 | PTN | MP2K2 | | PH | Notch1 | IGFALS | S100A13 | CILP2 |
| 0.680886 | NADK | H2A3 | PTN | | CDON | PH | Notch1 | IGFALS | S100A13 | CILP2 |
| 0.656626 | NADK | H2A3 | | MP2K2 | CDON | PH | Notch1 | IGFALS | S100A13 | CILP2 |
| 0.682776 | NADK | | PTN | MP2K2 | CDON | PH | Notch1 | IGFALS | S100A13 | CILP2 |
| 0.679972 | | H2A3 | PTN | MP2K2 | CDON | PH | Notch1 | IGFALS | S100A13 | CILP2 |
| 0.683841 | NADK | H2A3 | PTN | MP2K2 | CDON | PH | Notch1 | IGFALS | S100A13 | CILP2 |

TABLE 51
Combinations of markers from Table 6 includes *NADK, H2A3, PTN,
MP2K2, CDON, PH, Notch1, IGFALS, S100A13, CILP2 -5 year model

| AUC | * | CDCP1 | NDST1 | TIM-1 | TBCA | ASB9 | OPG | SVEP1 | PARC |
|---|---|---|---|---|---|---|---|---|---|
| 0.683841 | * | | | | | | | | |
| 0.704107 | * | CDCP1 | | | | | | | |
| 0.707758 | * | | NDST1 | | | | | | |
| 0.685272 | * | | | TIM-1 | | | | | |
| 0.687931 | * | | | | TBCA | | | | |
| 0.689101 | * | | | | | ASB9 | | | |
| 0.691237 | * | | | | | | OPG | | |
| 0.691609 | * | | | | | | | SVEP1 | |
| 0.686942 | * | | | | | | | | PARC |
| 0.68724 | * | | | | | | | | |
| 0.685182 | * | | | | | | | | |
| 0.694419 | * | | | | | | | | |
| 0.685197 | * | | | | | | | | |
| 0.691088 | * | | | | | | | | |
| 0.695672 | * | | | | | | | | |
| 0.692159 | * | | | | | | | | |

| AUC | ATS13 | YLK-40 | sRAGE | calgranulin B | IL18 | NFL | NPTXR |
|---|---|---|---|---|---|---|---|
| 0.683841 | | | | | | | |
| 0.704107 | | | | | | | |
| 0.707758 | | | | | | | |
| 0.685272 | | | | | | | |
| 0.687931 | | | | | | | |
| 0.689101 | | | | | | | |
| 0.691237 | | | | | | | |
| 0.691609 | | | | | | | |
| 0.686942 | | | | | | | |
| 0.68724 | ATS13 | | | | | | |
| 0.685182 | | YLK-40 | | | | | |
| 0.694419 | | | sRAGE | | | | |
| 0.685197 | | | | calgranulin B | | | |
| 0.691088 | | | | | IL18 | | |
| 0.695672 | | | | | | NFL | |
| 0.692159 | | | | | | | NPTXR |

*Includes CDON, CILP2, H2A3, IGFALS, MAP2K2, NADK, NOTCH1, PH, PTN, S100A13

TABLE 52
Combinations of markers from Table 6 includes #CDON, CILP2,
H2A3, IGFALS, MAP2K2, NADK, NOTCH1, PH, and PTN-5 year model

| AUC | # | CDCP1 | NDST1 | TIM-1 | TBCA | ASB9 | OPG | SVEP1 | PARC | ATS13 |
|---|---|---|---|---|---|---|---|---|---|---|
| 0.703956 | # | CDCP1 | | | | | | | | |
| 0.706464 | # | | NDST1 | | | | | | | |
| 0.684355 | # | | | TIM-1 | | | | | | |
| 0.686021 | # | | | | TBCA | | | | | |
| 0.687415 | # | | | | | ASB9 | | | | |
| 0.690587 | # | | | | | | OPG | | | |
| 0.688516 | # | | | | | | | SVEP1 | | |
| 0.685939 | # | | | | | | | | PARC | |
| 0.685774 | # | | | | | | | | | ATS13 |
| 0.684552 | # | | | | | | | | | |
| 0.694396 | # | | | | | | | | | |
| 0.684144 | # | | | | | | | | | |
| 0.690289 | # | | | | | | | | | |
| 0.692092 | # | | | | | | | | | |
| 0.690925 | # | | | | | | | | | |

| AUC | YLK-40 | sRAGE | calgranulin B | IL18 | NFL | NPTXR | NOTCH1 |
|---|---|---|---|---|---|---|---|
| 0.703956 | | | | | | | NOTCH1 |
| 0.706464 | | | | | | | NOTCH1 |
| 0.684355 | | | | | | | NOTCH1 |
| 0.686021 | | | | | | | NOTCH1 |
| 0.687415 | | | | | | | NOTCH1 |
| 0.690587 | | | | | | | NOTCH1 |
| 0.688516 | | | | | | | NOTCH1 |
| 0.685939 | | | | | | | NOTCH1 |
| 0.685774 | | | | | | | NOTCH1 |
| 0.684552 | YLK-40 | | | | | | NOTCH1 |
| 0.694396 | | sRAGE | | | | | NOTCH1 |
| 0.684144 | | | calgranulin B | | | | NOTCH1 |
| 0.690289 | | | | IL18 | | | NOTCH1 |
| 0.692092 | | | | | NFL | | NOTCH1 |
| 0.690925 | | | | | | NPTXR | NOTCH1 |

#Includes CDON, CILP2, H2A3, IGFALS, MAP2K2, NADK, NOTCH1, PH, and PTN

TABLE 53
Univariate performance for each marker
from Table 6 alone-5 year model

| Target_Names | AUC |
|---|---|
| NPTXR | 0.522951 |
| MP2K2 | 0.573402 |
| NFL | 0.53876 |
| H2A3 | 0.56117 |
| IL-18 | 0.583848 |
| CDON | 0.608496 |
| calgranulin B | 0.569012 |
| sRAGE | 0.538212 |
| CILP2 | 0.574274 |
| YKL-40 | 0.545559 |
| ATS13 | 0.600425 |
| PARC | 0.550091 |
| Notch 1 | 0.550853 |
| SVEP1 | 0.593491 |
| PH | 0.571486 |
| OPG | 0.602114 |
| ASB9 | 0.59645 |
| S100A13 | 0.517915 |
| TBCA | 0.531442 |
| TIM-1 | 0.574049 |
| NADK | 0.572587 |
| IGFALS | 0.570391 |
| PTN | 0.614296 |
| NDST1 | 0.641926 |
| CDCP1 | 0.623381 |

What is claimed is:

1. A method of determining whether a subject is at risk for developing dementia within a 20 year period, comprising detecting a level of CILP2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

2. A method of detecting levels of N biomarker proteins in a sample, comprising obtaining the sample from the subject, and detecting the level of each of the N biomarker proteins in the sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

3. A method of determining whether a subject is at risk for developing dementia within a 20 year period, comprising detecting a level of PTN biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

4. A method of determining whether a subject is at risk for developing dementia within a 20 year period, comprising detecting a level of PH biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

5. A method of determining whether a subject is at risk for developing dementia within a 20 year period, comprising detecting a level of Notch1 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

6. A method of determining whether a subject is at risk for developing dementia within a 20 year period, comprising detecting a level of NADK biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

7. A method of determining whether a subject is at risk for developing dementia within a 20 year period, comprising detecting a level of CDON biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

8. A method of determining whether a subject is at risk for developing dementia within a 20 year period, comprising detecting a level of MP2K2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

9. A method of determining whether a subject is at risk for developing dementia within a 20 year period, comprising detecting a level of H2A3 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

10. A method of determining whether a subject is at risk for developing dementia within a 20 year period, comprising detecting a level of IGFALS biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, S100A13, NPTXR, TBCA, CDCP1, sRAGE, SVEP1, NDST1, ASB9, NFL, PARC, TIM-1, calgranulin B, YKL-40, IL-18, ATS13, and OPG.

11. A method of determining whether a subject is at risk for developing dementia within a 20 year period, comprising detecting a level of S100A13 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, IGFALS, NPTXR, TBCA, CDCP1, sRAGE, SVEP1, NDST1, ASB9, NFL, PARC, TIM-1, calgranulin B, YKL-40, IL-18, ATS13, and OPG.

12. The method of any one of the preceding claims, wherein N is 2 to 25, or N is 3 to 25, or N is 4 to 25, or N is 5 to 25, or N is 6 to 25, or N is 7 to 25, or N is 8 to 25, or N is 9 to 25, or N is 10 to 25, or N is 11 to 25, or N is 12 to 25, or N is 13 to 25, or N is 14 to 25, or N is 15 to 25.

13. The method of any one of the preceding claims, wherein N is 2, N is 3, or N is 4, or N is 5, or N is 6, or N is 7, or N is 8, or N is 9, or N is 10, or N is 11, or N is 12, or N is 13, or N is 14, or N is 15, or N is 16, or N is 17, or N is 18, or N is 19, or N is 20, or N is 21, or N is 22, or N is 23, or N is 24, or N is 25.

14. The method of any one of the preceding claims, wherein each of the N biomarker proteins is selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

15. The method of any one of the preceding claims, wherein at least one of the N biomarker proteins is selected from CILP2, PTN, PH, Notch1, NADK, CDON, MP2K2, H2A3, IGFALS, and S100A13.

16. The method of any one of the preceding claims, wherein at least 2, at least 3, at least 4, at least 5, at least 6, at least 7, at least 8, at least 9, or at least 10 of the N protein biomarkers are selected from CILP2, PTN, PH, Notch1, NADK, CDON, MP2K2, H2A3, IGFALS, and S100A13.

17. The method of any one of the preceding claims, wherein two of the N biomarker proteins are CILP2 and H2A3, or two of the N biomarker proteins are CILP2 and IGFALS, or two of the N biomarker proteins are CILP2 and MP2K2, or two of the N biomarker proteins are CILP2 and NADK, or two of the N biomarker proteins are CILP2 and Notch1, or two of the N biomarker proteins are CILP2 and PH, or two of the N biomarker proteins are CILP2 and PTN, or two of the N biomarker proteins are CILP2 and S100A13, or two of the N biomarker proteins are CILP2 and CDON.

18. The method of any one of the preceding claims, wherein two of the N biomarker proteins are PTN and PH, or two of the N biomarker proteins are PTN and S100A13, or two of the N biomarker proteins are PTN and Notch1, or two of the N biomarker proteins are PTN and NADK, or two of the N biomarker proteins are PTN and MP2K2, or two of the N biomarker proteins are PTN and IGFALS, or two of the N biomarker proteins are PTN and H2A3, or two of the N biomarker proteins are PTN and CDON.

19. The method of any one of the preceding claims, wherein two of the N biomarker proteins are PH and Notch1, or two of the N biomarker proteins are PH and NADK, or two of the N biomarker proteins are PH and CDON, or two of the N biomarker proteins are PH and MP2K2, or two of the N biomarker proteins are PH and H2A3, or two of the N biomarker proteins are PH and IGFALS, or two of the N biomarker proteins are PH and S100A13.

20. The method of any one of the preceding claims, wherein two of the N biomarker proteins are Notch1 and NADK, or two of the N biomarker proteins are Notch1 and CDON, or two of the N biomarker proteins are Notch1 and MP2K2, or two of the N biomarker proteins are Notch1 and H2A3, or two of the N biomarker proteins are Notch1 and IGFALS, or two of the N biomarker proteins are Notch1 and S100A13.

21. The method of any one of the preceding claims, wherein two of the N biomarker proteins are NADK and CDON, or two of the N biomarker proteins are NADK and MP2K2, or two of the N biomarker proteins are NADK and H2A3, or two of the N biomarker proteins are NADK and IGFALS, or two of the N biomarker proteins are NADK and S100A13.

22. The method of any one of the preceding claims, wherein two of the N biomarker proteins are CDON and MP2K2, or two of the N biomarker proteins are CDON and H2A3, or two of the N biomarker proteins are CDON and IGFALS, or two of the N biomarker proteins are CDON and S100A13.

23. The method of any one of the preceding claims, wherein two of the N biomarker proteins are MP2K2 and H2A3, or two of the N biomarker proteins are MP2K2 and IGFALS, or two of the N biomarker proteins are MP2K2 and S100A13.

24. The method of any one of the preceding claims, wherein two of the N biomarker proteins are H2A3 and IGFALS, or two of the N biomarker proteins are H2A3 and S100A13.

25. The method of any one of the preceding claims, wherein two of the N biomarker proteins are IGFALS and S100A13.

26. The method of any one of the preceding claims, wherein the sample is a blood sample, a plasma sample, or a serum sample.

27. The method of any one of the preceding claims, wherein the subject is 39, 40, 41, 42, 43, 44, 45, 46, 47, 48, 49, 50, 51, 52, 53, 54, 55, 56, 57, 58, 59, 60, 61, 62, 63, 64, or 65 years of age or older.

28. The method of any one of the preceding claims, wherein detecting is performed using mass spectrometry, an aptamer based assay and/or an antibody based assay.

29. The method of any one of the preceding claims, wherein the method comprises contacting biomarker proteins of the sample or samples with a set of biomarker capture reagents, wherein each biomarker capture reagent of the set of biomarker capture reagents specifically binds to a different biomarker protein being detected.

30. The method of claim 29, wherein each biomarker capture reagent is an antibody or an aptamer.

31. The method of claim 30, wherein each biomarker capture reagent is an aptamer.

32. The method of claim 31, wherein at least one aptamer is a slow off-rate aptamer.

33. The method of claim 32, wherein at least one slow off-rate aptamer comprises at least one, at least two, at least three, at least four, at least five, at least six, at least seven, at least eight, at least nine, or at least 10 nucleotides with modifications.

34. The method of claim 32 or claim 33, wherein each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes.

35. The method of any one of claims 28-34, wherein the level of each biomarker protein measured is determined from a relative florescence unit (RFU) or a protein concentration.

36. The method of any one of the preceding claims, wherein determining the risk for developing dementia within a 20 year period is based on input of the levels of the N biomarker proteins measured in a statistical model.

37. The method of claim 36, wherein the determining comprises analyzing the levels of the N biomarker protein using an accelerated failure time (AFT) model with a Weibull distribution.

38. The method of claim 36 or 37, wherein the model has an area under the curve (AUC) selected from at least 0.65, at least 0.66, at least 0.67, at least 0.68, at least 0.69, at least 0.7, at least 0.75, at least 0.8, at least 0.85, at least 0.9, or at least 0.95.

39. The method of any one of claims 36-38, wherein the model provides an absolute risk probability of a dementia diagnosis within 20 years.

40. The method of any one of claims 36-38, wherein the model provides a relative risk probability calculation of dementia diagnosis within 20 years.

41. The method of claim 40, wherein the relative risk is a value range used to predict the development of dementia within 20 years.

42. The method of claim 41, wherein the relative risk is selective from low to high.

43. The method of any one of claims 40-42, wherein the range of relative risk is 0.25-6.67.

44. The method of any one of claims 36-43, wherein the model provides the absolute probability or the relative risk prediction for developing dementia within a 20 year period based on the level of each of the proteins selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

45. The method of any one of the preceding claims, wherein the subject is identified as at risk for developing dementia within the 20 year period.

46. The method of claim 45, further comprising administering a treatment to the subject.

47. The method of claim 46, wherein the treatment comprises implementing a healthy diet, exercise, social and cognitive stimulation, and/or lowering vascular disease risk factors.

48. The method of any one of claims 45-47, further comprising monitoring the subject at one or more additional time points to determine the risk of developing dementia within the 20 year period.

49. The method of claim 45, wherein the subject is stratified in a preventive therapeutic trial.

50. The method of claim 45, wherein the subject is administered a second diagnostic test.

51. The method of claim 50, wherein the second diagnostic test is an APOE diagnostic test, optionally wherein second diagnostic test is an APOE genotype test, optionally wherein the APOE genotype test determines ε2, ε3, and ε4 alleles.

52. The method of claim 45, wherein the subject at risk for developing dementia within the 20 year period is identified for a purpose of determining a life and/or financial plan.

53. The method of claim 45, wherein one or more additional biomarkers associated with dementia are identified in a subject at risk for dementia or having dementia.

54. A method of determining whether a subject is at risk for developing dementia within a 5, 10, and/or 15 year period, comprising detecting a level of CILP2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

55. A method of determining whether a subject is at risk for developing dementia within a 5, 10, and/or 15 year period, comprising detecting a level of PTN biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

56. A method of determining whether a subject is at risk for developing dementia within a 5, 10, and/or 15 year period, comprising detecting a level of PH biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

57. A method of determining whether a subject is at risk for developing dementia within a 5, 10, and/or 15 year period, comprising detecting a level of Notch1 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

58. A method of determining whether a subject is at risk for developing dementia within a 5, 10, and/or 15 year period, comprising detecting a level of NADK biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

59. A method of determining whether a subject is at risk for developing dementia within a 5, 10, and/or 15 year period, comprising detecting a level of CDON biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

60. A method of determining whether a subject is at risk for developing dementia within a 5, 10, and/or 15 year period, comprising detecting a level of MP2K2 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

61. A method of determining whether a subject is at risk for developing dementia within a 5, 10, and/or 15 year period, comprising detecting a level of H2A3 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

62. A method of determining whether a subject is at risk for developing dementia within a 5, 10, and/or 15 year period, comprising detecting a level of IGFALS biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, S100A13, NPTXR, TBCA, CDCP1, sRAGE, SVEP1, NDST1, ASB9, NFL, PARC, TIM-1, calgranulin B, YKL-40, IL-18, ATS13, and OPG.

63. A method of determining whether a subject is at risk for developing dementia within a 5, 10, and/or 15 year period, comprising detecting a level of S100A13 biomarker protein and a level of each of N biomarker proteins in a sample from the subject, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker proteins is selected from CILP2, PTN, PH, Notch1, NADK, CDON, MP2K2, H2A3, IGFALS, NPTXR, TBCA, CDCP1, sRAGE, SVEP1, NDST1, ASB9, NFL, PARC, TIM-1, calgranulin B, YKL-40, IL-18, ATS13, and OPG.

64. The method of any one of claims 54-63, wherein N is 2 to 25, or N is 3 to 25, or N is 4 to 25, or N is 5 to 25, or N is 6 to 25, or N is 7 to 25, or N is 8 to 25, or N is 9 to 25, or N is 10 to 25, or N is 11 to 25, or N is 12 to 25, or N is 13 to 25, or N is 14 to 25, or N is 15 to 25.

65. The method of any one of claims 54-64, wherein N is 2, N is 3, or N is 4, or N is 5, or N is 6, or N is 7, or N is 8, or N is 9, or N is 10, or N is 11, or N is 12, or N is 13, or N is 14, or N is 15, or N is 16, or N is 17, or N is 18, or N is 19, or N is 20, or N is 21, or N is 22, or N is 23, or N is 24, or N is 25.

66. The method of any one of claims 54-65, wherein each of the N biomarker proteins is selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

67. The method of any one of claims 54-65, wherein at least one of the N biomarker proteins is selected from CILP2, PTN, PH, Notch1, NADK, CDON, MP2K2, H2A3, IGFALS, and S100A13.

68. The method of any one of claims 54-65, wherein at least 2, at least 3, at least 4, at least 5, at least 6, at least 7, at least 8, at least 9, or at least 10 of the N protein biomarkers are selected from CILP2, PTN, PH, Notch1, NADK, CDON, MP2K2, H2A3, IGFALS, and S100A13.

69. The method of any one of claims 54-65, wherein two of the N biomarker proteins are CILP2 and H2A3, or two of the N biomarker proteins are CILP2 and IGFALS, or two of the N biomarker proteins are CILP2 and MP2K2, or two of the N biomarker proteins are CILP2 and NADK, or two of the N biomarker proteins are CILP2 and Notch1, or two of the N biomarker proteins are CILP2 and PH, or two of the N biomarker proteins are CILP2 and PTN, or two of the N biomarker proteins are CILP2 and S100A13, or two of the N biomarker proteins are CILP2 and CDON.

70. The method of any one of claims 54-65, wherein two of the N biomarker proteins are PTN and PH, or two of the N biomarker proteins are PTN and S100A13, or two of the N biomarker proteins are PTN and Notch1, or two of the N biomarker proteins are PTN and NADK, or two of the N biomarker proteins are PTN and MP2K2, or two of the N biomarker proteins are PTN and IGFALS, or two of the N biomarker proteins are PTN and H2A3, or two of the N biomarker proteins are PTN and CDON.

71. The method of any one of claims 54-65, wherein two of the N biomarker proteins are PH and Notch1, or two of the N biomarker proteins are PH and NADK, or two of the N biomarker proteins are PH and CDON, or two of the N biomarker proteins are PH and MP2K2, or two of the N biomarker proteins are PH and H2A3, or two of the N biomarker proteins are PH and IGFALS, or two of the N biomarker proteins are PH and S100A13.

72. The method of any one of the preceding claims, wherein two of the N biomarker proteins are Notch1 and NADK, or two of the N biomarker proteins are Notch1 and CDON, or two of the N biomarker proteins are Notch1 and MP2K2, or two of the N biomarker proteins are Notch1 and H2A3, or two of the N biomarker proteins are Notch1 and IGFALS, or two of the N biomarker proteins are Notch1 and S100A13.

73. The method of any one of claims 54-65, wherein two of the N biomarker proteins are NADK and CDON, or two of the N biomarker proteins are NADK and MP2K2, or two of the N biomarker proteins are NADK and H2A3, or two of the N biomarker proteins are NADK and IGFALS, or two of the N biomarker proteins are NADK and S100A13.

74. The method of any one of claims 54-65, wherein two of the N biomarker proteins are CDON and MP2K2, or two of the N biomarker proteins are CDON and H2A3, or two of the N biomarker proteins are CDON and IGFALS, or two of the N biomarker proteins are CDON and S100A13.

75. The method of any one of claims 54-65, wherein two of the N biomarker proteins are MP2K2 and H2A3, or two of the N biomarker proteins are MP2K2 and IGFALS, or two of the N biomarker proteins are MP2K2 and S100A13.

76. The method of any one of claims 54-65, wherein two of the N biomarker proteins are H2A3 and IGFALS, or two of the N biomarker proteins are H2A3 and S100A13.

77. The method of any one of claims 54-65, wherein two of the N biomarker proteins are IGFALS and S100A13.

78. The method of any one of claims 54-77, wherein the sample is a blood sample, a plasma sample, or a serum sample.

79. The method of any one of claims 54-78, wherein the subject is 39, 40, 41, 42, 43, 44, 45, 46, 47, 48, 49, 50, 51, 52, 53, 54, 55, 56, 57, 58, 59, 60, 61, 62, 63, 64, or 65 years of age or older.

80. The method of any one of claims 54-79, wherein detecting is performed using mass spectrometry, an aptamer based assay and/or an antibody based assay.

81. The method of any one of claims 54-80, wherein the method comprises contacting biomarker proteins of the sample or samples with a set of biomarker capture reagents, wherein each biomarker capture reagent of the set of biomarker capture reagents specifically binds to a different biomarker protein being detected.

82. The method of claim 81, wherein each biomarker capture reagent is an antibody or an aptamer.

83. The method of claim 82, wherein each biomarker capture reagent is an aptamer.

84. The method of claim 83, wherein at least one aptamer is a slow off-rate aptamer.

85. The method of claim 84, wherein at least one slow off-rate aptamer comprises at least one, at least two, at least three, at least four, at least five, at least six, at least seven, at least eight, at least nine, or at least 10 nucleotides with modifications.

86. The method of claim 84 or claim 85, wherein each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes.

87. The method of any one of claims 80-86, wherein the level of each biomarker protein measured is determined from a relative florescence unit (RFU) or a protein concentration.

88. The method of any one of claims 54-87, wherein determining the risk for developing dementia within a 5, 10, and/or 15 year period is based on input of the levels of the N biomarker proteins measured in a statistical model.

89. The method of claim 88, wherein the determining comprises analyzing the levels of the N biomarker protein using an accelerated failure time (AFT) model with a Weibull distribution.

90. The method of claim 88 or 89, wherein the model has an area under the curve (AUC) selected from at least 0.65, at least 0.66, at least 0.67, at least 0.68, at least 0.69, at least 0.7, at least 0.75, at least 0.8, at least 0.85, at least 0.9, or at least 0.95.

91. The method of any one of claims 88-90, wherein the model provides an absolute risk probability of a dementia diagnosis within 5, 10, and/or 15 years.

92. The method of any one of claims 88-90, wherein the model provides a relative risk probability calculation of dementia diagnosis within 5, 10, and/or 15 years.

93. The method of claim 92, wherein the relative risk is a value range used to predict the development of dementia within 5, 10, and/or 15 years.

94. The method of claim 93, wherein the relative risk is selective from low to high.

95. The method of any one of claims 92-94, wherein the range of relative risk is 0.25-6.67.

96. The method of any one of claims 88-95, wherein the model provides the absolute probability or the relative risk prediction for developing dementia within a 5, 10, and/or 15 year period based on the level of each of the proteins selected from CILP2, PTN, PH, Notch1, NADK, CDON, MP2K2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

97. The method of any one of the preceding claims, wherein the subject is identified as at risk for developing dementia within the 5, 10, and/or 15 year period.

98. The method of claim 97, further comprising administering a treatment to the subject.

99. The method of claim 98, wherein the treatment comprises implementing a healthy diet, exercise, social and cognitive stimulation, and/or lowering vascular disease risk factors.

100. The method of any one of claims 97-99, further comprising monitoring the subject at one or more additional time points to determine the risk of developing dementia within the 5, 10, and/or 15 year period.

101. The method of claim 97, wherein the subject is stratified in a preventive therapeutic trial.

102. The method of claim 97, wherein the subject is administered a second diagnostic test.

103. The method of claim 102, wherein the second diagnostic test is an APOE diagnostic test, optionally wherein second diagnostic test is an APOE genotype test, optionally wherein the APOE genotype test determines ε2, ε3, and ε4 alleles.

104. The method of claim 97, wherein the subject at risk for developing dementia within the 5, 10, and/or 15 year period is identified for a purpose of determining a life and/or financial plan.

105. The method of claim 97, wherein one or more additional biomarkers associated with dementia are identified in a subject at risk for dementia or having dementia.

106. The method of any one of claims 54-105, wherein the risk is determined within a 5 year period.

107. The method of any one of claims 54-105, wherein the risk is determined within a 10 year period.

108. The method of any one of claims 54-105, wherein the risk is determined within a 15 year period.

109. A kit comprising N biomarker protein capture reagents, wherein N is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, and wherein at least one of the N biomarker protein capture reagents specifically binds to a biomarker protein selected from CILP2, PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

110. The kit of claim 109, wherein N is 2 to 25, or N is 3 to 25, or N is 4 to 25, or N is 5 to 25, or N is 6 to 25, or N is 7 to 25, or N is 8 to 25, or N is 9 to 25, or N is 10 to 25, or N is 11 to 25, or N is 12 to 25, or N is 13 to 25, or N is 14 to 25, or N is 15 to 25.

111. The kit of claim 109 or 110, wherein N is 2, N is 3, or N is 4, or N is 5, or N is 6, or N is 7, or N is 8, or N is 9, or N is 10, or N is 11, or N is 12, or N is 13, or N is 14, or N is 15, or N is 16, or N is 17, or N is 18, or N is 19, or N is 20, or N is 21, or N is 22, or N is 23, or N is 24, or N is 25.

112. The kit of any one of claims 109 to 111, wherein each of the N biomarker protein capture reagents specifically binds to a different biomarker protein.

113. The kit of any one of claims 109 to 112, wherein each of the N biomarker protein capture reagents specifically binds to a biomarker protein selected from CILP2, PTN, PH, Notch1, NADK, CDON, MP2k2, H2A3, IGFALS, S100A13, NPTXR, TBCA, CDCP1, sRAGE, SVEP1, NDST1, ASB9, NFL, PARC, TIM-1, calgranulin B, YKL-40, IL-18, ATS13, and OPG.

114. The kit of any one of claims 109 to 113, wherein at least 1 of the N biomarker protein capture reagents specifically binds to a biomarker protein selected from CILP2, PTN, PH, Notch1, NADK, CDON, MP2K2, H2A3, IGFALS, and S100A13.

115. The kit of any one of claims 109 to 114, wherein at least 2, at least 3, at least 4, at least 5, at least 6, at least 7, at least 8, at least 9, or at least 10 of the N biomarker protein capture reagents specifically binds to a biomarker protein selected from CILP2, PTN, PH, Notch1, NADK, CDON, MP2K2, H2A3, IGFALS, and S100A13.

116. The kit of any one of claims 109 to 115, wherein two of the N biomarker protein capture reagents specifically bind CILP2 and H2A3, or two of the N biomarker protein capture reagents specifically bind CILP2 and IGFALS, or two of the N biomarker protein capture reagents specifically bind CILP2 and MP2K2, or two of the N biomarker protein capture reagents specifically bind CILP2 and NADK, or two of the N biomarker protein capture reagents specifically bind CILP2 and Notch1, or two of the N biomarker protein capture reagents specifically bind CILP2 and PH, or two of the N biomarker protein capture reagents specifically bind CILP2 and PTN, or two of the N biomarker protein capture reagents specifically bind CILP2 and S100A13, or two of the N biomarker protein capture reagents specifically bind CILP2 and CDON.

117. The kit of any one of claims 109 to 115, wherein two of the N biomarker protein capture reagents specifically bind PTN and PH, or two of the N biomarker protein capture reagents specifically bind PTN and S100A13, or two of the N biomarker protein capture reagents specifically bind PTN and Notch1, or two of the N biomarker protein capture reagents specifically bind PTN and NADK, or two of the N biomarker protein capture reagents specifically bind PTN and MP2K2, or two of the N biomarker protein capture reagents specifically bind PTN and IGFALS, or two of the N biomarker protein capture reagents specifically bind PTN and H2A3, or two of the N biomarker protein capture reagents specifically bind PTN and CDON.

118. The kit of any one of claims 109 to 115, wherein two of the N biomarker protein capture reagents specifically bind PH and Notch1, or two of the N biomarker protein capture reagents specifically bind PH and NADK, or two of the N biomarker protein capture reagents specifically bind PH and CDON, or two of the N biomarker protein capture reagents specifically bind PH and MP2K2, or two of the N biomarker protein capture reagents specifically bind PH and H2A3, or two of the N biomarker protein capture reagents specifically bind PH and IGFALS, or two of the N biomarker protein capture reagents specifically bind PH and S100A13.

119. The method of any one of claims 109 to 115, wherein two of the N biomarker protein capture reagents specifically bind Notch1 and NADK, or two of the N biomarker protein capture reagents specifically bind Notch1 and CDON, or two of the N biomarker protein capture reagents specifically bind Notch1 and MP2K2, or two of the N biomarker protein capture reagents specifically bind Notch1 and H2A3, or two of the N biomarker protein capture reagents specifically bind Notch1 and IGFALS, or two of the N biomarker protein capture reagents specifically bind Notch1 and S100A13.

120. The kit of any one of claims 109 to 115, wherein two of the N biomarker protein capture reagents specifically bind NADK and CDON, or two of the N biomarker protein capture reagents specifically bind NADK and MP2K2, or two of the N biomarker protein capture reagents specifically bind NADK and H2A3, or two of the N biomarker protein capture reagents specifically bind NADK and IGFALS, or two of the N biomarker protein capture reagents specifically bind NADK and S100A13.

121. The kit of any one of claims 109 to 115, wherein two of the N biomarker protein capture reagents specifically bind CDON and MP2K2, or two of the N biomarker protein capture reagents specifically bind CDON and H2A3, or two of the N biomarker protein capture reagents specifically bind CDON and IGFALS, or two of the N biomarker protein capture reagents specifically bind CDON and S100A13.

122. The kit of any one of claims 109 to 115, wherein two of the N biomarker protein capture reagents specifically bind MP2K2 and H2A3, or two of the N biomarker protein capture reagents specifically bind MP2K2 and IGFALS, or two of the N biomarker protein capture reagents specifically bind MP2K2 and S100A13.

123. The kit of any one of claims 109 to 115, wherein two of the N biomarker protein capture reagents specifically bind H2A3 and IGFALS, or two of the N biomarker protein capture reagents specifically bind H2A3 and S100A13.

124. The kit of any one of claims 109 to 115, wherein two of the N biomarker protein capture reagents specifically bind IGFALS and S100A13.

125. A kit comprising N biomarker protein capture reagents, wherein the kit comprises biomarker protein capture reagents for carrying out the method of any one of claims 1-108.

126. The kit of any one of claims 109 to 125, wherein each of the N biomarker protein capture reagents is an antibody or an aptamer.

127. The kit of claim 126, wherein each biomarker protein capture reagent is an aptamer.

128. The kit of claim 127, wherein at least one aptamer is a slow off-rate aptamer.

129. The kit of claim 128, wherein at least one slow off-rate aptamer comprises at least one, at least two, at least three, at least four, at least five, at least six, at least seven, at least eight, at least nine, or at least 10 nucleotides with modifications.

130. The kit of claim 128 or claim 129, wherein each slow off-rate aptamer binds to its target protein with an off rate (t½) of ≥20 minutes, ≥30 minutes, ≥60 minutes, ≥90 minutes, ≥120 minutes, ≥150 minutes, ≥180 minutes, ≥210 minutes, or ≥240 minutes.

131. The kit of any one of claims 109 to 130, for use in detecting the N biomarker proteins in a sample from a subject.

132. The kit of claim 131, for use in determining whether the subject is at risk for developing dementia within a 5, 10, 15 and/or 20 year period.
